(19) United States
(12) Patent Application Publication
(10) Pub. No.: US 2019/0262367 A1
(43) Pub. Date: Aug. 29, 2019

(54) ORAL FORMULATIONS MIMETIC OF ROUX-EN-Y GASTRIC BYPASS ACTIONS ON THE ILEAL BRAKE; COMPOSITIONS, METHODS OF TREATMENT, DIAGNOSTICS AND SYSTEMS FOR TREATMENT OF METABOLIC SYNDROME MANIFESTATIONS INCLUDING INSULIN RESISTANCE, FATTY LIVER DISEASE, HYPERLIPIDEMIA, AND TYPE 2 DIABETES

(71) Applicant: VOLANT HOLDINGS GMBH, Feusisburg (CH)

(72) Inventors: Joseph M. FAYAD, Las Vegas, NV (US); Jerome SCHENTAG, Amherst, NY (US)

(73) Assignee: VOLANT HOLDINGS GMBH, Feusisburg (CH)

(21) Appl. No.: 16/059,199

(22) Filed: Aug. 9, 2018

Related U.S. Application Data
(63) Continuation of application No. 15/833,360, filed on Dec. 6, 2017, now abandoned.
Application No. 15/833,360 is a continuation of application No. 15/151,358, filed on May 10, 2016, now abandoned.
Application No. 15/151,358 is a continuation of application No. 13/460,753, filed on Apr. 30, 2012, now abandoned.
Application No. 13/460,753 is a continuation-in-part of application No. 12/932,633, filed on Mar. 2, 2011, now Pat. No. 9,757,346.
(60) Provisional application No. 61/309,991, filed on Mar. 3, 2010.
(60) Provisional application No. 61/480,788, filed on Apr. 29, 2011.
(60) Provisional application No. 61/514,174, filed on Aug. 2, 2011.
(60) Provisional application No. 61/551,638, filed on Oct. 26, 2011.

(51) Int. Cl. A61K 31/7004; A61K 45/06; A61K 31/195; A61K 31/20; A61K 31/40; A61K 36/8998; A61K 9/00; A61K 9/28; A61K 31/155; A61K 9/48; A61K 31/4985; A61K 31/555; A61K 36/05; A61K 36/48

(52) U.S. Cl. CPC A61K 31/7004; Y02A 90/26; A61K 31/195; A61K 31/20; A61K 31/40; A61K 36/8998; A61K 9/0053; A61K 9/28; A61K 31/155; A61K 9/4891; A61K 31/4985; A61K 31/555; A61K 36/05; A61K 36/48; A61K 9/4866; A61K 9/288; Y02A 50/387; Y02A 50/463; A61K 45/06

(57) ABSTRACT

The invention provides compositions, methods, and diagnostics for the treatment of metabolic syndromes. In an additional aspect of the invention, compositions and methods of treatment are calibrated to mimic the ileal brake response resulting from surgical intervention, e.g. RYGB, to control metabolic syndrome manifestations and thereby reverse or ameliorate the cardiovascular damage (atherosclerosis, hypertension, lipid accumulation, and the like) resulting from progression of metabolic syndrome. Combination medicaments that act synergistically on the ileal brake and the manifestations of metabolic syndrome are also disclosed.

### RELATED APPLICATIONS

This application is a continuation-in-part application of U.S. application Ser. No. 12/932,633, filed Mar. 2, 2011 entitled “Compositions and Methods for Inducing Satiety and Treating Non-insulin Dependent Diabetes Mellitus, Prediabetic Symptoms, Insulin Resistance and Related Disease Slates and Conditions. This application also claims the benefit of priority from the United States provisional application nos. U.S. 61/480,788, filed 29 Apr. 2011, entitled “Long-Term Stimulation of Ileal Hormones By an Orally Delivered, Ileal Released Natural Product Aphoeline” U.S. 61/514,174, filed Aug. 2, 2011, entitled “The Gut CFO: the ileal hormones. Decreasing Insulin resistance, triglycerides, liver enzymes, signaling caloric intake, using caloric reserve, and turning body to health with every meal” and U.S. 61/551,638, filed Oct. 26, 2011, entitled “Oral formulations Mimetic Roux-en-Y gastric bypass actions on the ileal brake; Compositions, Methods of Treatment, Diagnostics and Systems for treatment of metabolic syndrome manifestations including insuling resistance, fatty liver disease, hyperlipidemia, and type 2 diabetes. Each of said applications listed above is incorporated by reference in its entirety herein.

### FIELD OF THE INVENTION

The invention provides pharmaceutical compositions, methods for the treatment, and diagnostics and computer-implementable systems that relate to the treatment of an array of the manifestations of metabolic syndromes, including Type 2 diabetes, hyperlipidemia, weight gain, obesity, insulin resistance, hypertension, atherosclerosis, fatty liver diseases and certain chronic inflammatory states that lead to these manifestations. In an additional aspect of the invention, compositions and methods of treatment-(which may entail concomitant pharmacological and surgical intervention e.g. Roux-en-Y gastric bypass (RYGB)) activate the ileal brake, which acts in the gastrointestinal tract and the liver of a mammal to control metabolic syndrome manifestations and thereby reverse or ameliorate the cardiovascular damage (atherosclerosis, hypertension, lipid accumulation, and the like) resulting from progression of metabolic syndrome.

In other aspects, the invention provides compositions, methods of treatment, diagnostics, and related systems useful in stabilizing blood glucose and insulin levels, control of hyperlipidemia, control of inflammation in organs tissues and blood vessel walls and treating gastrointestinal disorders.

Thus, the invention provides methods of treatment and pharmaceutical compositions that can be used to prevent, reduce the likelihood of, or delay the onset of, a metabolic syndrome in art obese but otherwise healthy subject, can also be used to treat obese subjects who suffer from one or move metabolic syndromes or consequences thereof. One aspect of the invention teaches that a novel formulation of glucose in dosages of approximately 10 grams or less per day, has both short and long term beneficial effects on patients with Type 2 diabetes. Glucose is normally considered to be damaging to Type 2 diabetes, so it is very novel to use small amounts of specially formulated glucose, applied to a distal location of the intestine by the unique release properties of this formulation, to ameliorate not only the hyperglycemic manifestations of Type 2 diabetes, but also to control the entire associated metabolic syndrome that begins with obesity in the pre-diabetic phase of the disease. This invented medicament can lower their insulin resistance, lower triglycerides, reduce body weight, reduce HBA1c, and lower chronic inflammation, all in the manner of RYGB surgery, whose teachings gave insight into the discovery of this medicament. By means of careful study of enabling biomarker studies, it was apparent that said medicament acts on the same anatomical location and produces the same biochemical pathways as RYGB surgery, the biological target of both being the L-cells of the ileum and distal intestine.

In certain embodiments, the present invention relates to compositions and methods useful for selective modulation of appetite in the manner of RYGB surgery. For example, the present invention also relates to ileal brake hormone releasing substances, and more specifically to the discovery and use of an oral formulation of ileal brake hormone releasing substances which contain a combination of naturally occurring substances which are particularly adapted to treating non insulin dependent diabetes mellitus, pre-diabetic symptoms, insulin resistance and related disease states and conditions of the gastrointestinal tract, diagnostic applications and biological transport of medicaments. Accordingly, the present invention, also relates to methods of using a novel formulation for the treatment of disease states, disorders and/or conditions, or manifestations of metabolic syndrome. It should be added that there is no single treatment for metabolic syndrome in all of its manifestations, while both RYGB and the Brake formulation encompass the widest array of beneficial treatment tints tar discovered.

In one embodiment, the present invention is directed to a method of enhancing the regeneration or remodeling of target organs and tissues of patients with metabolic syndrome disease in need, wherein the treatment is oral mimicry of RYGB actions which thereby produces the endogenous process of regeneration or remodeling of target organs and tissues. In one embodiment, the present invention is directed to a method of enhancing the regeneration or remodeling of target organs and tissues of patients with metabolic syndrome disease in need thereof, wherein the primary treatment is a cell transplant or a stem cell transplant or graft of cells and/or tissue, wherein said method enhances the implanted cells or tissues by oral mimicry of RYGB actions according to the methods otherwise disclosed herein.

### BACKGROUND OF THE INVENTION

One of the factors thought to act as a result of activation of the ileal brake is glucagon-like peptide-1 (7-36) amide (GLP-1), which is processed from proglucagon throughout the small bowel and in the distal small bowel (ileum), and to a lesser extent in the ascending colon, as well as in the central nervous system. GLP-1 has powerful actions on the gastrointestinal tract. Infused in physiological amounts, GLP-1 potently inhibits pentagastrin-induced as well as meal-induced gastric acid secretion. It also inhibits gastric emptying rate and pancreatic enzyme secretion. Similar inhibitory effects on gastric and pancreatic secretion and motility may be elicited in humans upon perfusion of the ileum with carbohydrate- or lipid-containing solutions. Concomitantly, GLP-1 secretion is greatly stimulated during intestinal perfusion experiments, and it has been speculated that GLP-1 may be at least partly responsible for this so-called “ileal-brake” effect.

Within the central nervous system, GLP-1 has a satiating effect, since administration of GLP-1 into the third cerebral ventricle reduces short-term food intake (and meal size). while administration of GLP-1 antagonists have the opposite effect. The administration of graded doses of human GLP-1 produced plasma GLP-1 concentrations within physiological ranges and resulted in the reduction of intake of food in non-obese, healthy male subjects.

GLP-1 is formed and secreted in parallel in the intestinal mucosa along with glicentin (corresponding to PG (1 69), with the glucagon sequence occupying residues Nos. 33 61); small amounts of C-terminally glycine-extended but equally bioactive GLP-1 (737), (PG (78 108)); intervening peptide-2 (PG (111 122) amide); and GLP-2 (PG (126 158)). A fraction of glicentin is cleaved further into GRPP (PG (1 30)) and oxyntomodulin (PG (33 69)).

GLP-1 is also effective in selectively stimulating insulin secretion in patients when the blood glucose is ≥90 mg/dl. Thus, it has the advantage of lowering blood glucose primarily during the prandial phase and does not carry the risk of hypoglycemia if administered without insulin or secretagogues. Additionally, through action at the pancreatic alpha-cells it potently inhibits the inappropriate glucagon secretion seen in Type 2 diabetes. Because of these actions it has pronounced blood glucose lowering effects, particularly in patients with Type 2 diabetes. Byetta® (exenatide) is an incretin mimetic and a GLP-1 receptor agonist, the advantage being a longer half-life in the body compared to native GLP-1. Administered subcutaneously. Byetta® mimics the actions of GLP-1 that occur naturally in the gastrointestinal tract and has emerged as an efficacious type 2 (non-insulin-dependent) diabetes therapy adjunct to one or more oral hypoglycemic agents. While there is general consensus that GLP-1 agonists are partially responsible for the actions of the ileal brake on satiety, it has been controversial whether GLP-1 is responsible for the beneficial actions of RYGB on weight loss, and in fact peripheral administration of GLP-1 agonists like Byetta (exenatide) and Victoza (liraglutide) are associated with modest weight loss (3-5 kg) that occurs slowly over months of treatment RYGB associated weight loss occurs more rapidly, and is associated with a marked decline in insulin and insulin resistance, the magnitude of which is not seen when GLP-1 is administered peripherally to patients with Type 2 diabetes. Some studies argue that calorie restriction alone can produce weight loss. (Isbell J M, Diabetes Care 2010; 33; 1438-1442). In their obese subjects, calorie restriction alone reduces weight over a very short period, but does not elevate GLP-1, or increase the 1^{st} phase insulin response to the meal, or decrease Ghrelin to the extent of RYGB. Thus, there is a controversial interpretation of the real effect of RYGB on body weight and Type 2 diabetes. None the less, it is argued that approximately 80% of type 2 diabetic patients who have RYGB surgery resolve their diabetes and insulin resistance even before they begin losing weight. The RYGB patients in these studies have elevations in GLP-1 to values not seen if they undergo caloric restriction alone. This and other discoveries led to the use of exogenous GLP-1 agonists as drugs to treat Type 2 diabetes, and several of these are on the market or in final stages of approval. In spite of their beneficial impact on Type 2 diabetes, the marketed GLP-1 agonists such as Byetta (exenatide) and Victoza (liraglutide) do not produce all of the beneficial actions that can cure type 2 diabetes, and the recent trend is to treat Type 2 diabetes with combinations of Insulin and GLP-1 agonists.

Peripherally injected GLP-1 drugs do not cure Type 2 diabetes in obese patients, while RYGB cures 80% of these same patients. So, it has been proposed that there are other effects of RYGB beyond GLP-1 and even beyond caloric restriction in combination with peripheral GLP-1 agonists. To this point in the work, there has not been a means of mimicry of the entire spectrum of effects of RYGB that can be observed, in patients who undergo the procedure and lose weight. It is believed from the improved response above and beyond that of exogenous GLP-1 alone, that there are additional endogenous substances that must be involved in bringing the body into weight and metabolic balance and resolving type 2 diabetes, but until the present invention of an oral mimetic of RYGB, these have not been advanced into practice. In fact, although there is marked improvement in HBA1c with GLP-1 agonists, metabolic syndrome complications of hyperlipidemia, atherosclerosis and inflammation are not as effectively treated, or completely resolved, by administration of GLP-1 substances as drugs in comparison to RYGB. GLP-1 drugs are not yet approved for, nor marketed as weight loss products. By contrast, surgical treatment of the type 2 diabetes patient with Roux-en-Y gastric bypass (RYGB) produces all of the beneficial effects on patients with type 2 diabetes AND weight loss and control of the manifestations of metabolic syndrome, and is increasingly viewed by physicians as curative of the entire spectrum of manifestations associated with metabolic syndrome. This leads to the completely novel idea that metabolic syndrome manifestations have a single root cause and RYGB mimicry would become a single treatment for all the manifestations. It therefore became necessary to invent a means to mimic all of the actions of RYGB to produce beneficial action on aspects of metabolic syndrome not controlled by GLP-1 agonists or any of the other available medicaments alone. We disclose herein a formulation and method of use to treat all of these manifestations of metabolic syndrome with a single oral treatment, in a dosage that will be generally free of adverse effects.

Peptide YY (PYY), a 36-amino-acid peptide, is secreted primarily from L-cells residing in the intestinal mucosa of the ileum and large intestine. PYY, which belongs to a family of peptides including neuropeptide Y (NPY) and pancreatic polypeptide, is released into the circulation as PYY (PYY (1-36) and PYY(PYY (3-36); the latter is the major form of PYY in gut mucosal endocrine cells and throughout the circulation. Plasma PYY levels begin to rise within fifteen minutes after the ingestion of food, plateau within approximately ninety minutes, and remain elevated for up to six hours. Exogenous administration of PYY (PYY (3-36) reduces energy intake and body weight in both humans and animals. Via Y2 receptors, the satiety signal mediated by PYY inhibits NPY neurons and activates pro-opiomelanocortin neurons within the hypothalamic arcuate nucleus. Peripheral PYY (PYY 3-36) binds Y2 receptors on vagal afferent, terminals to transmit the satiety signal to the brain. There are studies that imply a beneficial effect of PYY in combination with GLP-1 in animal models of weight loss. There also exist studies that demonstrate the desire for food and the sense of taste change significantly following RYGB. This is likely related to orchestra of gut-derived hormonal and signaling changes following the procedure. The bulk of evidence favors benefit to the PYY elevations that follow RYGB surgery, and to an oral formulation that would mimic this effect as well.

Insulin is the principal hormone responsible for the control of glucose metabolism. It is synthesized in the β cells of the islets of Langerhans as the precursor, proinsulin, which is processed to form C-peptide and insulin, and both are secreted in equimolar amounts into the portal circulation. Insulin has been used for treatment of diabetes for many years and is lifesaving for patients with Type 1 diabetes, where the impact of replacing deficiency of pancreatic insulin with peripheral insulin is beyond doubt. The value of additional insulin to the Type 2 diabetes patient, who already secretes large amounts of insulin, is less clear although most physicians use insulin when oral treatments do not control blood glucose. It is very interesting and perhaps counter-intuitive that RYGB cures type 2 diabetes and does so by lowering both insulin and glucose levels, producing a rapid decline in insulin resistance by HOMA-IR measurements. This decline in insulin resistance is associated with very early resolution of Type 2 diabetes, before meaningful weight loss. Type 2 diabetes patients who undergo RYGB surgery are off their insulin within a few days of surgery, before they have lost significant amounts of weight. Clearly, the unique RYGB cure of Type 2 diabetes does not require more insulin, in fact it appears to require much less, including cessation of both basal and prandial peripheral insulin requirements within a few (lays of RYGB.

It may be asked why RYGB surgery produces such a novel effect on not only Type 2 diabetes but also on the protean manifestations of metabolic syndrome, even before die RYGB patient has any significant weight loss. The discovery was associated with the controlling centers in the distal intestine, which are termed the L-cells. The actions of the L-cells have been used to describe a pathway biomarker pathway to resolution of Type 2 diabetes and metabolic syndromes, and the pathway in general has been called the Ileal Brake. The original description of the ileal brake was physiological, and at the time not much was known of the various biomediators of its action. It was not anticipated that the ileal brake controlled the onset or resolution of Type 2 diabetes or metabolic syndrome. Furthermore, there was no need to evoke the ileal brake as a means of curing metabolic syndromes because at the time we were all focused on treating glucose elevations, lipid elevations, and heart attacks as caused by clots in coronary arteries. So, the discovery of an ileal brake sensor received little attention except to catalyze commercialization of GLP-1 agonists. The ileal brake was not considered important because GLP-1 drugs were administered peripherally and there was no need to evoke a GI-pancreas-Liver explanation for progression of Type 2 diabetes. There was no need to evoke a metabolic syndrome discussion because we were satisfied treating each manifestation as a separate disease. There was no need to consider a GI hormone regulatory pathway for the use of GLP-1 peripherally, it was only when RYGB effects could not be explained beyond the weightless effect that we sought an explanation for the cure of Type 2 diabetes. We discovered the key role of the distal ileum of the GI tract. It was these discoveries that led to the new understanding that RYGB was a common solution for all of the manifestations of metabolic syndrome, and it was very surprising to link them to the rapid resolution of insulin resistance, indeed which occurred within days of RYGB surgery. Furthermore, mimicry of the entire spectrum of actions of RYGB on the ileal Brake with an oral formulation is very novel, although we refer to it in the course of the invention of the Supply Side Model.

Oral mimicry of the ileal brake pathways as discovered by RYGB surgery has now been studied in patients as disclosed herein. Oral formulations targeting the ileal brake offer a fresh approach to the treatment of Type 2 diabetes, obesity and other metabolic syndrome manifestations. It is the best means of oral mimicry of the resolution of Type 2 diabetes after RYGB. With regard to the impact of RYGB on Type 2 diabetes, we proposed a Supply Side model to describe Type 2 diabetes progression from the ingestion of glucose load to the impact of various oral treatments and insulin on the cardiovascular complications that are so common in Type 2 diabetes. The Supply Side Model of Type 2 diabetes, and the system involved in discovery of the impact of the ileal brake on Type 2 diabetes was first disclosed in US20110097807A2, which is incorporated by reference in its entirety here wherein there was clearly an impact of glucose supply on the progression of Type 2 diabetes, a beneficial effect of RYGB on Type 2 diabetes, and for the first time it was proposed to treat Type 2 diabetes with small amounts of precisely formulated glucose via actions on the ileal brake in the same manner as RYGB surgery. In the Supply Side Model, the most beneficial approach to treatment of Type 2 diabetes and its complications was RYGB surgery, while the second most active approach to Type 2 diabetes was an oral formulation of a small amount of glucose applied to the ileal brake alone or in combination with currently available anti-diabetes drugs such as DPP-IV inhibitors.

There has been other work on the ileal brake response to stimulation. For example, U.S. Pat. Nos. 5,753,253 and 6,267,988 disclosed that since satiety feedback from die ileum is more intense per amount of sensed nutrient than from proximal bowel (jejunum), timing the release of a satiety-inducing agent to predominate in ileum will also enhance the satiety response per amount of agent ingested. Thus, both the spread and predominant site of delivery (ileum) will maximize the effect, so that a small amount of released nutrient will be sensed as though it were a large amount, creating a high satiating effect U.S. Pat. Nos. 5,753,253 and 6,267,988 disclose administration of a satiety-inducing agent with a meal and at a time of around 4-6 hours before rite next scheduled meal.

U.S. Pat. No. 7,081,239 discloses manipulating the rate of upper gastrointestinal transit of a substance in a mammal, as well as methods of manipulating satiety and post-prandial pyramidal visceral blood flow. The methods of treatment disclosed in U.S. Pat. No. 7,081,239 can be administered up to a period of 24 hours prior to ingestion of the food, nutrient and/or drug, but most preferably are administered between about 60 to 5 minutes before ingestion. U.S. Pat. No. 7,081,239 notes that in prolonged treatment of postprandial diarrhea or intestinal dumping, there is at least a potential for an adaptive sensory feedback-response that can allow treatment to be discontinued for a number of days without a recurrence of the disorders.

Despite the aforementioned knowledge regarding the role of ileal hormones in digestion and insulin secretion, the need continues to exist for improved therapies that harness the additional anti-metabolic syndrome aspects of the “ileal-brake” effect, beyond the limited exploitation of the peripheral administration of GLP-1 and/or insulin pathway to treat or prevent the onset of Type 2 diabetes or obesity-related disorders. There Ls increasing evidence that the action of the ileal brake is beyond satiety, and more specifically, the regulation of digestion related inflammation is a novel effect of the ileal brake. This pathway is a new explanation for metabolic syndrome manifestations inducting but not limited to progressive obesity and the complications of type 2 diabetes in humans. The growing prevalence of type 2 diabetes, obesity and obesity-related disorders makes this need particularly acute.

Type 2, or noninsulin-dependent diabetes mellitus (NIDDM) typically develops in adulthood. Type 2 diabetes is associated with resistance of glucose-utilizing tissues like adipose tissue, muscle, and liver, to the actions of insulin. Initially, the pancreatic islet beta cells compensate by secreting excess insulin. Eventual islet failure results in decompensation mid chronic hyperglycemia. Conversely, moderate islet insufficiency can precede or coincide with peripheral insulin resistance.

There are several classes of drugs that are useful for treatment of Type 2 diabetes: 1) alpha-glucosidase inhibitors which block and delay carbohydrate absorption, 2). Bile acid sequestrates that ace thought to diminish hepatic gluconeogenesis, 3) basal insulin secretagogues (sulfonylureas), which directly stimulate insulin release, carrying the risk of hypoglycemia; 4) prandial insulin secretagogues (meglitinides) which potentiate glucose-induced insulin secretion, and must be taken before each meal, and also carry risk of hypoglycemia; 5) biguanides, including metformin, which attenuate hepatic gluconeogenesis (which is paradoxically elevated in diabetes); 6) insulin sensitizers, for example the thiazolidinedione derivatives rosiglitazone and pioglitazone, which improve peripheral responsiveness to insulin, but which have side effects like weight gain, edema, and occasional liver toxicity; 7) Dopamine agonists which are drought to reduce hypothalamic dopaminergic tone and insulin resistance; 8) DPP-IV inhibitors which are responsible for the breakdown of DPP-IV, the principle enzyme responsible for GLP-1 degradation; 9) GLP-1 mimetics which are peripherally administered replacements for GLP-1, as noted above; 10) Amylinomimetics which are peripherally administered replacements of amylin, a neuroendocrine hormone cosecreted with insulin by the β-cells that slows gastric emptying, suppresses post-prandial glucagon secretion, and centrally modulates appetite) 11) basal and bolus insulin injections, which may be necessary in the later stages of Type 2 diabetes when the islets have either failed or lay dormant under chronic hyperstimulation.

Insulin resistance can also occur without marked hyperglycemia, and is generally associated with atherosclerosis, obesity, hyperlipidemia, and essential hypertension. This cluster of abnormalities constitutes the “metabolic syndrome” or “insulin resistance syndrome”. Insulin resistance is also associated with fatty liver, which can progress to chronic inflammation, nonalcoholic steatohepatitis, fibrosis, and cirrhosis. Cumulatively, insulin resistance syndromes, including but not limited to diabetes, underlies many of the major causes of morbidity and death of people over age 40.

The present understanding and treatment of metabolic syndrome is highly fragmented, with the choice of one or more popular medications for each of its components. There are drugs for each manifestation that treat only lit at particular biochemical aspect, (such as diabetes drugs for glucose, lipid control drugs for hyperlipidemia, obesity drugs for weight control, and the like). Surprisingly, there are currently no modern approaches to treat all of the manifestations of metabolic syndrome as a unit or constellation. Because each of the available treatments has certain disadvantages and some reverse the beneficial effects of others, indeed it was a novel approach to find a treatment for all of these manifestations with a single oral medicament, and it was even more surprising to discover that the home for metabolic syndromes was the supply of glucose and the controller was the ileal brake. Thus metabolic syndromes of all types could be viewed holistically, with a common source, controllers in place that regulate many aspects of glucose supply in diet, clear links to other nutritional components, and once again a curative surgical procedure (RYGB) that points to the actions of oral treatments designed to mimic its actions on the L-cells in the distal small bowel. Stimulation of these cells, which grow tolerant to dietary glucose overload, wakes up the ileal brake and re-balances the Supply of Nutrients and thus insulin demand pathways disclosed in U.S. Ser. No. 12/911,497 filed Oct. 25, 2010; Published as US 2011/097807 A1 on Apr. 28, 2011, which is incorporated by reference herein.

The gastrointestinal tract is not heretofore known as the primary driver of metabolic syndrome, even though it is possible to account for inflammation, obesity, hyperlipidemia and fatty liver disease arising all from the interaction between the gastrointestinal tract, the pancreas and the liver. Indeed there is evidence that the metabolic syndrome symptomotology begins with dietary components such as glucose, according to the teachings of the supply side model of diabetes as disclosed in United States patent application publication no. US2011/0097807-A1 published Apr. 28, 2011, which is incorporated by reference herein. Drugs acting directly on the ileal brake of the GI tract are highly active against the entire spectrum of metabolic syndrome manifestations, but in particular those that are associated with insulin resistance as an early manifestation. Examples would be pre-diabetes, obesity, and triglyceride dominant hyperlipidemia. In these conditions the glucose load is the primary driver of insulin resistance, and the defect that leads to obesity is the down regulation of the L-cell response to increasing dietary glucose. The body does not reject more glucose in the diet as the L-cells are down regulated, but this increasing dietary supply leads to the need to store the excess as fat. Insulin resistance is the first systemic manifestation of increasing glucose load and down regulation of the ileal brake. It is die purpose of the present application to disclose in detail a formulation and method for treatment of the entire array of man infestations of metabolic syndrome drat are linked to increasing insulin resistance, thereby obviating the long term inflammatory and vascular complications such as morbid obesity, atherosclerosis, myocardial infarction, stroke and later stages of Type 2 diabetes that involve loss of pancreatic ability to secrete insulin. RYGB surgery restores the homeostasis and these manifestations are avoided or at least delayed in onset. Accordingly, formulations and compositions are disclosed that treat all of the major manifestations of insulin resistance, tatty liver diseases, increased triglycerides and other lipids, and obesity.

Despite the existence of various anti-diabetes and glucose control drugs, diabetes remains a major and growing public health problem. Even more concerning recent large-scale randomized controlled trials (ACCORD, ADVANCE, VADT) have created confusion with respect to the proper glucose target because of contradictory data on major cardiovascular events when lowering the glucose too aggressively through algorithms that favor aggressive intensification strategies with secretagogues and insulin. Therein, the problems of hypoglycemia and weight gain inherent to secretagogues and insulin were confounding to any benefits of blood glucose lowering. No clear data exist to determine if preferential treatment with weight neutral or gut hormone-based regimens (i.e. GLP-1) would have resulted in clear improvements in both microvascular and macrovascular complications. Long-term randomized clinical trial evidence demonstrating that treating with GLP-1 agonists yield improved cardiovascular benefits would lend significant credence to the concept that treating with an agent that corrects multiple physiologic hormone signals would not only benefit the management of blood glucose, but the overall cardiovascular status. Similar to Type 2 diabetes, despite many lipid lowering drugs, vascular disease continues to increase in scope and the number of patients with complications also increases. Obesity is increasing rapidly in-spite of weight loss foods and stimulatory drugs. What are needed are not necessarily new drug therapies, which often are accompanied by significant side effects, but rather a method of treatment as an alternative or supplement that addresses the underlying metabolic syndrome and associated insulin resistance. Because it is known that all of the metabolic syndrome man infestations are ameliorated by RYGB surgery, it was desirous to produce each and all of these beneficial events by evoking the mechanistic pathway involved in the reduction of metabolic syndrome by RYGB surgery. Because this mechanistic pathway has recently been discovered by the inventors, it was soon possible to create an orally available formulation of the same substances that produce beneficial actions in the mimicry of RYGB action on the ileal brake. Indeed both of these produce beneficial actions on metabolic syndrome by activation of the ileal brake, which is primarily located in the distal small intestine in the ileum. The formulation that is active, called Brake or Aphoeline in some configurations is a unique combination of natural substances which are food components such as lipids and simple sugars (e.g. mono- and disaccharides, preferably glucose or dextrose). Most of these substances have been listed as GRAS (Generally Regarded As Safe) substances, which after specific formulation for release at the ileum, may be administered as an ileal brake hormone releasing substance, and target the dietary associated inflammatory condition which leads to metabolic syndrome and its consequences. There is a particular need to provide a new orally active approach to treatment of all of the manifestations of metabolic syndrome, which effectively addresses the primary defects of inflammation, obesity, insulin resistance and hyperlipidemia without side effects, so that the therapeutic substance can be administered to those who are in the pre-diabetic stages, or who exhibit pre-diabetic symptoms, so as to forestall or preclude the onset of type 2 diabetes or other complications of metabolic syndrome. It is much easier to resolve obesity and insulin resistance early with an oral formulation, and this would place the use of RYGB later in the course of the disease where more dramatic procedures are easier to justify.

When glucose is absorbed from the early portion of the duodenum, the glucose quickly reaches the beta cells of the pancreas and enters these pancreatic cells via the glut 2 glucose transporter. The amount of glucose in the blood plasma is directly proportional to the glucose being transported into the beta cells.

When insulin is released into the body, it exerts an effect at the cellular level throughout the entire body, but more specifically in the liver, the muscle tissues, and the fat or adipose tissues. The effects can occur in a “short acting” way that stimulates the glucose uptake in muscles and fat cells, thereby increasing the synthesis of glycogen in muscle and liver, inhibiting glucose secretion in the liver, and increasing amino acid uptake, or in a “long term” way which increases protein synthesis and stimulates certain gene expression in all cells. Insulin works by binding with insulin receptors on a cell surface. Once coupled, kinase enzymes push glut 4, the major glucose transport receptor, to attach to the cell surface for driving the glucose intracellularly.

It is generally known that the surface of muscles and fat cells have other receptors that can drive the glucose intracellularly without insulin. These receptors work with IGF-1 and IGF-2 hormones. There is also believed to be an undefined IRR receptor structurally similar to the receptors working with IGF-1 and IGF-2 hormones located on the cell surface but die correlating hormone has not yet been found. In general, the body should maintain a substantial equilibrium, that is, the amount of insulin secreted should be equal to the amount of insulin needed to keep the blood glucose level steady.

One problem that can be experienced is when insulin is not being adequately produced, typically because the pancreas, and mom specifically the beta cells, have been destroyed or are devitalized, as typically seen in Type 1 diabetes, where the output of insulin is decreased or absent. A second problem is where insulin interactions, that is between the insulin, the insulin receptors, and the cells, are hindered by a multitude of cellular and inflammatory factors so that the action is not an efficient use of the insulin available, and as a result, much more insulin is needed to achieve the same goal of driving the glucose intracellularly. This latter condition is far more common and it is currently termed Type 2 diabetes based on the observation that there is no lack of available insulin in the body. It is this type of insulin inefficiency that the present method and composition are directed to. Insulin resistance or insulin insensitivity encompasses the majority of the population dealing with diabetes; Type A, a genetic defect of the insulin receptors (i.e., leprechaunism, Rabson Mendhall syndrome, and lipodystrophy); Type B, an autoimmune type with an antibody to the insulin receptors; and Type 2, a post membrane receptor resistance, that includes the metabolic syndrome manifestations of obesity, hypertension, non insulin dependent diabetes, aging, and polycystic ovary syndrome.

The commonly accepted theory for these two types of insulin resistant afflictions is that the glucoses are not being transported into the cells due to an autoimmune antibody (Type B) or some sort of post receptor resistance. As a result, glucoses outside of the cells build up. The pancreas, attempting to equilibrate the level of glucose and insulin, causes insulin production to increase. Even though more insulin is being produced, glucose is not being transported into the cells. Initially, the increase in insulin is capable of overcoming die insulin resistance but this requires a much higher level of insulin production. This stage is considered the pre-diabetic stage where insulin is high but glucose is normal. Ultimately, the pancreas is not capable of keeping up with the high insulin and precursor proinsulin production rate that is required, thereafter causing the glucose levels to spike, with the person eventually becoming officially classified as diabetic.

The common non-invasive treatment for diabetics is to start and maintain a proper diet and exercise routine. Second, doctors may prescribe medication such as (i) sulfonylureas to stimulate additional secretion of insulin, which can speed up the exhaustion of the pancreas; (ii) metformin may be prescribed to improve the efficiency of insulin action and also improve on the clearance of glucose in liver and peripheral tissues, therefore decreasing the level of glucose and insulin as well.

While pre-di abetics have been treated at times with the same medications, the side effects of rite medications made it difficult for the patient to improve their health since the foregoing treatments were designed for full diabetics. In other circumstances, the medications that may produce weight loss (i.e. exenatide, liraglutide) are not permitted for the management of pre-diabetes or obesity.

### SUMMARY OF THE INVENTION

The present invention relates inter alia to the following oral formulations and methods and provides the following objectives;

Oral agent biochemical mimickry of the biochemical hormone profile of RYGB surgery;

Oral formulations of nutrients and methods which mimic RYGB surgery;

Oral formulations of nutrients and methods that reawaken and/or modulate the down, regulated ileal brake in the manner of RYGB surgery;

Oral formulations of nutrients and methods that act on enterogastric hormone releasing pathways by stimulation of L-cell pathways in the jejunum, and ileum;

Oral formulations of nutrients and methods that selectively modulate appetite and feeding response in obese type 2 diabetic patients;

Oral formulations of nutrients including sugars and/or lipids and methods that re-awaken ileal brake hormone responsiveness in obese type 2 diabetic patients with fatty liver disease and insulin resistance;

Oral formulations of nutrients and methods that control fatty deposits in in livers of humans with obesity or type 2 diabetes;

Oral formulations of nutrients and methods that lower insulin resistance in subjects with obesity, pre-diabetes and type 2 diabetes;

Oral formulations of nutrients, including: sugars and/or lipids and methods that target release of these nutrients in the ileum to activate the ileal brake, thereby treating insulin resistance, fatty liver disease, hyperlipidemia and type 2 diabetes;

Oral formulations of nutrients, including sugars and/or lipids and methods that target release of these nutrients in the ileum to activate the ileal brake, thereby treating metabolic syndrome manifestations including insulin resistance, fatty liver disease, hyperlipidemia and type 2 diabetes;

Oral formulations of nutrients, including sugars and/or lipids and methods that re-awaken dormant, ileal brake response in patients with metabolic syndrome manifestations including insulin resistance, fatty liver disease, hyperlipidemia, and type 2 diabetes;

Oral formulations of nutrients, including sugars and/or lipids and including probiotic bacteria that alter normal intestinal flora populations and control underlying endotoxemia;

Oral formulations of nutrients, including sugars and/or lipids and methods that are beneficial on the supply side of type 2 diabetes treatment regiments;

Oral formulations of nutrients, including sugars- and/or lipids and methods that provide control of non-alcoholic fatty liver disease by activating the ileal break hormone releasing cells.

Thus, according to the present invention, in one aspect, the invention provides a system and method describing the use of novel oral medicament mimickry of the beneficial teachings of the effect of RYGB surgery on the ileum, thereby providing a treatment for the spectrum of insulin resistance associated metabolic syndromes. The integrated approach to these types of metabolic syndromes uses a single agent oral treatment that re-awakens the responsiveness of the endogenous ileal brake in obese patients wherein it is in a quiescent state. Thereby, one oral treatment can be offered for the full range of manifestations of metabolic syndromes including insulin resistance, hyperlipidemia, weight gain, obesity, hypertension, atherosclerosis, fatty liver diseases and certain chronic inflammatory states, wherein said oral treatment method comprises: testing of biomarkers; testing of breath, blood or body fluid biomarkers and selection of pharmaceutical compositions to resolve one or more of the metabolic syndrome conditions including but not limited to chronic inflammatory states, hyperlipidemia, weight gain, obesity, insulin resistance, hypertension, atherosclerosis, and fatty liver. These methods of treatment and compositions can entail personalized treatments and can use the results of biomarker testing such as HBA1c, glucose, GLP-1, PYY, GLP-2, Proinsulin, CRP, hsCRP, triglycerides, oxyntomodulin, endotoxin, IL-6. All of these biomarkers are affected by the novel treatment used for metabolic syndrome manifestations, and all are affected by RYGB surgery. Testing thus far establishes a ratio of potency between said oral medicament and RYGB. Notably, these personalized treatment and pharmaceutical compositions can be selected using a Glucose Supply Side computerized algorithm and system, wherein said Glucose Supply Side treatment method for diabetes consists of an algorithm (incorporated herein in its entirety) ranking favorable attributes of pharmaceutical compositions acting by minimizing excess glucose inside cells, and minimizing the amount of glucose that reaches target cells of the metabolic syndrome afflicted patient. The supply side algorithm provides for novel combinations of treatments including oral stimulation of the ileal brake hormones with a specifically formulated composition. It further provides for combination of the composition acting on ileal brake hormones with drugs that act on glucose, lipids, inflammation, blood pressure, obesity and other manifestations of the metabolic syndrome that afflicts the patient. More specifically, the invention claims the same or lower dose of statin products plus Brake for lipid control, the same or lower dose of DPP-IV inhibitors plus Brake for glucose control, and the same or lower doses of anti-obesity drugs such as lorcaserin for weight control.

In certain aspects, the aforementioned personalized treatments and pharmaceutical compositions may be selected by comparison of biomarker behavior patterns between patients' response to Roux-en-Y bariatric surgery and their response to oral dosing with pharmaceutical formulations comprised of sugars, lipids or amino acids which activate the ileal brake response of the ileum in a manner similar to RYGB surgery.

Significantly, the present invention provides a formulation and a drug delivery strategy that mimics the surgical re-alignment of the intestines to deliver food component substances to distal locations of the intestine. For example, in certain embodiments, RYGB Surgery and an orally administered pharmaceutical composition of the invention produce substantially the same effects on the ileal brake, even with respect to subtle and unexpected aspects like a rapid reduction in insulin resistance and regulation of the gut driven inflammation. In a purely illustrative example, an orally administered dosage of approximately 2 to 1.0 grams, about 2.5-3 to 10 grams, about-7.5 to about 10 grams, preferably 10 grams of active ingredient of a pharmaceutical composition of the invention can have an aggregate positive effect on ileal brake parameters equal to approximately 25% to approximately 80% or more of the aggregate positive effect on such parameters realized by RYGB Surgery. It is notable that these actions far exceed the action of GLP-1 given separately, and dearly evoke different and additional mechanisms and pathways for complete action against metabolic syndromes of type 2 diabetes and other associated conditions. The oral medicament will mimic the beneficial aspects of the ileal brake in the same manner as RYGB, but it will not be associated with loss of as much weight as RYGB. That is because RYGB surgery decreases the size of the stomach and thereby limits intake of food by a second, profoundly important pathway.

Thus, in one embodiment, the invention provides a method of treatment comprising administering to a subject in need thereof an ileum hormone-stimulating amount of an ileal brake hormone releasing substance which releases In vivo substantially in the subject's ileum, wherein (1) the subject suffers from, or is at risk of developing, a metabolic syndrome selected from the group consisting of hyperlipidemia, weight gain, obesity, insulin resistance, hypertension, atherosclerosis, fatty liver diseases and certain chronic inflammatory states (2) optionally, prior to or concurrent with administration, the concentration of one or more of the subject's metabolic syndrome biomarkers is measured and the ileal brake hormone releasing substance or dosage of ileal brake hormone releasing substance is selected based on the biomarker level, and (3) wherein the ileal brake hormone releasing substance comprises at least one microencapsulated glucose, lipid, or amino acid and activates the subject's ileal brake in the manner of RYGB surgery.

Orally administered pharmaceutical compositions of the invention mimic the full range of actions of RYGB surgery on the ileal brake. Mimicry of all of the actions of the ileal brake in this manner using compositions and methods according to the present invention are able to substantially inhibit and in many cases actually cure many patients of their Type 2 diabetes. It is clear that unexpected and surprising benefits of the present invention occur in the control of atherosclerosis, fatty liver, obesity, and many other chronic inflammatory states that are characteristic of metabolic syndromes in the developed world, liven more specifically, the formulation for treatment of metabolic syndrome comprises the micro-encapsulation of glucose, lipids and components of diet formulated to release these active compositions at pH values between about 6.8 and about 7.5, which allows substantial release and targets the action of said medicaments at the ileal brake in the distal intestine. Conventional formulation strategies used for pharmaceuticals never target release at pH values above 6.8. It has only been recently discovered by the inventors (using the “Smart Pill” as invented by Schentag in U.S. Pat. No. 5,279,607 herein incorporated by reference) that pH values above 7.0 are found in the GI tract, and they are characteristic of the ileum in the area ascribed to L-cells and the ileal brake. The encapsulated compositions disclosed are a preferred medicament to reduce dietary glucose associated chronic inflammation, the primary driver of metabolic syndrome and eventual development of obesity and type 2 diabetes. Use of the encapsulated compositions according to the present invention decreases appetite for glucose in the Supply Side model, beneficial to the patient with metabolic syndrome, and thereby lowers both insulin resistance and inflammation and is of benefit to the treatment of patients with metabolic syndrome, according to the results of testing of targeted biomarkers. Accordingly, methods of treatment of the invention may or may not include concomitant or even subsequent RYGB surgery, as control of metabolic syndrome in preferred practice of the invention would be possible with oral use of said medicaments, reserving RYGB surgery for cases beyond the control of said encapsulated compositions alone.

In a preferred embodiment of the invention, oral dosing with about 2,000 to about 10.000 milligrams, preferably about 3,000 to about 10,000 milligrams, about 7,500 to about 10.000 milligrams of a pharmaceutical formulation comprising microencapsulated glucoses, lipids, and/or amino acids activates the ileal brake in a dose increasing magnitude and treats one or more of the following components of metabolic syndrome: hyperlipidemia, weight gain, obesity, insulin resistance, hypertension, atherosclerosis, fatty liver diseases and chronic inflammatory states. In various embodiments according to the present invention, the disclosed formulations and compositions have been described as Aphoeline which is trademarked. Hereinafter, certain aspects of tins composition may be referred to by its trademark Brake™. Compositions of the invention may be used alone or in combination with medicaments ordinarily used to treat specific manifestations of metabolic syndromes such as diabetes, hyperlipidemia, atherosclerosis, hypertension; obesity, insulin resistance, or chronic inflammation. The benefit of Combination is a broader spectrum action for treatment of metabolic syndrome than the single agent, and additional potency of the combination over its components. For example, compositions and methods of treatment of the invention may employ co-administration of a drug such as a biguanide antihyperglycemic agent (e.g. metformin); DPP-IV inhibitors (e.g. Vildagliptin, Sitagliptin; Dutogliptin, Linagliptin and Saxagliptin); TZDs or Thiazolidinediones (which are also known to be active on PPAR), e.g. pioglitazone, rosiglitazone, Troglitazone, aleglitazar and the PPAR-sparing agents MSDC-0160, MSDC-0602; alpha glucosidase inhibitor including but not limited to acarbose (including delayed release preparations of Acarbose, Miglitol, and Voglibose); Glucokinase Activators including but not limited to TTP399 and the like; HMG-CoA reductase inhibitors, (examples of similar agents, thought to act on the defined statin pathway or by HMG-CoA reductase inhibition, include atorvastatin, simvastatin, lovastatin, cerevastatin, pravastatin pitavastatin); angiotensin 11 inhibitors (All inhibitors) (e.g. Valsartan, Olmesartan, Candesartan, Irbesartan, losartan, Telmisartan and the like); a phosphodiesterase type 5 inhibitor (PDE5 inhibitor) such as sildenafil (Viagra), vardenafil (Levitra) and Tadalafil (Cialis®); Anti-obesity compositions that may benefit from combination with Brake™ include Lorcaserin and Topiramate; Combinations that will act beneficially on gas tin intestinal flora include pH encapsulated pro-biotic organisms that release the live bacteria hi the ileum, at pH 7.0 to 7.4, these pH encapsulated probiotic bacteria may be combined further with treatments for irritable bowel disease such as linaclotide or even with antibiotics where the goal is to restore bacterial flora after disruption by potent antibiotic therapy.

In certain embodiments, compositions of the invention act hi the gastrointestinal tract and on the ileal brake to limit glucose supply and to lower all aspects of metabolic syndrome manifestations. Tints, the combination of Brake and a lipid lowering drug such as colesevelam acts on the lipid content of the blood in the same manner as colesevelam and Brake individually, with the potential to lower the dosage of one or both of the components because of this synergy; While illustrative, the selection of a combination including colesevelam is not meant to be exhaustive and it is readily apparent that additional Colesevelam mimetic medicaments can be added to the pharmaceutical composition without departing from the practice of oral treatments for metabolic syndrome that combine oral mimetics of Roux-en-Y surgery effects on the ileal brake in conjunction with conventional anti-diabetes medicaments of the class represented by colesevelam.

As summarized above, the invention includes the combination of Brake and a Glucose Supply Side method for the treatment of Type 2 diabetes mellitus and metabolic syndrome manifestations associated with Type 2 diabetes mellitus, wherein said Glucose Supply Side method has its primary action on the ileal brake and comprises the administration to a human or non-human mammal in need thereof of any of the pharmaceutical compositions in any combination and each in any dosage according to the results of testing of biomarkers demonstrating action of the medicaments chosen on the ileal brake. For example, the invention provides a method for the treatment of Type 2 diabetes mellitus and conditions associated with diabetes mellitus, using a Glucose Supply Side algorithm, wherein said method comprises testing of each patient for genomic markers of response to Glucose Supply Side selected pharmaceutical compositions, and then using the results of genomic testing to individualize the dosage of said compound using genomic markers of the Glucose Supply Side and of the patients individual metabolism of said composition alone or in combination with the results of the Glucose Supply Side breath test biomarkers. Such systems and methods of treatment of the invention can include an input/output (I/O) device coupled to a processor; a communication system coupled to the processor; and a medical computer program and system coupled to the processor, the medical system configured to process medical data of a user and generate processed medical information, wherein the medical data includes one or mom of anatomical data, diabetes associated biomarkers, test specimen data, biological parameters, health information of the user, wherein the processor is configured to dynamically control operations between the communication system and the medical system.

The invention also provides an analyzer coupled to xerogel-based substrates for concentration-dependent analyte detection, the analyzer including a xerogel-based sensor coupled to a processor configured to analyze the specimen and generate the processed medical information, wherein analysis of the specimen includes correlating parameters of the specimen with the medical data.

Further, the invention provides a system for providing metabolic syndrome component management, comprising: a sensor unit measuring concentrations of analytes; an interface unit; one or more processors coupled to the interface unit; a memory for storing data and instructions which, when executed by the one or more processors, causes the one or more processors to receive data associated with monitored analyte concentrations for a predetermined rime period substantially hi real time, retrieve one or more therapy profiles associated with the monitored analyte concentrations, and generate one or more modifications to the retrieved one or more therapy profiles based on the data associated with the monitored analyte concentrations.

In alternative embodiments, the inventors have discovered that the once-daily administration, preferably once-daily administration of an ileum-targeting, delayed and/or controlled release dosage form containing an ileal brake hormone releasing substance to a fasting subject—at a time of around four and one-half to around ten to twelve hours, preferably around six to around nine hours prior to the subject's next intended meal (most preferably at bedtime)) or in AM—produces all of the beneficial actions of the deal brake, including lowering of insulin resistance, control of glucose, and lowering of inflammation in the subject for a period of around twelve hours and preferably twenty-four hours or more (effect can be cumulative depending on the duration of taking the dosage). These beneficial actions in treatment of the manifestations of metabolic syndrome are sustained for long time periods, as present experience exceeds one year of use in some patients to be described. Alternatively, a dosage may be administered at least twice daily, preferably once before bedtime and once within the first two hours (preferably first hour) of waking. Alternatively three dosages may be administered—once in the morning, once in the afternoon and once before bedtime. While not wishing to be bound by any theory, the inventors believe that the Therapeutic substance stimulates the “ileal-brake” effect at a particularly advantageous point during a subject's feeding cycle and thereby induces the beneficial actions on type 2 diabetes and other metabolic syndromes for an extended period of time (for at least about three hours, at least about six hours, at least about twelve hours or as long as twenty-four hours or longer). Benefits continue if the medication is taken daily in proper dosage, and surprisingly, the beneficial effects persist for a period of time after the medication is stopped. Compositions and methods of treatment of the invention therefore also prove particularly useful in the treatment or prevention of overweight, overeating, obesity and obesity-related disorders, as well as the treatment of non insulin dependent diabetes mellitus, pre-diabetic symptoms, metabolic syndrome and insulin resistance, as well as disease states and conditions which occur secondary to diabetes, pre-diabetes, metabolic syndrome and insulin resistance, as well as polycystic (fibrous) ovaries, arteriosclerosis and fatty liver, as well as cirrhosis. The present methods also may be used to increase muscle mass and decrease fat in a subject.

Notably, compositions and methods of treatment of the invention modulate ileal hormone, blood insulin and glucose levels relatively consistently in a variety of tested human subjects and can therefore be used to diagnose the presence of new or established disorders related to absolute or relative deficiency or excessive secretions of one or more hormones of the ileal break, and relative response to the stimuli in the overweight or obese, or in obese related disorders or likely onset of obesity or obesity-related disorders. Compositions according to the present invention may also be used to increase blood concentrations of insulin-like growth factor I and II (IGF1 and IGF2) in a patient.

Accordingly, in one embodiment, the invention provides a method of treatment of type 2 diabetes or metabolic syndromes in a subject by once-daily administration to the subject of a delayed and/or controlled release dosage form. The dosage form is administered while the subject is in the fasted state and at a time of around six to around nine hours prior to the subject's next intended meal. The dosage form comprises an enterically-coated, ileum hormone-stimulating amount of ileal brake hormone releasing substance and releases the majority of the ileal brake hormone releasing substance in vivo upon reaching the subject's ileum.

In some embodiments as a separate effect of administration of compositions of the present invention, satiety is induced in a subject who is overweight, or suffers from obesity or an obesity-related disorder, as determined by the BMI of the subject or patient In another embodiment, the invention provides a method of treatment comprising reducing and/or stabilizing a subject's blood glucose and insulin levels, decreasing insulin resistance, by once-daily administration to the subject of a delayed and/or controlled release oral dosage form with the target site being the ileal brake. The dosage form is administered while the subject is in the fasted state and at a time of around six to around nine hours prior to the subject's next intended meal. The dosage form comprises an enterically-coated, ileum hormone-stimulating amount of ileal brake hormone releasing substance and releases the majority of the ileal brake hormone releasing substance in vivo upon reaching the subject's ileum.

In still another embodiment, the invention provides a method of treating a subject suffering from a gastrointestinal disorder by administering to the subject a delayed and/or controlled release oral dosage form comprising an enterically-coated, ileum hormone-stimulating amount of an ileal brake hormone releasing substance. The dosage form is administered while the subject is in the fasted state and at a time of around four and one-half to ten hours, more preferably around six to around nine hours prior to the subject's next intended meal. The dosage form comprises an enterically-coated, ileum hormone-stimulating amount of ileal brake hormone releasing substance and releases the majority of the ileal brake hormone releasing substance in vivo upon reaching the subject's ileum.

In still other preferred embodiments, the invention provides methods for control of metabolic syndrome and its various detrimental actions, through specific biochemical pathways that stabilize blood glucose and insulin levels, and treating gastrointestinal and hepatic inflammatory disorders comprising once-daily administration to a subject in need thereof of a delayed and/or controlled release composition which may comprise an emulsion or a microemulsion containing an ileum hormone-stimulating amount of ileal brake hormone releasing substance. The composition is administered while the subject is in the fasted state and at a time of around four to ten, preferably around six to around nine hours prior to the subject's next intended meal. The composition releases the majority of the ileal brake hormone releasing substance in vivo upon reaching the subject's ileum, the site of its intended effect.

In preferred embodiments of the aforementioned methods of treatment of the invention, the dosage form is administered once-daily at bedtime, or in AM.

By administering the dosage form to a subject in the fasted state at around four to ten, around six to around nine hours prior to the subject's next intended meal, and delivering substantially all of the ileal brake hormone releasing substance to the ileum, methods and compositions of the invention achieve improved levels of plasma gastrointestinal hormones and prove useful in the treatment or prevention of one or more of obesity, obesity-related disorders, and gastrointestinal disorders, as well as metabolic syndrome and/or type II diabetes mellitus. The benefit of obtaining at least twenty-four hour appetite suppression and improved blood glucose and insulin levels from a single oral dosage of an inexpensive ileal brake hormone releasing substance increases the likelihood that the subject will adhere to the methods of treatment for an extended time (improved patient compliance), thereby achieving a maximum health benefit. Further, compositions and methods of the invention utilize ileal brake hormone releasing substances that are free of the safety and cost concerns associated with pharmacological and surgical intervention, and can induce long-term control of appetite, inflammation, insulin resistance and hyperlipidemia.

In another embodiment, the invention provides a delayed and/or controlled release oral dosage form comprising an effective amount of an ileal brake hormone releasing substance, preferably D-glucose or dextrose in an amount effective when released in the ileum to stimulate or inhibit the release of hormones in that portion of the small intestine of a subject or patient. This dosage form is administered in accordance with, and achieves the advantages of, the aforementioned methods of treatment of the invention. In addition, die present invention provides a method for diagnosing metabolic syndrome (glucose intolerance) and/or type II diabetes in a patient or subject.

Thus, the invention provides methods of stimulating or inhibiting the hormones (depending on the hormone) of the ileum in an easy and reproducible or standardized way which did not exist prior to the present method. Pursuant to the present application, the testing on a large scale of the ileal release to study and classify the variation or pathology of the hormone releases as such release relates to control of metabolic syndromes or type 2 diabetes and related pathological states and conditions, and the effect these hormones have on the rest of the metabolic and hormonal status of the body is another aspect of the invention. Thus, the present method allows the introduction of one or more dosages in oral dosage form to the ileum of the patient which can be standardized sufficiently to allow the creation of a normal reference range for the hormonal stimulation. It has been discovered that the present invention can be used to probe different diseases stemming from the relative or absolute increase or decrease of the ileal hormones, not only in treating the overweight/obesity metabolic syndrome axis but a number of other gastrointestinal diseases as otherwise described herein.

The present method also can be used to diagnose and treat a number of gastrointestinal disorders and/or conditions which may occur as a consequence of infection, medical treatment or diseases of atrophy, including atrophic gastritis, post chemotherapy disorder, intestinal motility disorder (gut dysmobility), mild reflux, chronic pancreatitis, malnutrition, malabsorption, voluntary or involuntary long term starvation, post infectious syndrome, short bowel syndrome, irritable bowel, malabsorption, diarrheal states, post chemotherapy gastrointestinal disorder, post infectious syndrome, radiation enteritis, chronic pancreatitis, celiac disease, fatty liver disease, cirrhosis, radiation, inflammatory bowel disease and Crohn's disease, among others.

In another embodiment, the invention may be used to improve the health of the liver, improve the pancreas health, as well as the health of the intestine, and to decrease/ameliorate fatty liver, to increase the size of pancreatic beta cells (hyperplasia) in the pancreas as well as increase the size of the absorptive villae of the small bowel.

In another embodiment, the method of preparation of the pills can be used in combination with traditional bioactive agents (medication); delivery by itself or together with die core to deliver the content specifically to the ileum for targeted therapy avoiding side effects and increasing the yield of the therapy, such as specialized antibiotics, antispasmodic agents, non-specific chelating agents, antibacterial agents, probiotic bacteria that are normal components of the intestinal tract, antidiabetes agents, statin drugs, anti-obesity drugs, anti-inflammatory drugs, Crohn's disease drugs, drugs for treatment of Alzheimer's disease, drugs for treatment of multiple sclerosis, and laxatives among numerous others, including natural plant oils such as olive oil, corn oil, vegetable and animals oils, fats, such as animal fats, butter and vegetable fat, oils and fats from seeds and nuts, stimulants including caffeine, herbs, teas, ingredients that increase post receptor activities after cellular level, selected extracts or food products and chemicals, natural or otherwise, including metabolites.

In another embodiment, the invention provides a method for diagnosing metabolic syndrome (glucose intolerance) and/or type 31 diabetes in a patient the present invention approaches the problem of metabolic syndrome in a natural physiological manner by stimulating hormones in the ileum which act synergistically for a period of at least about 12 hours and preferably at least about 24 hours. It does this most preferably using natural and safe nutritional components in healthful, pleasant compositions which are preferably coated using a polymeric, preferably aqueous pH-sensitive/dissolution/release of contents of formulation occurs at a pH of the ileum, or a pH of approximately 7-8, preferably 7.2-8.0, about 7.4-8.0, about 7.5-8.0) shellac nutrateric coming to effect a natural physiological response within the subject's ileum with favorable results. The present invention represents a change in the nature of treatment for metabolic syndrome to a more wholesome, natural physiological process, completely distinguishable over pharmaceutical or synthetic approaches.

In other particular embodiments, orally administering an ileal hormone stimulating effective amount of a glucose such as dextrose or other ileal brake hormone releasing substance as otherwise described herein, optionally combined with one or more of other advantageous substances such as alfalfa leaf, chlorella algae, chlorophyllin and barley grass juice concentrate, and further formulated with a delayed release base adapted to release die composition in the lower gut, in particular the ileum, has been shown to result in normalized blood glucose and insulin levels, in particular, in subjects where there previously was shown to be an absence of elevated blood glucose but the subjects exhibited high insulin levels, that is, pre-diabetic symptoms, administering the supplement caused a decrease in insulin levels back to a normal range while glucose levels remained normal (reduced and/or stabilized). In other words, the body system achieved substantial equilibrium, with substantially no side effects reported. The result was similar to what can be achieved administering drugs such as Metaformin and IGF-1, with relatively few, if any, side effects.

Without being limited by way of theory, it is believed that by stimulating the ileal hormones contained in the lower gut, the inventive substance drives the glucose intracellularly by either (i) stimulating the production or increasing the level of IGF-1 and/or IGF-2 that will act on their own receptors, (ii) direct action on IGF-1 and/or IGF-2 receptors, or (in) stimulating one or more intestinal hormones, including a new intestinal hormone that will act on its own receptors as per the IRR receptors.

Accordingly, in another embodiment, the invention provides a method of treating noninsulin dependent diabetes mellitus, pie-diabetic symptoms, metabolic syndrome, increasing glucose tolerance and/or decreasing insulin resistance by reducing insulin levels in the bloodstream comprising administering a ileal brake hormone releasing substance composition containing an effective amount of a glucose, such its dextrose or other ileal brake hormone releasing substance as otherwise defined herein, optionally and preferably combined with one or more of alfalfa leaf, chlorella algae, chlorophyllin and barley grass juice concentrate or sodium alginate, alone or in combination with the other ingredients and further formulated with a delayed release base adapted to release the composition in the lower gut (ileum), that is, in a delayed and/or controlled release dosage form. The dosage form may comprise the ileal brake hormone releasing substance in a unit or partial dose form and have an enteric coating, including a nutrateric coating (e.g., containing shellac as a polymeric material, hypromellose, as an emulsifier, thickener and suspending agent and triacetin as an emulsifier). Alternatively, the ileal brake hormone releasing substance (preferably D-glucose or dextrose) and optionally, one or more of alfalfa leaf, chlorella algae, chlorophyllin and barley grass juice concentrate may be combined with binders, diluents, additives and other pharmaceutical additives such as one or more of a filler, compressibility enhancer (e.g., corn starch or lactose), lubricant (stearic acid), extrusion agent (magnesium stearate), silicon dioxide (dispersing agent), and enteric coated or nutrateric coated with a coating which dissolves at the pH of the ileum and includes one more polymeric components as otherwise described herein.

In another embodiment, the invention provides a method which comprises equilibrating a subject's insulin level to compliment a blood glucose level, preferably by once-daily administration to the subject of a delayed and/or controlled release oral dosage form of the invention.

In still another embodiment, the invention provides a method of treating a subject exhibiting pre-diabetic symptoms comprising administering a ileal brake hormone releasing substance composition containing an effective amount (generally, at least in part, to reduce insulin) of a glucose such as dextrose (glucose) or other ileal brake hormone releasing substance as otherwise described here, either alone, or preferably in combination with one or more of alfalfa leaf, chlorella algae, chlorophyllin and barley grass juice concentrate, in a delayed and/or controlled release dosage form, adapted to release the composition in the lower gut, the combination providing an insulin reducing effect so as to equilibrate the amount of insulin produced to correspond to the amount of blood glucose. The dosage form may comprise the ileal brake hormone releasing substance in a unit or partial dose form and having an enteric coating.

By administering the ileal brake hormone releasing substance to a person who exhibits noninsulin dependent diabetes mellitus, pre-diabetic symptoms, and/or insulin resistance, reduced levels of insulin are produced so as to avoid the “over-working” of the pancreas, thereby reducing stress on the pancreas which may forestall, for example, in someone exhibiting pre-diabetes symptoms, the onset of full blown diabetes. Thus, the present invention also has the advantage of reducing the likelihood that a patient or subject with metabolic syndrome or noninsulin dependent diabetes mellitus (type II diabetes) will see these conditions advance to insulin dependent diabetes mellitus (type 1 diabetes).

Other aspects of the invention relate to compositions which comprise an effective amount of an ileal brake hormone releasing substance as otherwise described herein, preferably glucose or dextrose which is formulated in delayed and/or controlled release dosage form in order to release an effective amount of ileal brake hormone releasing substance in the ileum of the patient or subject to whom compositions according to the present invention are administered, generally, at least 50% of the total amount of the ileal brake hormone releasing substance present, and preferably at least about 70%, at least about 75%, at least about 80%, at least about 85%, at least, about 90%, and al least about 95% or more of the ileal brake hormone releasing substance present in the composition. In the case of D-glucose or dextrose as the ileal brake hormone releasing substance, it is preferred that at least about 2.5 grams, at least about 3 grams, at least about 7.5 grams and more preferably about 10-12.5 grams or more of glucose be released in the patients or subject's ileum in order to stimulate ileal hormone release.

Compositions according to the present invention comprise effective amounts of ileal brake hormone releasing substance, preferably Diglucose or dextrose, which may be combined with at least one delayed or controlled release component such as a delayed/controlled release polymer or compound such as a cellulosic material, including, for example, ethyl cellulose, methyl cellulose, hydroxymethylcellulose, hydroxypropylcellulose, polyvinylpyrrolidone, cellulose acetate trimellitiate (CAT), hydroxypropylmethyl cellulose phthalate (HPMCP), polyvinyl acetate phthalate (PVAP), cellulose acetate phthalate (CAP), shellac, copolymers of methacrylic acid and ethyl acrylate, copolymers of methacrylic acid and ethyl acrylate to which a monomer of methylacrylate has been added during polymerization, a mixture of amylose-butan-1-ol complex (glassy amylose) with Ethocel® aqueous dispersion, a coating formulation comprising an inner coating of glassy amylose and an outer coating of cellulose or acrylic polymer material, pectins (of various types), including calcium pectinate, carageenins, aligns, chondroitin sulfate, dextran hydrogels, guar gum, including modified guar gum such as borax modified guar gum, beta-cyclodextrin, saccharide containing polymers, e.g., a polymeric construct comprising a synthetic oligosaccharide-containing biopolymer including methacrylic polymers covalently coupled to oligosaccharides such as cellobiose, lactulose, raffinose and stachyose, or saccharide-containing, natural polymers including modified mucopolysaccharides such as cross-linked pectate; methacrylate-galactomannan, pH-sensitive hydrogels and resistant starches, e.g., glassy amylose. Other materials include methylmethacrylate or copolymers of methacrylic acid and methylmethacrylate having a pH dissolution profile that delays release in vivo of the majority of the ileal brake hormone releasing substance until the dosage form reaches the ileum may also be used. Such materials are available as Eudragit® polymers (Rohm Pharma, Darmstadt, Germany). For example, Eudragit® L100 and Eudragit® S100 can be used, either alone or in combination. Eudragit® L100 dissolves at pH 6 and upwards and comprises 48.3% methacrylic acid units per g dry substance; Eudragit® S100 dissolves at pH 7 and upwards and comprises 29.2% methacrylic acid units per g dry substance. Generally, the encapsulating polymer has a polymeric backbone and acid or other solubilizing functional groups. Polymers which have been found suitable for purposes of the present invention include polyacrylates, cyclic acrylate polymer, polyacrylic acids and polyacrylamides. A particularly preferred group of encapsulating polymers are the polyacrylic acids Eudragit® L and Eudragit® S which optionally may be combined with Eudragit® RL or RS. These modified acrylic acids are useful since they can be made soluble at a pH of 6 or 7.5, depending on the particular Eudragit chosen, and on the proportion of Eudragit® S to Eudragit® L, RS, and RL used in the formulation. By combining one of both of Eudragit® L and Eudragit® S with Eudragit® RL and RS (5-25%), it is possible to obtain a stronger capsule wall and still retain the capsule's pH-dependent solubility.

A delayed and/or controlled release oral dosage form used in the invention can comprise a core containing an ileum hormonal-stimulating amount of an ileal brake hormone releasing substance along with carriers, additives and excipients that is coated by an enteric coating. In some embodiments, the coating comprises Eudragit® L100 and shellac, or food glaze Eudragit® S100 in the range of 100 parts L100:0 parts S100 to 20 parts L100:80 parts S100, more preferably 70 parts L100:30 parts S100 to 80 parts L100:20 parts S100. In preferred alternatives, the preferred coating is a numeric coating which dissolves at the pH of the ileum (about 7-8, about 7.2-8.0, about 7.4-8.0, about 7.5-8.0) comprising a shellac, and emulsifiers such as triacetone and hypromellose, among others. Alternative nutrateric coatings include ethyl cellulose, ammonium hydroxide, medium chain triglycerides, oleic acid, and stearic acid. As the pH at which the coating begins to dissolve increases, the thickness necessary to achieve ileum-specific delivery decreases. For formulations where the ratio of Eudragit® L100:S100 is high, a coat thickness of the order 150-200 μm can be used. For coatings where the ratio Eudragit® L100:S100 is low, a coat thickness of the order 80-120 μm can be used in the present invention.

In still further embodiments, the present invention relates to a method of improving muscle functions and coordination in a patient in need thereof comprising administering an effective amount of a composition according to the present invention in a patient in need thereof, optionally in combination with a bioactive agent. Additional methods according to the present invention relate to improving the action of traditional anti-diabetes medications, including DPP-IV inhibitors, among others, that suppress GLP-1 inhibition/destruction and work to potentiate GLP-1 levels stimulated by compositions according to the present invention. The agents act in a synergistic manner to produce favorable results in diabetes (especially including type II) treatment:

In additional embodiments of the present invention, a method of treating impairment to or improving basal membrane structure of gastrointestinal tract comprises administering an effective amount of a compound according to the present invention to a patient in need thereof optionally in combination with a bioactive agent. This method may be used to treat, inhibit or reduce the likelihood of multiple sclerosis in a patient or to enhance recovery from injury which occurs secondary to radiation, chemotherapy or other toxins.

The present method also relates to a method of treating or reducing the likelihood of liver disease such as fatty liver, non-alcoholic fatty liver disease and various forms of hepatitis, including steatohepatitis and autoimmune hepatitis, as well as other types of hepatitis in a patient comprising administering an effective amount of a compound according to the present invention to a patient in need thereof, optionally in combination with a bioactive agent. Hepatitis includes hepatitis from viral infections, including Hepatitis A, B, C, D and E, Herpes simplex, Cytomegalovirus, Epstein-Barr virus, yellow fever virus, adenoviruses; non-viral infections, alcohol, toxins, drugs, ischemic hepatitis (circulatory insufficiency); pregnancy; autoimmune conditions, including Systemic Lupus Erythematosis (SLE); metabolic diseases, e.g. Wilson's disease, hemochromatosis and alpha one antitrypsin deficiency; and non-alcoholic steatohepatitis.

In still a further embodiment, the present invention relates to a treatment or inhibition of hyperlipidemia, especially hyperlipidemia associated with high triglycerides comprising administering to a patient in need thereof an effective amount of a compound according to the present invention, optionally in combination with a bioactive agent, in preferred embodiment a statin or statin-like drug substance.

Further embodiments are directed to one of more of the following aspects of the invention:

Oral mimetic compositions of RYGB surgery and methods of the present invention that cause the release of ileal brake hormones from the L-cells of the distal intestine, whereby effective dosages of the oral RYGB mimetics promote or accelerate pathway driven cellular level regeneration and remodeling of target organs and tissues in a mammal, principally a human;

The oral mimetic compositions of RYGB and methods of the present invention where the regenerated and or remodeled target is the pancreas in a patient with diabetes or pre-diabetes;

The oral mimetic compositions of RYGB and methods of the present invention where the regenerated and or remodeled target is the liver in a patient with NAFLD; NASH, cirrhosis, Hepatitis or HIV infection;

The oral mimetic compositions of RYGB and methods of the present invention where the regenerated and or remodeled target is the heart in a patient with ASHD, CHF, or ASCVD;

The oral mimetic compositions of RYGB and methods of the present invention where the regenerated and or remodeled target is the Gastrointestinal tract, principally the small intestine, in a patient with malabsorption, immune mediated injury such as coeliac disease, IBS, crohn's disease, or ulcerative colitis;

The oral mimetic compositions of RYGB and methods of the present invention where the regenerated and or remodeled target is the lung in a patient with COPD, asthma, or pulmonary fibrosis.

The oral mimetic compositions of RYGB and methods of the present invention where the regenerated and or remodeled target is the brain, in a patient with alzheimers disease or viral dike illnesses including but not limited to MS, ALS or the like;

The oral mimetic compositions of RYGB and methods of the present invention wherein patients with T2D have improved control of glucose and insulin resistance as a direct result of cellular level regeneration or remodeling of the pancreas;

The oral mimetic compositions and methods of the present invention wherein patients with T1D have improved control of glucose and insulin resistance as a direct result of cellular level regeneration or remodeling of the pancreas;

The oral mimetic compositions and methods of the present invention wherein patients with hepatic diseases have a reduction in NAFLD and hepatic inflammation as a direct result of cellular level regeneration or remodeling of the liver;

The oral mimetic compositions and methods of the present invention wherein patients with heart diseases, congestive heart failure, myocarditis and cardiomyopathy have a reduction in atherosclerosis and associated ischemic injury as a direct result of cellular level regeneration or remodeling of the heart and associated cardiovascular system;

The oral mimetic compositions and methods of the present invention wherein patients with malabsorptive gastrointestinal diseases such as coeliac, IBD, Crohn's disease and the like have a reduction in malabsorption and/or inflammation of intestinal mucosa and associated injury as a direct result of cellular level regeneration or remodeling of the gastrointestinal intimal surfaces;

The oral mimetic compositions and methods of the present invention wherein patients with lung diseases have a reduction in inflammation or fibrosis and associated ischemic injury as a direct result of cellular level regeneration or remodeling of the lungs;

The oral mimetic compositions and methods of the present invention wherein patients with brain diseases have a reduction in inflammation or abnormal amyloid accumulation and associated loss of neuron mass as a direct result of cellular level regeneration or remodeling of the brain;

The oral mimetic compositions of RYGB wherein the active compound responsible for cellular level regeneration or remodeling is Brake™ (an oral ileal brake hormone releasing composition as otherwise described herein), a specific formulation targeting release of ileal brake hormones from the L-cells of the distal small intestine;

The oral mimetic compositions of RYGB wherein Brake™ composition (an oral ileal brake hormone releasing composition as otherwise described herein) is combined with a second active pharmaceutical to produce an enhanced degree of cellular level regeneration or remodeling beyond that of Brake alone, and said oral combination of active pharmaceuticals can be used to treat disease states and/or conditions including any of T2D, T1D, Obesity, Hyperlipidemia, ASHD, CHF, COPD, Diabetic complications such as Neuropathy, Alzheimer's disease, or any end organ manifestation of metabolic syndrome or the associated systemic inflammation;

A method of stimulating cellular level regeneration of target organs and tissues by administering an oral mimetic of RYGB surgery to a human patient in need thereof, wherein the oral mimetic of RYGB surgery can be used alone or in combination to treat any condition that is improved by RYGB surgery and the associated cellular level regeneration of target organs and tissues;

An oral ileal brake hormone releasing composition comprising a compound for stimulating long-term release of ileal hormones in combination with at least one additional bioactive or pharmaceutical agent.

An oral ileal brake hormone releasing composition wherein the bioactive or pharmaceutical agent is a hepatitis C anti-viral agent, an anti-diabetes agent including a DPP-IV inhibitor, a proton pump inhibitor an anti-obesity agent or an agent which reduces Hyperlipidemia in a patient or subject.

An oral ileal brake hormone releasing composition wherein the compound for stimulating is a composition comprising an effective amount of pH encapsulated glucose, optionally with other components which deliver effective amounts of glucose into the ileum to influence the ileal brake and the release of hormones in the ileum including as described herein.

An oral ileal brake hormone releasing composition comprising an effective amount of pH encapsulated lipids in an effective amount to stimulate the GPR-120 receptor on the L-cells of the jejunum and ileum.

In an additional embodiment, the present invention also relates to a method of enhancing the regeneration or remodeling of target organs and tissues of patients with metabolic syndrome diseases in need thereof, wherein the treatment is oral mimicry of RYGB actions and thereby produces the endogenous process of regeneration or remodeling of target organs and tissues.

In still a further embodiment, the present invention relates to a method of enhancing the regeneration or remodeling of target organs and tissues of patients with metabolic syndrome diseases in need thereof, wherein the primary treatment is a cell transplant or a stem cell transplant or the like, and the enabling treatment to benefit retention of the implanted cells or tissues is oral mimicry of RYGB actions as described hereinabove.

These and other aspects of the invention are explained further in the following detailed description of the invention.

### BRIEF DESCRIPTION OF THE FIGURES

Figures for Examples 1-4

FIG. 1 is a scatter plot of blood levels (ng/ml) of GLP-1, GLP-2, C-peptide, GLP-1 (total) (determined by radioimmunoassay (RIA)), PYY, blood glucose (BS), GLP-1 (total) (with plasma), and insulin for live subjects tested in the experiment described in Example 1.

FIG. 2 illustrates four-month weight loss of the subject described in the experiment of example 2. Significant weight loss using the presently claimed compositions was evidenced. Further data (not presented) also evidenced consistent significant reduction/stabilization in glucose levels pursuant to the ingestion of a composition according to the present invention within about a 4 hour to 10 hour period.

FIGS. 3A and 3B show the total stimulation above the baseline as a consequence of administration as a function of time to subjects. 3A is the total stimulation above the baseline for Case 1. 3B is the total stimulation above the baseline for Case 2.

FIG. 4 discloses a table A and a table B containing the statistical correlations undertaken in connection with the experiments of example 3.

FIGS. 5A-J discloses twelve-hour values of blood levels above baseline of (5A) GLP-1 (pM). (5B) GLP-1 (with patient I as outlier and removed from graph), (5C) Glucose (blood glucose, mg/dl), (5D) C-peptide (ng/ml), (5E) Insulin (μIu/ml), GLP-1 (total) (RIA), (5F)PYY (3-36, pg/ml), (5G) leptin (ng/ml), (5H) Glucagon (pg/ml), (5I) IGF-1 (ng/ml) and (5J) IGF-II (ng/ml) for subjects F, G, H, I and J tested in the experiment described in Example 3. The IGF and other parameters were measured to try to explain the decrease of insulin resistance seen as well as the simultaneous decrease in both the insulin and glucose showing a significant potential for treating diabetes as well as prediabetes and an increase in muscle mass, and reduction in fat mass.

FIGS. 6A-F shows the results of GLP-1 response to a formulation according to the present invention for five patients tested. The graphs presented represent the total GLP-1 (pM) stimulation per hours comparing prior art levels in response to a mixed meal (triangles) and the results obtained from the use of the present invention in 5 patients. Note that the stimulation of the hormones by the present invention occurs between approximately hours 4 and 10 or more (after ingestion). FIG. 6F represents outlier results for patient I.

FIGS. 7A-E shows the results of PYY response in individuals following the ingestion of a formulation according to the present invention. As can be seen from the results presented in these figures, PYY stimulation (pg/ml) is the same pattern as other hormones of the ileal break with a maximum intensity between about 4 to 10 hours, even though the cephalic phase is more prominent than is GLP-1 (pM). The overall stimulus is consistent with the stimulation by the formulation of the present invention.

FIGS. 8A-J shows the results of glucose, insulin and C-peptide response in five groups of individuals following the ingestion of a formulation according to the present invention. 8A shows the results of glucose (mg/dl), insulin (μlu/ml) and C-peptide (ng/nil) response in individuals with normal glucose and mild elevation of insulin; 8B shows the results of glucose, insulin and C-peptide response in individuals with elevated glucose and normal to reduced/low levels of insulin; 8C shows the results of glucose, insulin and C-peptide response in individuals with elevated levels of glucose and insulin; 8D shows the results of glucose, insulin and C-peptide response in individuals with normal glucose and elevated fasting insulin and 8E shows the results of glucose, insulin and C-peptide response in individuals with normal glucose and mild insulin increase. FIG. 8F shows that with elevated blood glucose and insulin there is a continual seesaw between insulin stimulation and suppression. 8G and 8H show that with normal glucose and elevated fasting insulin there is a decrease in glucose and insulin over time. 8I and 8J show that with normal glucose and mild insulin increase there is insulin reduction with decrease in blood glucose.

FIG. 9 is a chart showing the change in levels of various blood components during testing, with Table 1 showing the data, for the following subject: white male, 35 years old with a BMI of 29 (overweight). Note that the following is applicable, where relevant for FIGS. 9-28: GLP-1 (pM, RIA), GLP-2 (ng/ml), Glucose-(mg-dl), Crpeptide (ng/ml), insulin (μlu/ml), GLP-1 (total) (RIA), PYY (3-36, pg/ml), leptin (ng/ml), Glucagon (pg/ml), IGF-I (ng/ml) and IGF-11 (ng/ml).

FIG. 10 is a chart showing the change in levels of various blood components during testing, with Table 2 showing the data, for the following subject: white male, 33 years old with a BMI of 23 (normal);

FIG. 11 is a chart showing the change in levels of various blood components during testing, with Table 3 showing the data, for the following subject: white male, 46 years old with a BMI of 29 (overweight);

FIG. 12 is a chart showing the change in levels of various blood components during testing, with Table 4 showing the data, for the following subject: white female, 50 years old with a BMI of 26 (overweight);

FIG. 13 is a chart showing the change in levels of various blood components during testing, with Table 5 showing the data, for the following subject: white male, 23 years old with a BMI of 40 (obese);

FIG. 14 is a chart showing the change in levels of various blood components during testing, with Table 6 showing the data, for the following subject: white male, 33 years old with a BMI of 32 (obese);

FIG. 15 is a chart showing the change in levels of various blood components during testing, with Table 8 showing the data, for the following subject: white male, 61 years old with a BMI of 34 (obese);

FIG. 16 is a chart showing the change in levels of various blood components during testing, with Table 9 showing the data, for the following subject: white male, 29 years old with a BMI of 26 (overweight);

FIG. 17 is a chart showing the change in levels of various blood components during testing, with Table 10 showing the data, for the following subject: black female, 44 years old with a BMI of 37 (obese);

FIG. 18 is a chart showing the change in levels of various blood components during testing, with Table 11 showing the data, for the following subject: black male, 18 years old with a BMI of 29 (overweight);

FIG. 19 is a chart showing the change in levels of various blood components during testing, with Table 12 showing the data, for the following subject: white female, 58 years old with a BMI of 22 (normal);

FIG. 20 is a chart showing the change in levels, of various blood components during testing, with Table 13 showing the data, for the following subject: white female, 45 years old with a BMI of 30 (obese);

FIG. 21 is a chart showing the change in levels, of various blood components during testing, with Table 14 showing the data, for the following subject: white male, 68 years old with a BMI of 29 (overweight):

FIG. 22 is a chart showing the change in levels of various blood components during testing, with Table 15 showing the data, for the subject tested;

FIG. 23 is a chart showing the change in levels of Various blood components during testing, with Table 16 showing the data, for the subject tested;

FIG. 24 is a chart showing the change in levels of various blood components during testing, with Table 1 showing the data, for the following subject: black female, 24 years old with a BMI of 44 (obese);

FIG. 25 is a chart showing the change in levels of various blood components during testing, with Table 18 showing the data, for the tested subject;

FIG. 26 is a chart showing the change in levels of various blood components during testing, with Table 19 showing the data, for the following subject: white male, 48 years old with a BMI of 26 (overweight);

FIG. 27 is a chart showing the change in levels of various blood components during testing, with Table 20 showing the data, for the following subject: Hispanic female, 47 years old with a BMI of 22 (normal);

FIG. 28 is a chart showing the change in levels of various blood components during testing, with Table 21 showing the data, for the following subject: white female, 57 years old with a BMI of 37 (obese).

Figures for Further Examples

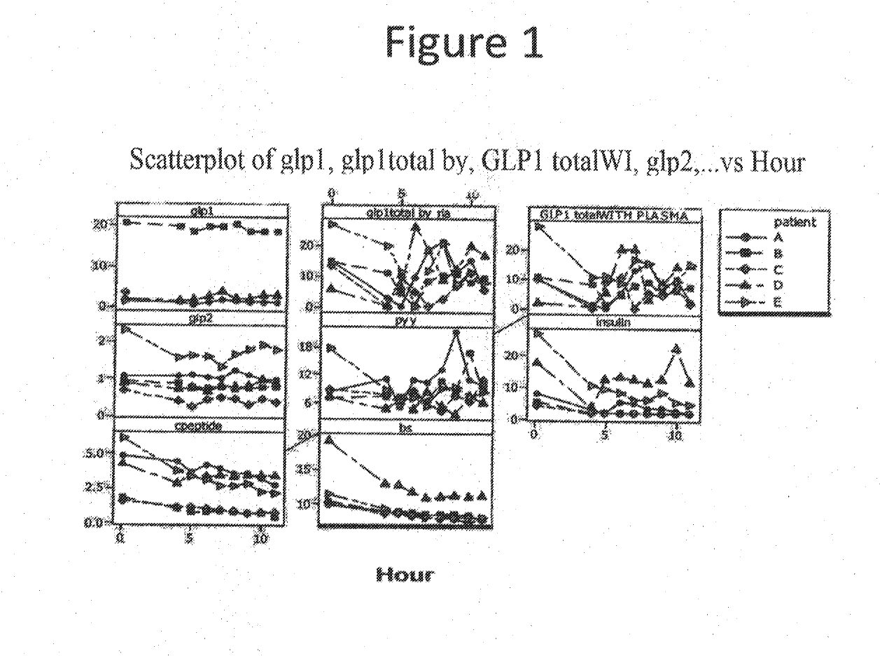

FIG. 1B (Further Examples) resting Results for GLP-1 and GLP-2 by Formulation Aphoeline 0 and Aphoeline 1.

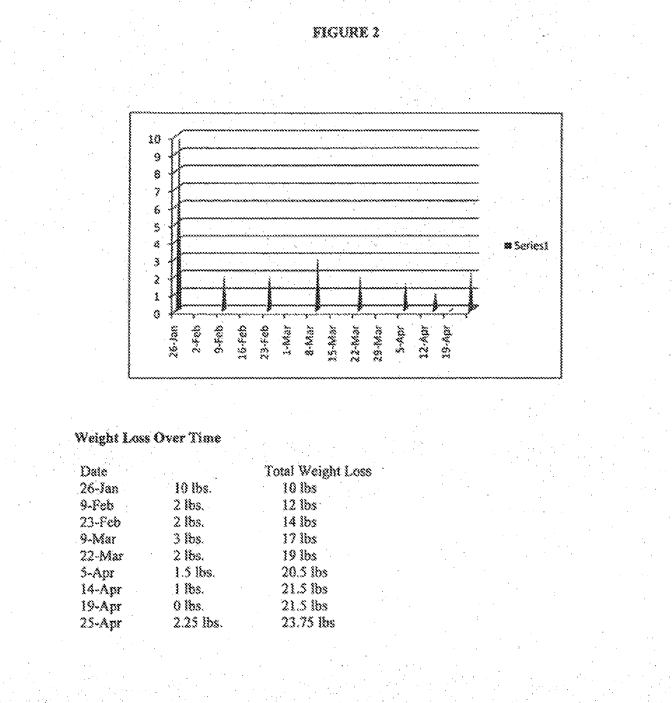

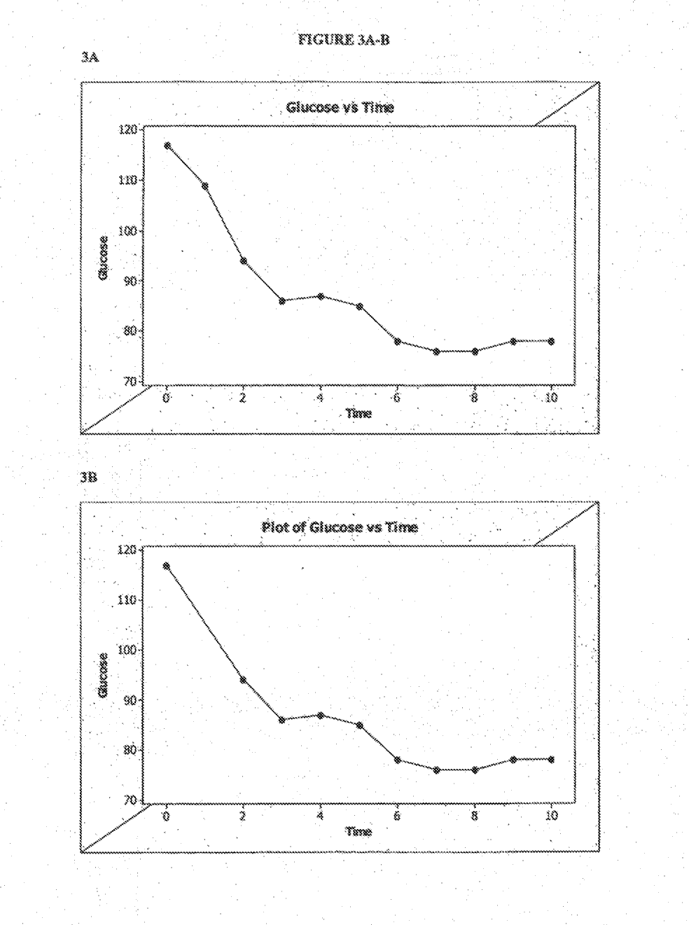

FIG. 2E (Further Examples) Testing Results for EGF1 and IGF2 by Formulation Aphoeline 0 and Aphoeline 3.

FIG. 3E (Further Examples) Testing Results for Glucose and Insulin by Formulation Aphoeline 0 and Aphoeline 1.

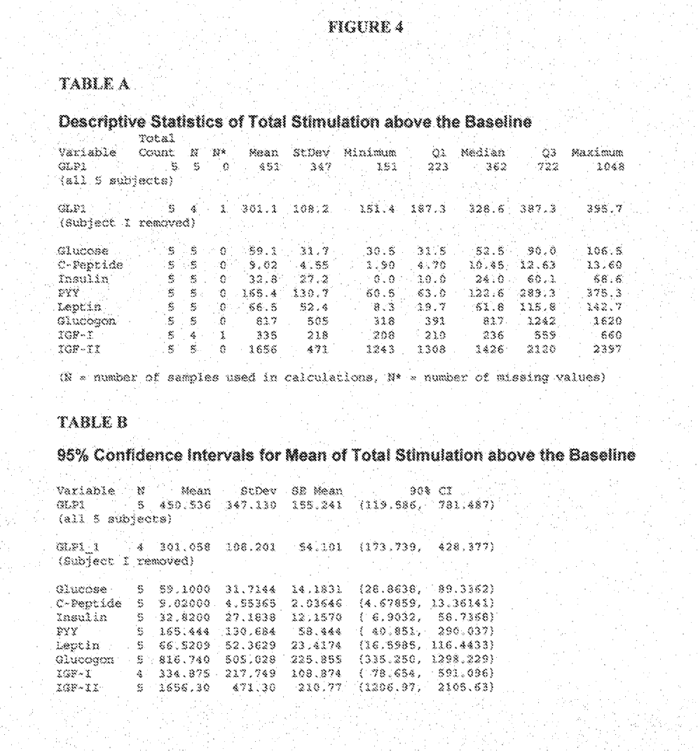

FIG. 4E (Further Examples) Testing Results for EGF1 and IGF2 by Formulation Aphoeline 0 and Aphoeline 1.

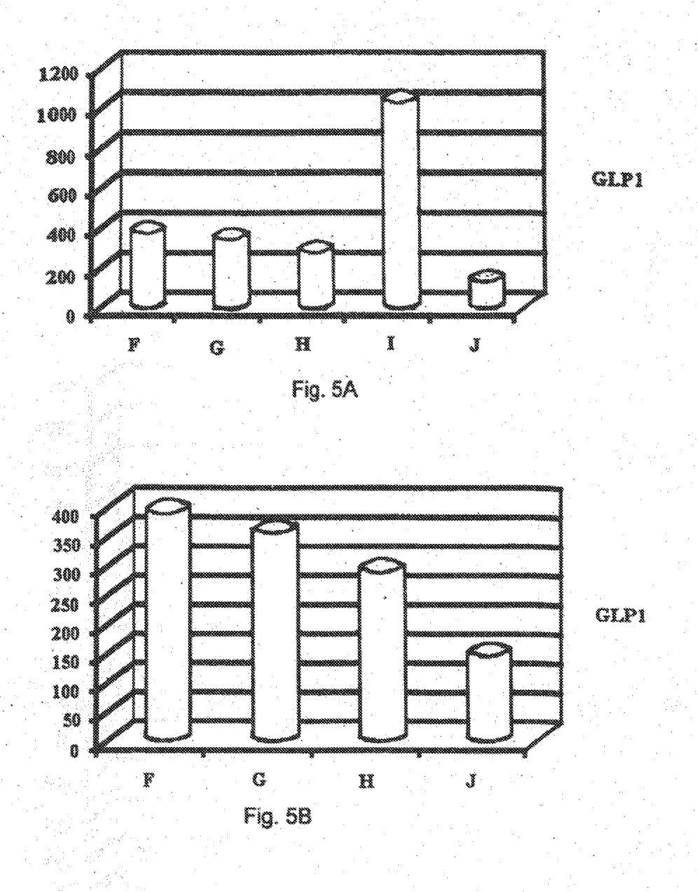

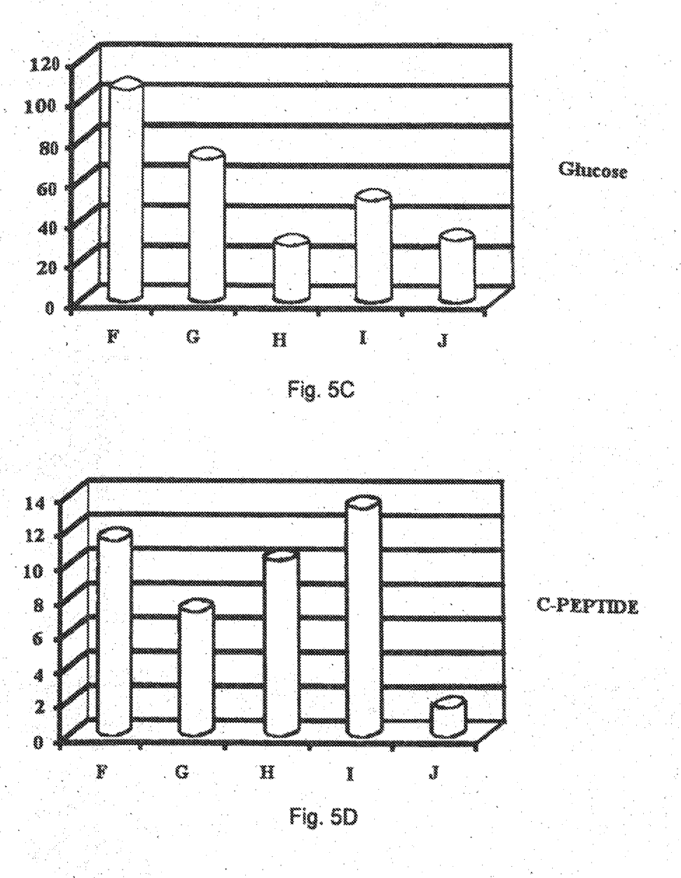

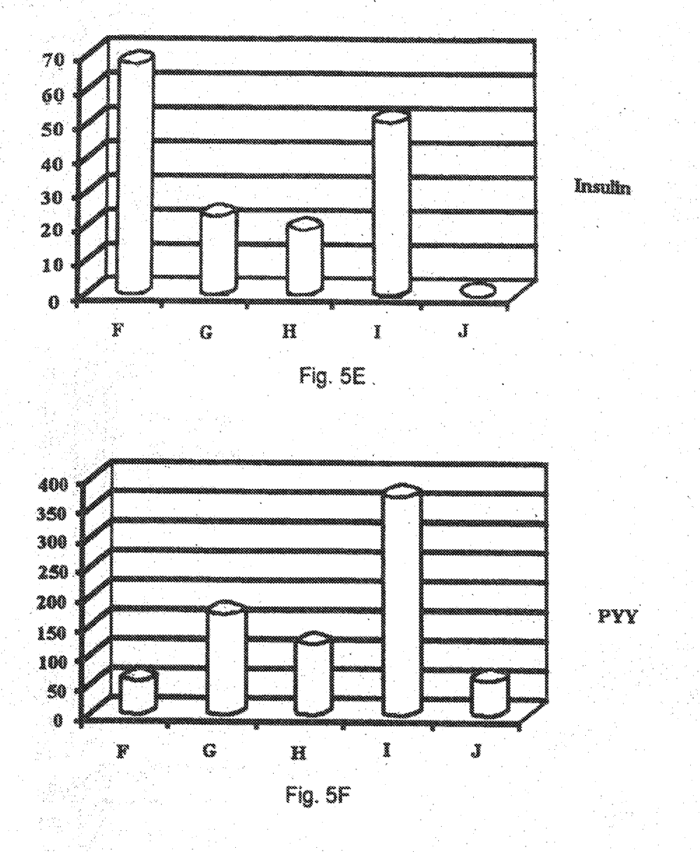

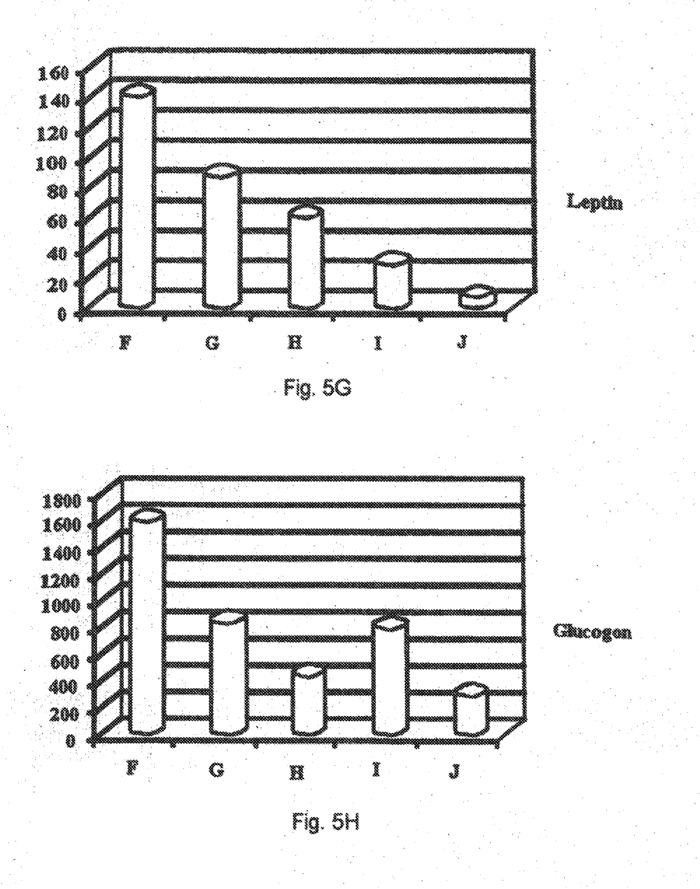

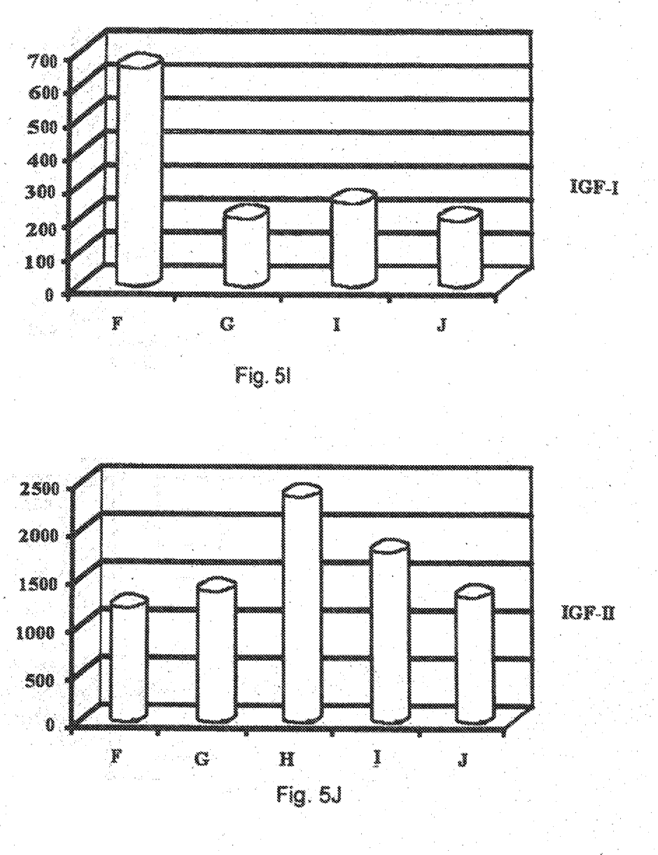

FIG. 5E (Further Examples) Average Levels for Aphoeline 0 Group.

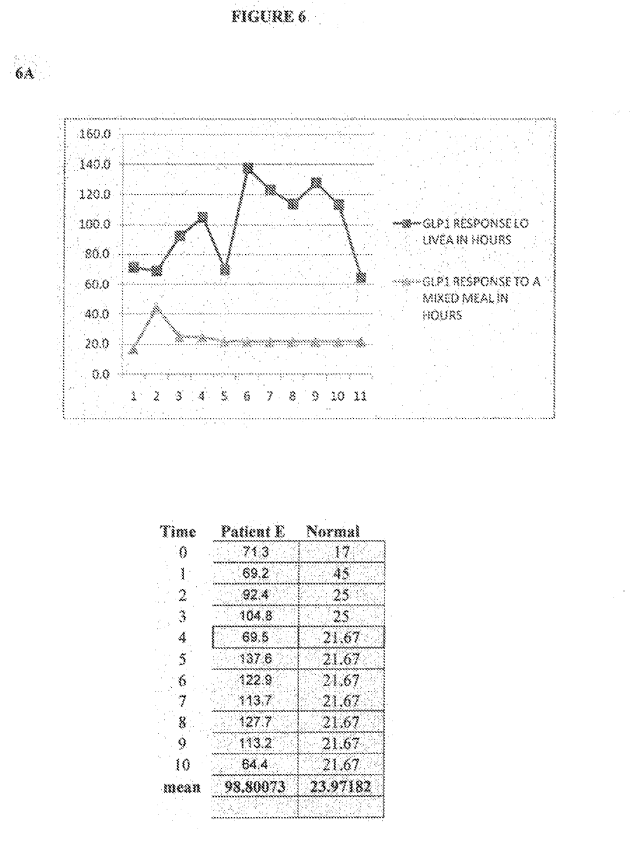

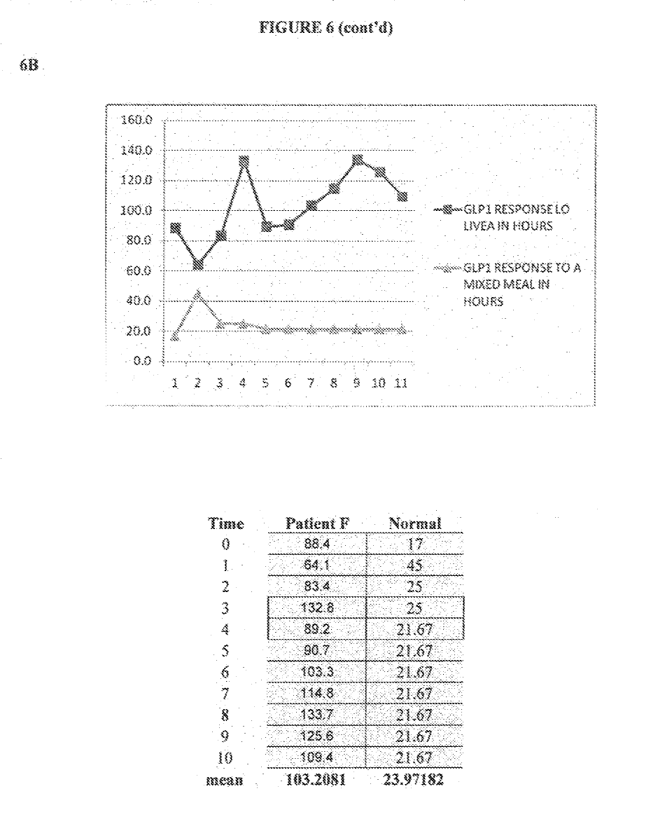

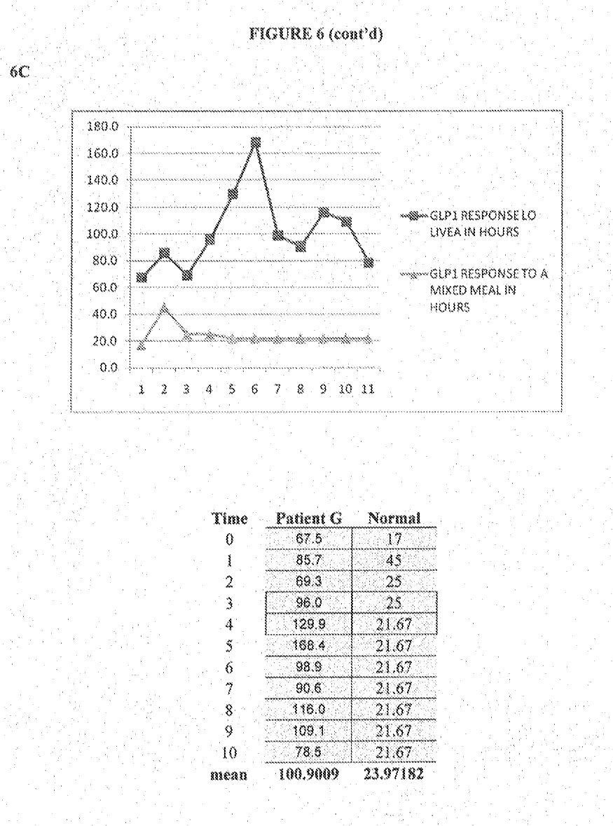

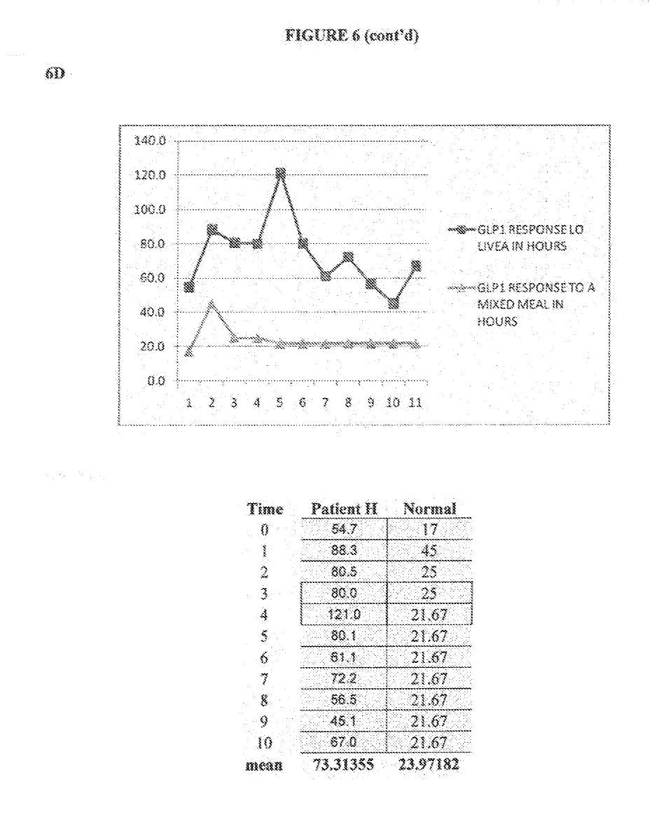

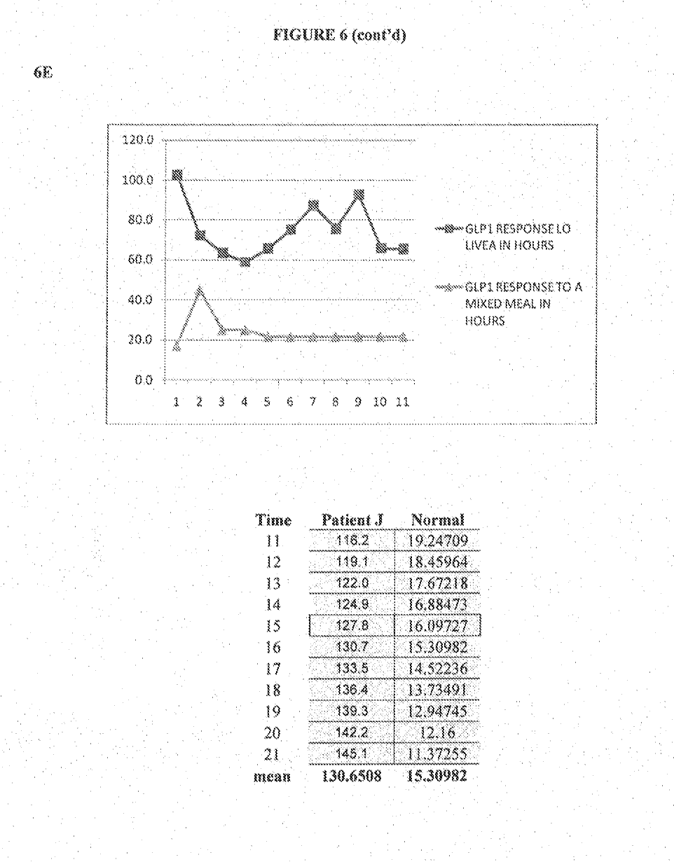

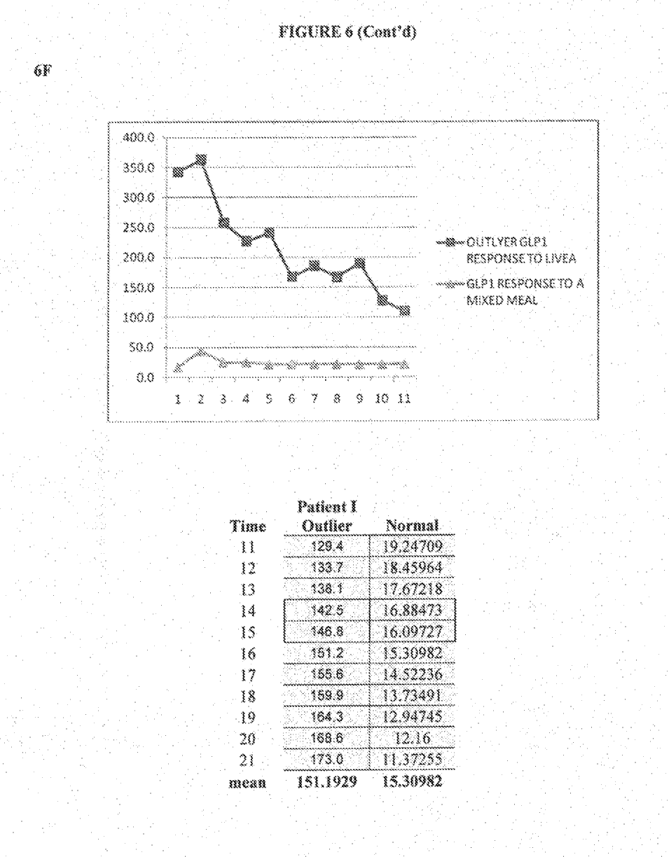

FIG. 6E (Further Examples) Average Levels for Aphoeline 1 Group,

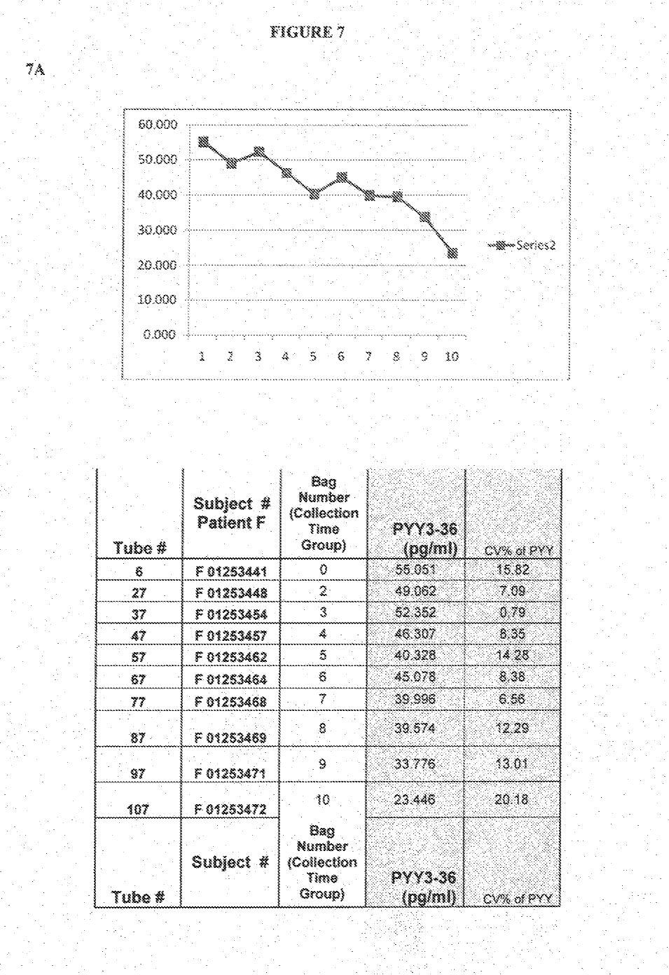

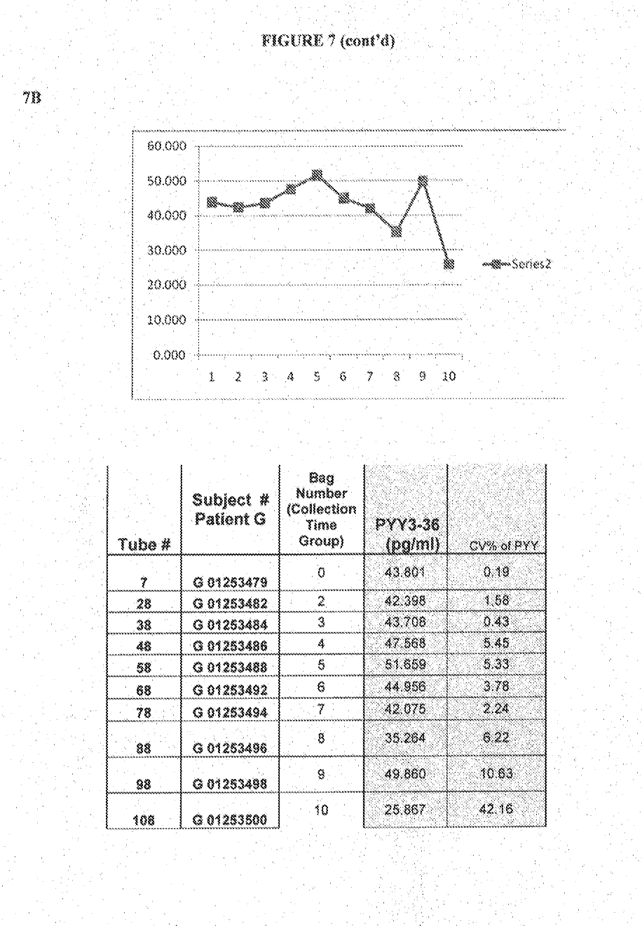

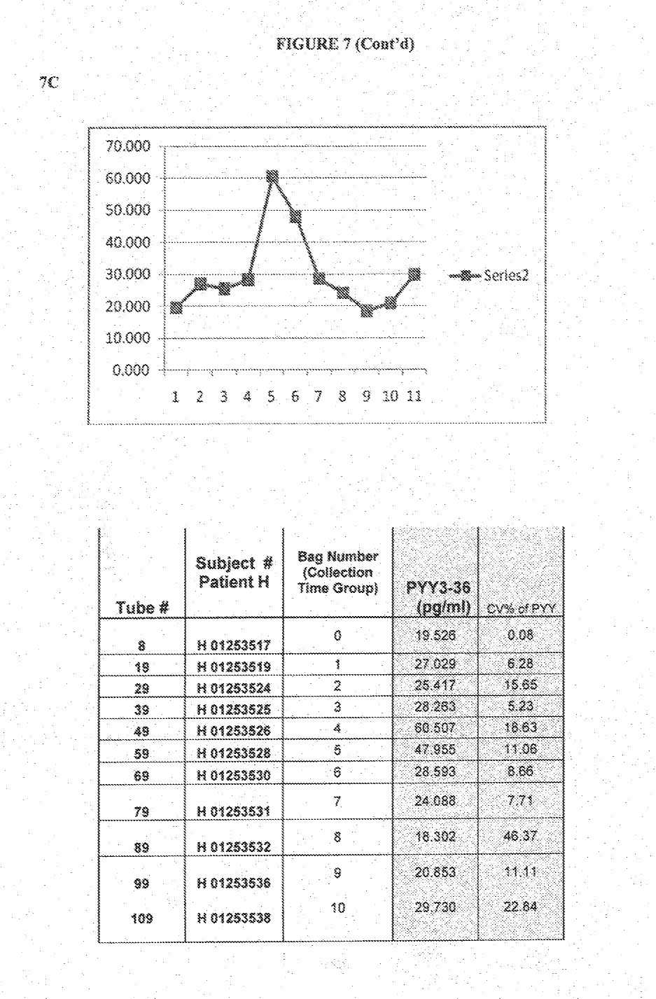

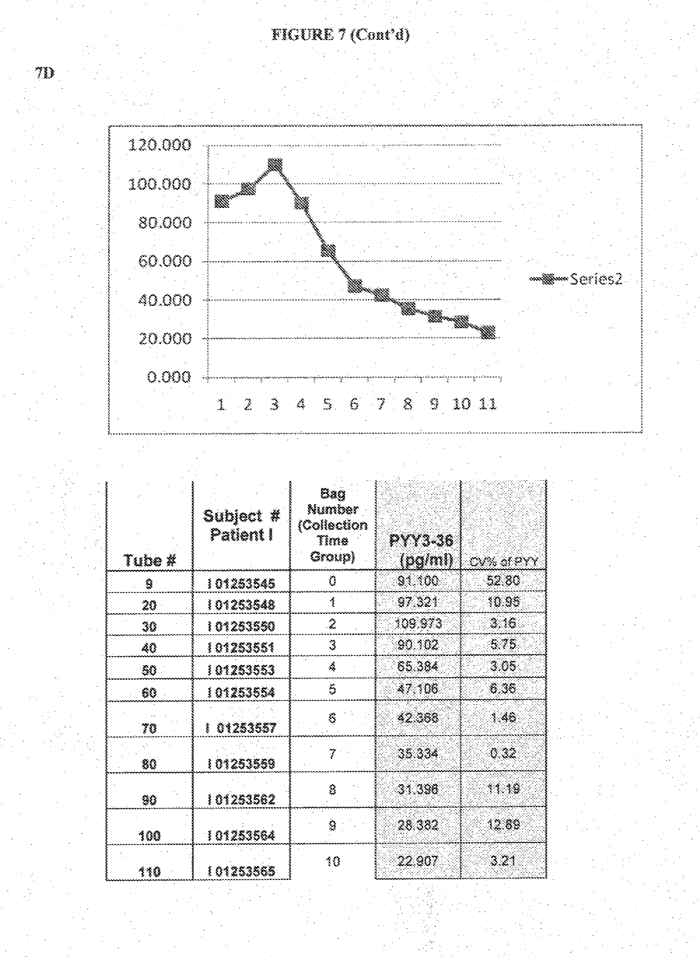

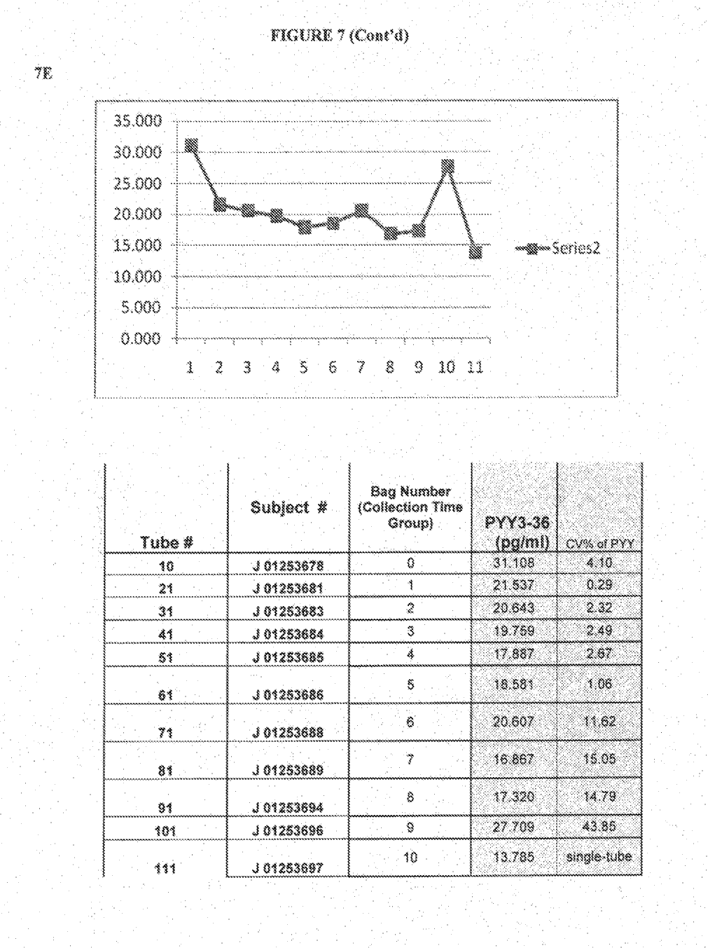

FIG. 7E (Further Examples) Glucose concentrations for subjects with elevated Glucose/Insulin concentrations.

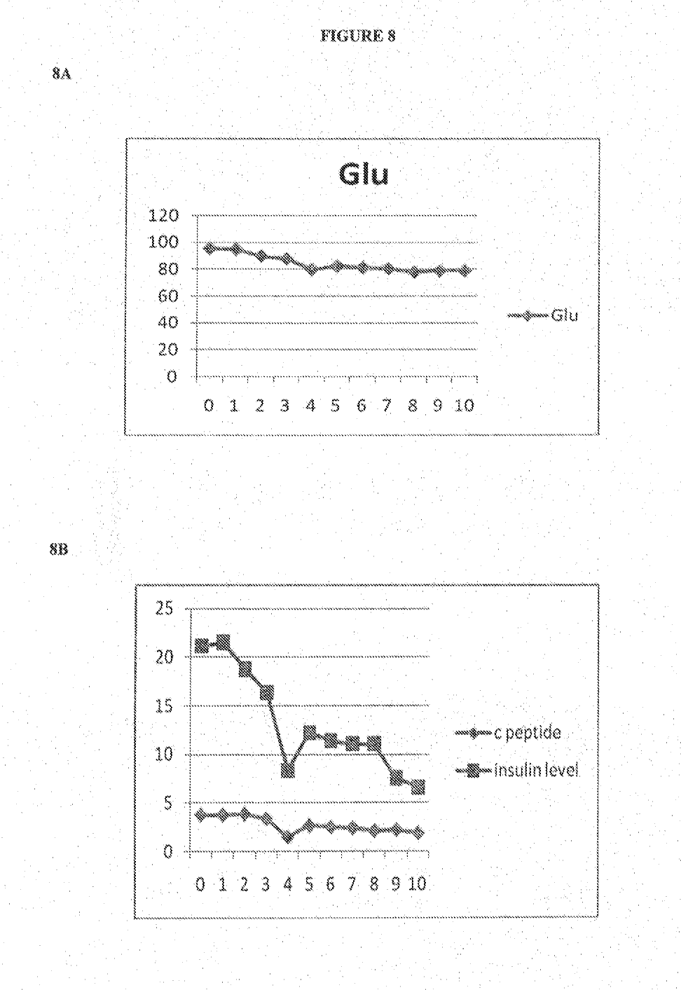

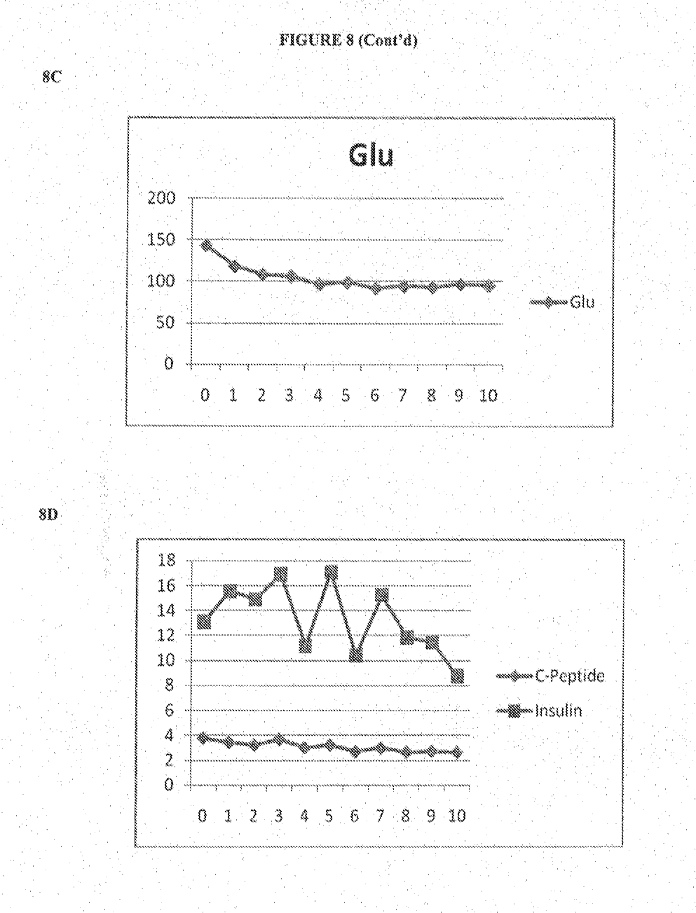

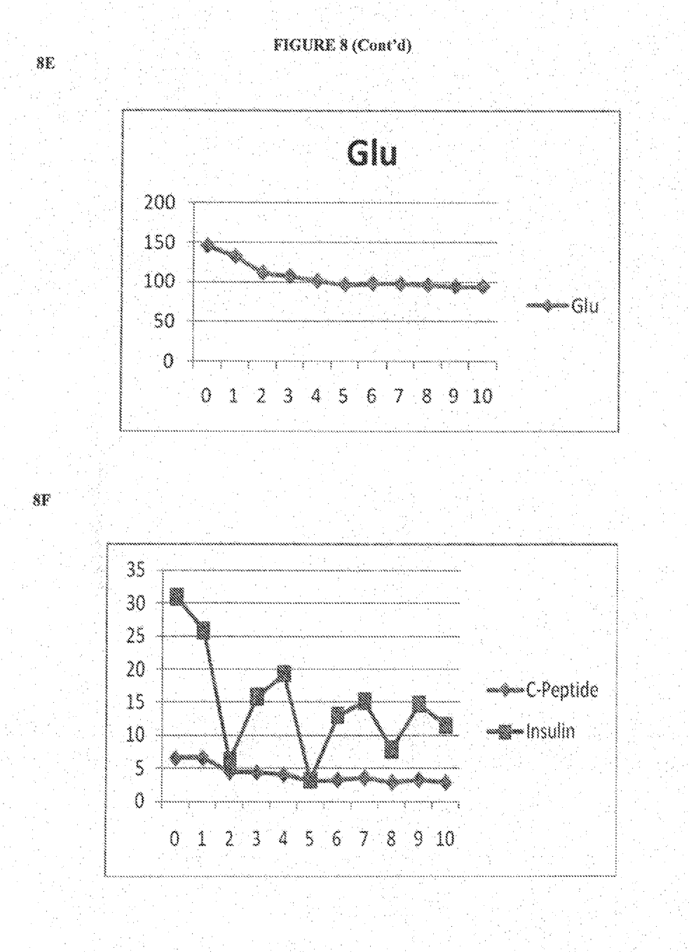

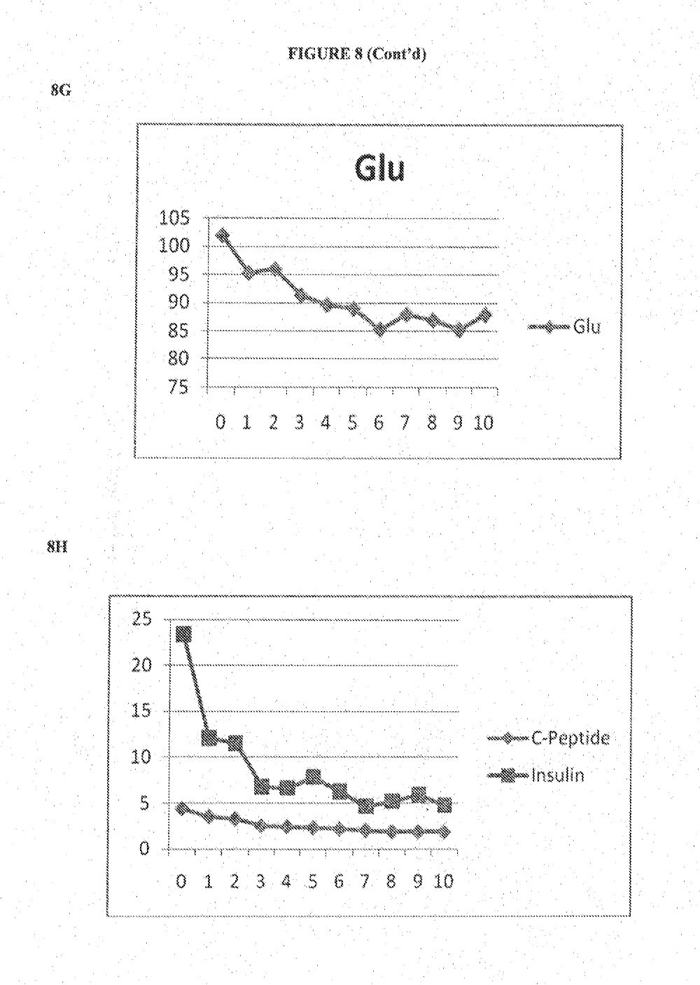

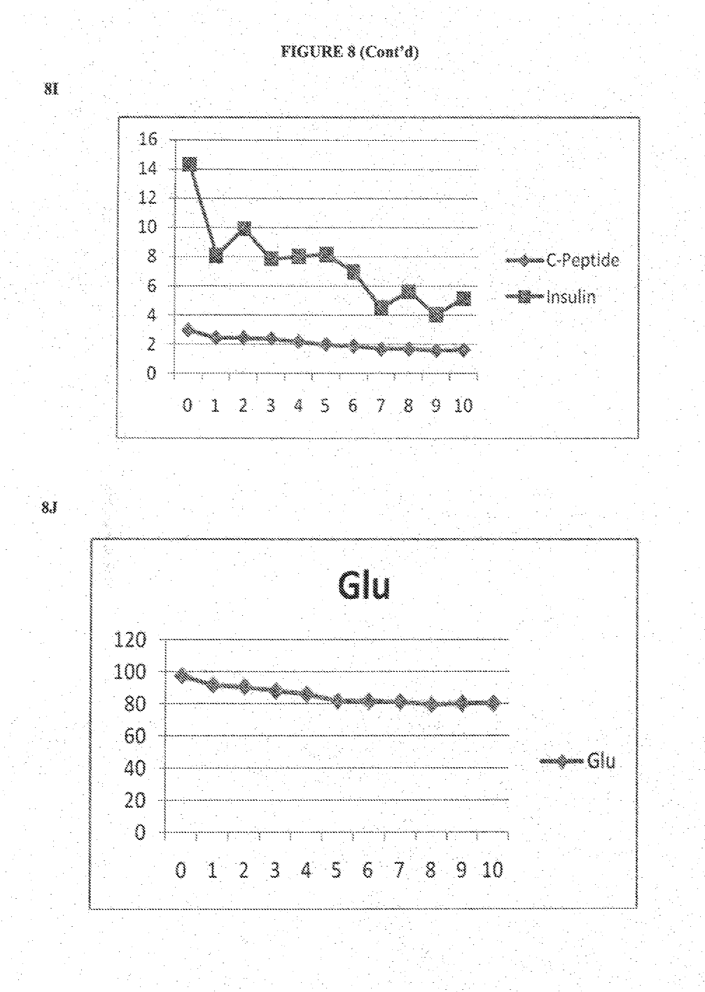

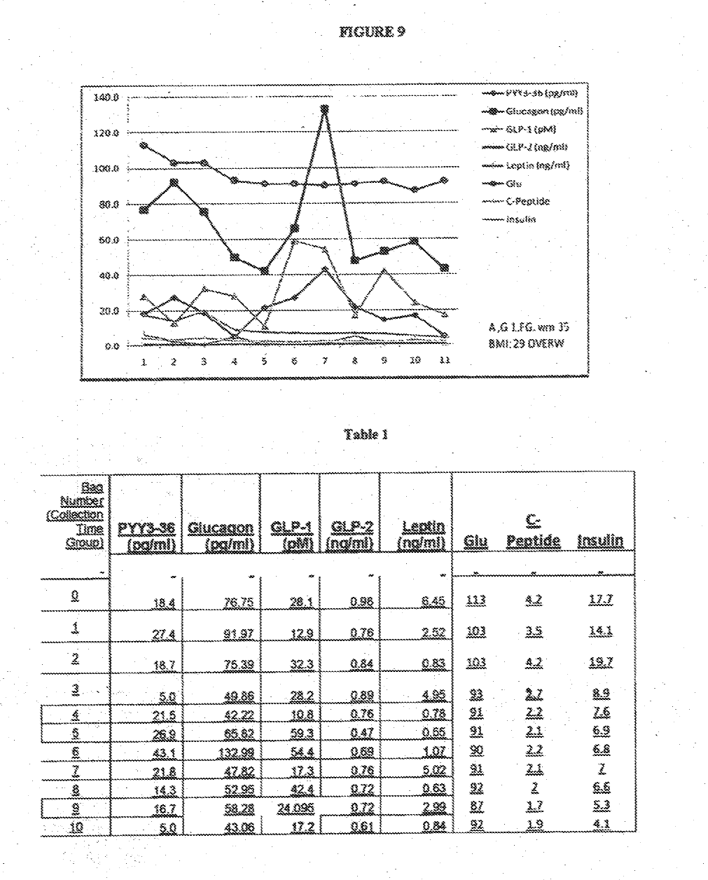

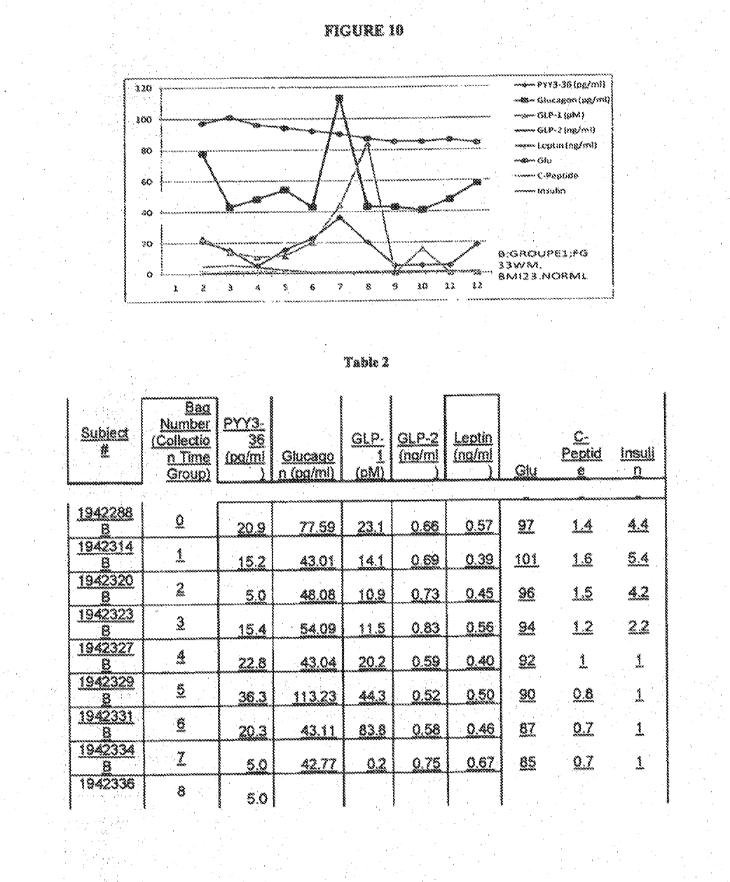

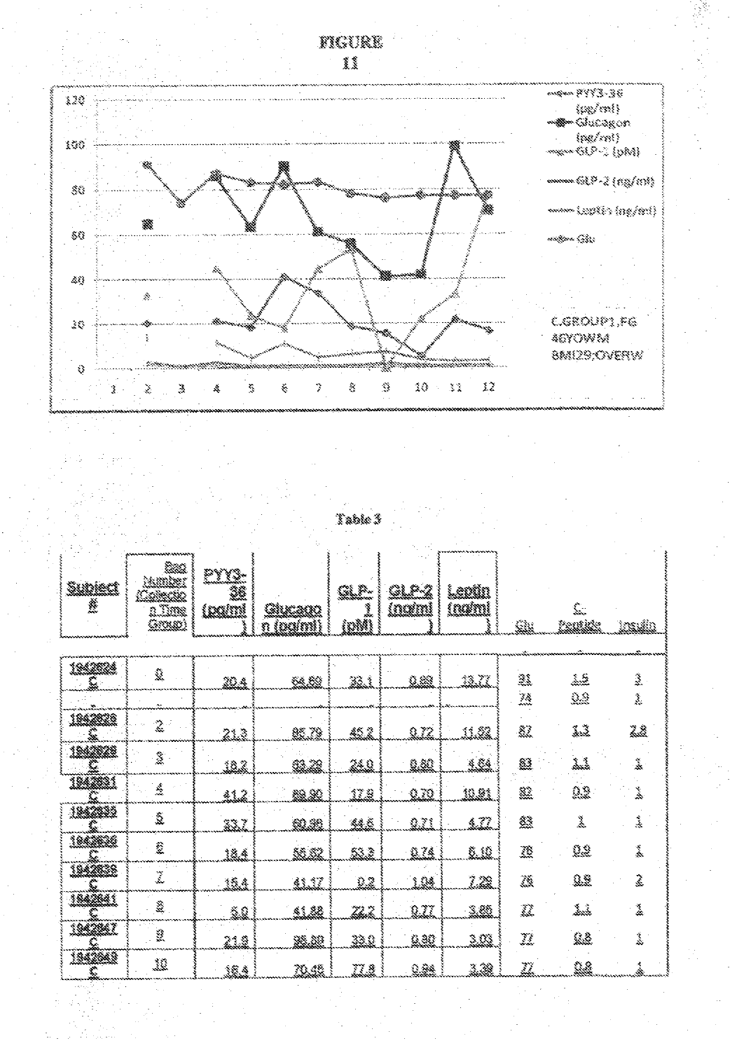

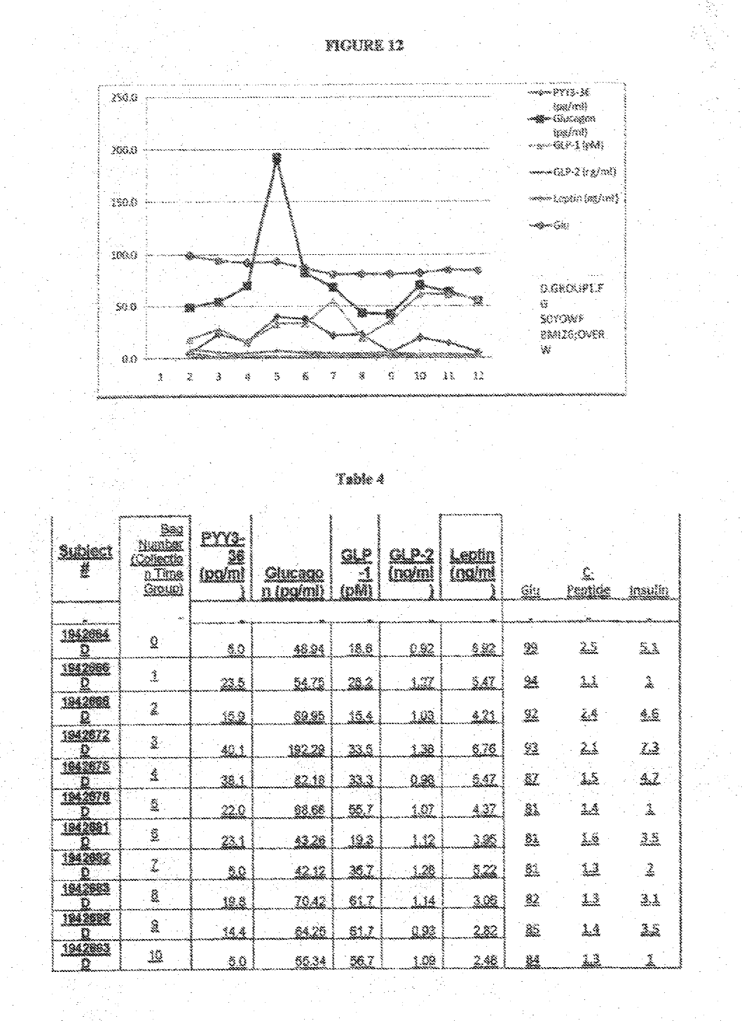

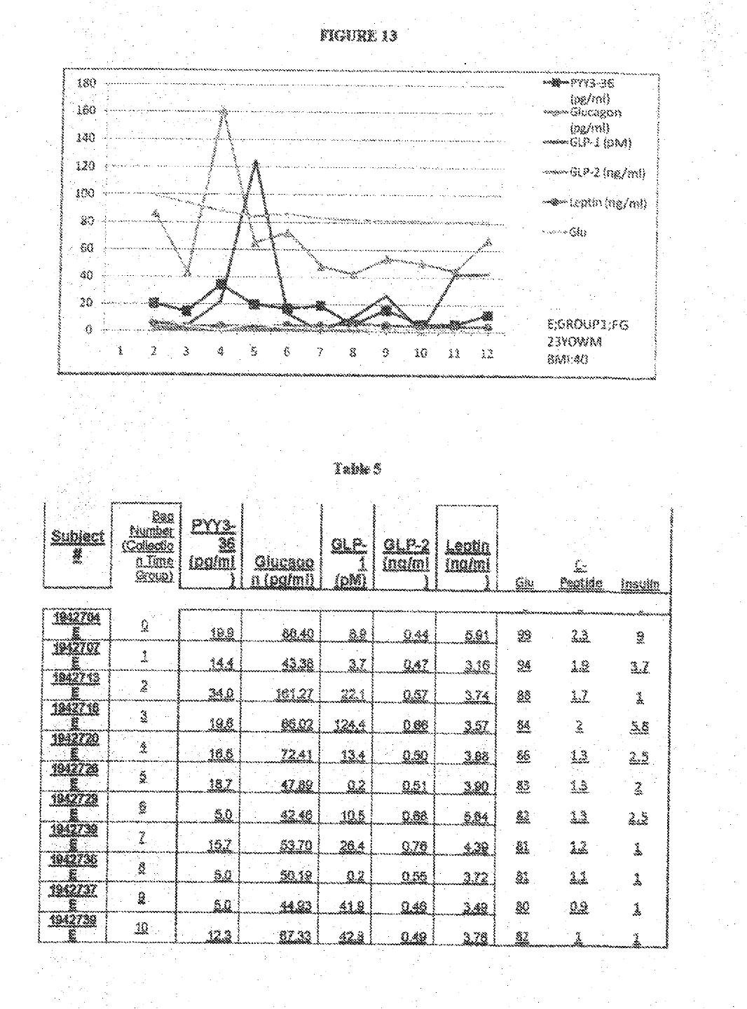

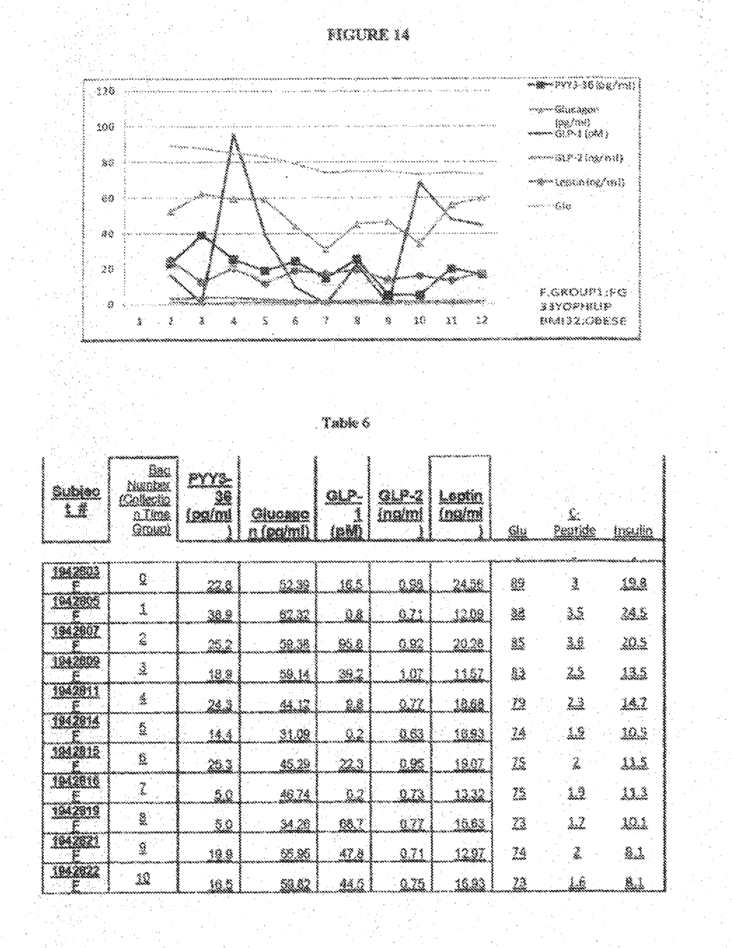

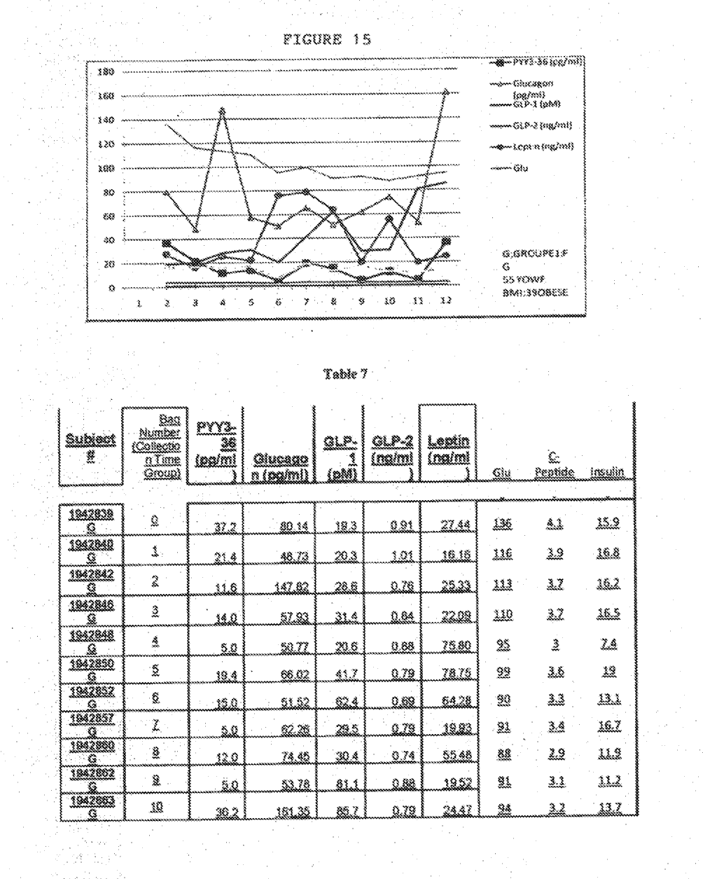

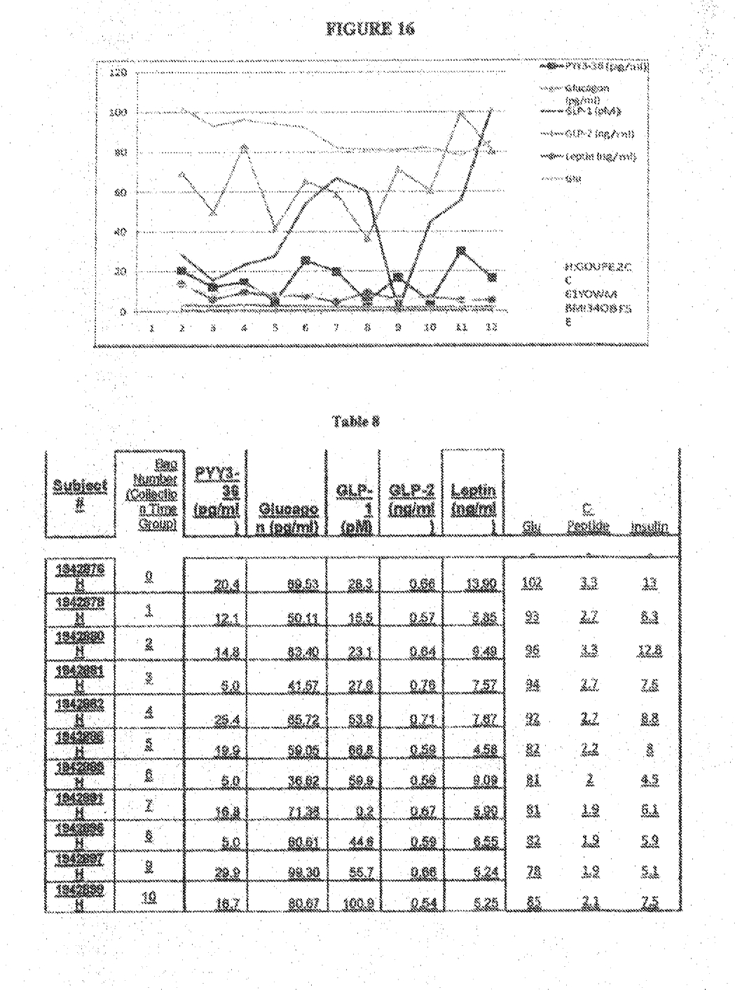

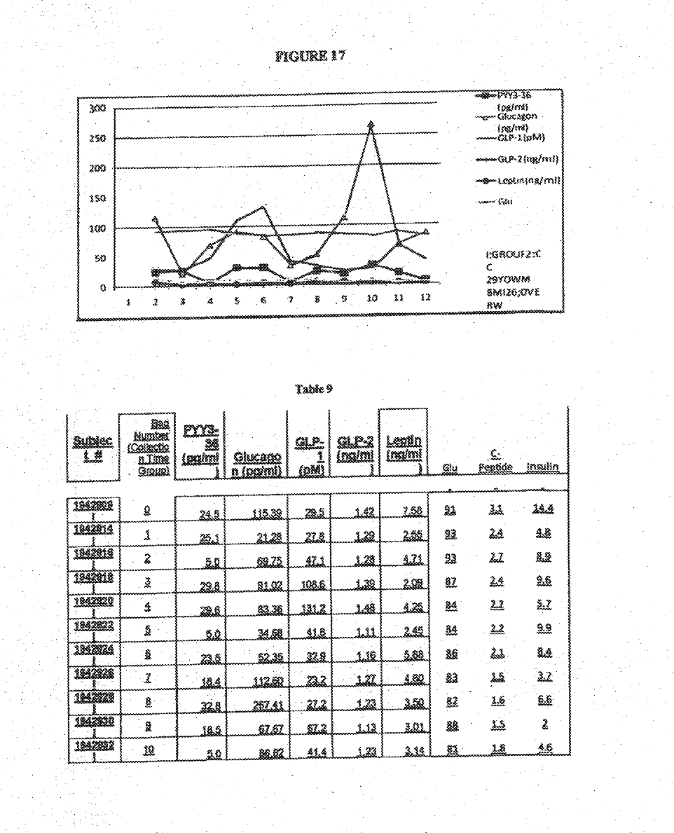

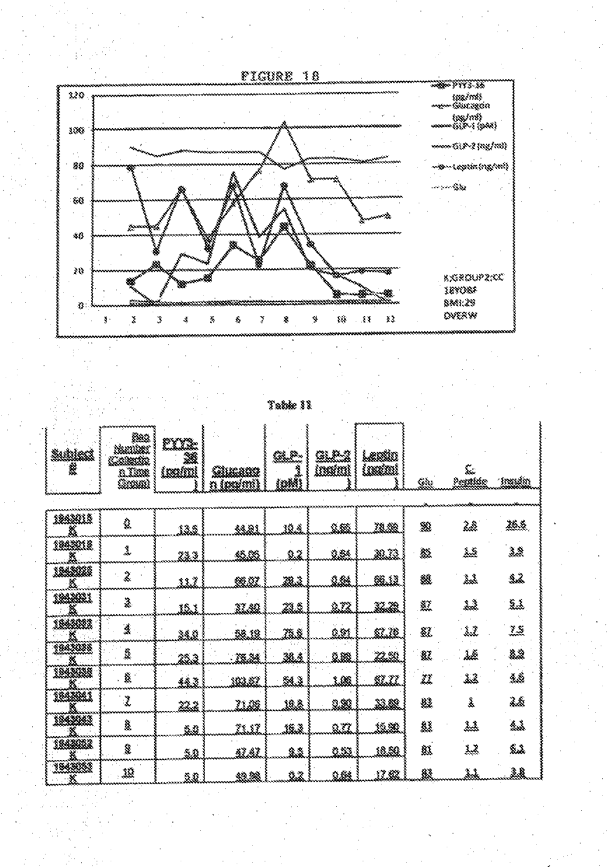

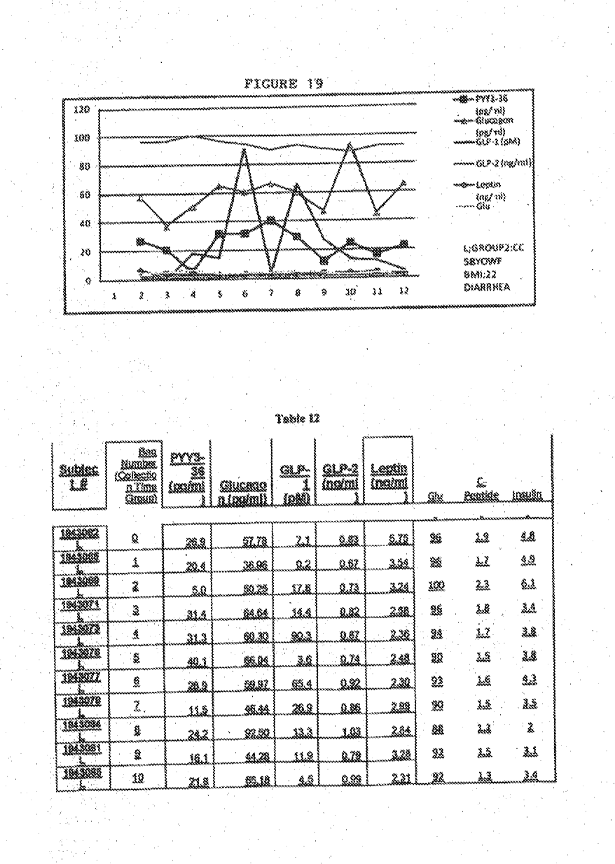

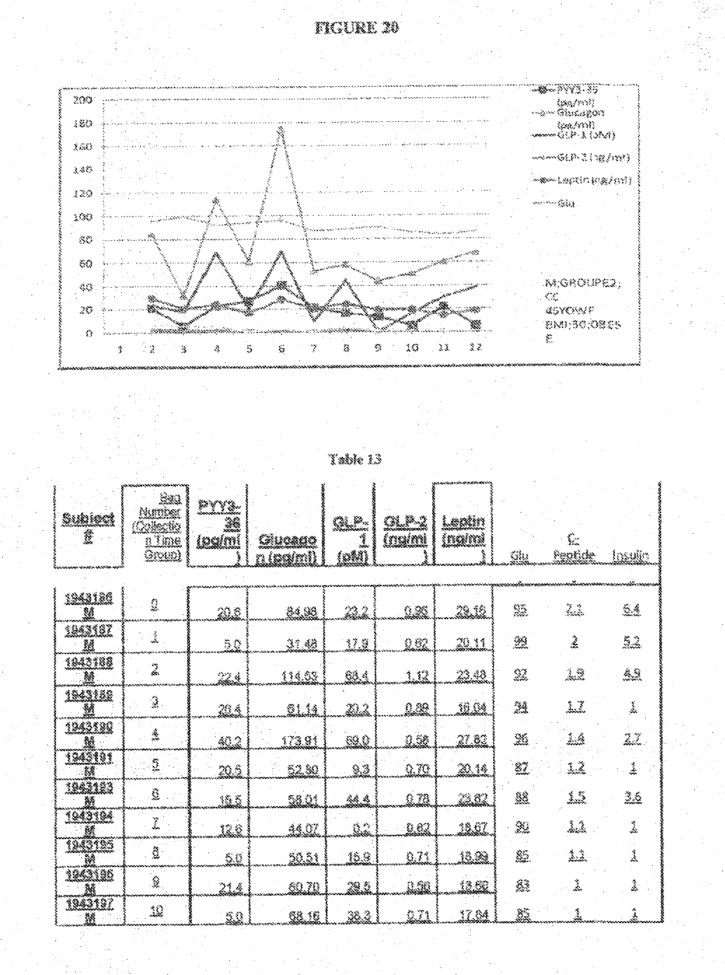

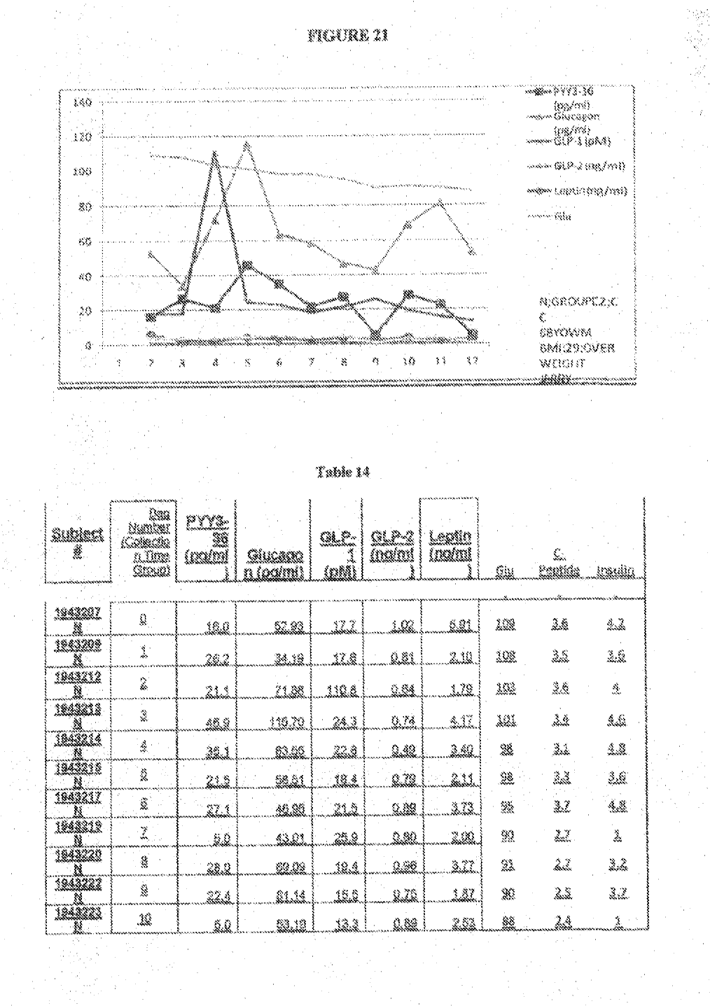

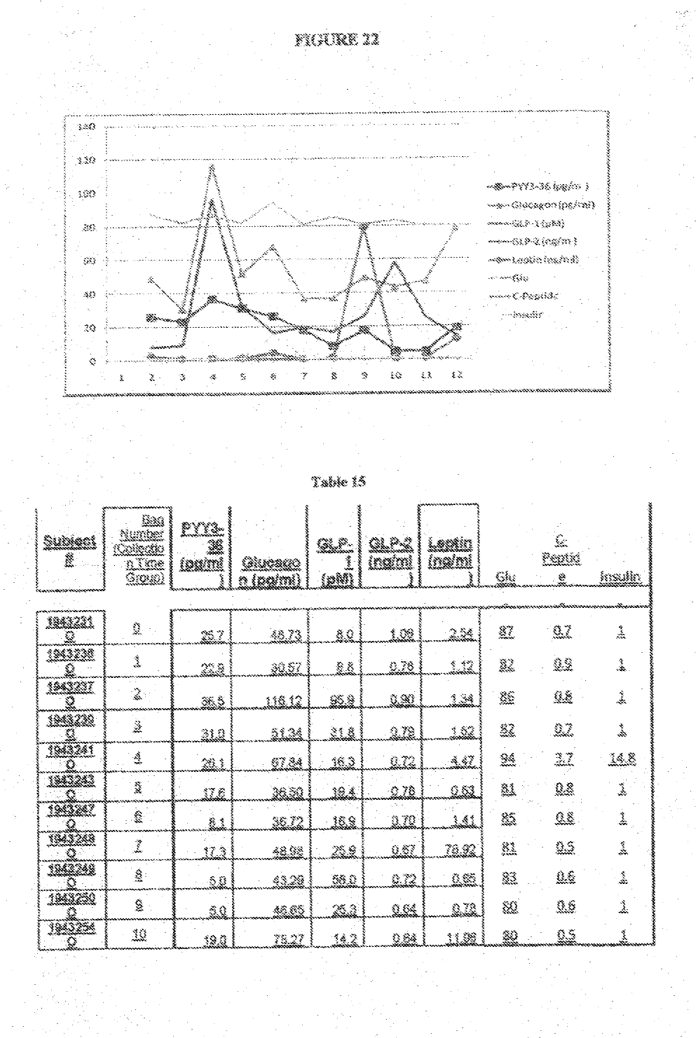

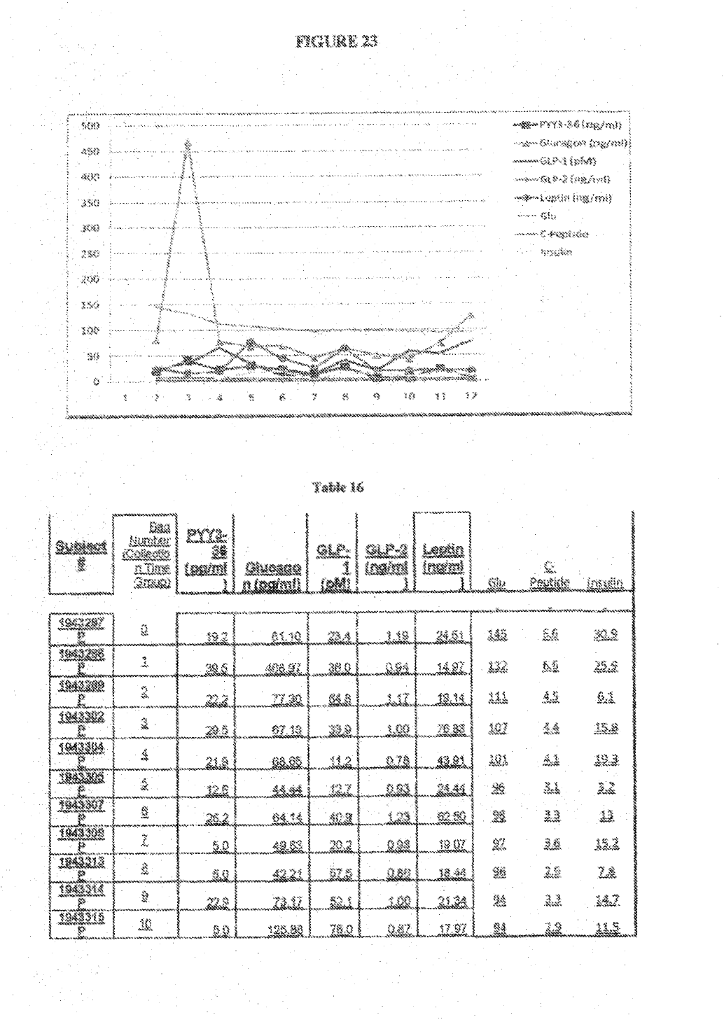

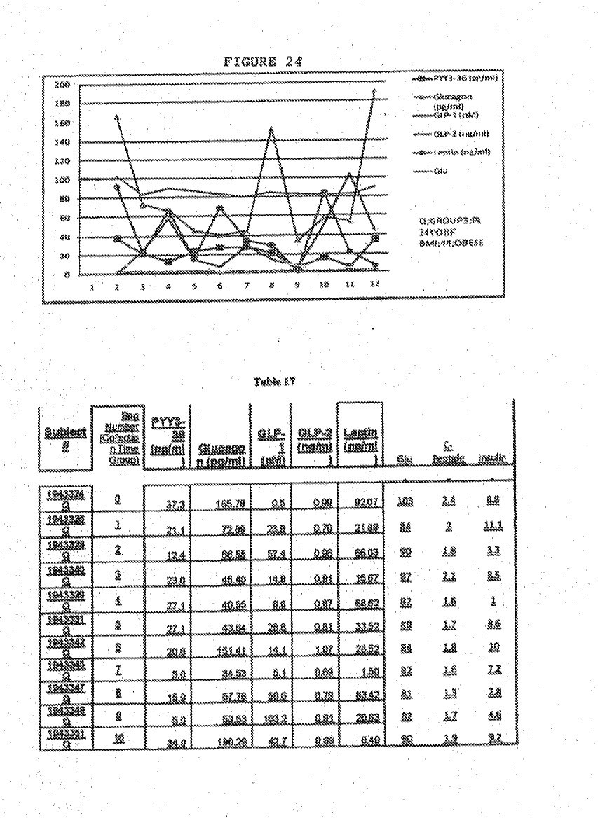

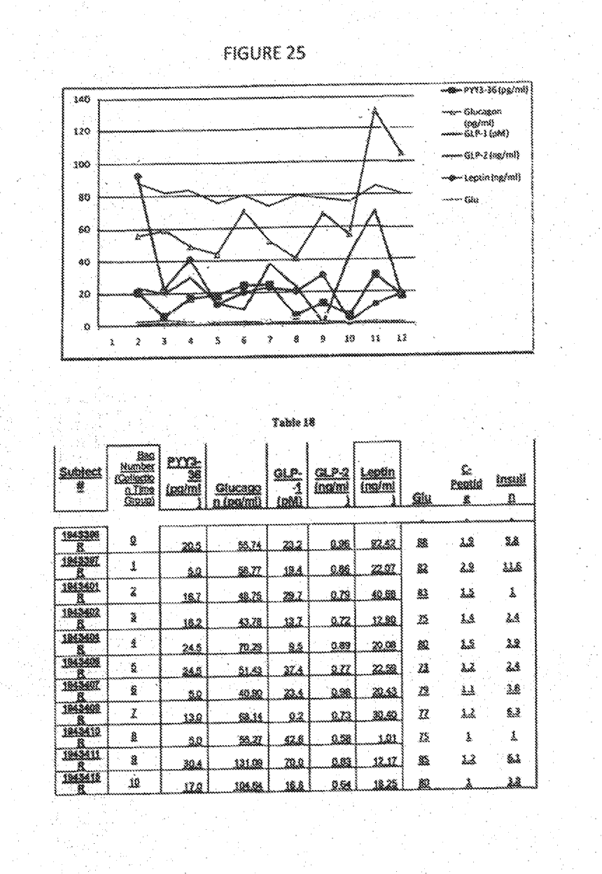

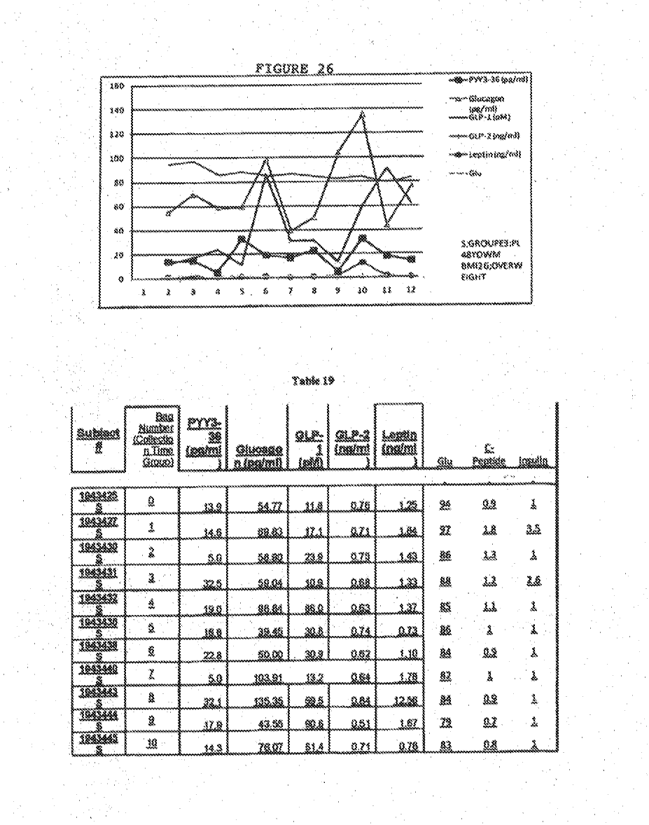

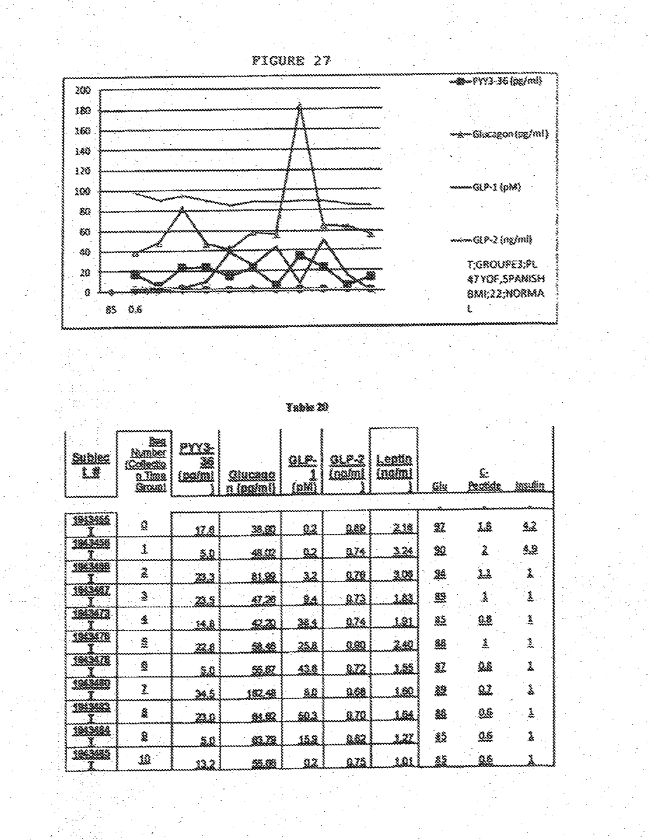

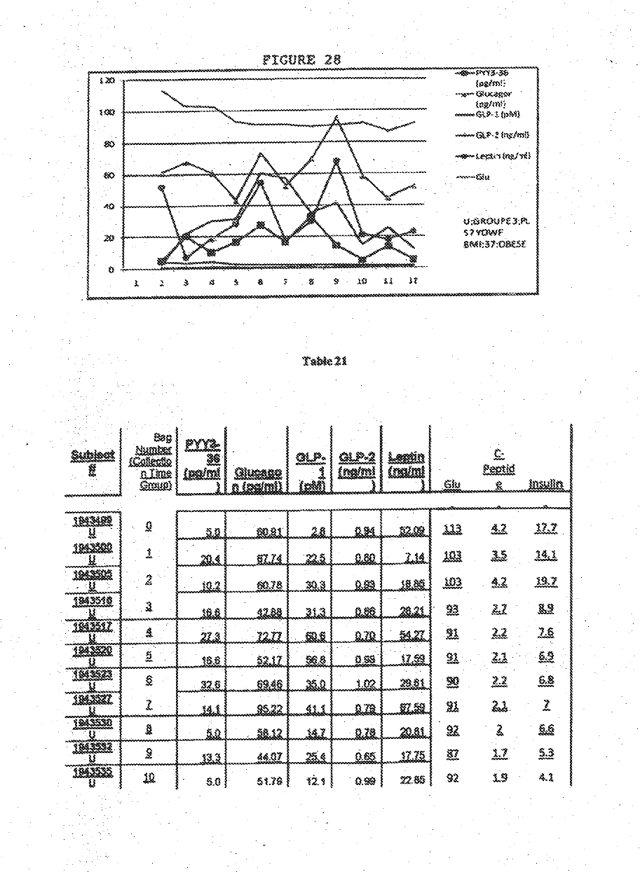

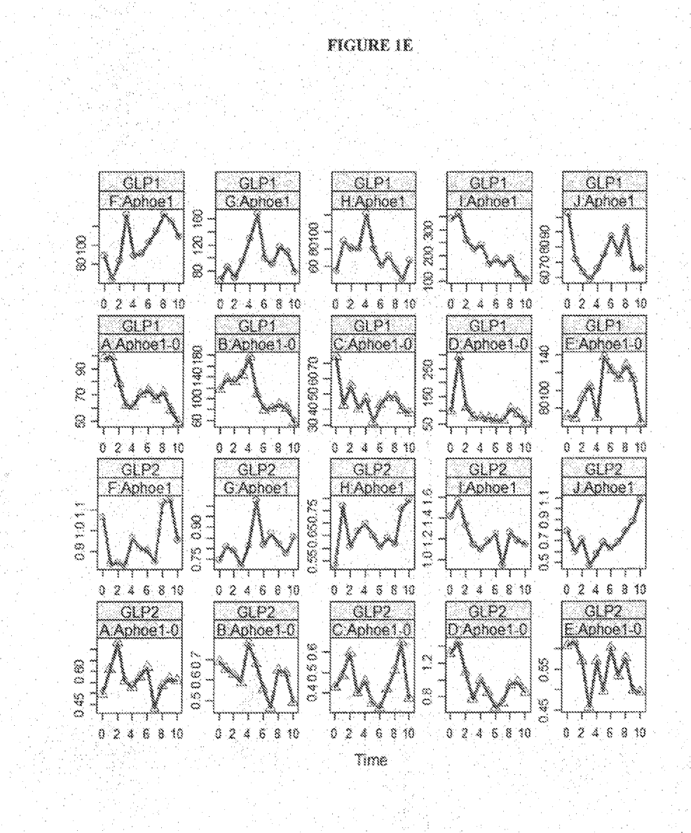

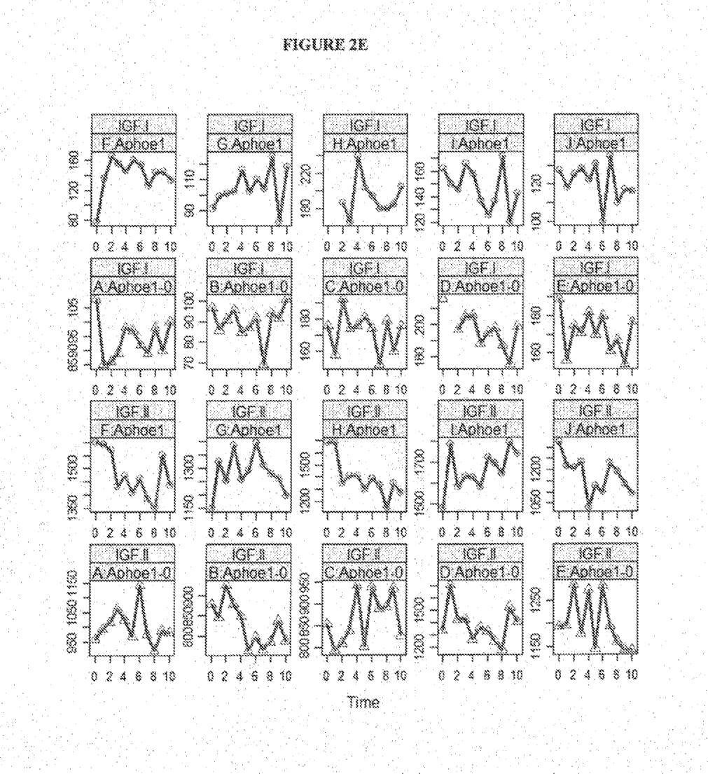

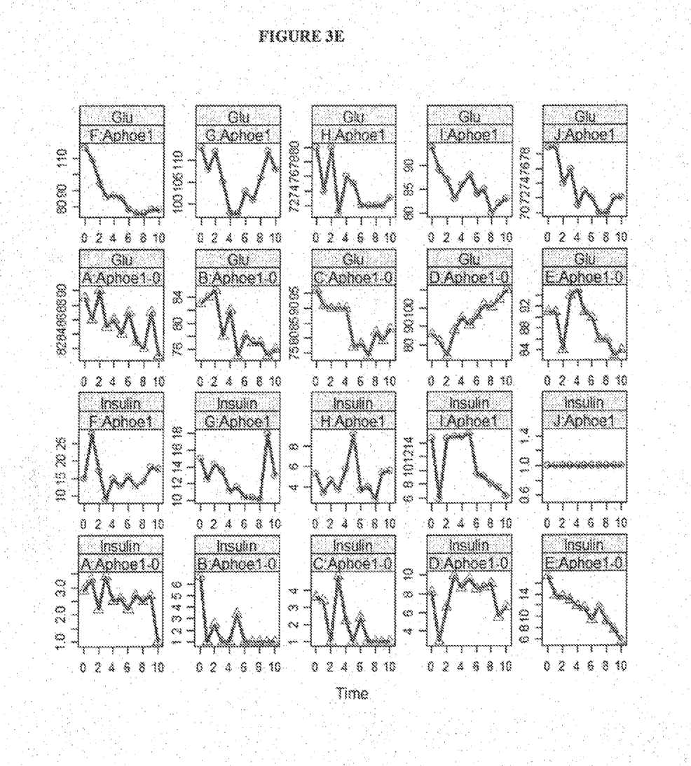

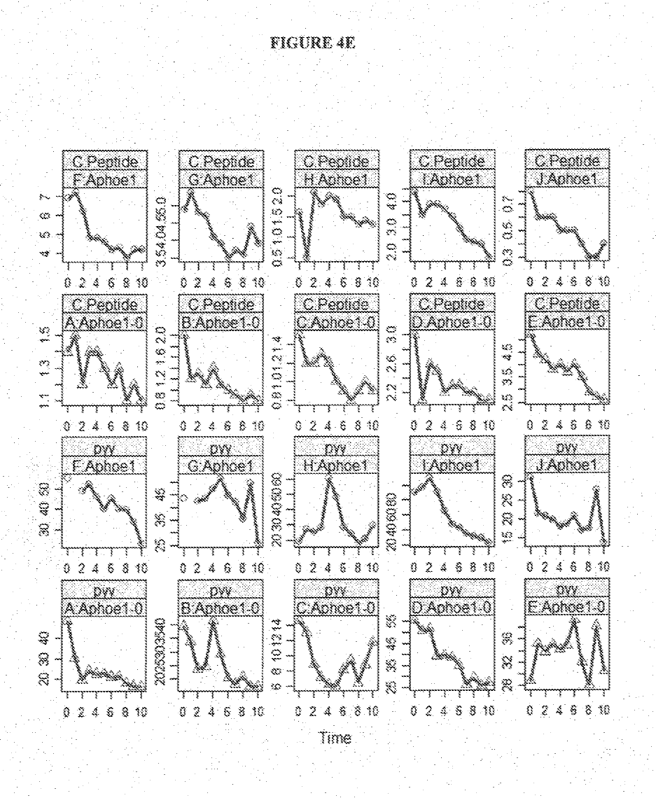

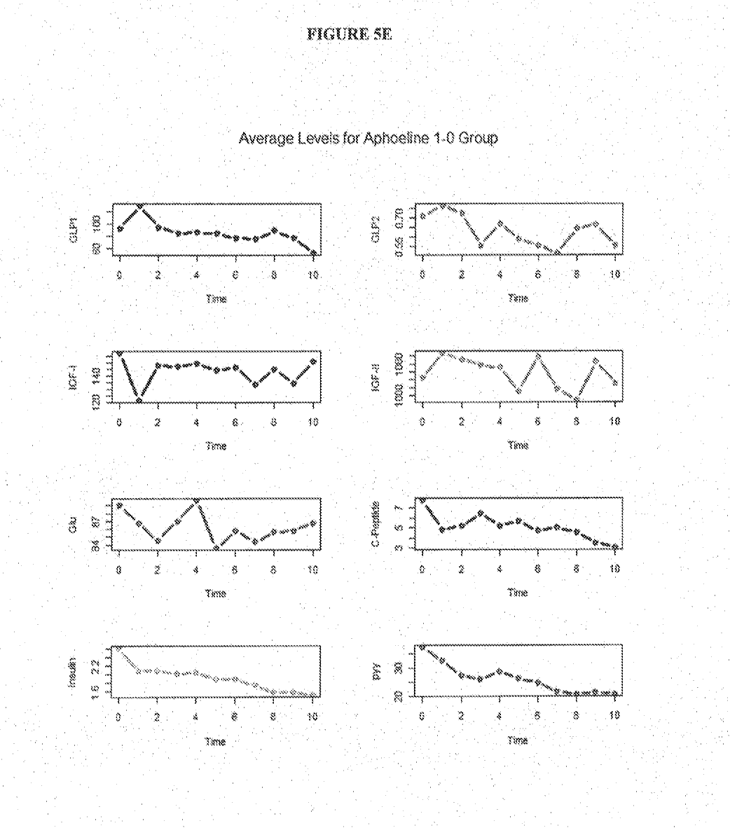

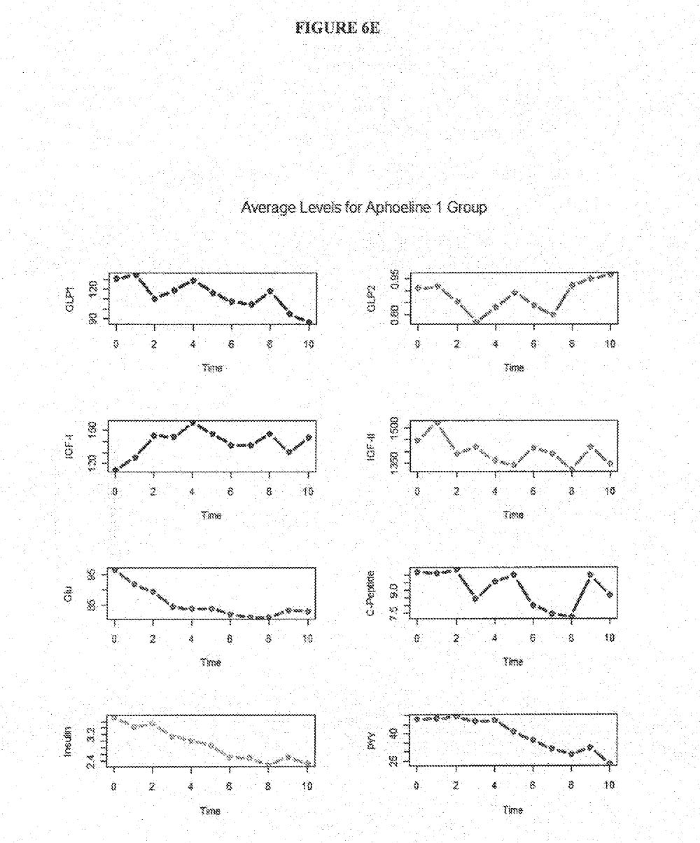

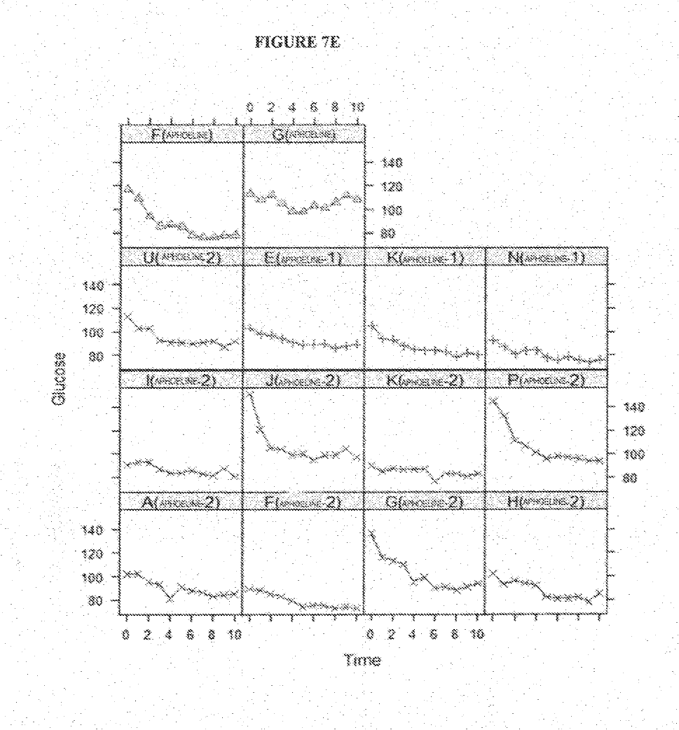

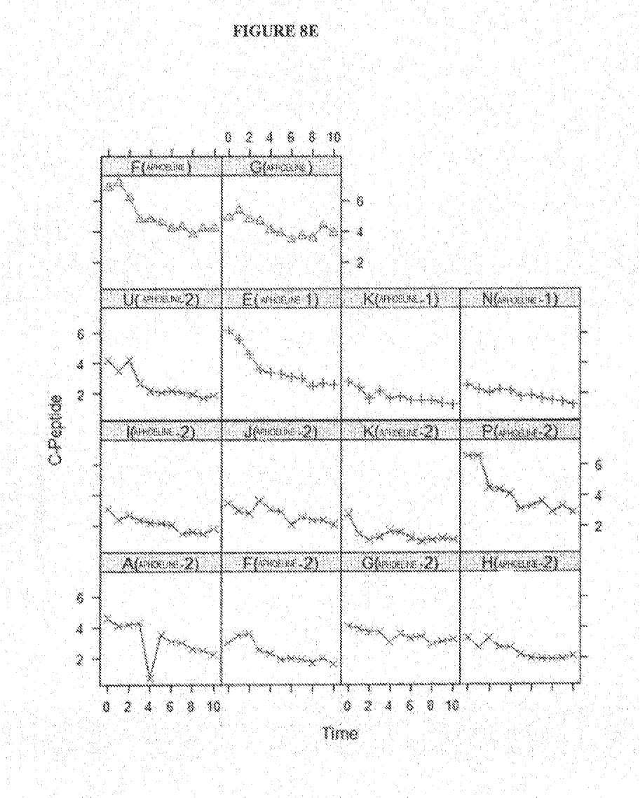

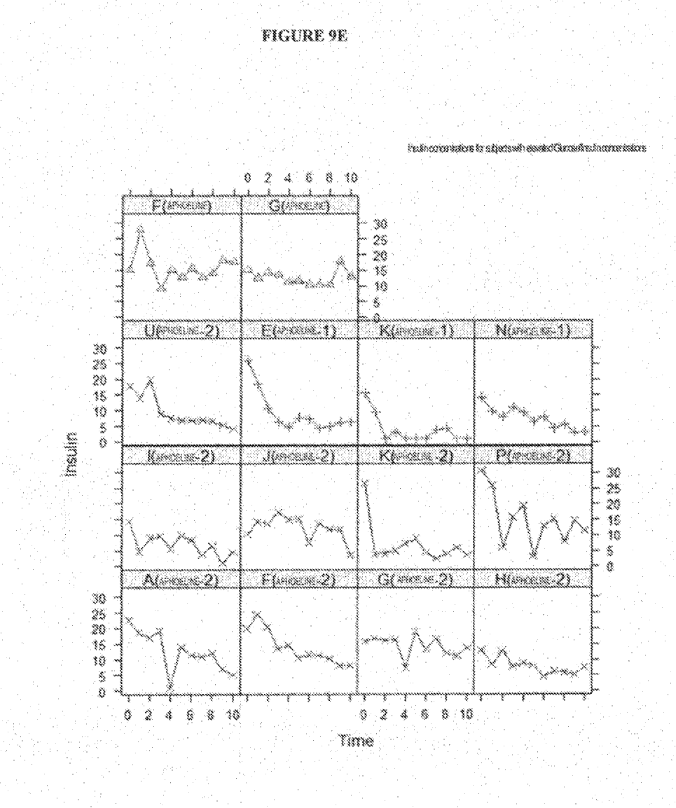

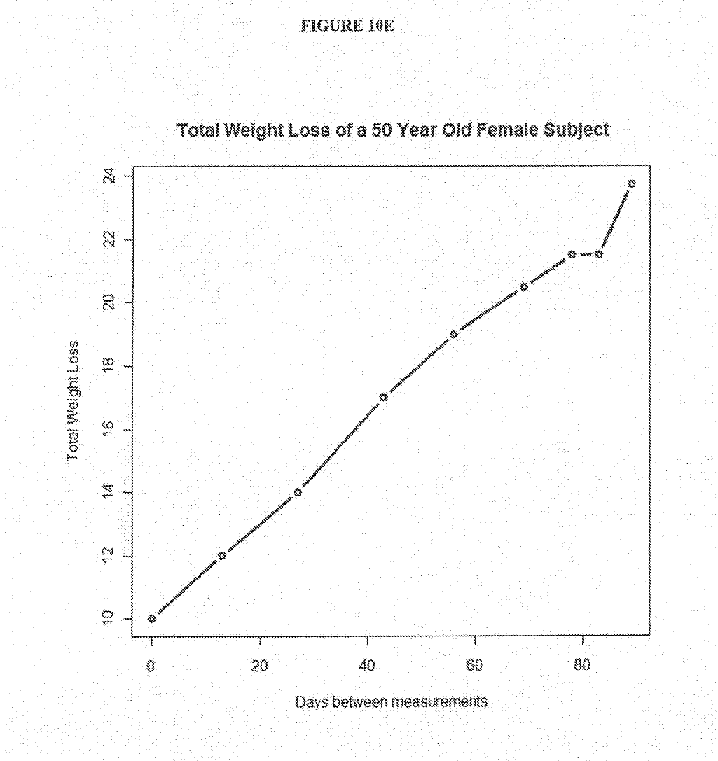

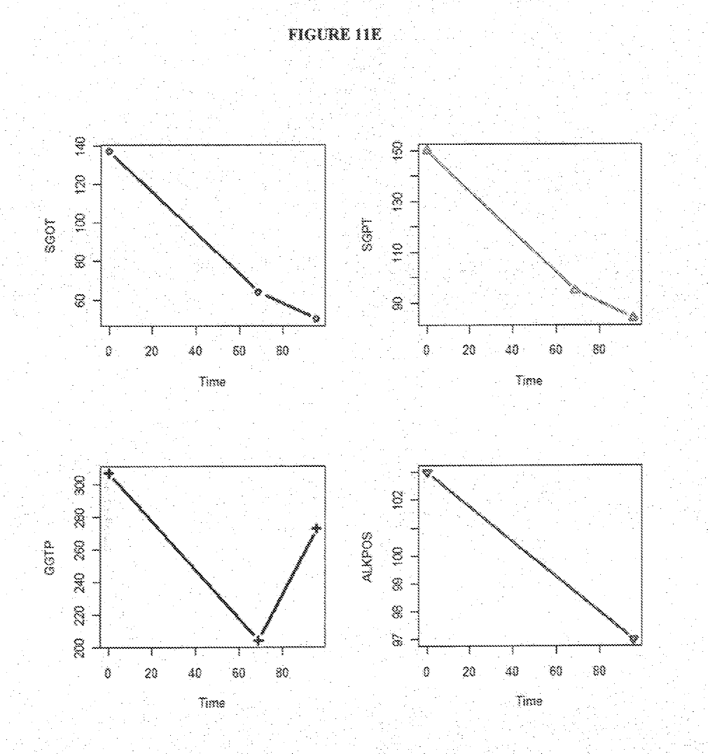

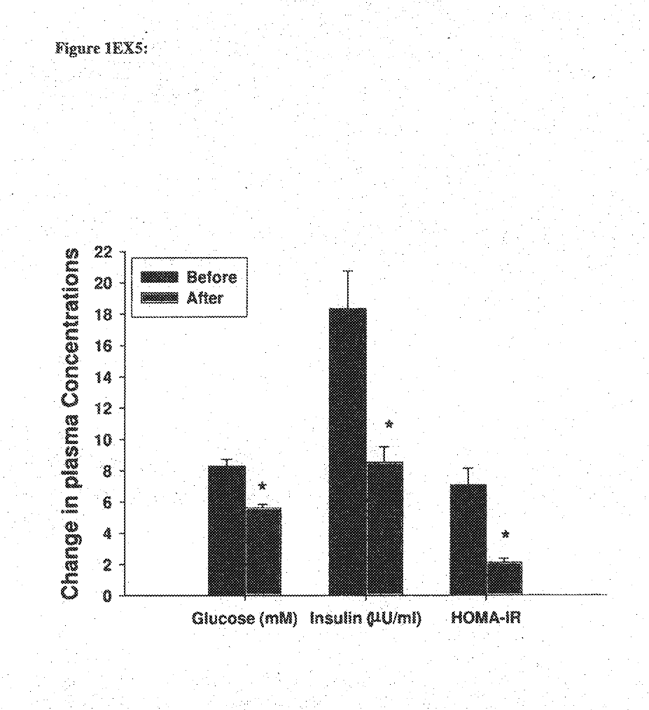

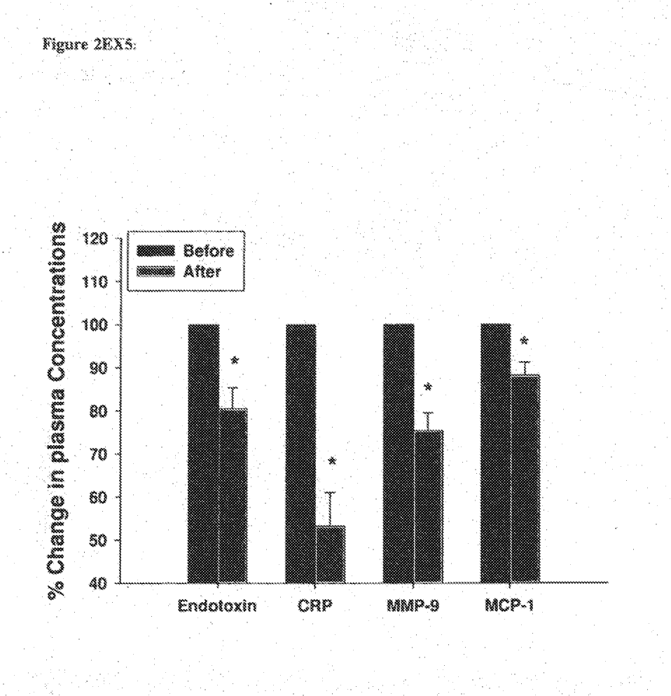

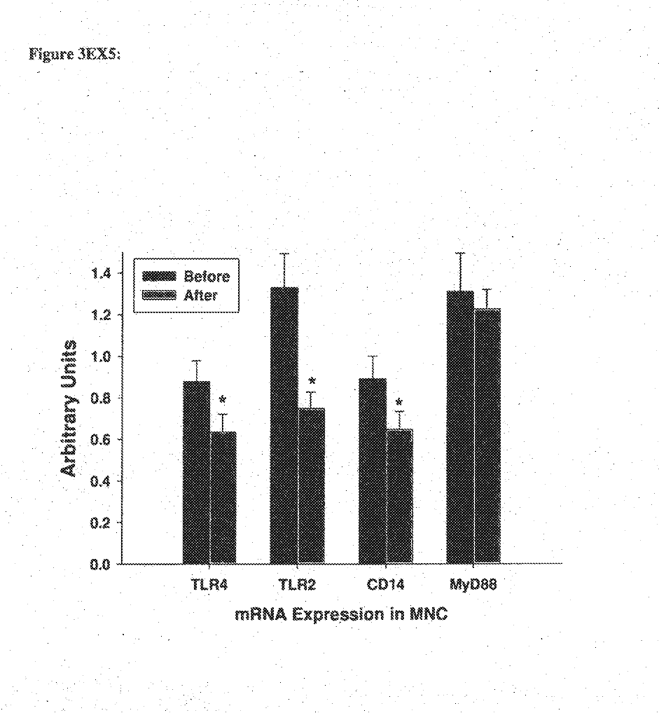

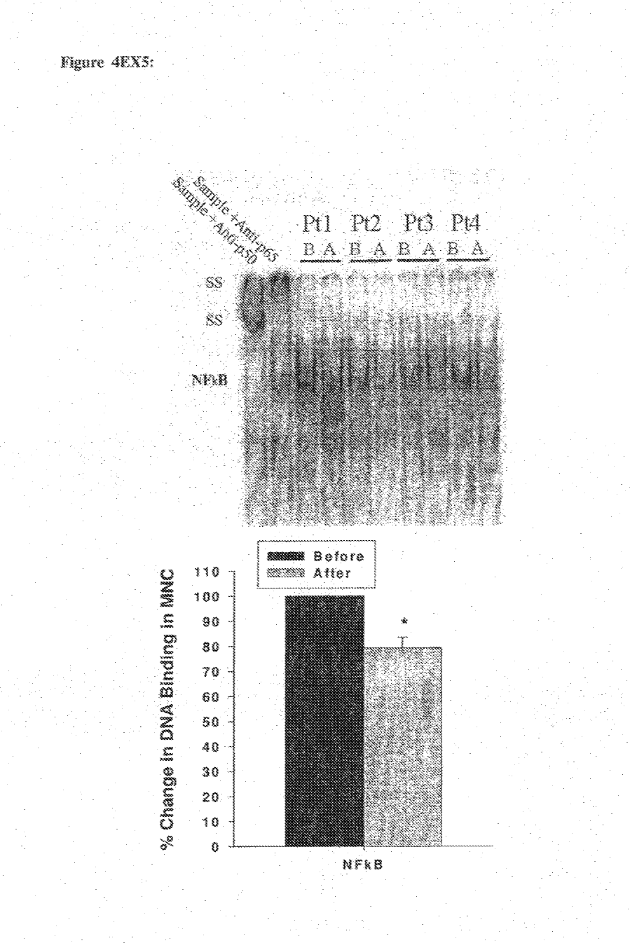

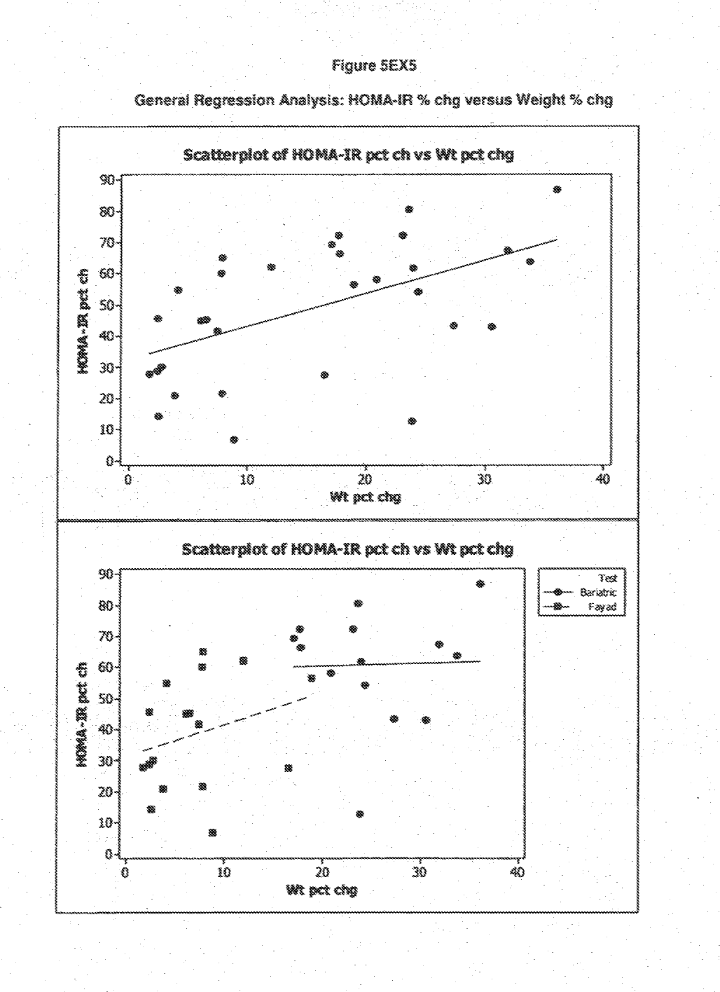

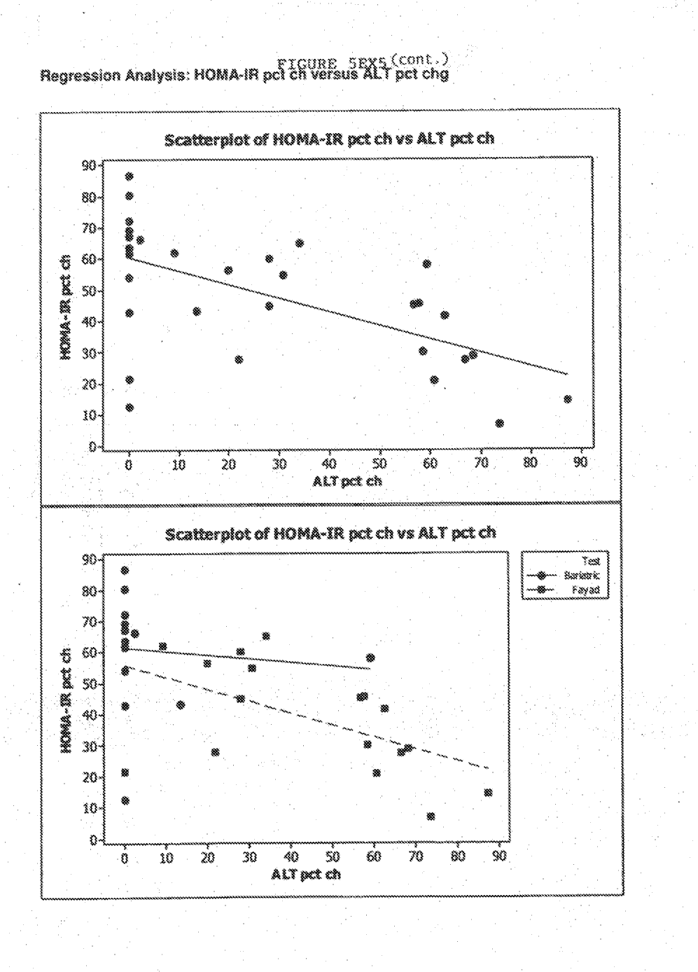

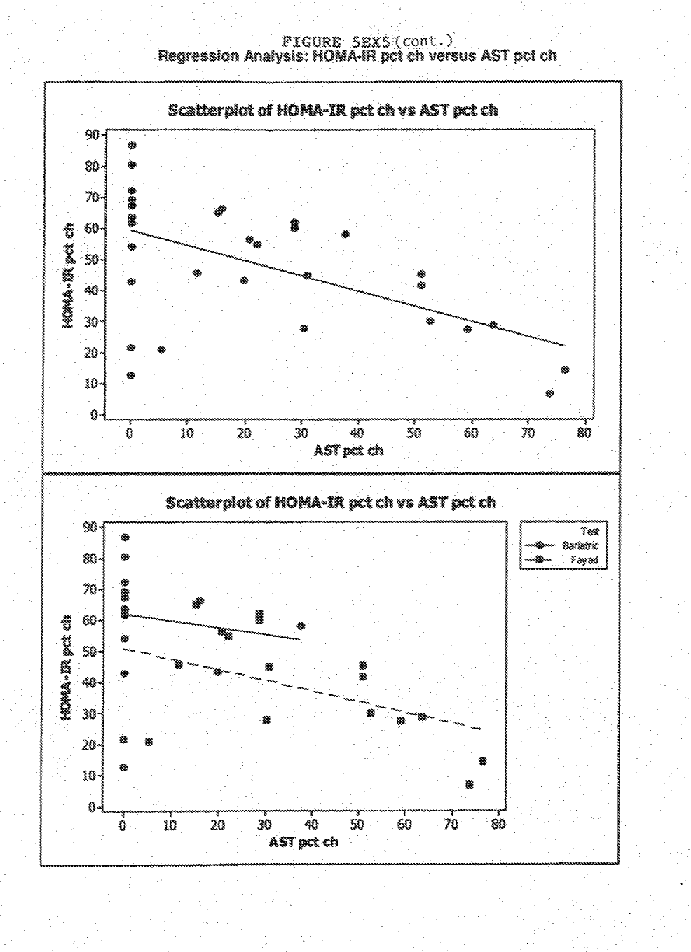

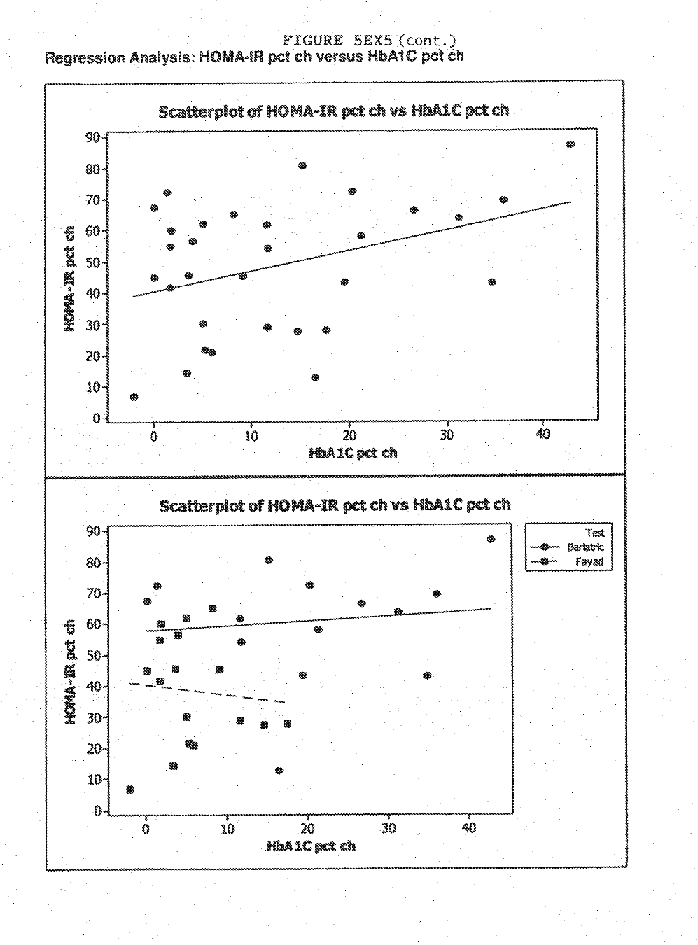

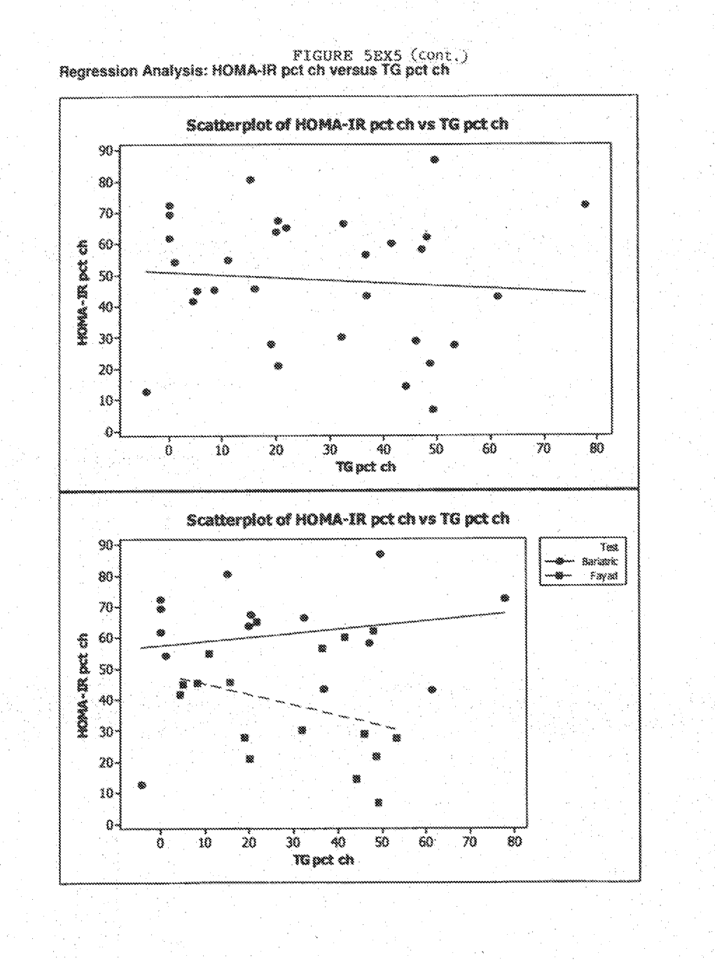

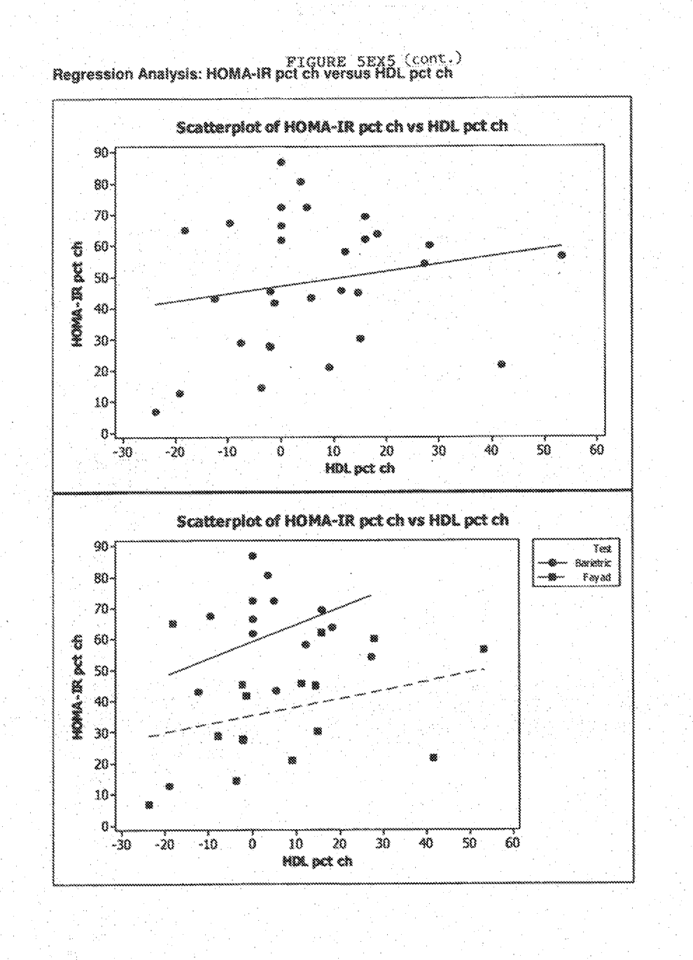

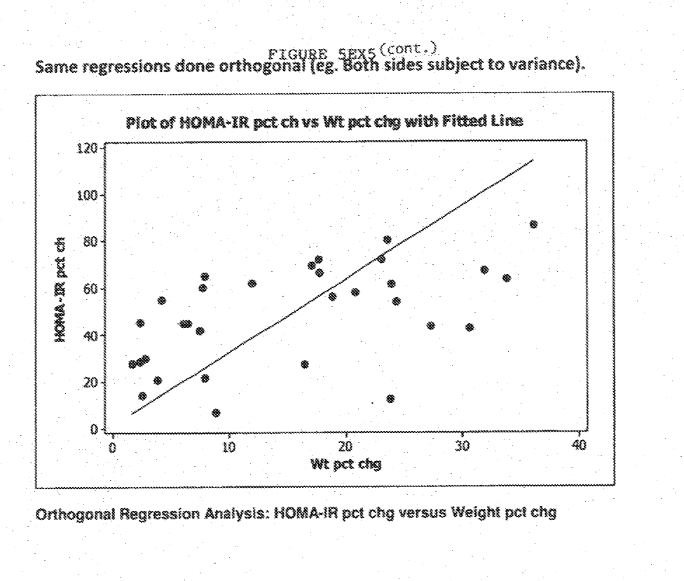

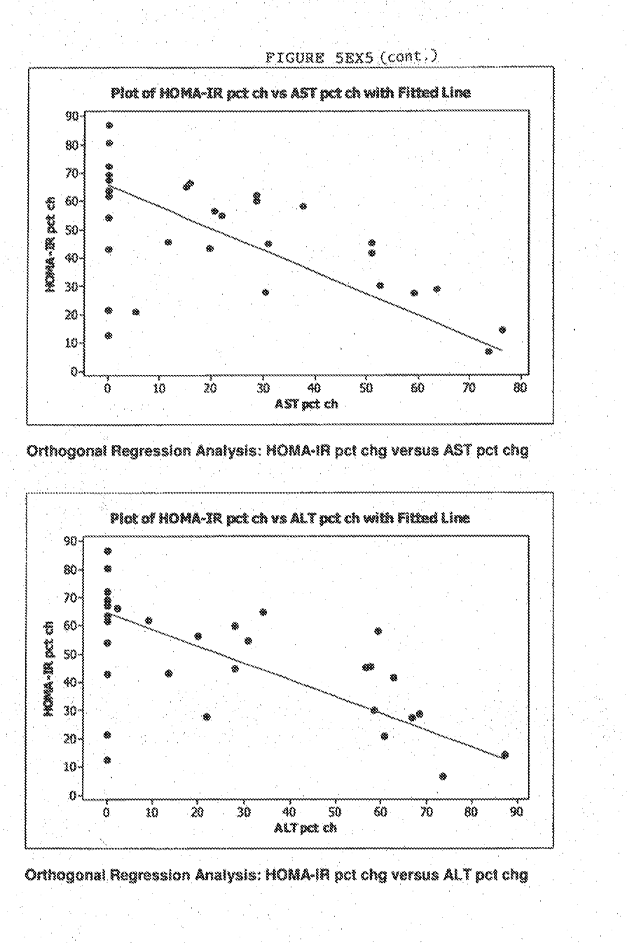

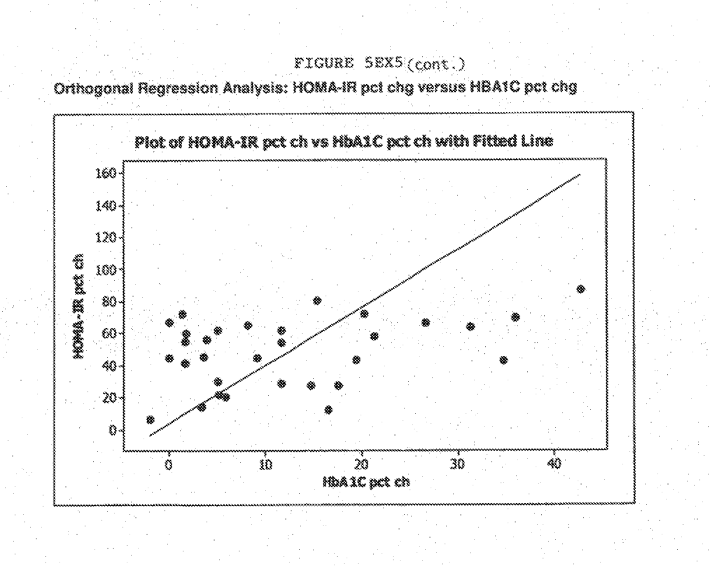

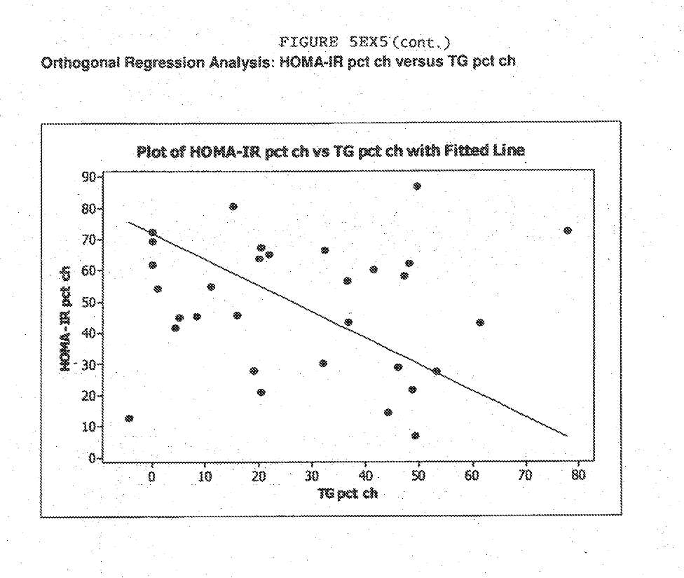

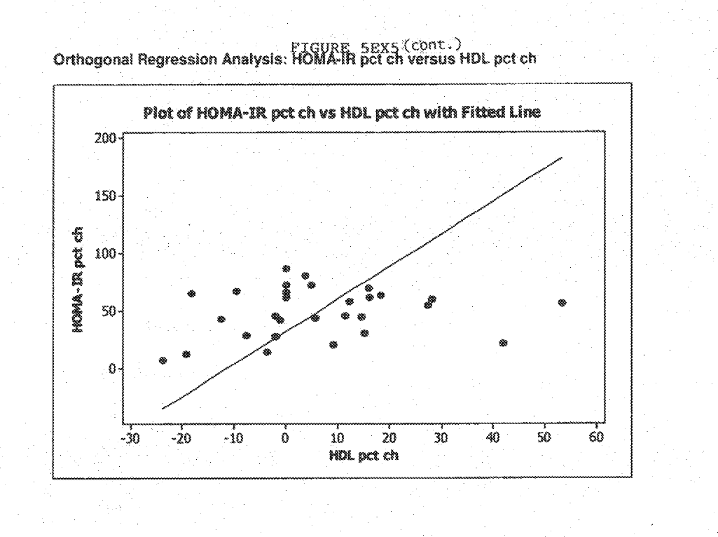

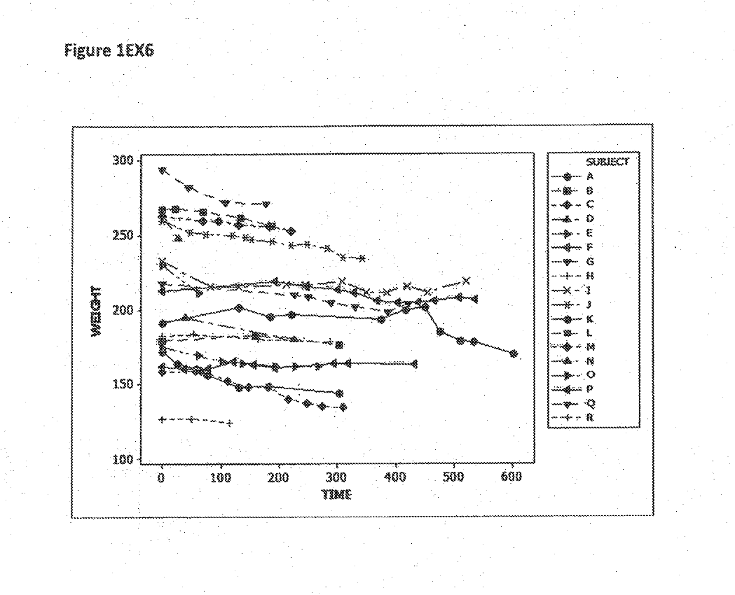

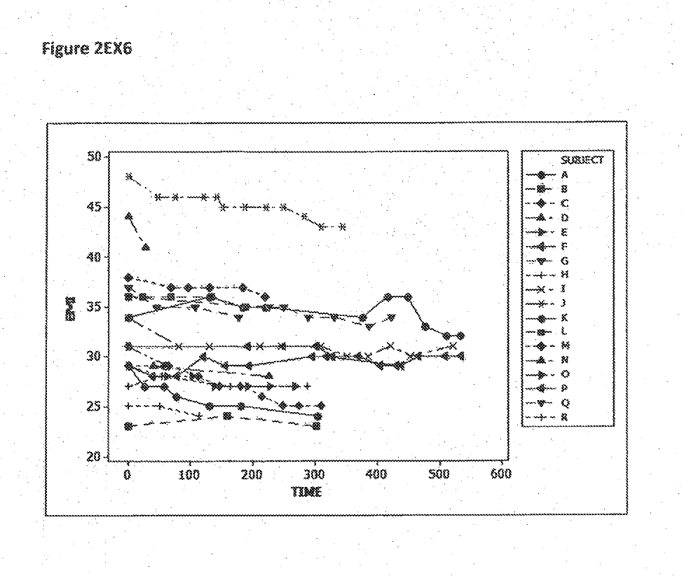

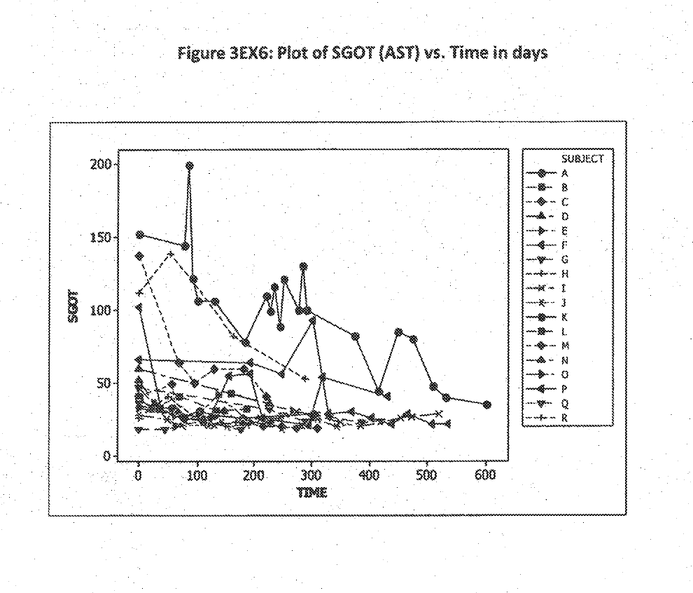

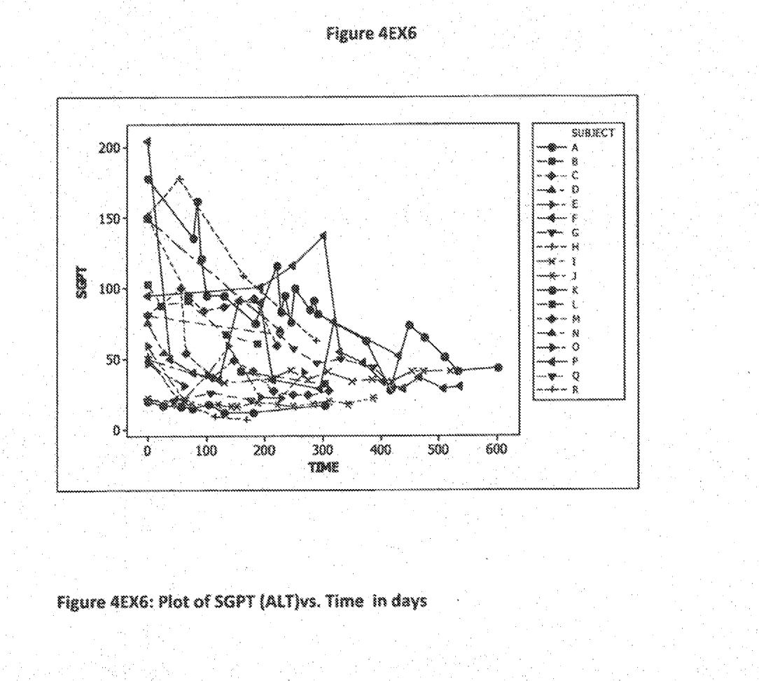

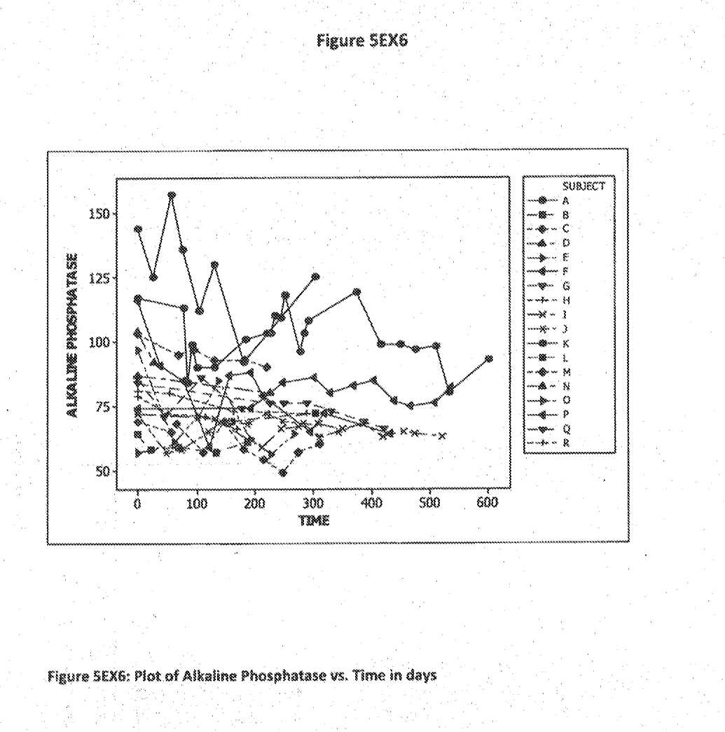

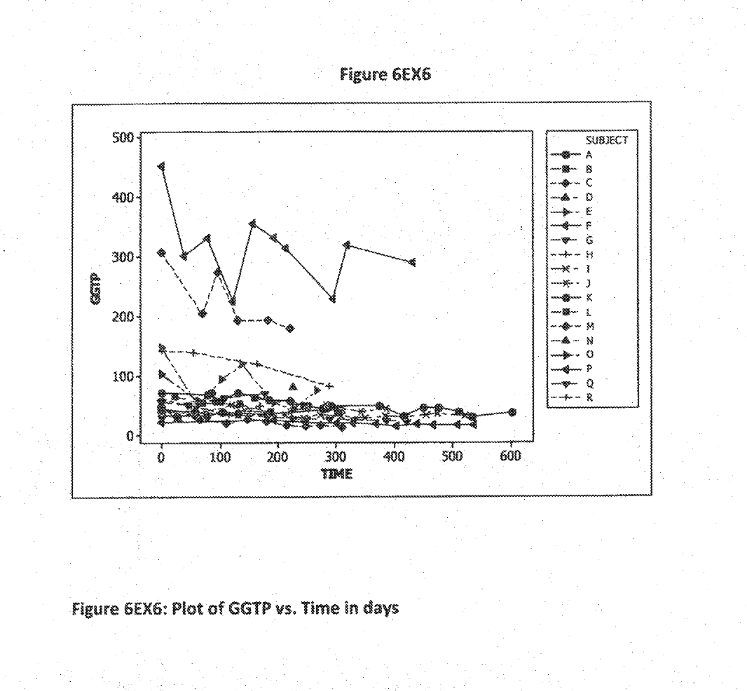

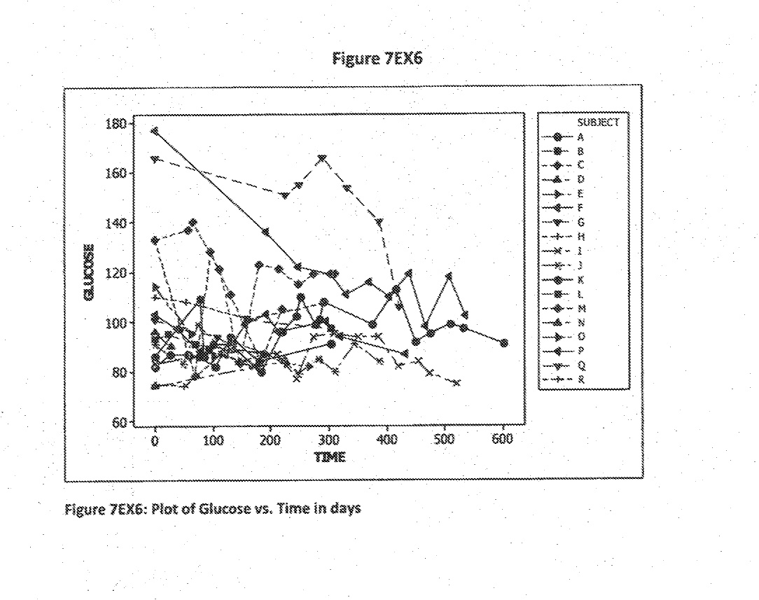

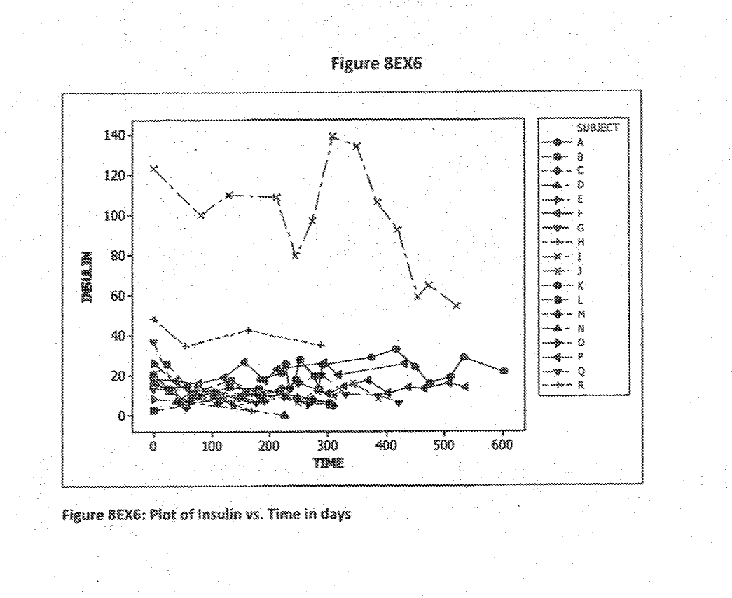

FIG. 8E (Further Examples) C-Peptide concentrations for subjects with elevated Glucose/Insulin concentrations.

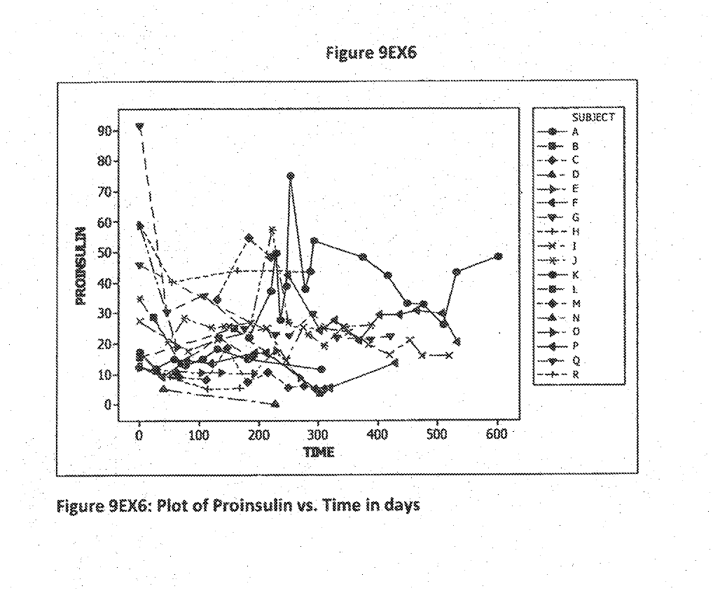

FIG. 9E (Further Examples) Insulin concentrations for subjects with elevated Glucose/Insulin concentrations.

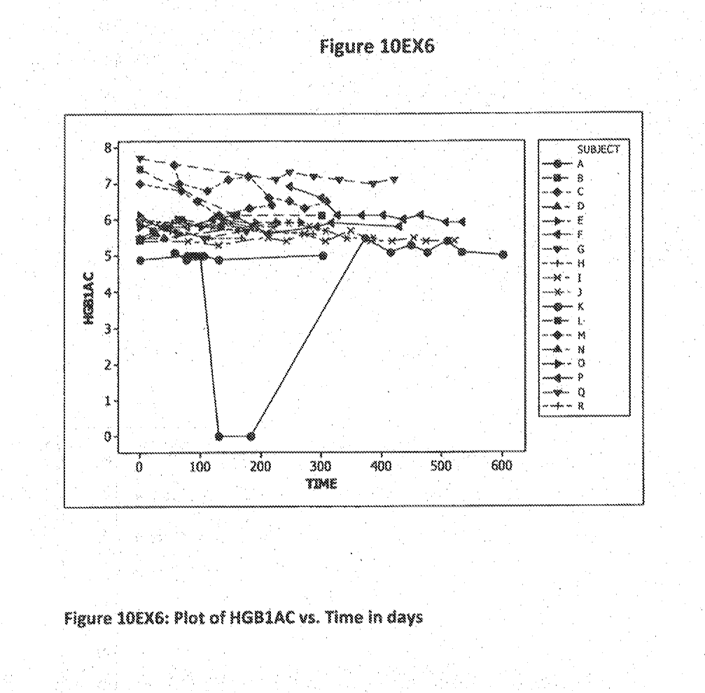

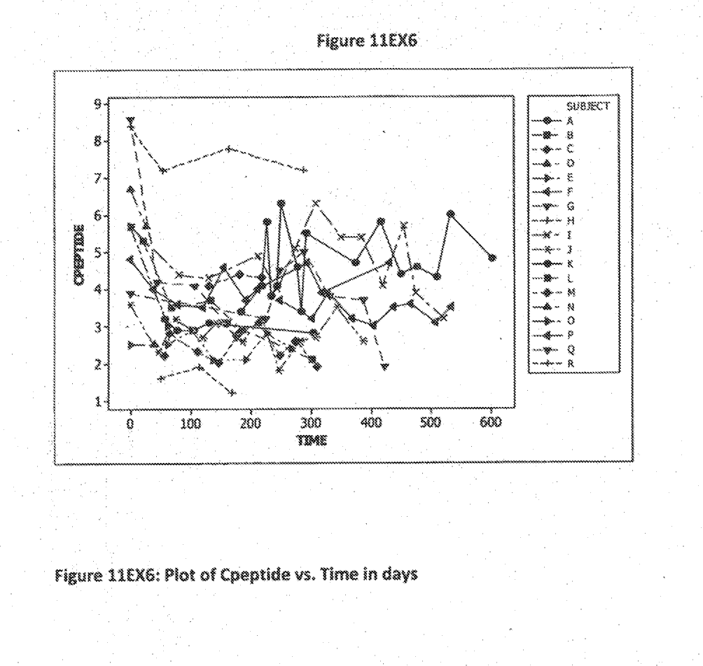

FIG. 10E shows the total weight loss observed for a subject on Aphoeline 1 (a 5.0 year old female) as a function of days between measurements, and FIG. 11 shows levels of liver enzymes in the same patient at the times of measurements. For this subject, Aphoeline 1 clearly has a positive and significant effect on liver enzymes. Total weight loss for a 50 year old white female with an initial blood glucose fasting of 220, ending with a fasting blood glucose of 110 mg/dL.

FIG. 11E shows the levels of liver enzymes for a steatohepatitis patient.

Figures for Example 5

FIG. 1EX5: Change in plasma concentrations of glucose and insulin, and calculated HOMA-IR in obese T2DM patients before and six months following RYGB (NM 5). Data are presented as Mean±SE. *P<0.05 by Paired t-test.

FIG. 2EX5; Change in TLR4, TLR2, CD14 and MyD88 expression m MNC from obese T2DM patients before and six months following RYGB (N=12). Data are presented as Mean±SE. *P<0.05 by Paired t-test.

FIG. 3EX5: Representative EMS A (A) and percent change (B) for NFkB DNA binding activity in MNC from 3 obese T2DM patients (Pt) before (8) and six-months after (A) RYGB (N=12). Data are presented as Mean±SE. *P<0.05 by Paired t-test Active NFkB complex band was determined by the addition of anti-p65 or anti-p50 (components of the active NFkB complex) to the reaction mixture containing nuclear extracts from Pt1-B sample causing the supershifting (SS) of the NFkB complex NFkB band but no other nonspecific (NS) bands.

FIG. 4EX5: Representative EMSA (A) and percent change (B) for NFkB DNA binding activity in MNC from obese T2DM patients (Pt) before (B) and six months after (A) RYGB (N=12). Data are presented as Mean & SE. *P<0.05 by Paired t-test.

FIG. 5EX5: provides the results of additional regression analyses of data taken from RYGB surgery patients. The data compilations presented in the FIG. 5 illustrate that a dosage of approximately 10 grams of active ingredient of a pharmaceutical composition of the invention can have an aggregate positive effect on ileal brake parameters equal to approximately 25% to approximately 80% of the aggregate positive effect on such parameters realized by RYGB Surgery.

Figures for Example 6

FIG. 1EX6: plot of weight in pounds versus time in days.

FIG. 2EX6: plot of BMI versus time in days.

FIG. 3EX6; plot of SGOT (AST) versus time in days.

FIG. 4EX6; plot of SGPT (ALT) versus time in days.

FIG. 5EX6: plot of alkaline phosphatase versus time in days.

FIG. 6EX6: plot of GGTP versus time in days.

FIG. 7EX6; plot of glucose versus time in days.

FIG. 8EX6; plot of insulin versus time in days.

FIG. 9EX6: plot of proinsulin versus time in days.

FIG. 10EX6: plot of HGB1AC versus time in days.

FIG. 11EX6: plot of C peptide versus time in days.

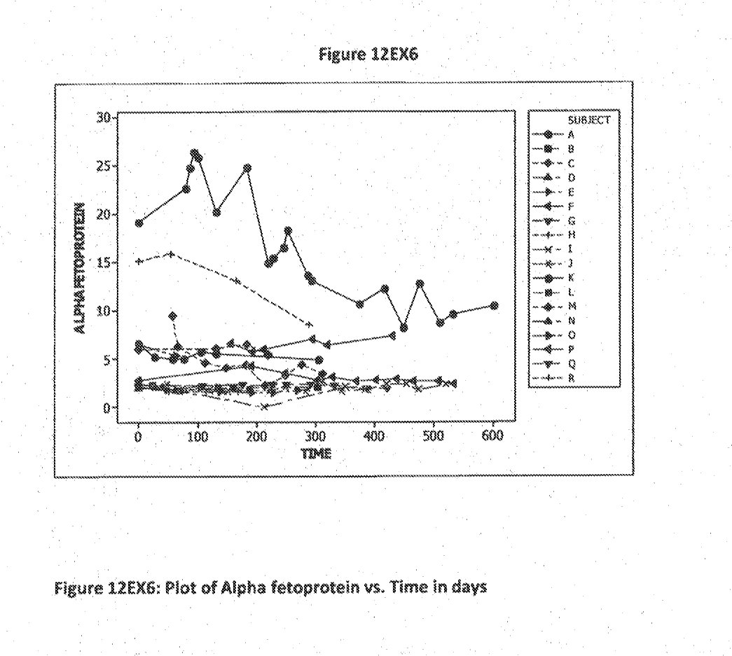

FIG. 12EX6: plot of alpha fetoprotein versus time in days.

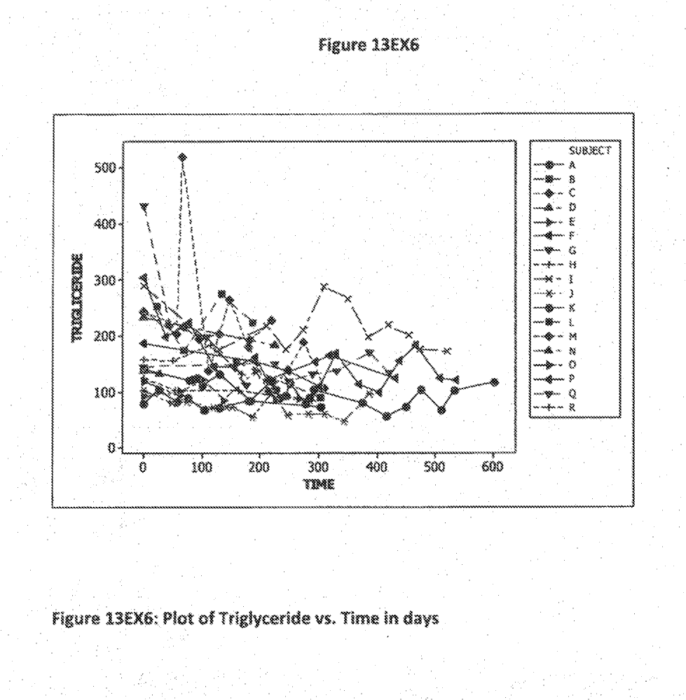

FIG. 13EX6; plot of triglyceride versus time in days.

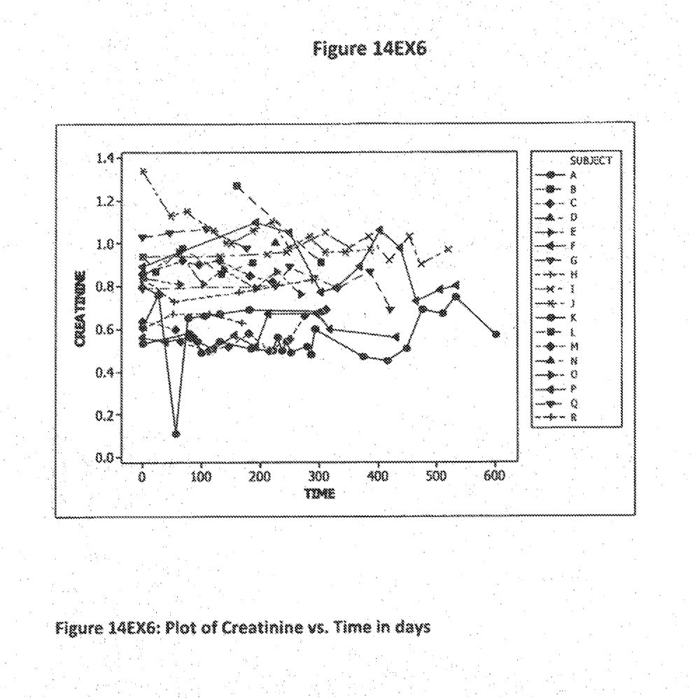

FIG. 14EX6: plot of creatinine versus time in days.

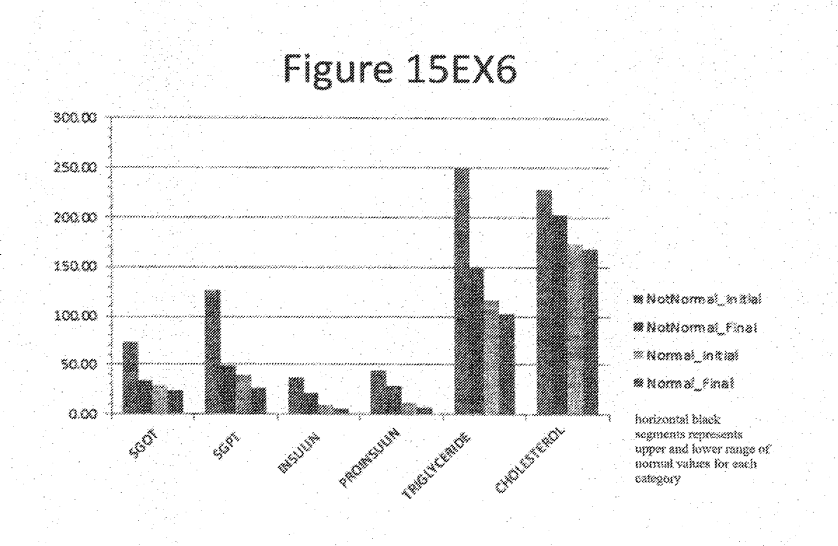

FIG. 15EX6: averages Normal vs; Not-Normal patients

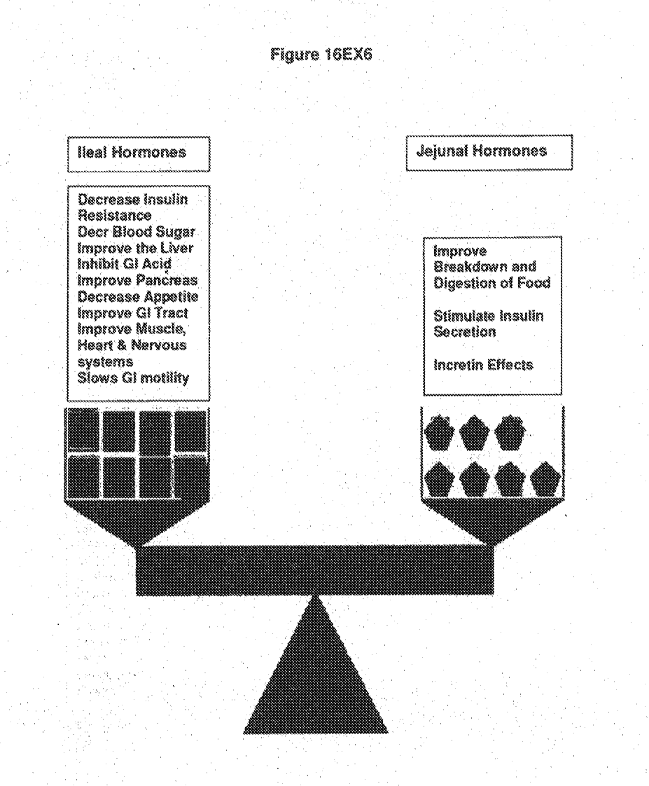

FIG. 16EX6: conceptual illustration of the effects of ileal and jejunal hormones.

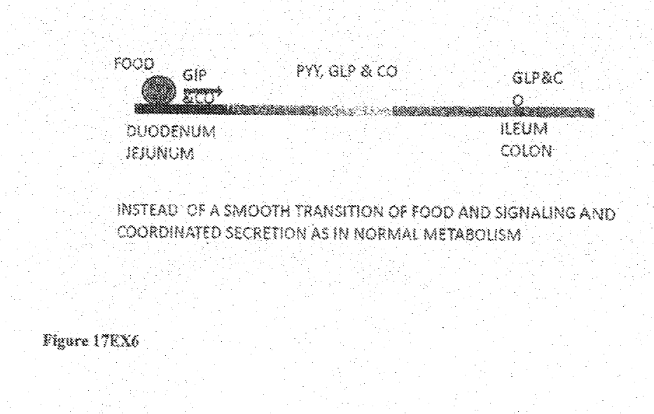

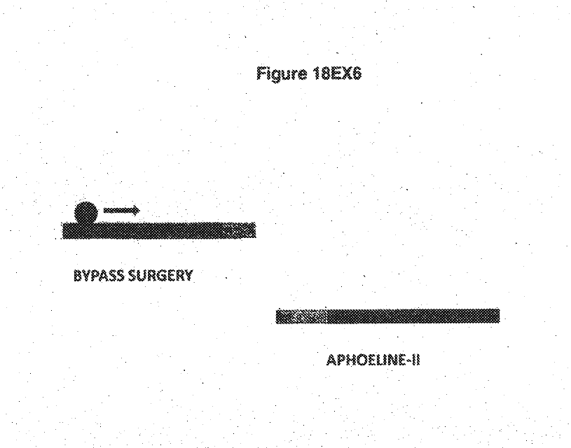

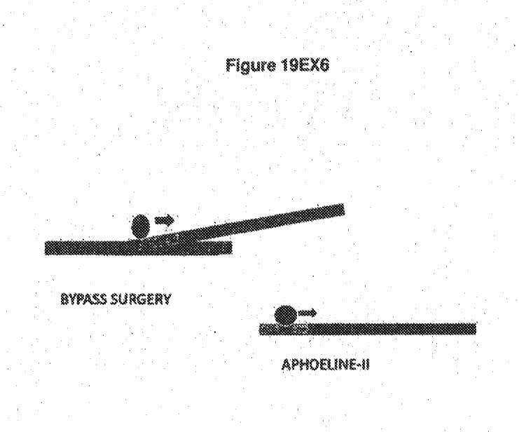

FIG. 17EX6: conceptual illustration of PYY, GFP, and CO effects. In altered metabolism the balance will shift toward glucose absorption, increased insulin production and poor or no stimulation of the ileal hormones, therefore poor signaling that would otherwise lower systemic inflammation and obesity, which causes additional insulin resistance, fatty liver and obesity, instead of a smooth transition of food and signaling and coordinated secretion.^{(FIG. 18)}. Both gastric bypass as well as oral ileal stimulation with Aphoeline or Brake™ will restore some physiological signaling^{(FIG. 19)}.

FIG. 18EX6: additional conceptual illustration of altered metabolism effects.

FIG. 19EX6: conceptual illustration of the effects of gastric bypass surgery and Aphoeline-II.

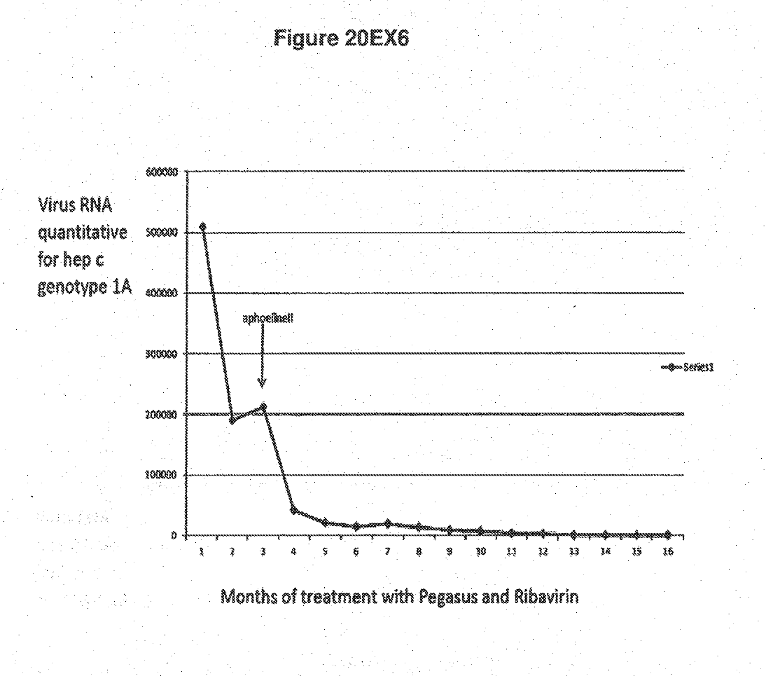

FIG. 20EX6: plot of Aphoeline response to hepatitis C in a CT Genotype 1A.

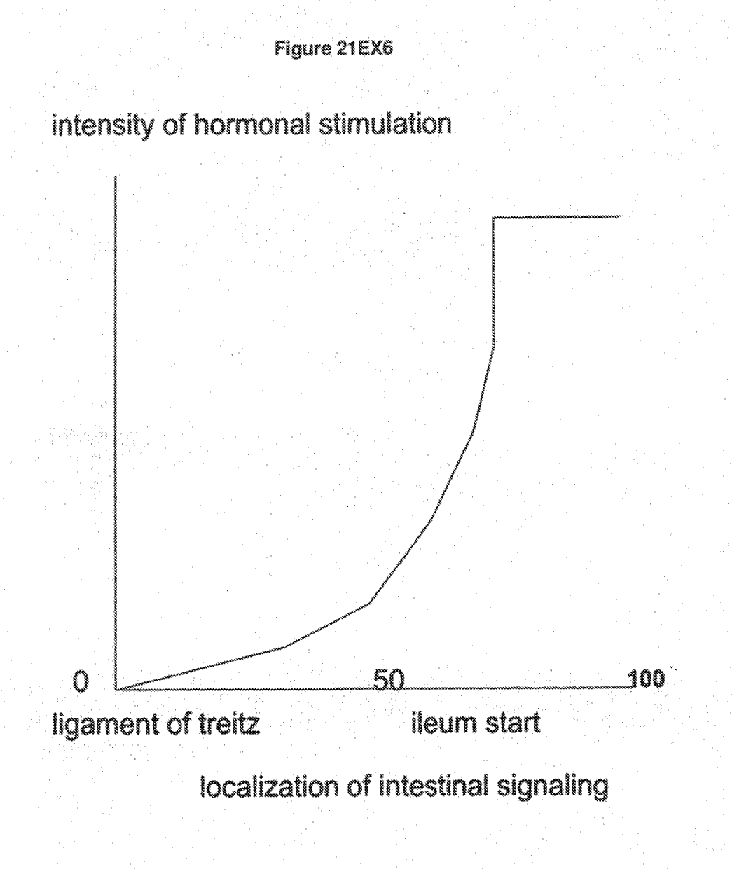

FIG. 21EX6: presents a theoretical graph of intestinal signaling levels from the L-cells along the intestine and colon.

FIG. 1EX7 shows the GLP″-1 concentration following 400-500 kcal Meal Challenge or Brake.

FIG. 2EX7A shows a regression analysis of HOMA-IR percent change vs. AST percent change.

FIG. 2EX7B shows a regression analysis of HOMA-IR percent change vs. ALT percent change.

FIG. 2EX7C shows a regression analysis of HOMA-IR percent change vs. AST percent change.

FIG. 2EX7D shows a regression analysis of HOMA-IR percent change vs. HbA1C percent change.

FIG. 2EX7E shows a regression analysts of HOMA-IR percent change versus TG percent change.

FIG. 2EX8 shows that the balance between absolution, and signaling of satiety and maintenance of the body is in equilibrium and factors affecting drat balance.

FIG. 2EX9 shows that in altered metabolism the balance will shift toward, the absorption, insulin production and poor or no stimulation of the ileal hormones, therefore poor signaling of satiety and body caloric reserve and usage, resulting in insulin resistance, fatty liver and obesity. Obesity is a natural state in a setting of excessive availability of readily absorbed, dense and high nutritional content foods, typical of the modern western diet. Even alter obesity is fully developed it is reversible; Both RYGB and oral ileal stimulation of ileal hormones with Brake will restore some physiological signaling.

### DETAILED DESCRIPTION OF THE INVENTION

The present invention approaches the problem of insulin resistance in a natural physiological manner by stimulating hormones in the lower gut, that is, the ileum, which act synergistically to reduce insulin resistance, so as to promote a substantial equilibrium between the amount of insulin produced and the amount of blood glucose, it does this using natural ileal brake hormone releasing components in healthful, pleasant compositions which am preferably coated using a polymeric, preferably nutrateric coating to release effective ileal brake hormone releasing substances within the ileum of a patient or subject and effect a natural physiological response within the subject's ileum with favorable results. The present invention represents a change in the nature of treating an insulin imbalance in a subject, using a more wholesome, natural physiological process, completely distinguishable over pharmaceutical or synthetic approaches. Use of this formulation to release L-cell derived regulatory substances into the portal blood supply to the liver avoids the disadvantages of peripherally administered analogues of similar L-cell derived regulatory substances. The present invention may also be used treat noninsulin dependent diabetes mellitus, pre-diabetes syndrome, metabolic syndrome, glucose intolerance and insulin resistance as well as a number of gastrointestinal tract disorders or conditions as otherwise described herein. The following definitions are used to describe the present invention and apply unless otherwise indicated.

The term “patient” or “subject” is used throughout the specification within context to describe an animal, generally a mammal and preferably a human, to whom treatment, including prophylactic treatment, with the compositions and/or methods according to the present invention, is provided. For treatment of a particular condition or disease state which is specific for a specific animal such as a human patient, the term patient refers to that specific animal.

The term “effective” is used herein, unless otherwise indicated, to describe an amount of a compound, composition or component and for an appropriate period of time which, in context, is used to produce or effect an intended result, whether that result relates to the treatment of a disorder or condition associated with the present invention or alternatively, is used to produce another compound, agent or composition. This term subsumes all other effective amount or effective concentration terms which are otherwise described in the present application. In many instances, with the administration of D-glucose (dextrose) as a ileal brake hormone releasing substance in compositions and methods according to the present invention, an effective amount of D-glucose ranges from about 500 mg, to about 12.5 grams or more, preferably about 10 grams used on a daily basis.

The term “nutritional substance” is used synonymously with “pharmaceutical composition” and “ileal brake hormone releasing substance” in certain contexts herein and refers to the substance which produces the intended effect in the ileum of a patient or subject pursuant to the present invention. A “nutritional substance” includes, but is not limited to, proteins and associated amino acids, fats including saturated fats, monosaturated fats, polyunsaturated fats, essential fatty acids, Omega-3 and Omega-6 fatty acids, trans fatty acids, cholesterol, fat substitutes, carbohydrates such as dietary fiber (both soluble and insoluble fiber), starch, sugars (including monosaccharides, fructose, galactose, glucose, disaccharides, lactose, maltose, sucrose, and alcohol), polymeric glucoses including inulin and polydextrose, natural sugar substitutes (including brazzein, Curculin, erythritol, fructose, glycyrrhizin, glycyrrhizin, glycerol, hydrogenated starch hydrosylates, isomalt, lactitol, mabinlin, maltitol, mannitol, miraculin, monellin, pentadin, sorbitol, stevia, tagatose, thaumatin, and xylitol), sahlep, and halwa root extract. D-glucose (dextrose) is a preferred ileal brake hormone releasing substance. Ileal brake hormone releasing substances include all compositions that yield the aforementioned nutrients upon digestion or that contain such nutrients, including polymeric forms of these nutrients.

Additional ileal brake hormone releasing components which may be included in compositions according to the present invention include, barley grass, known to be a rich source of highly metabolizable vitamins and minerals such as vitamins A, B1, B2, B6, B12 and C, potassium, magnesium, and zinc. In addition, barley grass also has a high concentration of the enzyme superoxide dismutase (SOD), which has been shown to have high levels of antioxidant activity. Barley grass is believed to be an important nutrient in the regulation of the digestive process because the micronutrients, enzymes (e.g., SOD), and fiber contained in barley grass are believed to improve intestinal function.

Alfalfa fresh or dried leaf tea is also usable in the invention, to promote appetite, and as a good source of chlorophyll and fiber. Alfalfa contains biotin, calcium, choline, inositol, iron, magnesium, PABA, phosphorus, potassium, protein, sodium, sulfur, tryptophan (amino acid), and vitamins A, B complex, C, D, B, K, P, and U. Alfalfa supplements are recommended for treating poor digestion, and were shown to lower cholesterol levels in animal studies. Alfalfa is categorized as Generally Regarded as Safe (GRAS) by the FDA. Dosages can range from 25-1500 mg, preferably 500-1000 mg dried leaf per day.

Chlorella is yet another substance usable in the invention in combination with the ileal brake hormone releasing substance (preferably D-glucose or dextrose), being a genus of unicellular green algae, grown and harvested in tanks, purified, processed and dried to form a powder. Chlorella is rich in chlorophyll, carotenes, and contains the full vitamin B complex, vitamins E and C, and has a wide range of minerals, including magnesium, potassium, iron and calcium. Chlorella also provides dietary fiber, nucleic acids, amino acids, enzymes, CGF (Chlorella Growth Factor) and other substances. Dosages can range from 300-1500 mg/day.

Chlorophyllin is yet another ileal brake hormone releasing substance, being a known food additive and has been used as an alternative medicine. Chlorophyllin is a water-soluble, semi-synthetic sodium/copper derivative of chlorophyll, and the active ingredient in a number of internally-taken preparations intended to reduce odors associated with incontinence, colostomies and similar procedures, as well as body odor in general. It is also available as a topical preparation, purportedly useful for treatment and odor control of wounds, injuries, and other skin conditions, such as for radiation burns.

Sodium alginate may also be used as a nutritional substance, preferably in combination with D-glucose or dextrose.

The term “ileum” is used to describe the third (of three) portion of the small intestine just before the small intestine becomes the large intestine in the gastrointestinal tract. The ileum is the final section of the small intestine in higher vertebrates, including mammals. The ileum follows the duodenum and jejunum in the small intestine, and is separated from the “Cecum” by the ileocecal valve (ICV), in humans, the ileum is about 2-4 meters long, and the pH usually ranges between 7 and 8 (neutral or slightly alkaline). The function of the ileum is mainly to absorb vitamin B12 bile salts and whatever products of digestion were not absorbed by the jejunum. The wall itself is made up of folds, each of which has many tiny finger-like projections known as “villi” on its surface. In turn, the epithelial cells which line these villi possess even larger numbers of microvilli. Therefore, the ileum has an extremely large surface area both for the adsorption of enzyme molecules and for the absorption of products of digestion. The DNES (diffuse neuroendocrine system) cells that line the ileum contain lesser amounts of the protease and carbohydrase enzymes (gastrin, secretin, and cholecystokinin) responsible for the final stages of protein and carbohydrate digestion. These enzymes are present in the cytoplasm of the epithelial cells.

The term “delays the release in vivo of the majority of the ileal brake hormone releasing substance until the dosage form reaches the subject's ileum” means: (1) that not less than around 50% by weight, not less than around 70% by weight, more preferably not less than around 80% by weight, and more preferably not less than around 90% and in certain instances substantially all of the ileal brake hormone releasing substance remains unreleased in vivo prior to the dosage form's arrival al a subject's ileum; and (2) that not less than around 50%, not less than around 70% by weight, more preferably not less than around 80% by weight, and more preferably not less than around 90%, of the ileal brake hormone releasing substance is remains released in vivo by the time when the dosage form enters the subject's ileum. In preferred aspects of the invention this amount is at least about 1 gram, at least about 2.5 grams, at least about 3 grams, at least about 5 grams, at least about 7.5 grams, preferably about 10 grams to about 12-12.5 grams or more (about 12.5 to about 20 grams, especially of polymeric materials such as polydextrose or those compounds of higher molecular weight) of the ileal brake hormone releasing substance and in particular, glucose, is released within the small intestine in the ileum in order to stimulate ileum hormones and related hormones and effect the intended result associated with lowering the manifestations of metabolic syndrome and/or influencing one or more of insulin resistance (decrease resistance), blood glucose (decrease m/stabilize glucose levels), glucagon secretion (decrease), insulin release (decrease and/or stabilize release and/or levels), ileum hormone release (increase) or other hormone release, in particular, one or more of GLP-1, glicentin, C-terminally glycine-extended GLP-1 (7 37), (PG (78 108)); C-peptide, intervening peptide-2 (PG (111 122) amide); GLP-2 (PG (126 158), GRPP (PG (1 30)), oxyntomodutin (PG (33 69), and other peptide fractions to be isolated, PYY (1-36), PYY (3-36), cholecystokinin (CCK), gastrin, enteroglucagon, secretin, as well as leptin, IGF-1 and IGF-2, and preferably, one or more, two or more, three or more, four or more, five or more, six or more, seven or more, or all of GLP1, GLP2, C-peptide, PYY (1-36 and/or 3-36), glucagon, leptin, IGF-1 and IGF-2.

The term “ileum hormones” includes all hormones that are associated with intraluminal food substances stimulating the release of said hormones, could lie associated with action of the ileal brake and associated feedback, from the ileum or ileum-related stimulation of insulin secretion or inhibition of glucagon secretion. “Ileum hormones” therefore include, but are not limited to, GLP-1, glicentin, C-terminally glycine-extended GLP-1 (7 37), (PG (78 108)); intervening peptide-2 (PG (111 122)amide); GLP-2 (PG (126 158), GRPP (PG (1 30)), oxyntomodulin (PG (33 69), and other peptide tractions to be isolated, PYY (PYY 1-36) and (PYY 3-36), cholecystokinin (CCK), gastrin, enteroglucagon and secretin.

The term “ileum hormone-stimulating amount of a nutritional substance” means any amount of a nutritional substance that is effective to induce measurable hormone release in the ileum, and induce feedback from the ileum or ileum-related stimulation of insulin secretion or inhibition of glucagon secretion, or other effect such as shutting down or decreasing insulin resistance and increasing glucose tolerance. Consequently, an “ileum hormone-stimulating amount of a nutritional substance” can vary widely in dosage depending upon factors such as the specific nutrient at issue, the desired effect of administration, the desired goal of minimizing caloric intake, and the characteristics of the subject to whom the ileal brake hormone releasing substance is administered. For example, at least about 500 mg of D-glucose is used, and a particularly preferred ileum hormonal-stimulating amount of D-glucose includes between about 7.5-8 g to about 12-12.5 g (preferably around 10 g).

The term “gastrointestinal disorder” includes diarrheal states, malabsorption in the upper gut (i.e., chronic pancreatitis, celiac disease), fatty liver, atrophic gastritis, short bowel syndrome, radiation enteritis, irritable bowel disease, Crohn's disease, post infectious syndrome, mild reflux, certain gut dysmotility, post chemotherapy disorder, malnutrition, malabsorption, and voluntary or involuntary long term starvation. Ille present invention may be used to treat each of these conditions, alone or secondary to the treatment or resolution of symptoms associated with noninsulin dependent diabetes mellitus, pre-diabetic symptoms, metabolic syndrome and insulin resistance.

Dosage forms used in methods of the invention can be in a form suitable for oral use, for example, as tablets, troches, lozenges, suspensions, micro suspensions, dispersible powders or granules, emulsions, micro emulsions, hard or soft capsules. Useful dosage forms include osmotic delivery systems as described in U.S. Pat. Nos. 4,256,108; 5,650,170 and 5,681,584, multiparticulate systems as disclosed in U.S. Pat. No. 4,193,985; systems in which the nutritional substance is coated with a mixed film of a hydrophobic organic compound-enteric polymer as disclosed in U.S. Pat. No. 6,638,534; systems such as diose described in U.S. Pat. Nos. 7,081,239; 5,900,252; 5,603,953; and 5,573,779; enteric-coated dry emulsion formulations (e.g., Journal of Controlled Release, vol. 107, issue 1 20 Sep. 2005, Pages 91-96), and emulsions such as the emulsion system of Olibra® and those disclosed in U.S. Pat. No. 5,885,590. Those of ordinary skill in the prior art know how to formulate these various dosage forms such that they release the majority of their nutritional substance in a subject's ileum as otherwise described herein.

Exemplary dosage forms that will release the majority of the ileal brake hormone releasing substance in vivo upon reaching the ileum include oral dosage forms such as tablets, troches, lozenges, dispersible powders or granules, or a hard or soft capsules which are formed by coating the ileal brake hormone releasing substance with an enteric coating (e.g., an enteric cellulose derivative, an enteric acrylic copolymer, an enteric maleic copolymer, an enteric polyvinyl derivative, or shellac). Preferred enteric coatings have a pH dissolution profile that delays the release in vivo of the majority of the ileal brake hormone releasing substance until the dosage form reaches the ileum. Enteric coatings can consist of a single composition; or can comprise two or more compositions, e.g., two or more polymers or hydrophobic organic compound-enteric polymer compositions as described in U.S. Pat. No. 6,638,534).

A “material having a pH dissolution profile that delays release in vivo of the majority of the ileal brake hormone releasing substance until the dosage form reaches the ileum” includes but is not limited to cellulose acetate trimellitate (CAT), hydroxypropylmethyl cellulose phthalate (HPMCP), polyvinyl acetate phthalate (PVAP), cellulose acetate phthalate (CAP), shellac, copolymers of methacrylic acid and ethyl acrylate, copolymers of methacrylic acid and ethyl acrylate to which a monomer of methylacrylate has been added during polymerization, a mixture of amylose-butan-1-ol complex (glassy amylose) with Ethocel® aqueous dispersion (Milojevic et al., Proc. Int. Symp. Contr. Rel Bioact. Mater. 20, 288, 1993), a coating formulation comprising an inner coating of glassy amylose and an outer coating of cellulose or acrylic polymer material (Allwood et al. GB 9025373.3), calcium pectinate (Rubenstein et al., Pharm. Res., 10, 258, 1993) pectin, chondroitin sulfate (Rubenstein et al. Pharm. Res. 9, 276, 1992), resistant starches (PCT WO 89/11269), dextran hydrogels (Hovgaard, et al., 3rd Eur. Symp. Control. Drug Del., Abstract Book, 1994, 87) modified guar gum such as borax modified guar gum, (Rubenstein and Gliko-Kabir, S. T. P. Pharma Sciences 5, 41-46, 1995), beta-cyclodextrin (Sidke et al., Eu. J. Pharm. Biopharm. 40 (suppl), 335, 1994), saccharide containing polymers, e.g., a polymeric construct comprising a synthetic oligosaccharide-containing biopolymer including methacrylic polymers covalently coupled to oligosaccharides such as cellobiose, lactulose, raffinose and stachyose, or saccharide-containing, natural polymers including modified mucopolysaccharides such as cross-linked pectate (Sintov and Rubenstein PCT/US 91/03014); methacrylate-galactomannan (Lehmann and Dreher, Proc. Int. Symp. Control. Rel. Bioact. Mater. 18, 331, 1991) and pH-sensitive hydrogels (Kopecek et al., J. Control Rel 19, 121, 1992), and resistant starches, e.g., glassy amylose.

Methylmethacrylate or copolymers of methacrylic acid and methylmethacrylate are preferred materials having a pH dissolution profile that delays release in vivo of the majority of the ileal brake hormone releasing substance until the dosage form reaches the ileum. Such materials are available as Eudragit® polymers (Rohm Pharma, Darmstadt, Germany). For example, Eudragit® L100 and Eudragit® S100 can be used, either alone or in combination, Eudragit® L100 dissolves at pH 6 and upwards and comprises 48.3% methacrylic acid units per g dry substance; Eudragit® S100 dissolves at pH 7 and upwards and comprises 29.2% methacrylic acid units per g dry substance. Generally, the encapsulating polymer has a polymeric backbone and acid or other solubilizing functional groups. Polymers which have been found suitable for purposes of the present invention include polyacrylates, cyclic acrylate polymer, polyacrylic acids and polyacrylamides. Another preferred group of encapsulating polymers are the polyacrylic acids Eudragit® L and Eudragit® S which optionally may be combined with Eudragit® RL or RS. These modified acrylic acids are useful since they can be made soluble at a pH of 6 or 7.5, depending on the particular Eudragit chosen, and on the proportion of Eudragit® S to Eudragit® L, RS, and RL used in the formulation. By combining one or both of Eudragit® L and Eudragit® S with Eudragit® RL and RS (5-25%), it is possible to obtain a stronger capsule wall and still retain the capsule's pH-dependent solubility. In additional preferred aspects of the invention, a coating of shellac (which also includes one or more emulsifiers such as hypromellose and/or triacetin) which is chosen to have a suitable pH-dependent dissolution profile for release die contents of a dosage form such as a tablet within the ileum of a patient or subject may be used. This type of coating provides a nutrateric approach to delayed and/or controlled release using naturally occurring, non-synthetic components.

A delayed and/or controlled release oral dosage form used in the invention can comprise a core containing an ileum hormonal-stimulating amount of a ileal brake hormone releasing substance that is coated by an enteric coating. In some embodiments, the coating comprises Eudragit® L100 and shellac, dr food glaze Eudragit® S100 in the range of 100 parts L100:0 parts S100 to 20 parts L100:80 parts S100, more preferably 70 parts L100:30 parts S100 to 80 parts L100:20 parts S100. As the pH at which the coating begins to dissolve increases, the thickness necessary to achieve ileum-specific deli very decreases. For formulations where the ratio of Eudragit® L100:S100 is high, a coat thickness of the order 150-200 μm can be used. For coatings where the ratio Eudragit® L100:S100 is low, a coat thickness of the order 80-120 μm can be used. Dosage forms used in methods of the invention can include one or more pharmaceutically acceptable carriers, additives, or excipients. The term “pharmaceutically acceptable” refers to a carrier, additive or excipient which is not unacceptably toxic to the subject to which it is administered. Pharmaceutically acceptable excipients are described at length by E. W. Martin, in “Remington's Pharmaceutical Sciences”, among others well-known in the art, pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7); wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk glucoses, as well as high molecular weight polyethylene glycols and the like.

Emulsions and microemulsions may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the ileal brake hormone releasing substance, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

Techniques for formulating the aforementioned useful dosage forms are either disclosed in the references cited above or are well-known to those of ordinary skill in the art.

“Stabilizing a subject's blood glucose and insulin levels” means lowering the subject's blood glucose and insulin levels to healthy levels within normal or close to normal ranges.

The terms “obesity” and “overweight” are generally defined by body mass index (BMI), which is correlated with total body fat and estimates the relative risk of disease. BMI is calculated by weight in kilograms divided by height in meters squared (kg/m2). Normal BMI is defined as a BMI of about 183 to 24.9 kg/m2. Overweight is typically defined as a BMI of 25-29.9 kg/m2, and obesity is typically defined as a BMI of at least 30 kg/m2. See, e.g., National Heart, Lung, and Blood institute, Clinical Guidelines on the Identification, Evaluation, and Treatment of Overweight and Obesity in Adults. The Evidence Report, Washington, D.C.: U.S. Department of Health and Human Services, NIH publication no. 98-4083 (1998). Obesity and its associated disorders are common and very serious public health problems in the United States and throughout the world. Upper body obesity is the strongest risk factor known for type 2 diabetes mellitus and is a strong risk factor for cardiovascular disease. Obesity is a recognized risk factor for hypertension, atherosclerosis, congestive heart failure, stroke, gallbladder disease, osteoarthritis, sleep apnea, reproductive disorders such as polycystic ovarian syndrome, cancers of the breast, prostate, and colon, and increased incidence of complications of general anesthesia. Obesity reduces life-span and carries a serious risk of the co-morbidities listed above, as well as disorders such as infections, varicose veins, acanthosis nigricans, eczema, exercise intolerance, insulin resistance, hypertension hypercholesterolemia, cholelithiasis, orthopedic injury, and thromboembolic disease (Rissanen et al., Br. Med. J. 301: 835-7 (1990)). Obesity is also a risk factor for the group of conditions called insulin resistance syndrome, or “Syndrome X” and metabolic syndrome. The present compositions are useful for treating obesity, and favorably impact the conditions which often occur secondary to obesity.

“Obesity-related disorder” includes all of the diseases and disorders mentioned in the preceding definition of “obesity”.

“Once-daily administration to the subject of a delayed and/or controlled release dosage form” includes self-administration of the dosage form by the subject.

“Dietary components” in the phrase “wherein the nutritional substance comprises a micro-encapsulation of glucose, lipids and dietary components” means any natural substance which either itself evidences impact on the ileal brake, or alternatively, enhances the impact that glucose and/or lipids have on the ileal brake, such components including other complex carbohydrates and nutritional components as otherwise described herein including, for example, alfalfa leaf, chloretlla algae, chlorophyllin and barley juice concentrate, among a number of other agents.

As summarized above, the invention provides methods for, the treatment of metabolic syndromes including hyperlipidemia, weight gain, obesity, insulin resistance, hypertension, atherosclerosis, fatty liver diseases and certain chronic inflammatory states. These methods can entail the testing of biomarkers; testing of breath, blood or body fluid biomarkers and selection of pharmaceutical compositions to resolve one or more of the metabolic syndrome conditions including but not limited to hyperlipidemia, weight gain, obesity, insulin resistance, hypertension, and atherosclerosis, fatty liver and chronic inflammatory states.

Thus, the invention provides a method of treatment of metabolic syndromes, wherein personalized treatments and pharmaceutical compositions are selected using the results of biomarker testing such as HbA1c, glucose, GLP-1, PYY, GLP-2, Proinsulin, CRP, hsCRP, endotoxin, IL-6. Personalized treatment and pharmaceutical compositions can be selected using a Glucose Supply Side computerized algorithm and system, wherein said Glucose Supply Side treatment method for diabetes consists of an algorithm (incorporated herein in its entirety) ranking favorable attributes of pharmaceutical compositions acting by minimizing excess glucose inside cells, and minimizing the amount of glucose that reaches target cells of the metabolic syndrome afflicted patient.

The invention also provides a method of treatment of metabolic syndromes, wherein personalized treatment and pharmaceutical compositions are selected by comparison of biomarker behavior patterns between patients having responded to Roux-en-Y bariatric surgery and their own response to oral dosing with pharmaceutical formulations comprised of carbohydrates, lipids or amino acids which activate the ileal brake response of the ileum in a manner similar to Roux-en-Y surgery. The method specifically entails orally administered pharmaceutical compositions that mimic, the action of Roux-en-Y gastric bypass surgery on the ileal brake. Even more specifically, the formulation for treatment of metabolic syndrome comprises the micro-encapsulation of glucose, lipids and components of diet formulated to release these active compositions at pH values between 6.5 and 7.5, which targets the action of said medicaments at the ileal brake in the distal intestine. The encapsulated compositions disclosed are a preferred medicament to decrease appetite for glucose, and thereby lower inflammation and benefit to the treatment of patients with metabolic syndrome, according to the results of testing of targeted biomarkers.

In a preferred embodiment of a method of treatment of metabolic syndromes according to the invention, oral dosing with about 2,000 to 10,000, about 2500-3,000 to, 10.000, about 7,500-10,000 milligrams of a pharmaceutical formulation of microencapsulated sugars, lipids, and/or amino acids activates the ileal brake in a dose increasing magnitude and treats one or more of the following components of metabolic syndrome: hyperlipidemia, weight gain, obesity, insulin resistance, hypertension, atherosclerosis, fairy liver diseases and chronic inflammatory states. The name of this medicament is BRAKE™.

In another embodiment, the invention provides a pharmaceutical formulation for treatment of metabolic syndrome, wherein the microencapsulated activation of the ileal brake is produced at a pH of about 6.5 to about 7.5 and involves the release of about 2,000 to about 10.000, about 2,500-3,000 to 10,000, about 7,500 to 10,000 milligrams of glucose, fructose, dextrose, sucrose or other glucose compositions active on the ileal brake in mammals at dosages between about 2,000 and about 10,000 milligrams, and as presented above.

In another embodiment, the invention provides a pharmaceutical formulation wherein the microencapsulated activation of the ileal brake is produced by approximately pH 6.5 to 7.5 release of about 2,000 to about 6,000, about 2,500-3,000 to about 10,000 milligrams of dextrose and about 2,000-4,000 milligrams of a lipid such as olive oil, corn oil, palm oil, omega3 fatty acid or other suitable lipid substances active on rite ileal brake of mammals.

In one embodiment, a pharmaceutical formulation for treatment of metabolic syndrome of the invention can achieve the microencapsulated activation of the ileal brake at about pH 6.5 to 7.5 by release of about 2,000 to about 10,000, about 2,500-3,000 to about 10,000, about 7,500-10,000 milligrams given once, twice or three times daily.

In another embodiment, a method of treatment of metabolic syndrome according to the invention involves oral treatment and includes use of pharmaceutical formulations as described above that activate the ileal brake and which act in the gastrointestinal, tract and the liver of a mammal to control metabolic syndrome manifestations and thereby reverse or ameliorate the cardiovascular damage (atherosclerosis, hyper tension, lipid accumulation, and the like) resulting from progression of metabolic syndrome.

In another preferred embodiment, a composition or a method of treatment of metabolic syndromes according to the invention involves an oral formulation mimetic of RYGB and includes use of said oral formulation with medicaments ordinarily used for treatments of manifestations of metabolic syndrome including but not necessarily limited to diabetes, hyperlipidemia, atherosclerosis, hypertension, obesity, insulin resistance, or chronic inflammation. The added combination pharmaceutical agent can be, by way of specific example, metformin, sitagliptin, saxagliptin, methotrexate, olanzapine, donepezil, memantine, atorvastatin, simvastatin, lovastatin, olmesartan, Enalapril, lisinopril, candesartan, irbesartan. Such compositions are the first to combine treatment of all of the primary metabolic syndrome manifestations into one product given once or twice daily to patients with all or many of the manifestations of metabolic syndrome.

In a preferred example, a composition of the invention can act to limit hepatic gluconeogenesis in the same manner as metformin, as well as add many other actions beneficial to the treatment of metabolic syndrome. The class of compounds related to and including metformin is called biguanide antihyperglycemic agents. While metformin is illustrative, and the combination product therefrom is called MetaBrake, the list of biguanides is not exclusive beyond metformin, and additional metformin mimetic or biguanide medicaments can be added to the formulations of the invention without departing from the practice of treatments for metabolic syndrome that combine oral mimetics of Roux-en-Y surgery effects on the ileal brake in conjunction with conventional anti-diabetes medicaments of the class represented by metformin. When used together with biguanide medicaments with particular metformin, the dosage required to lower glucose, lipids, obesity and inflammation may be reduced. When combined into an oral dosage form of Brake and a biguanide such as metformin, each tablet would contain about 500 mg of ileal hormone releasing substances and 25-50 mg of metformin, hi this manner the total dose of metformin per day would-be about 75 mg to about 150 mg and the ileal hormone releasing substance would be less than about 1500 mg, yet the combined product would control glucose, lower body weight, control triglycerides and lower systemic inflammation, actions that are somewhat beyond diose of metformin alone.

In one aspect of a composition or a method of treatment of metabolic syndromes according to the invention, the added combination pharmaceutical agent is from the class of DPP-IV inhibitors, including but not limited to formulations whereby the composition acts in the same way as DPP-IV inhibitors and the like. Examples of similar orally administered agents, thought to act by inhibition of DPP-IV, include Alogliptin, Vildagliptin, Sitagliptin, Dutogliptin, Linagliptin and Saxagliptin. While illustrative, this list is not meant to be exhaustive and it is readily apparent to persons skilled in the art of diabetes care that additional DPP-IV inhibitors can be added to the formulations of the invention without departing from the practice of preparing oral treatments for metabolic syndrome that combine oral mimetics of RYGB surgery effects on the ileal brake in conjunction with conventional anti-diabetes medicaments of the class represented by DPP-IV inhibitors. When used together with so called DPP-IV inhibitors, the dosage required to lower glucose, lipids, obesity and inflammation may be reduced to the benefit of reduction of the side effects of DPP-IV inhibitors, in particular the pancreatitis, which is presumed to be related to dosage of DPP-IV inhibitor chosen for treatment. When combined into an oral dosage form of Brake and a DPP-IV inhibitor such as sitagliptin, by way of example, each tablet would contain about 500 mg of ileal hormone releasing substances and 5 mg of sitagliptin. In this manner the total dose of sitagliptin per day would be less than 100 mg, yet the combined product would, in a completely novel way, control glucose, lower body weight, control triglycerides and lower systemic inflammation in a similar manner as RYGB surgery. This combination product of Brake and sitagliptin, called JanuBrake would be given once or twice daily and be suitable for consumer use of sitagliptin with an increased safety profile over that of sitagliptin alone. Similar gains in potency at lower doses, broad array of treatment responses in metabolic syndrome, and safety advantages over the statin alone would be seen with each of the DPP-IV inhibitors reduced to practice, and the disclosure of invention of a synergistic combination encompasses all DPP-IV inhibitor combinations with Brake prepared in this manner for these purposes.

In another aspect of a composition or a method of treatment of metabolic syndromes according to the invention, the added combination pharmaceutical agent is from the class of insulin sensitizers, also known as TZDs or Thiazolidinediones which are also known to be active on PPAR, Examples of similar agents, thought to act on the defined insulin sensitizer pathway, include pioglitazone, rosiglitazone, rivoglitazone, aleglitazar and the PPAR-sparing agents MSDC-0160, MSDC-0602. While illustrative, this list is not meant to be exhaustive and it is readily apparent to persons skilled in the art that additional insulin sensitizers, thiazolidinediones or PPARs or PPAR-sparing medicaments can be added to the formulations of the invention without departing front the practice of oral treatments for metabolic syndrome that combine oral mimetics of Roux-en-Y surgery effects on the ileal brake in conjunction with conventional anti-diabetes medicaments of the class represented by insulin sensitizers.

In another aspect, of a composition or a method of treatment of metabolic syndromes according to the invention, the added combination pharmaceutical agent is an alpha glucosidase inhibitor including but not limited to acarbose. The pharmaceutical thereby acts in the gastrointestinal tract, combining the effects on the ileal brake hormone release with the interruption of glucose absorption in the same way as acarbose, with fewer adverse effects, and to specifically include delayed release preparations of Acarbose, Miglitol, Voglibose and the like.

A composition or a method of treatment of metabolic syndromes according to the invention can also include the additional use of colesevelam, or can involve the use of a composition that acts in the gastrointestinal tract and on the ilal brake to limit glucose supply and to lower the lipid content of the blood in the same manner as colesevelam. While illustrative, the selection of a combination including colesevelam is not meant, to be exhaustive and it is readily apparent that additional Colesevelam mimetic medicaments can be added to the pharmaceutical composition of the invention without departing from the practice of oral treatments for metabolic syndrome that combine oral mimetics of Roux-en-Y surgery effects on the Beal brake in conjunction with conventional anti-diabetes medicaments of the class represented by colesevelam.

In another aspect of a composition or a method of combination treatment of metabolic syndromes according to the invention, the added combination pharmaceutical agent is from the class of statins, also known as cholesterol synthesis inhibitors or HMG-CoA reductase inhibitors. Examples of similar agents, thought to act on the defined statin pathway or by HMG-CoA reductase inhibition, include atorvastatin, simvastatin, lovastatin, ceruvastatin, pravastatin. While illustrative, this list of available statin drags is not meant to be exhaustive and it is readily apparent to persons skilled in the art that additional statins can be added to the formulations of the invention without departing from the practice of oral treatments for metabolic syndrome that combine oral mimetics of Roux-en-Y surgery effects on the ileal brake in conjunction with conventional anti-hyperlipidemic medicaments of the class represented by statins. When used together with so called statins, the dosage required to lower lipids and triglycerides may be reduced to the benefit of reduction of the side effects of statins, in particular the myopathy, which is known in the art to be related to higher dosages such as 80 mg of simvastatin. When combined into an oral dosage form of Brake and a statin such as atorvastatin, by way of example, each tablet would contain 500 mg of ileal hormone releasing substances and 1-2 mg of atorvastatin. In this manner the total dose of atorvastatin per day would be less than 20 mg, yet the combined product would control glycose, lower body weight, control triglycerides and lower systemic inflammation. This product, called LipidoBrake would be given once or twice daily and be suitable for consumer use of atorvastatin with an improved safety profile over that of atorvastatin alone. Similar gains in potency at lower doses, a broad array of treatment responses in metabolic syndrome, and safety advantages over the statin alone would be seen with each of the statins reduced to practice, and the disclosure encompasses all statin combinations with Brake prepared in this manner for these purposes.

In another aspect of a composition or a method of combination treatment of metabolic syndromes according to the invention, the added combination pharmaceutical agent is from the class of angiotensin AII inhibitors, also known as AII inhibitors. Examples of similar AH inhibitor agents, thought to act on the defined hypertension pathway, include Valsartan, Olmesartan, Candesartan, Irbesartan, Losartan, Telmisartan and the like. While illustrative, this list is not meant to be exhaustive and it is readily apparent to persons skilled in the an that additional AH inhibitors can be added to the formulations in claim 5 without departing from the practice of oral treatments for metabolic syndrome that combine oral mimetics of Roux-en-Y surgery effects on the ileal brake in conjunction with conventional anti-hypertensive medicaments of the class represented by AII inhibitors.

A composition or a method of combination treatment of metabolic syndromes according to the invention can use: added combination pharmaceutical agents that include a PDE5 inhibitor such as sildenafil (Viagra), vardenafil (Levitra) and Tadalafil (Cialis) phosphodiesterase type 5 inhibitor, often shortened to PDE5 inhibitor, is a drug used to block the degradative action of phosphodiesterase type 5 on cyclic GMP in the smooth muscle cells lining the blood vessels supplying the corpus cavernosum of the penis. These drugs are used in the treatment of erectile dysfunction. While illustrative, this list is not meant to be exhaustive and it is readily apparent to persons skilled in the art that additional medicaments active in the treatment of erectile dysfunction can be added to the formulations of the invention without departing from the practice of oral treatments for metabolic syndrome that combine oral mimetic of the Roux-en-Y surgery effect on the ileal brake in conjunction with conventional PDE5 inhibitors used in the treatment of erectile dysfunction.

A composition or a method of combination treatment of metabolic syndromes according to the invention can also use an added combination pharmaceutical agent such as methotrexate, Lorcaserin, topiramate, olanzapine (Zyprexa), risperidone or Ziprasidone, an added combination pharmaceutical agent that is active in the treatment of obesity and metabolic syndrome that leads to onset of Alzheimer's disease, including but not limited to Donepezil, (Aricept) a centrally acting reversible acetylcholinesterase inhibitor, memantine (Namenda), an NMDA receptor blocker involved with the action of glutamate or known inhibitors of beta amyloid protein formation.

A composition or a method of combination treatment of metabolic syndromes according to the invention can also use an added combination pharmaceutical agent such as an ACE inhibitor including but not limited to members of this class illustrated by captopril lisinopril, enalapril, quinapril, perindopril, trandolapril, a GPR119 agonist, including but not limited to the following candidates in early phase human trials: Arena/Ortho McNeil APD597; Metabolex MBX-2982; Prosidion/OSI PSN821 and the like, one or more of the active compositions used to treat HIV associated diseases, one or more of the active compositions used to treat Hepatitis B, C or other forms of chronic Hepatitis, or the method or composition my also include the rise of an intestinal pro-biotic mixture of bacteria formulated to release at pH between about 6.5 and about 7.5, which replaces the bacterial flora of the intestine at the location of the ileum.

In one embodiment of a composition or a method of treatment of metabolic syndromes according to the invention, the added combination pharmaceutical agent acts as a mimetic of the incretin pathway to lower glucose in the same or similar way as exenatide, including orally administered and parenterally administered sustained release preparations of exenatide and the like. Examples of similar agents, thought to act on the defined. GLP-1 pathway, include liraglutide, Lixisenatide. and taspoglutide. While illustrative, this list is not meant to be exhaustive and it is readily apparent to persons skilled in the art of diabetes care that additional GLP-1 pathway mimetics that are not DPP-IV inhibitors can be added to this list without departing from the practice of oral treatments for metabolic syndrome that combine oral mimetics of Roux-en-Y surgery effects on the ileal brake in conjunction with conventional anti-diabetes medicaments of the class represented by incretin pathway mimetics.

In another embodiment of a composition or a method of treatment of metabolic syndromes according to the invention, the added combination pharmaceutical agent can also act in the same way as insulin formulated for oral administration, including orally administered sustained release preparations of insulin and the like. Micro-spheres or nano-spheres formed of polymers or proteins such as insulin are well known to those skilled in the art, and can be tailored for passage through the gastrointestinal tract directly into the blood stream. Alternatively, the compound can be incorporated into cholestosomes, bio-erodible polymers, and/or micro-spheres/nano-spheres, or composites of these delivery vehicles. See, for example, U.S. Pat. Nos. 4,906,474, 4,925,673 and 3,625,214, and Jein, TIPS 19:155-157 (1998), the contents of which are hereby incorporated by reference. Examples of these oral formulations of insulin include HDV-1 insulin and oral insulin formulations by Emisphere, Biocon and Oramed. While illustrative, this list is not meant id be exhaustive and it is readily apparent to persons skilled in the art of diabetes care that additional formulations of oral insulin can be added to this list without departing from the practice of oral treatments for metabolic syndrome that combine oral mimetics of Roux-en-Y surgery effects on the ileal brake in conjunction with conventional anti-diabetes medicaments of the class represented by the oral insulin pathway mimetics.

In still another embodiment of a composition or a method of treatment of metabolic syndromes according to the invention, personalized treatment and pharmaceutical compositions can be selected for treatment of metabolic syndrome manifestations including, but not limited to diabetes mellitus obesity, insulin resistance, hypertension, hyperlipidemia, fatty liver disease, and chronic inflammation.

In still another embodiment of a composition or a method of treatment of metabolic syndromes according to the invention, the combination pharmaceutical formulation of an anti-diabetic drug and sugars, lipids and amino acids of Brake™ activates the ileal brake and thereby reduces insulin resistance, lowers blood glucose, lowers body weight in obesity, lowers systemic inflammation, lowers fatty liver disease and lowers triglycerides and other lipids in a patient with any or all of the components of metabolic syndromes.

In still another embodiment of a composition or a method of treatment of metabolic syndromes according to the invention, the combination pharmaceutical formulation of a lipid lowering drug and sugars, lipids and amino acids of BRAKE activate the ileal brake and thereby reduces insulin resistance, lowers blood glucose, lowers body weight in obesity, lowers systemic inflammation, lowers fatty liver disease and lowers triglycerides and other lipids in a patient with any or all of the components of metabolic syndromes.

In still another embodiment of a composition or a method of treatment of metabolic syndromes according to the invention, the combination pharmaceutical formulation of an anti-obesity drug and sugars, lipids and amino acids of BRAKE activates the ileal brake and thereby reduces insulin resistance, lowers blood glucose, lowers body weight in obesity, lowers systemic inflammation, lowers fatty liver disease and lowers triglycerides and other lipids in a patient with any or all of the components of metabolic syndromes.

In still another embodiment of a composition or a method of treatment of metabolic syndromes according to the invention, the combination pharmaceutical formulation of an anti-inflammatory drug such as methotrexate and sugars, lipids and amino acids of BRAKE activate the ileal brake to produce beneficial immunoregulatory actions and thereby reduces insulin resistance, lowers blood glucose, lowers body weight in obesity, lowers systemic inflammation, lowers fatty liver disease and lowers triglycerides and other lipids in a patient with any or all of the components of metabolic syndromes.

In still another embodiment of a composition or a method of treatment of metabolic syndromes according to the invention, the combination pharmaceutical formulation of an anti-hypertensive drug with sugars, lipids and amino acids of BRAKE activates the ileal brake and thereby reduces insulin resistance, lowers blood glucose, lowers body weight in obesity, lowers systemic inflammation, lowers fatty liver disease and lowers triglycerides and other lipids in a patient with any or all of the components of metabolic syndromes.

In still another embodiment of a composition or a method of combination treatment of metabolic syndromes according to the invention, the combination pharmaceutical formulation of an anti-atherosclerosis drug, and sugars, lipids and amino acids of BRAKE activates the ileal brake and thereby reduces insulin resistance, lowers blood glucose, lowers body weight in obesity, lowers systemic inflammation, lowers fatty liver disease and lowers triglycerides and other lipids in a patient with any or all of the components of metabolic syndromes.

In still another embodiment of a composition or a method/of treatment of metabolic syndromes according to the invention, personalized treatment and pharmaceutical compositions are selected for treatment of metabolic syndrome manifestations of Erectile Dysfunction that act on the ileal brake and thereby reduces insulin resistance, lowers blood glucose, lowers body weight in obesity, lowers systemic inflammation, lowers fatty liver disease and lowers triglycerides and oilier lipids in a patient with any or all of the components of metabolic syndromes.

In still another embodiment of a composition or a method of combination treatment of metabolic syndromes according to the invention, personalized treatment and pharmaceutical compositions are selected for treatment of metabolic syndrome manifestations of chronic obstructive pulmonary disease, or COPD, that act on the ileal brake and thereby reduces insulin resistance, lowers blood glucose, lowers body weigh in obesity, lowers systemic inflammation, lowers fatty liver disease and lowers triglycerides and other lipids in a patient with any or all of the components of metabolic syndromes.

In still another embodiment of a composition or a method of treatment of metabolic syndromes according to the invention, personalized treatment and pharmaceutical compositions are selected for treatment of metabolic syndrome manifestations of Rheumatoid Arthritis, or RA, that act on the ileal brake and thereby reduces insulin resistance, lowers blood glucose, lowers body weight in obesity, lowers systemic inflammation, lowers fatty liver disease and lowers triglycerides and other lipids in a patient with any or all of the components of metabolic syndromes.

In still another embodiment of a composition or a method of combination treatment of metabolic syndromes according to the invention, personalized treatment and pharmaceutical compositions are selected for treatment of metabolic syndrome manifestations of Alzheimer's disease, with or without component Type 2 Diabetes that act on the ileal brake and thereby reduces insulin resistance, lowers blood glucose, lowers body weight in obesity, lowers systemic inflammation, lowers fatty liver disease and lowers triglycerides and other lipids in a patient with any or all of the components of metabolic syndromes.

In still another embodiment of a composition or a method of combination treatment of metabolic syndromes according to the invention, personalized treatment and pharmaceutical compositions are selected for treatment of metabolic syndrome manifestations of Multiple Sclerosis that act on the ileal brake and thereby reduces insulin resistance, lowers blood glucose, lowers body weight in obesity, lowers systemic inflammation, lowers fatty liver disease and lowers triglycerides and other lipids in a patient with any or all of the components of metabolic syndromes.

In still another embodiment of a composition or a method of combination treatment of metabolic syndromes according to the invention, personalized treatment and pharmaceutical compositions are selected for treatment of metabolic syndrome manifestations of Crohn's Disease that act on the ileal brake and thereby reduces insulin resistance, lowers blood glucose, lowers body weight in obesity, lowers systemic inflammation, lowers fatty liver disease and lowers triglycerides and other lipids in a patient with any or all of the components of metabolic syndromes.

In still another embodiment of a composition or a method of combination treatment of metabolic syndromes according to the invention, personalized treatment and pharmaceutical compositions are selected for treatment of metabolic syndrome manifestations of Non-Alcoholic Fatty Liver Disease (NAFLD) that act on the ileal brake and thereby reduces insulin resistance, lowers blood glucose, lowers body weight in obesity, lowers systemic inflammation, lowers fatty liver disease and lowers triglycerides and other lipids in a patient with any or all of the components of metabolic syndromes.

In still another embodiment of a composition or a method of treatment of metabolic syndromes according to the invention, personalized treatment and pharmaceutical compositions are selected for treatment of metabolic syndrome manifestations of Hepatitis that act on the ileal brake and thereby reduces insulin resistance, lowers blood glucose, lowers body weight in obesity, lowers systemic inflammation, lowers fatty liver disease and lowers triglycerides and other lipids in a patient with any or all of the components of metabolic syndromes.

In still another embodiment of a composition or a method of combination treatment of metabolic syndromes according to the invention, personalized treatment and pharmaceutical, compositions are selected for treatment of metabolic syndrome manifestations of HIV diseases that act on the ileal brake and thereby reduces insulin resistance, lowers blood glucose, lowers body weight in obesity, lowers systemic inflammation, lowers fatty liver disease and lowers triglycerides and other lipids in a patient with any or all of the components of metabolic syndromes.

The invention also provides a process for the combination oral treatment of metabolic syndromes including but not limited to Type 2 diabetes mellitus and conditions associated with diabetes mellitus, wherein said process comprises testing of breath biomarkers which include oxygen, glucose, acetoacetate, betahydroxybutyrate and other suitable free fatty adds and ketone bodies well known in the art; testing isoprostane and other metabolites of prostaglandins or any other analytes that are considered markers of oxidative stress; Nitrous oxides, methyl nitrous oxide metabolites; cytokines, proteins, GLP-1. GLP-2, PYY, proinsulin, insulin, incretins, peptides, adiponectin, C-Reactive Protein, hsCRP, endotoxin, procalcitonin, troponin, electrolytes, and other markers of the inflammatory pathways or those of cardiovascular injury. The processes specifically incorporate the testing of these and other biomarkers and use the results to select pharmaceutical compositions that act on the ileal brake and incorporate other currently available pathway specific biomarkers for metabolic syndrome manifestations. While illustrative, this list of medicaments for combination oral treatment is not meant to be exhaustive and it is readily apparent to persons skilled in the art of diabetes care that additional biomarkers and combinations of medicaments can be added to this list without departing from the practice of testing of biomarkers and using these results to select personalized treatments for patients with metabolic syndromes.

For example, in such practices of the invention of combination treatments for metabolic syndrome manifestations that include an active medicament and the disclosed formulations that act as ileal brake hormone releasing agents, the condition to be treated is Type 2 diabetes, Type 1 diabetes, Rheumatoid Arthritis, Obesity, Alzheimer's disease, Crohn's disease, Multiple Sclerosis, Irritable Bowel syndrome (IBS), COPD, Psoriasis, HIV or AIDS, Non-Alcoholic Fatty Liver Disease, Hepatitis C, Congestive Heart Failure, Atherosclerosis, Chronic Inflammation-, Hypertension, Hyperlipidemia, Erectile Dysfunction

In certain embodiments of a pharmaceutical composition of the invention used in die treatment of metabolic syndrome according to the practices of the invention disclosed herein, the composition includes a necessary amount of Vitamins A, D, E or B12, or a necessary daily amount of Aspirin, ranging between about 81 to about 325 mg, or a necessary amount, of omega-3, as derived from fish oils, or a necessary amount of micro-encapsulated food grade chocolate, either as dark chocolate, milk chocolate or white chocolate, each alone or as mixed components, hi other embodiments, a pharmaceutical composition of the invention includes the substances disclosed herein and the remainder of the dosage form comprises mixtures of food components of sugars, lipids and amino acids and acts in the same way as pH encapsulated glucose, releasing at a pH of about 6.8 to about 7.5 to lower appetite, selectively modify taste and thereby change taste preferences for foods and nutrients, regulate the immune system and lower systemic inflammation and restore normal compositions of bacteria in metabolic syndromes and associated conditions. Examples of active compositions include combinations of pH encapsulated microparticulates of different pH release for glucose, combined with immediate release DPP-IV inhibitors, TZD compounds, ACE inhibitors. All inhibitors, Incretin pathway mimetics, PDE5 inhibitors, pH encapsulated probiotic organisms, Statins, antibiotics, and GLP-1 mimetics. While illustrative, this list of combinations and pH release encapsulated compounds is not meant to be exhaustive and it is readily apparent to persons skilled in the art of metabolic syndrome treatment that additional pH encapsulated compounds and additional classes of supply side beneficial substances can be added to this list without departing from the practice of testing of biomarkers and using these results to select personalized treatments for patients with metabolic syndrome.

In another aspect, the invention provides a Glucose Supply Side method for the treatment of Type 2 diabetes mellitus and metabolic syndrome component conditions associated with Type 2 diabetes mellitus. The Glucose Supply Side method comprises the administration to a human or non-human mammal in need thereof of any of the pharmaceutical compositions described above in any combination and each in any dosage according to the results of testing of biomarkers. While illustrative, this list of combinations is not meant to be exhaustive and it is readily apparent to persons skilled in the treatment of metabolic syndromes, that additional combinations and medicaments can be added to this list without departing from the practice of testing of biomarkers and using these results to select personalized treatments for patients with metabolic syndrome.

In one embodiment of a method for the treatment of Type; 2 diabetes mellitus and conditions associated with diabetes mellitus, using a system Glucose Supply Side algorithm and method according to the invention, the method comprises testing of each patient for genomic markers of response to Glucose Supply Side selected pharmaceutical compositions, and then using the results of genomic testing to individualize the dosage of said compound using genomic markers of the Glucose Supply Side and of the patients individual metabolism of said composition alone or in combination with the results of the Glucose Supply Side breath test biomarkers

In another embodiment of a method for treatment of diabetes mellitus and conditions associated with diabetes mellitus in a human patient according to the invention and using the Glucose Supply Side algorithm incorporated by reference, the practice of said method comprises identifying said patient by inspection of medical records of care and results of tests.

In another aspect, the Glucose Supply Side method and associated process use: an input/output (I/O) device coupled to a processor; a communication system coupled to the processor; and a medical computer program and system coupled to the processor, the medical system configured to process medical data of a user and generate processed medical information, wherein the medical data includes one or more of anatomical data, diabetes associated biomarkers, test specimen data, biological parameters, health information of die user, wherein the processor is configured to dynamically control operations between the communication system and the medical system.

The operations of the communication system can include one or more of a mobile device, wireless communication device, cellular telephone/Internet Protocol (IP) telephone, Wi-Fi telephone, server, personal digital assistant (PDA), and portable computer (PC). Also, the biological parameters can include one or more of current and historical biological information of the user comprising one or more of weight, height, age, temperature, body mass index, medical analyses results, body fluid analyses, blood analyses results, breath testing results, electrical activity of a body of the user, heart activity, heart rate, and blood pressure. Health information used in the processes can include one or more of current and historical health information of the user, wherein the health information includes one or more of dietary data, types of food consumed, amounts of food consumed, medications consumed; times of food consumption, physical activity exercise regimen, work schedule, activity schedule, and sleep schedule.

Additionally, the communication system can be configured to communicate one or more of the medical data and the processed medical information to a remote device located one or more of on the user, in a home, in an office, and at a medical treatment facility, the remote device including one or more of a processor-based device, mobile device, wireless device, server, personal digital assistant (PDA), cellular telephone, wearable device, and portable computer (PC). Also, the processed medical information can be used for one or more of observation, research study, real time monitoring, periodic monitoring, correlation, diagnosis, treatment, database archival, communication, command, and control.

The communication process may be configured to communicate alert information in response to the processed medical information, wherein the alert information includes one or more of a message, a visual alert, an audible alert, and a vibratory alert communicated to the user, wherein the alert information includes one or more of voice data, text, graphics data, and multimedia information. Further, the communication process may be configured to process medical data comprises correlating one or more of the medical data and processed medical information with categorical data of the user, wherein the categorical data includes one or more of data of an age category of the user, data of a body type of the user, and parametric data of the user. The processor can be configured to convert one or more of the medical data and the processed medical information from a first form to a second form.

A system of the invention useful in the implementation of the processes described above can comprise a memory device coupled to the processor, wherein the memory device is configured for storing one or more of the medical data and the processed medical information. The system can comprise a positioning device coupled to the processor, the positioning device automatically determining a location of the user and outputting information of the location, wherein the positioning device is a Global Positioning System (GPS) receiver, wherein the location includes one or more of a latitude, a longitude, an altitude, a geographical position relative to a land-based reference. The r/o device may be configured to provide communication via a network comprising a wired network and a wireless network. The system may include a port configured to receive one or more of a specimen from a body of the user and a substrate including the specimen. Further, the system may also comprise an analyzer coupled to xerogel-based substrates for concentration-dependent analyte detection, the analyzer including a xerogel-based sensor coupled to a processor configured to analyze the specimen and generate the processed medical information, wherein analysis of the specimen includes correlating parameters of the specimen with the medical data.

The specimen used in processes and systems of the invention can be a biological sample, which could include breath, saliva or any fluid or tissue from a patient, wherein the processed medical information includes one or more of a chemical analysis of the specimen.

A device of the invention comprises the components of the invention's system as described above and can comprise at least one auxillary port for coupling to at least one other device. The device may include a medicament delivery system coupled to the processor, the delivery system including at least one reservoir that contains at least one composition, the delivery system configured to administer at least one composition for use in treating the user, wherein the composition is administered under control of the processor and the processed medical information. The delivery system is configured to automatically administer die composition or medicament. Also, the delivery system may be configured to administer the composition under manual control of the user.

Processed medical information employed in the processes, systems, and devices of the invention may include a mathematical expression for choice of medicament among a plurality of dosages, wherein the composition is administered under at least one of the plurality of dosages when personalized for the cam of the diabetes patient. The processed medical information includes information of the at least one composition, wherein the information of the at least one composition includes one dr more of composition identification information, an amount released, and a time of release. The processor may configure to generate and receive control signals.

In certain embodiments of the invention, personalizing one or more diabetes treatment profiles associated with a monitored analyte concentration in a specimen includes retrieving a current analyte pharmacokinetic rate of change information, calculation of a modified analyte rate of change information based on the received analyte data associated with monitored analyte concentration; mid generating one or more modifications to the medicament composition from the pharmacokinetic calculations performed thereon.

In certain embodiments of a device of the invention, the processor generates the control signals one or more of automatically and in response to an input from the user. Control signals may be configured to control one or more of devices coupled to the user, devices implanted in the user and devices coupled to the processor. Such control signals may control administration of at least one medicament composition or combinations thereof.

In a still further embodiment of the invention, the invention provides a system for providing metabolic syndrome component management, comprising: a sensor unit measuring concentrations of analytes; an interface unit; one or more processors couple to the interface unit; a memory for storing data and instructions which, when executed by the one or more processors, causes the one or more processors to receive data associated with monitored analyte concentrations for a predetermined time period substantially in real time, retrieve one or more therapy profiles associated with the monitored analyte concentrations, and generate one or more modifications to the retrieved one or more therapy profiles based on the data associated with the monitored analyte concentrations.

In a still further embodiment, of the invention, the invention provides a providing preferred embodiments of metabolic syndrome treatment, comprising: an analyte monitoring system configured to monitor analyte related levels of a patient substantially in real time; a medication delivery unit operatively for wirelessly receiving data associated with the monitored analyte level of the patient substantially in real time from the analyte monitoring system; and a data processing unit operatively coupled to the one or more of the analyte monitoring system or the medication delivery unit, the data processing unit configured to retrieve one or more therapy profiles associated with the monitored analyte related levels, and generate one or more modifications to the retrieved one or more therapy profiles based on the personalized treatment processes associated with the monitored analyte measurements.

In an embodiment of a system of the invention, the “Highest Risk” for cardiovascular injury and complications from diabetes corresponds to a composite glucose supply and insulin demand SD score generally less than 1.0. Medicaments such as excessive insulin (SD 0.62-0.79) and secretagogues (SD 0.69-0.81) have the lowest scores and the lowest potential benefits. Medicaments such as alpha-glucosidase inhibitors (SD 1.25), TZD's (SD 1.27-1.35), and metformin (SD 2.20) are associated with the SD scores above 1.0 and teach the greatest potential benefits in the Glucose Supply Side computerized algorithm.

In an embodiment of a system of the invention, the Glucose Supply Side system gauge is segmented into at least one category including “Low Risk”, and “High Risk.”

In an embodiment of a system of the invention, a Cardiovascular risk score is incorporated that is composed of other medicaments that affect the rate of disease progression; such risks are accelerated in a quantitative manner by some of these medicaments. Acceleration can be measured by biomarkers according to the teachings of the Supply Side System.

In another embodiment of a system of the invention; a Cardiovascular risk score is incorporated that is composed of other medicaments that affect the rate of disease progression; such risks are attenuated in a quantitative manner by some of these medicaments. Attenuation can be measured by means of biomarkers according to the teachings of the Supply Side System. A Cardiovascular risk score may be composed of other medical events that quantify the rate of cardiovascular injury progression in metabolic syndrome using an algorithm and one or more biomarkers of cardiovascular progression in a model and system, wherein such risks are attenuated or accelerated in a quantitative manner by some of the disclosed treatments. Acceleration and attenuation can be measured by means of biomarkers and used to adjust dosages or personalize treatment to individual patients.

The invention is described further in the following examples of the following Experimental-Section, which are illustrative and are not limiting.

### EXPERIMENTAL SECTION

In the Examples described hereinafter, the same table-numbers may be used, in different examples. For example, Examples 1-4 contain a “Table 1”, and Example 5 contains a different. (able which is also designated as “Table 1”. When an example refers to a table number, it means the table contained within that example.

### Example 1

### Healthy Human Volunteer Study

### Formulation 1

600 mg/capsule glucose

1000 mg capsule

10% Eudragit coating

Plasticizer (propylene glycol, triethyl acetate and water)

Magnesium stearate

Silicon Dioxide

A single formulation as described for formulation 1 above was-administered to five healthy adult human volunteers fasting in the morning at bedtime. Each of the volunteers was in the fasted state (i.e., none had eaten within two hours of the formulation administration). Blood levels (ng/ml) of GLP-1, GLP-2, C-peptide, GLP-1 (total) (determined by radioimmunoassay (RIA)), PYY, blood glucose (BS), GLP-1 (total) (with plasma), and insulin for each of the volunteers were measured just prior to administration of the above formulation and every four hours after administration until the eleventh hour after administration of the formulation.

Based on the data obtained for the live individuals tested as above, it was concluded that for all subjects except for one, that blood levels of GLP-1 (total) (RIA), GLP-1 (total) (with plasma), GLP-2, PYY, insulin, C-peptide, and blood glucose peaked at around 6-10 hours after administration of formulation 1. The peak levels of GLP-1 (total) (RIA), GLP-1 (total) (with plasma), GLP-2, and PYY correlated with the peak levels of insulin, C-peptide, and blood glucose, especially for subjects D and B. This suggests that there is an inverse correlation between these two groups and therefore the stimulation of the first grouping causing a reduction of levels of the second grouping.

Further, blood glucose and insulin levels dropped as the result of the stimulation of GLP-1, GLP-2, C-peptide, PYY, and insulin.

After the experiment described in this example, some patients continued to take formulation 1 above for an extended period of time and experienced a beneficial weight loss and as well as in one patient significant control of blood glucose and insulin levels.

Levels of blood glucose, ileal brake derived hormones and their response to food stimulation could be assessed and abnormalities in the ileal brake responsiveness could be evaluated (GLP-1, GLP-2, PYY). This indicates that methods of the invention can be used to diagnose whether a subject suffers from a disorder associated with an abnormality in their ileal brake hormones to respond to food, blood glucose or insulin levels. For example, a standard dosage form comprising an enterically-coated, ileum hormone-stimulating amount of a ileal brake hormone releasing substance could be administered to a subject, the subject's levels of ileal hormones blood glucose and insulin as well as ileal hormones including GLP-1, GLP-2, PYY, IGF-1, IGF-2 and leptin could be measured at regular intervals subsequent to administration of the ileal brake hormone releasing substance. Measured levels of the ileal hormones (e.g., GLP-1, GLP-2, PYY, IGF-1, IGF-2), as welt as blood glucose and insulin could be compared to healthy levels of ileal brake hormones, blood glucose and insulin determined by administering an equivalent enterically-coated, ileum hormone-stimulating amount of a ileal brake hormone releasing substance to a control subject.

Further, this example and the following examples establish that compositions such as formulation 1 above, among others, when administered while the subject is in the fasted state and at a time of about 3 to 12 hours, preferably about six to about nine horn's prior to the subject's next intended meal, provide an ileum hormone-stimulating amount of a ileal brake hormone releasing substance.

### Example 2

### Obese Subject Study

FIG. 2 illustrates four-month weight loss and blood glucose levels of a subject who took a single capsule according to formulation 1 once-daily in the fasted state at bedtime (about six to about nine hours prior to the subject's next intended meal) for a period of about four months. As illustrated in FIG. 2; the subject achieved a significant decrease in weight (about 24 pounds) at the end of about four months. The subject's blood glucose levels also improved significantly over the course of formulation 1 administration. Over the course of the four month period, the subject experienced periods of decreased appetite that lasted as long as 12 hours or longer, and enjoyed a substantial overall caloric intake reduction. By the end of the four month period, the subject would no longer be diagnosed as obese and had blood glucose levels that were well within acceptable ranges.

### Example 3

Formulation II

| | Amount | Range |
|---|---|---|
| Blend: | | |
| Alfalfa Leaf | 3.00 | 1-10+ |
| Chlorella Algae | 3.00 | 1-10+ |
| Chlorophyllin | 3.00 | 1-10+ |
| Barley Grass Juice Concentrate | 3.00 | 1-10+ |
| Dextrose | 1429.00 | 500-3000+ |
| Other Tablet Ingredients: | | |
| Coating * | 388.40 | 125-750+ |
| Corn Starch NF | 80.00 | 25-160+ |
| Hypromellose USP | 32.40 | 10-65+ |
| Stearic Acid NF (Vegetable Grade) | 19.50 | 6.5-35+ |
| Triacetin FCC/USP | 19.30 | 6.5-40+ |
| Magnesium Stearate NF/FCC | 7.00 | 2.5-15+ |
| Silicon Dioxide FCC | 2.50 | 0.75-5.0+ |

* Depending upon the composition used, 10% by weight Aqueous Nutrateric Enteric Coating (from Colorcon, Inc., Aphoeline-0) in the examples) as described below (for formulation III), 10% by weight Aqueous Shellac (Mantrose Haeuser, Inc. Aphoeline-1), 8% by weight Aqueous Indian Shellac (Aphoeline-2) was used to coat the formulations.

Formulation II was provided by mixing the actives with corn starch, stearic acid, magnesium stearate and silicon dioxide and pressing into a tablet, and coating the tablet, with shellac (either 10% or 8% shellac), triacetin and the hypromellose. A Eudragit coating could alternatively be used, similar to that which coats formulation 1, as described above.

Based upon the results in examples 1 and 2, the inventors embarked upon a project to create a vehicle which can be given orally and deliver the ileal brake hormone releasing substance to the ileum to stimulate the ileal brake. The following data (appearing in attached FIGS. 3-8) reports the results of the experiment conducted on the formulation (I composition. A number of formulations of pills with different coatings and structures and at times sub coatings were also used and tested and analyzed such that formulation 33 resulted. With the initial results, it was apparent that the pill composition and content indicate a logical pattern consistent with the hypothesis of stimulating the ileal brake hormonal pathways to control the manifestations of metabolic syndromes. The experiments were also performed to answer the issue of consistency of effect and the results obtained suggested that the approach was amenable for standardization and usage as a therapeutic composition, as well as a diagnostic tool in the future, the extra results showed improvement of the blood glucose and on subsequent testing of insulin and C-peptide showed that stimulation of insulin and C-peptide did not folly explain the theory involved in decreasing insulin resistance. Leptin, IGF-1 and IGF2 were measured and our results evidence that the stimulation of those factors contribute to the stabilization of blood glucose and reduction in insulin resistance observed.

The experiment was performed on volunteers as part of the testing of the different compositions, and structure of the pill in order to determine the best stimulation. The present example reports the results of the live patients that took formulation II as well as the graphs associated with it (FIGS. 3-8). Informed consent was obtained prior to administering the composition to five tasting volunteers, allowing them water only ad libitum throughout the day. They were given the recommended daily dose of formulation II after being examined by a physician and their vitals deemed appropriate for the test. A base line level blood level was obtained at hour 0 then hourly thereafter till hour 10. The blood was collected by a registered nurse, labeled accordingly and coded by a professional national lab, prepared according to the instruction of another out of state specialized national lab including cold centrifuge immediately upon receipt of the sample. The labeled coded samples were stored in dry ice refrigerated and shipped to 3 different specialty national labs for analysis and measurement of the metabolic and hormonal levels. The data was forwarded as per code numbers to the local national lab and encoded appropriately to match the volunteers for analysis. Analysis was performed and graphs were drawn accordingly. No unusual event occurred; Applicants were surprised with the results of one individual for the extremely high level of GLP-1 that did not follow the same pattern as the others. Even though it was advantageous to maintain that individual within the data to enhance the statistics, Applicants removed that data from the data presented.

Applicants note that the other pill compositions tested showed similar but less significant stimulation and a slight modification in pattern, in accordance with the expected formulation release and stimulation of the pills. The subjects were monitored at all times by a registered nurses and a physician. The results appear in FIGS. 3-8. Those figures clearly evidence that the compositions of the present invention had a favorable impact on blood glucose, reduced insulin resistance, and had favorable impact on glucagon, GLP-1, blood glucose, C-peptide, insulin, PYY, leptin, IGF-1 and IGF-2. Note that the IGF-1 and IGF-2 parameters may help explain some of the significant difference in muscle mass preservation observed and red need fat mass using the present compositions. The results for GLP-1 (FIG. 6) suggest favorable body composition (reduce fat/increased muscle), changes which matches to a certain extent the levels achieved with RYGB surgery without the attendant complications and side effects of such surgery. The results for PYY (FIGS. 7A-E) follow a similar stimulation pattern with earlier stimulation coupled with sustained stimulation at the level of about 3-8 hours and maximum intensity of 4 to 10 hours after the ingestion of the present composition. The patterns are predictable and amenable to standardization and are indicative of ileal peptide stimulation which contributes to appetite suppression.

Regarding the response of glucose, c-peptide and insulin to the composition of the present invention, that dam is summarized in FIG. 8A-J. Given the wide variation and the response of glucose/insulin interaction, the inventor divided the patients into categories with different starting points to determine if there is any difference in the action of the present compositions on the different groups (normal glucose/mild elevation insulin; elevated glucose/normal to low insulin levels; elevated glucose and elevated insulin; normal glucose/elevated fasting insulin and normal glucose/mild insulin increase). The principal effect of the present compositions is homeostasis; regulation of blood glucose and insulin is in a manner consistent with the suppression/reduction of insulin resistance and an increase in glucose tolerance (by up-regulating ileal hormones, IGF-1, IGF-2). In the first group (normal glucose/mild elevation insulin, FIG. 8A-B), the insulin levels are suppressed with a slight decrease in glucose levels, consistent with suppression of insulin resistance. The second group (elevated blood glucose/normal to low insulin levels, FIG. 8C-D) demonstrated that in the absence of insulin stimulation is similar to a typical stimulation of insulin in type 2 diabetes, with the peak of stimulation of insulin stimulation occurring early in the process, but with insulin declining later in the process, evidencing homeostasis and a reduction in insulin resistance and enhanced glucose tolerance over time. The third group (elevated blood glucose and insulin, FIG. 8E-F) demonstrates the continual seesaw between insulin stimulation and suppression as it relates to suppression of insulin resistance as insulin trended down over time with insulin evidencing bouts of stimulation within a cycle. The fourth group (normal glucose/elevated fasting insulin, FIG. 8G-H) evidenced decline in glucose and insulin consistently over time (significant insulin decline with 3-4 hours after administration of composition). In the fifth group (normal glucose/mild insulin increase, FIGS. 8I-J), insulin reduction with decrease in blood glucose further evidenced suppression of insulin resistance.

In this set of experiments, the inventor was able to stimulate hormones of the ileal break using a safe, effective oral formulation comprising ileal brake hormone releasing substances with enteric release (delayed/controlled release) helps to curb appetite in a natural way without the side effects of prior art methods. The experiments evidenced a coherent pattern of hormone release that can serve as a diagnostic tool for testing the ileal break hormones for insufficiencies, excesses or other abnormalities. Also shown is the fact that the present invention stimulates IGF1 and IGF2 and leptin as well as decreasing/suppressing insulin resistance and enhancing glucose tolerance, giving it excellent prospects for treating NIDDM (type II diabetes mellitus), prediabetes, metabolic syndrome and insulin resistance. By stimulating the ileal hormones pursuant to the present invention, the present invention represents an enhancer of well-being, muscle mass preservation or production. Further, the present invention also is able to stimulate glucagon, glucagon-like (enteroglucagon, etc.).

### Example 4

An experiment was undertaken using two different formulas (including formula H, above, in order to determine the maximum yield of the pills given to subjects. The subjects were divided in groups of 7, and different pills compositions were given to each.

The object was to investigate and measure multiple parameters besides blood glucose, such as glucose homeostasis to include insulin, eupeptide, glucose, IGF-1, IGF-2, glucagon, as well as leptin. The composition of the pills was developed in such a way so as to decrease the number of pills from an initial 16 to 7. The pills were given orally while fasting, and the blood work was drawn hourly for all parameters and each tube was coded for, both time, and patient. The blood product was handled by a professional staff prepared as required by the different tests, and the samples sent to two different national labs drat provided results in coded numbers.

Once decoded and analysed for each patient, the results were taken as the average response to the different parameters for the different patients, considering that some of these subjects presented with either an abnormal insulin level, abnormal glucose level or both.

The two pills composition used during this testing were as follows (ingredients per tablet, in mg), Formula II (as above) in Example 3:

| | Amount | Range |
|---|---|---|
| Proprietary Blend: | | |
| Alfalfa Leaf | 3.00 | 1-10+ |
| Chlorella Algae | 3.00 | 1-10+ |
| Chlorophyllin | 3.00 | 1-10+ |
| Barley Grass Juice Concentrate | 3.00 | 1-10+ |
| Dextrose | 1429.00 | 500-3000+ |
| Other Tablet Ingredients: | | |
| Aqueous Shellac | 388.40 | 125-750+ |
| Corn Starch NF | 80.00 | 25-160+ |
| Hypromellose USP | 32.40 | 10-65+ |
| Stearic Acid NF (Vegetable Grade) | 19.50 | 6.5-35+ |
| Triacetin FCC/USP | 19.30 | 6.5-40+ |
| Magnesium Stearate NF/FCC | 7.00 | 2.5-15+ |
| Silicon Dioxide FCC | 2.50 | 0.75-5.0+ |

Formulation II was provided by mixing the actives with corn starch, stearic acid, magnesium stearate and silicon dioxide into a tablet, and coating the tablet with the shellac, triacetin and the hypromellose. The shellac was either a European shellac (Aphoeline-1) or an Indian shellac (Aphoeline 2), as described above.

Formula III used a coating composed of 2% clear polyvinyl alcohol (PVA) coating plus 14% of a nutrateric coating (Aphoeline-O). The clear coating was made up of polyvinyl alcohol, talc, polyethylene glycol, polysorbate 80; the nutrateric coating was made up of ethyl cellulose, ammonium hydroxide, medium chain triglycerides, oleic acid, and stearic acid. The proprietary blend of active ingredients comprised sodium alginate and dextrose, 1150 gm (85% by weight of Formula III).

Protocol Testing.

All subjects were volunteers that signed an informed consent in regard to the GRAS compliant supplement which was to be administered. Each subject presented fasting, with the last oral intake having occurred the night prior. Baseline lab work was completed, including blood glucose, insulin and, c-peptide, as well as other hormones. Samples were collected by licensed professionals, and handled by professional lab technicians. The sample tubes were labeled according to a preset protocol, for anonymity and shipped in frozen containers as specified by the contracted, licensed labs for testing.

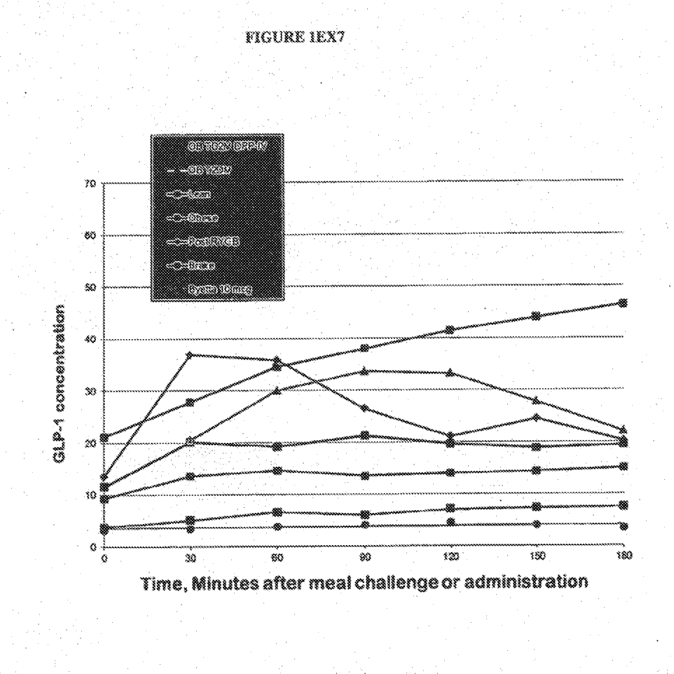

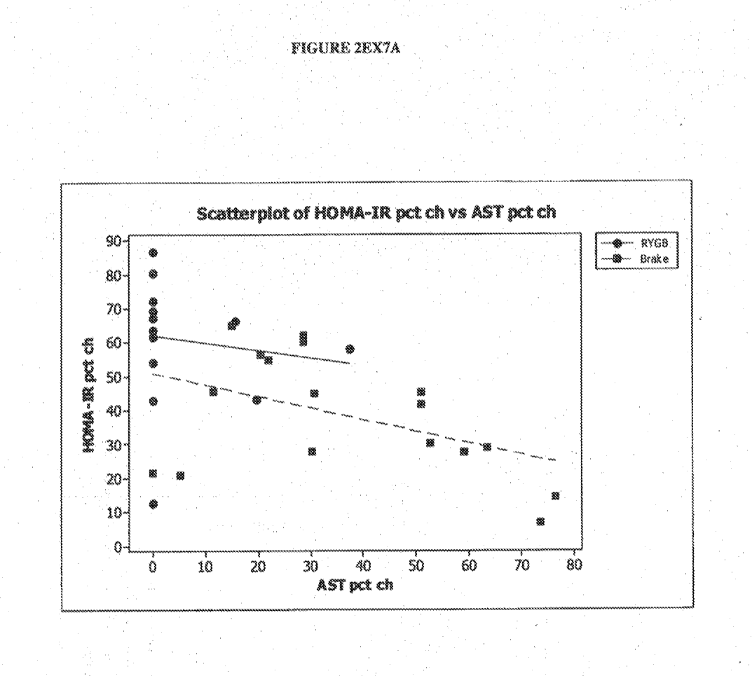

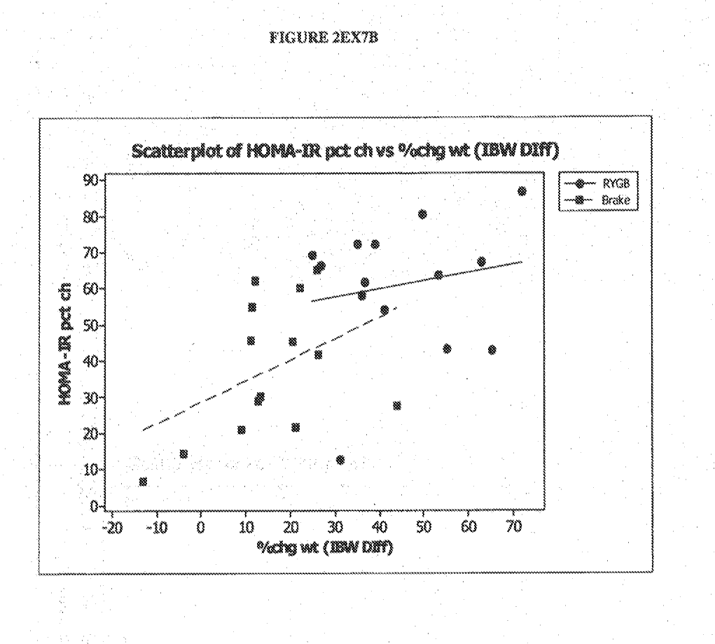

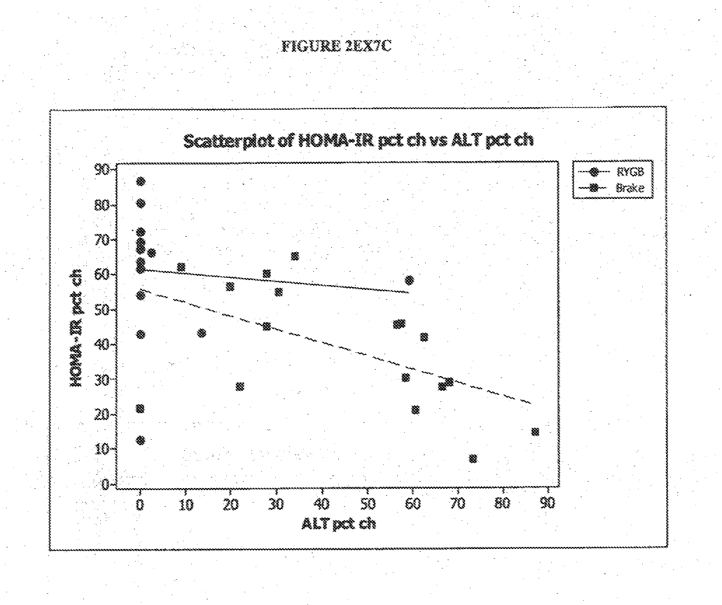

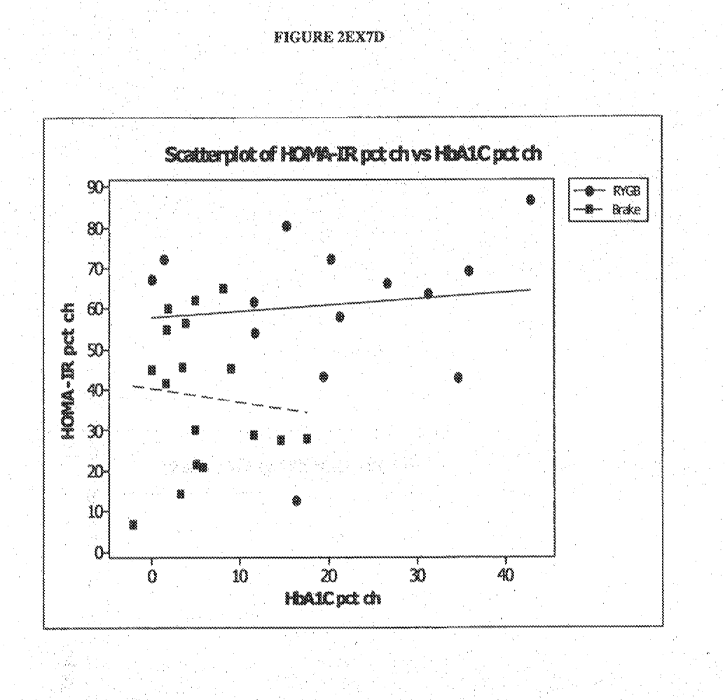

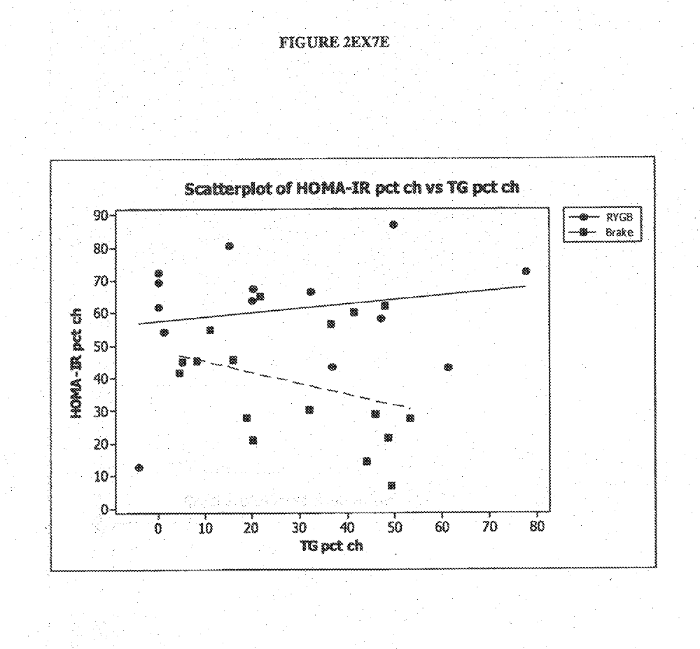

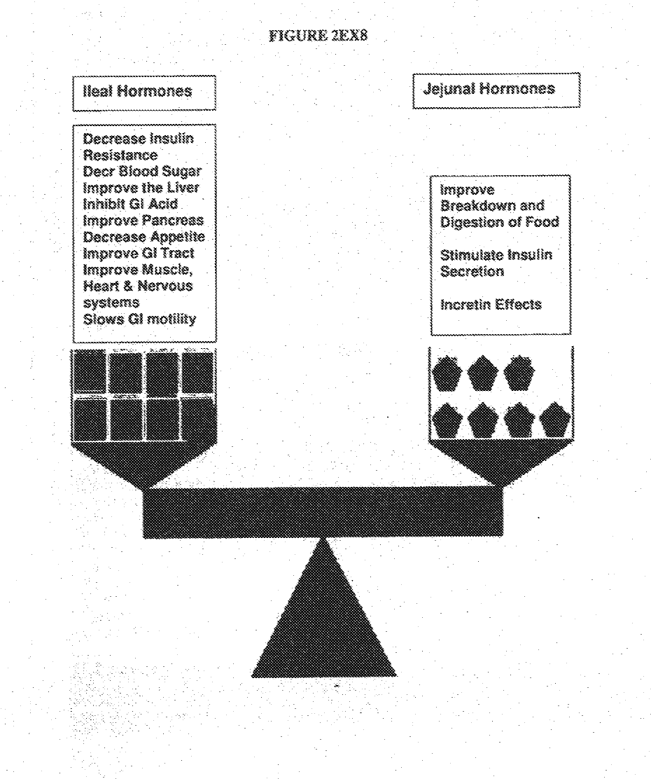

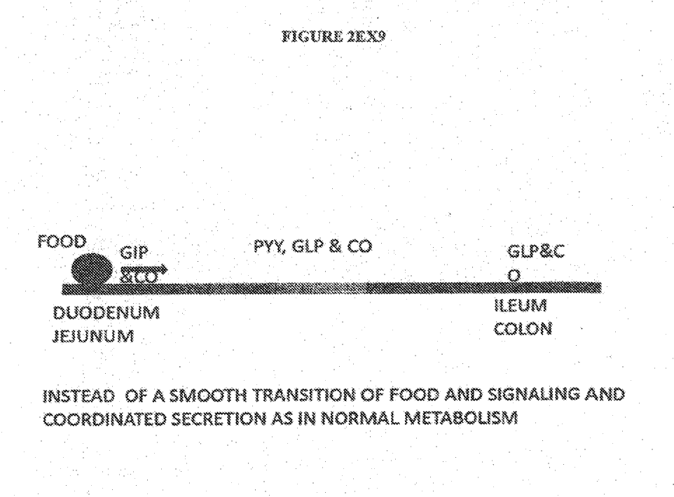

Sampling was done on an hourly basis, before and after the oral ingestion of the supplement. Vitals were taken before each draw. No food or drink was allowed prior or during the test, though water was allowed adlib. The results were compiled in the enclosed Tables, illustrated by the enclosed charts comprising FIGS. 9-28 and Tables 1-21.

The subjects selected were part of a much larger group, with only those that were found to have abnormal insulin or abnormal blood glucose or both included. There were no significant changes in levels of insulin, glucose or c-peptide for the rest of the group.

As evidenced by the figures and the corresponding tables, generally, blood glucose as well as insulin decreased and/or stabilized, in response to administering the ileal brake hormone releasing substance, which apparently results in a hormonal stimulation. This response appears to be greater the higher the starting value, indicating a significant decrease in insulin resistance. Also it can be noted that the more normal the value of insulin and glucose, the less significant are the changes to their values, indicating that the effect of the pill is self-limiting, that is, surprisingly, the ileal brake hormone releasing substance acts favourably to correct abnormal level shut does not pose a danger of decreasing blood glucose below normal, so there is no risk for hyperglycaemia. This makes the ileal brake hormone releasing substance particularly useful in persons who are only exhibit pre-diabetic symptoms, where drug therapy has not yet been indicated or is not preferred given the risk of side effects.

Established safe and effective dose ranges in humans for the ileal brake hormone releasing substance of the invention ranges from 500 to 12500 mg/day, preferably within the range of about 7,500 mg/day to about 12,000 mg/day, preferably about 10,000 mg/day. While not being limited by way of theory, the product therefore negates/reduces insulin resistance, thereby allowing blood glucose to enter the cells, with insulin at normal levels, as opposed to the abnormally high levels of insulin generated in the test subjects, and therefore decreasing insulin levels to baseline. This allows the body to use mote energy while decreasing the noxious effect of high insulin that promote obesity as well as the vicious cycle associated with high insulin levels, such as per metabolic syndrome, polycystic ovaries, arteriosclerosis, hypertension, fatty liver, etc.

The insulin production modulation achieved by administering the inventive formulating containing GRAS ingredients is believed to occur through the action of a stimulated hormone within the lower gut, which either acts through IGF like receptors or through a different receptor than foe receptor for IGF or insulin, possibly like receptor IRR. Since the ileal brake hormone releasing substance composition is not absorbed and appears to work through hormone stimulation, a new hormone from the same area could be stimulated as well that acts on a receptor, either its own or through IGF stimulation.

Accordingly, pursuant to foe present invention, it was discovered that, a ileal brake hormone releasing substance composed of GRAS compliant ingredients is effective in treating noninsulin dependent diabetes mellitus, pre-diabetic symptoms, and insulin resistance, with no side effects, by acting to suppress insulin resistance, lower/stabilize blood glucose, and therefore could be used in heating all form of insulin resistance as per NIDDM, polycystic ovary as well as type b insulin resistance.

### Discussion of Experimental Results: Examples 1-4

GLP-1, an insulinotropin, hormone released from the intestinal L cells in response to nutrient ingestion, has been extensively reviewed with respect to beta-cell function. GLP-1 is both a gut-derived hormone and a neurotransmitter synthesized in the brain. Early reports suggested that GLP-1 acts in the periphery to promote insulin secretion and affect glucose homeostasis, whereas central GLP-1 reduces food intake and body weight. However, current research indicates that in fact, GLP-1 in each location plays a role in these functions. There is substantial evidence for involvement of peripheral and brain GUM in food intake regulation and glucose homeostasis and proposes a model for the coordinated actions of GLP-1 at multiple sites. (19) However, GLP-1 receptors are abundant in many other tissues. Thus, the function of GLP-1 is not limited to the islet cells, and it has regulatory actions on many other organs. For example, it has been suggested that GLP-1 may have benefit in Congestive Heart Failure (20). GLP-1 has the ability to modulate myocardial glucose uptake and thereby make an impact on cardio protection, (This is for improving muscle function and heart) Glucose-insulin-potassium (GIK) infusions have been studied for decades, with conflicting results regarding benefit in acute myocardial infarction. Based on the same concepts, GLP-1 has recently been, demonstrated to be a more effective alternative in left ventricular (LV) systolic dysfunction (20).

A review of published, peer-reviewed medical literature (1987 to September 2008) on the extra pancreatic actions of GUM was performed (21). The extra pancreatic actions of GLP-1 include inhibition of gastric emptying and gastric acid secretion, (this is to help in decreasing acid secretion and prevention of cancer of the esophagus) thereby fulfilling the definition of GLP-1 as an enterogastrone. Other important extra pancreatic actions of GLP-1 include a regulatory role in hepatic glucose production, the inhibition of pancreatic exocrine secretion, cardio protective and cardio tropic effects, and the regulation of appetite, and stimulation of afferent sensory nerves. The primary metabolite of GLP-1, GLP-1 (9-36) amide, or GLP-1m, is the truncated product of degradation by dipeptidyl peptidase-4. GLP-1 has insulinomimetic effects on hepatic glucose production and cardiac function. Exendin-4 present in the salivary gland of the reptile, Gila monster (Heloderma suspectum), is a high-affinity agonist for the mammalian GLP-1 receptor. It is resistant to degradation by dipeptidyl peptidase-4, and therefore has a prolonged half-life. In conclusion, GLP-1 and its metabolite have important extra pancreatic effects particularly with regard to the cardiovascular system and insulinomimetic effects with respect to glucose homeostasis. These effects may be particularly important in the obese state. (21).

Given the above importance of GLP-1 and the effect of increasing its levels even higher, the use of a DPP-IV inhibitor in conjunction with an orally administered ileal brake hormone releasing substance as disclosed herein should work much better than die peripherally injectable GLP-1 medications that lack the primary portal concentration that control blood glucose and hepatic glucose release as well as insulin secretion and mesenteric fat use, acting in a physiological way will prevent complications and side effect and improve outcome. Therefore the use of Brake™ with DPP-IV inhibitors available on the market can target type 2 diabetes and prediabetes and serve as a pharmaceutical medicament more powerful and natural with fewer side effects in metabolic syndrome manifestations.

By contrast, food-related stimulation of GLP-1 is hypo-responsive or even absent in obese patients. The ileal brake is down regulated, Marks et al. also showed a remarkable absence of GLP-1 response to oral glucose in obese patients (22), indicating a down-regulation of the ileal brake pathway in the pathogenesis of obesity. On the other hand, obese patients who undergo bariatric surgery lose weight gradually by suppression of appetite. They also experience very positive impact on glucose levels in the blood and improvement in insulin resistance. One possible explanation for all of these effects is a remarkable reactivation of the dormant ileal brake pathway by bariatric surgery, just as would be expected from the experiments delivering high amounts of Brake or its components to the ileum via an enteric tube (23,24). Thus, the present invention also relates to its use as an alternative therapy or concomitant therapy or pretherapy or post therapy to bariatric surgery)

In 1996 it was postulated that this stimulation happens via neurotransmission (25), and to some extent involves GIP indirectly via neuron-stimulation of the ileal brake hormones. The effect could be inhibited by lowering neuron stimulation using blockers. Others have challenged these findings, and alternatively proposed that the ileal brake effects are mediated directly by the L-Cells that are found throughout the intestinal tract. In fact they argue that the effect on L-Cells coexists with the GIP hormones in the upper jejunum and with PYY in the lower gut.

Fractionation experiments with enteroglucagon resulted in isolation of GLP-1 and GLP-2. Because of its insulin activity GLP-1 is used to treat diabetics, and was noted to have significant weight loss properties. Analogues to GLP-1 made available for treatment of diabetes such as Exenatide (Byetta) are associated with favorable glucose control and appetite suppression associated weight loss. Other hormones in the ileal brake pathway, such as PYY analogues, were also made available and trials were also designed to use these in the treatment of human obesity.

Holst and colleagues (2006) published a detailed review on the action of GLP-1 on different parts of the body to include the muscles, nervous system, the heart as well as the pancreas the liver intestine and brain (26). GLP-1 was shown to be a powerful regulator of food intake in humans at physiological levels (27,28). GLP-2 targets growth and regeneration of the enteric organs, therefore acting as a growth factor hormone which serves in the recovery of the body from injury (32-37). This will help the body to recover from injury related to event such as chemotherapy, radiation, mechanical injuries such as surgeries or trauma, or infections. PYY was shown to induce satiety as well as to suppress acid secretion combined with GLP-1, and act on motility significantly (38,39). PYY was also tested by both injection and nasal administration, but was itself unsuccessful for prevention and treatment of obesity. Some studies suggest that stimulation of all the hormones of the ileum simultaneously worked synergistically to suppress appetite and regulate both glucose and insulin, and the result of this synergy was notable by its actions at lower doses and mainly on the portal system.

Beside the above we noted that triglyceride levels decreased even more significantly tan above liver enzymes indicating that the present invention can be used to target steatohepatitis as well as hyper triglyceride. On the topic of liver injury and fatty liver disease, one patient under treatment for Hepatitis C genotype 1a, experienced a reversal in the virus count during a conventional therapy with interferon and ribavirin therapy that usually is interpreted as resistance of the virus to treatment hack to a normal responsive trend, indicating a change in the patient's immune response to the therapy.

On another subject, a female patient under treatment for autoimmune hepatitis worsening liver enzymes and meld score on steroids and cellcept, improved her liver enzymes again indicating an improvement and change in the patient immune system indicating a more generalized indication for liver disease beyond the metabolic condition or another explanation that all liver diseases have a common important factor in response to any injury that relate to the way the liver respond to injury.

### Example 1-4 Summary

Injection of analogues of GLP-1 peripherally is a familiar approach in the treatment of diabetes, and produces appetite suppression in a manner similar to Aphoeline/Brake treatment. However, the properties of peripheral GLP-1 include a different biodistribution pattern and a short half-life of approximately 3 minutes. The majority of the dose does not enter the portal system as it would if GLP-1 was induced by GI tract stimulation and with peripheral administration less than 15% will go through the liver to the periphery. While exogenous use of enteric ileal brake hormones is demonstrated to have an effect on appetite suppression, the idea of using an oral formulation to reset, modulate or stimulate the endogenous ileal brake in the lumen of the GI tract has not been tried before, other than by RYGB surgery. The novel action of a formulation on the ileal brake pathway is of major advantage over peripheral subcutaneous injection, because this pathway is optimally activated LOCALLY in the distal small bowel. There are more substances than GLP-1 released with ileal brake activation, and when stimulated properly these ileal brake hormones act synergistically and in a highly complementary manner, which both avoids side effects associated with only one of them administered parenterally, and produces: an optimal exposure of the hormones to the pancreas, liver and the anterior GI tract. Thus the peripheral injection approach to use of GLP-1, although proven to have appetite suppression, is partly a delivery site problem. For example, subcutaneous injection of GLP-1 mimetic, at supra-physiological levels, does not confer the advantages of portal application of lowered amounts. Thus the liver and pancreas effects are not beneficial; only the brain appetite suppression axis is activated by peripheral subcutaneous injection. Furthermore there are GLP-1 receptors in non-target organs like the heart and kidney, and these may explain some, of the recently noted side effects of Exenatide. Thus the portal system is where most of the action is taking place, and activation of the local ileal brake pathways lead to the full complement of benefits beyond appetite suppression. With oral administration of Brake, there is appetite suppression, but also beneficial effects on glucose control, insulin pathways, re-set pancreatic glucose sensors, hepatic glycogen storage and glucose release, and mobilization of adipose tissue.

The actions controlled by Aphoeline/Brake and the biomediators released therefrom are in the GI tract itself all the way from the esophagus to the rectum. Another problem with peripheral GLP-1 is the development of antibodies to the peptide within one year and up to 40% of the treated patients with Exenatide. The other side effects of Exenatide include pancreatitis and renal failure associated with the treatment. These should not occur with local release of GLP-1 from use of Brake.

On reviewing the literature in regard to appetite control and obesity the mainstream approach has been caloric counting and exercise. Excessive caloric intake has been linked to a psychological problem. As a consequence, from the patient viewpoint they are either addicted to food without will power or the patient is not sufficiently active to compensate for the intake of calories (49). Though valid, these statements do not give an accurate picture of the problem afflicting the large proportion of patients that appear to be very balanced psychologically and despite their best efforts are not capable of losing weight. Some reviews suggest that people under stress tend to lose less weight than people under less stressful situations, ascribing cortisol as the etiological factor. Other studies using a rat model (48) suggest that obesity is predetermined and one will tend to go back to the genetic curve with age.

We do know that certain conditions, including diabetes, hypertension, insulin resistance, commonly used antidepressants and anti-psychotics are associated with weight gain. The effect of bariatric surgery on patients with obesity and concomitant diabetes also seem to be mediated thru the suppression of appetite centrally after local GI activation of the ileal brake pathway. There is the likelihood that use of Brake in combination with centrally active compounds that stimulate appetite, such as olanzapine (Zyprexa), will offset the weight gain disadvantage of these drugs, giving rise to combination products such as ZyprexaBrake. The mechanism of action is not psychological as oral caloric intake and energy expenditure, since patients with a RYGB surgery for obesity have improved appetite control compared to people that undergo a lap band surgery. The effectiveness of RYGB surgery is also related to the connection site of the bypass. Make it too short and severe malabsorption results, while if the loop is too long the patient does not lose weigh t. The site of the surgical connection clearly influences the activation of the ileal Brake. Another consistent observation is the favorable weight loss action of Liraglutide in spite of no major changes in patient behavior or lifestyle (29).

The other approach to the treatment of obesity is to try to bypass different systems like providing medications that work directly on the appetite control center by different medications that are available on the market. The different side effects that will have to be dealt with include hypertension, stroke, addiction, seizures, cardiac arrhythmias and coronary events, pulmonary hypertension, severe depression, suicide, and insomnia. Even when the patient loses weight, there is a rebound off medications associated with binge eating and the patient ends up being either recycled in the system for another course of therapy in weight control centers, or gaining more weight than he started with, putting him at risk that could be higher than the baseline due to the severe weight fluctuations over short periods of time.

Vildagliptin is a selective dipeptidyl peptidase IV inhibitor that augments meal-stimulated levels of biologically active glucagon-like peptide-1. Chronic Vildagliptin treatment decreases postprandial glucose levels and reduces hemoglobin A1C in type 2 diabetic patients. However, little is known about the mechanism(s) by which Vildagliptin promotes reduction in plasma glucose concentration. METHODS: Sixteen patients with type 2 diabetes (age, 48+/−3 yr.; body mass index, 34.4+/−1.7 kg/m2; hemoglobin A1c, 9.0+/−7-0.3%) participated in a randomized, double-blind, placebo-controlled trial. On separate days patients received 100 mg Vildagliptin or placebo at 1730 h followed 30 min later by a meal tolerance test (MTT) performed with double tracer technique (3-(3)H-glucose iv and 1-(14)C-glucose orally). RESULTS: After Vildagliptin, suppression of endogenous glucose, production (EGP) during 6-h MTT was greater than with placebo (1.02+/−0.06 vs. 0.74+/−0.06 mg·kg-1·min-1; P=0.004), and insulin secretion rate increased by 21% (P=0.003) despite significant reduction in mean plasma glucose (213+/−4 vs. 230+/−4 mg/dl; P=0.006). Consequently, insulin secretion rate (area under the curve) divided by plasma glucose (area under the curve) increased by 29% (P=0.01). Suppression of plasma glucagon during MTT was 5-fold greater with Vildagliptin (P<0.02). The decline in EGP was positively correlated (r=0.55; P<0.03) with the decrease in fasting plasma glucose (change=−14 mg/dl). CONCLUSIONS: During MTT, Vildagliptin augments insulin secretion and inhibits glucagon release, leading to enhanced suppression of EGP. During the postprandial period, single dose of Vildagliptin reduced plasma glucose levels by enhancing suppression of EGP. (40)

Other approaches to weight loss target absorption, create states of malabsorption, produce stool incontinence, and may result in fatty liver another undesirable effects (51).

Based on these premises leaders in the field started to emphasize a more natural GI tract based approach to weight loss that would involve all the endogenous mechanisms that regulate caloric intake and body weight. The goal was to lose more weight with fewer side effects, and the standard is RYGB Surgery. A recent review of approaches to this problem eloquently summarizes the field (17, 41-44). The focus is shifting to the ileal brake pathways that are using the body natural signals; the gut hormones for future research of obesity pharmacotherapy (45,46). It was discovered that RYGB should be the standard for comparison of the actions of Aphoeline/Brake, considering both, physiology and mechanistic pharmacology. The RYGB and the oral formulation were shown, for the first time, to be acting in a nearly identical manner. The only difference is greater weight loss tram RYGB, but that was to be expected because the size of the stomach is reduced dramatically in RYGB, while there is no change in stomach size when taking Brake.

Based on our clinical observations, there is a component of hunger and obesity that is visceral and unconscious. To a certain extent, these effects are unknown to the patient, making it very difficult for the person to control appetite. The person at the time will be trying to replace the lack of visceral perception with an alternative voluntary conscious awareness resulting in continuous monitoring of the calories and input output as well as calories used and activity at all time to control the weight. This is difficult, and often causes frustration to those attempting to lose weight in this manner. The action of the ileal Brake is involved in the Selective Modulation of Appetite, and control of appetite in this both conscious and unconscious state. To some extent, the lower GI tract influences appetite for foods that the body needs, and the tuning of these appetite pathways is controlled both by consumption and by expenditure. With respect to glucose control, the teachings of the supply side model amplify the understanding of these pathways and their contribution to long term weight and to control of Type 2 diabetes via diet and exercise. The surprising observation was the impact of the ileal brake hormones on control of type 2 diabetes, and the homology between RYGB and Brake.

Going back to the literature trying to figure out the different responses of the body to food between normal and overweight or obese patients, the only significant abnormality that was reported, is the response of the ileal break to the intake of the mixed meal (17, 22), and more specific to carbohydrates. Therefore it seems the natural appetite suppressive pathways become tolerant to the intake of carbohydrates. This partially explains the success of the Adkins diet, even though in this case there arene demonstrable differences in the anatomy or histology of those two groups, except in rare cases of severe morbid long term obesity associated with atrophy of the ileum. Given the fact that food delivered to that part of the intestine is capable of stimulating diose hormones independently of oral intake and the fact that the ileal stimulation during a mixed meal can be inhibited by suppressing the neurotransmission, raise the possibility that the problem seems to be about the transmission of the signal from gut to brain. It is possible that a reset of a carbohydrate-tolerant ileal brake pathway will re-set the appetite center and renews the feedback loop that interrupts eating, all without progression to a metabolic syndrome. Therefore if we are able to directly stimulate die ileum in the manner of RYGB with an orally administered formulation, we should be able to restore the ileal brake signal and at least give the patient some help in restoring visceral signals that measures the food intake.

These visceral signals are not only important to control of metabolic syndrome abnormalities but as reported in review articles (34,44) these hormones are extremely beneficial to the patient. Their absence during down-regulation could be what the patients am seeking unconsciously when they overeat. Since these hormones are also very important in the homeostasis of the insulin and glucose levels they will help tremendously in the use is of the reserves that are already present Finally there is new evidence that gut derived inflammation, itself an effect of food and intestinal bacteria, is regulated by the hormones released by the ileal brake pathway, and that for the first time RYGB surgery and oral administration of Brake control these long term inflammation pathways. When out of control, these pathways lead to metabolic syndrome manifestations such as atherosclerosis, and perhaps contribute to deposition of metabolic byproducts such as amyloid in the brain, thought to be an important pathway in Alzheimer's disease. In this manner use of Brake would improve atherosclerosis or Alzheimer's disease, beneficial effects already attributed to RYGB surgery.

By stimulating the hormones naturally with Aphoeline/Brake™ we are delivering the majority of the hormones where they belong in the portal system, where they have the most powerful impact on the pancreas and the liver. We were also encouraged by the fact that RYGB surgery for obesity is capable of stimulating those hormones in all patients, indicating that the innate ability of these hormones to respond is still present.

We set a goal to stimulate the ileal hormones with an oral formulation of GRAS ingredients, created to become an ileal brake hormone releasing substance that mimics the action of RYGB surgery. The data from a comparison of Aphoeline/Brake with RYGB are compelling and the stimulation of the ileal brake pathway seems independent of age or weight or diabetes. This establishes the intestine still functions despite obesity, and the problem seems to be in the down-regulation of the signaling from the ileum. (Another confirmation to that statement comes from the RYGB surgical procedure that in appropriate individual triggers the same process).

What we discovered from oral formulations given to modulate ileal brake hormone release, is that local stimulation of the ileum in this manner has a very powerful effect on the glucose and insulin homeostasis, leading to a rapid decline in of insulin resistance. Insulin resistance is the first major biomarker to change in response to either the oral use of Brake or to RYGB surgery. We discovered that the ileal brake pathway is not a means of further stimulating insulin, but rather a reduction of glucose supply side delivery leading to a reduction of insulin resistance that occurs well before the patient begins to lose weight. This is also consistent, with the data from RYGB surgery, where the reduction in insulin resistance occurs within a few hours of surgical anastomosis, again much earlier than any weight loss.

The more powerful effect on steatohepatitis, seen by decrease of the enzymes level to normal within 3-4 weeks of treatment with Aphoeline/Brake need to be studied on a much longer duration to confirm the trend and the gains, but it seems from the reduction in endotoxin, inflammation, insulin resistance and the trend to normalize triglyceride and cholesterol as well as to the surprising improvement of all parameters including platelets that die trend is true. Similar platelets trend is seen in cirrhotic patients (non-published data).

Based on the recent publication of Liraglutide and weight loss (29), the GLP-1 family of gut hormones will induce weight loss but in a different way than expected, the weight loss is slow and happens after other parameters start to improve. Weight loss is insidious, just like weight gain, and occurs on a rather unconscious level. The pathway is re-activated after being dormant and the distal caloric signals are now once again responded to in the ileum.

The advantage of having an oral stimulation of all the ileal hormones is the synergistic effect of the hormones that were meant to be stimulated together in a broad pathway beyond any individual component. The fact that these hormones are released in the portal system that seems to be the center of all metabolism except the muscles and the brain, the fact that the highest concentration of these hormones is in the portal system make our stimulation much less intrusive and more efficient than the peripheral administration of such hormones.

The mechanism for the suppression of insulin resistance needs further investigation. Although we showed that the IGF system is stimulated, we do not feel this is the only answer to the question; other peptides as well as other cellular receptors such as the RR receptors need to be investigated as part of the equation. In the next section, we prioritize our future work in tins direction.

### Additional Objectives Relating to Examples 1-4 (Figures Labeled with E)

### Project Description

Given that the most natural way to stimulate those hormones is the use of an oral formulation for gut stimulation of the ileal brake pathways, we devised a project and a product to both stimulate and then reset the ileal brake in patients. The major goals were:

1. To establish proof of concept with oral activation of the ileal brake pathways, whereby an oral pill containing food content that is protected with an enteric-coating mechanism, could deliver this food content: to the distal ileum, and thereby stimulate ileal brake hormones.
2. To demonstrate that stimulation of the ileal brake with this formulation is reproducible and can cause the released ileal hormones to reach significant levels physiologically in humans.
3. To determine a time related pattern of response to stimulation of the ileal brake and to use the local enteric stimulation as means of re-setting the ileal brake of obese patients.
4. To demonstrate stimulation of the ileal brake in overweight and obese patients.
5. To demonstrate that the increase in the hormones of the ileal brake cause weight loss in obese patients by regulating gut-brain signaling and therefore lower appetite.
6. To study the interactions between ileal brake hormones and systemic effects, such as control of blood glucose, insulin homeostasis, and appetite control.
7. To establish doses, administration times and optimal schedules for Aphoeline/Brake™ in treated patients with obesity.

This project was designed to reset a biological process regulating appetite. It tests an endogenous pathway that appears to be hypo-responsive obese patients. It is believed that a reset of the ileal brake mimics the effect of bariatric surgery in the obese patient, without exposing the obese patients to the risks of surgery. If successful, the product will use an existing pathway that protects from the harmful effects of metabolic syndrome, and the associated controls and feedback loops, avoiding complications and side effects. Use of Brake™ will help the body regain control of the intestinal factors that regulate ingested nutrients and weight. Furthermore, giving patients control of an unconscious pan of appetite control, a pathway dial is very difficult to deal with at the conscious level, will make it easier for them to follow a diet and lose weight There is no evidence that the hypo-responsive ileal brake in obese patients is an organic defect that cannot be subject to external regulation, although it is theoretically possible since some patients do not respond to bariatric surgery.

### Methodology

As a starting point we needed to calculate the amount of food needed to deliver to the ileum. For that purpose we decided to use carbohydrate as a starting solution. Carbohydrate is a significant stimulus to the ileal brake mechanism (12), and it was easy to monitor for any absorption or failure of the pill by checking the blood glucose level. Finally, absorption of carbohydrate stops much sooner than fat and gives us more room for the initial testing of the oral formulation.

Based on the above we have to calculate the right amount of calories to be delivered to the ileum. We decided to proceed with testing the minimal amount of carbohydrate needed to stimulate insulin and be visible in the blood stream; we termed this a minimal metabolic unit. The drought behind it was that if the upper gut was able to perceive it as food, the lower gut that is supposed to monitor malabsorption should be able to react to it as a signal of malabsorption. It was determined that the unit should be between 8 to 15 gm of carbohydrate. The amount used in the direct ileal stimulation experiments was around 15 gm (12).

The second task was to have the coating for the pill to deliver the carbohydrate to the ileum without proximal small bowel absorption. This required a slow release formulation to avoid an osmotic side effect.

Because of the amount of carbohydrate involved in re-setting the ileal brakes the goal was to decrease the number of the pills, starting at 18 and decreasing the number to a manageable level of 7 per day. The formulation and dose finding experiments started in 2003, and by 2008 we had arrived at 4-5 different formulations that withstood these in-vitro challenges and were ready for testing.

Three trials were conducted with pilot formulations to arrive at the components of Aphoeline (pursuant to the formulations, provided above). After informed consent front healthy volunteers, monitored at all times medically the pills were given after an overnight fasting state and blood work was drawn on an hourly basis for 10 to 12 hours testing. Measured were the peptide ileal brake associated hormones and their associated biomarkers: blood glucose, insulin, c-peptide, and in the last tests IGF-1, IGF-2. Patients were allowed to drink water ad libitum. The samples were drawn according to the recommendations of the various specialized labs by professional registered nurses, and the blood was handled on the premises by a reference lab (which one) immediately on withdrawal, each tube coded accordingly packaged on dry ice and shipped overnight to the specialty labs.

Patients were separated in different groups. The groups were handled sequentially. Each subject in the group was handled simultaneously with the other elements of his group at a separate drawing station with a registered nurse, according to the time schedule. Therefore group 1 was done all at one time at the different stations from one to seven, the time frame was kept by an independent monitor to try to assure punctuality.

Initially the groups were processed and paper were filled out with a short history and physical, consent were signed, and a heparin lock was placed by the nurse at the station, then a draw at zero time was done, time was marked then the pills was given to all individual of group at the same time. The same was done to the other groups sequentially. Blood was drawn thereafter as per protocol on an hourly basis on the clock for all members of the group simultaneously, at every draw the person and vitals were assessed and blood drawn from the heparin lock, after saline flush and after discarding the first cc s to avoid high heparin concentration. For testing the GLP-1, GLP-2, and PYY was as follows: EDTA (purple top) tubes with addition of 500 micro liters of Aprotinin and 10 micro liters of DPP IV per tube. Collect blood, centrifuge within 10 minutes in a 4 degree G centrifuge. Pour off supernatant (plasma) and immediately freeze. Label and code each tube separately according to a pre organized labeling system. The tubes were Stored and ship these specimens at −70 C. The Insulin, C-peptide and glucose were collected in SST tubes, spun and sent to the local national lab.

Heparin lock, after saline flush and after discarding the first cc s to avoid high heparin concentration. The blood was placed in 2 separate tubes from the same draw to assure redundancy and control, in Vacutainer tubes containing protease inhibitors (EDTA, Aprotinin, and DPP IV inhibitor) cocktails. After blood collection and centrifuged in refrigerated centrifuge, in those tubes, then transfer the 2.5 ml plasma to a container or combine two plasmas from the same subject at “same time point” into a 6 ml container. To freeze, labeled and code each tube separately according to a pre organized labeling system then ship in dry ice as soon as possible to the peptide labs measurement preferably overnight.

Results were reported from the reference lab and decoded back in standard excel format, and forwarded to us for analysis.

The hormone data set was statistically analyzed; the results are described in the next section.

### Results of Statistical Analyses

Aphoeline has been developed after testing a sequence of formulations and careful statistical analyses of the blood test results. Testing was done at three different times with three different formulations, as shown in Table 1:

TABLE 1
Time of testing and formulation

| Time | Formulation | SUBJECTS* |
|---|---|---|
| August 2008 | Aphoeline -1 | A, F, G, H, I, J, K, P, U |
| September 2008 | Aphoeline-1 | E, K, N |
| Oct. 26, 2008 | Aphoeline-1 | A, B, C, D, E |
| Oct. 26, 2008 | Aphoeline-1 | F, G, H, I, J |

*There were different subjects at different testing times [e.g., Subject A in August testing is not same as Subject A in October testing] Formulations described above.

### Results of Statistical Analysis

The R software package for statistical computing was used for all statistical analyses and data visualization.
1) Measurements of GLP1, GLP2, and IGF-I, IGF-II, Glucose, Insulin, C-Peptide and PYY for; each of the 10 subjects were plotted against time (FIGS. 1B, 2E, 3E, and 4E).
2) It can be seen from FIG. 3E (Further Examples) that [i] all 5 Aphoeline subjects [F, G, H, I, J] have elevated Glucose levels at time 0, [ii] except for subject G, the Glucose level monotonically decreases to normal levels; in the case of Subject G, Glucose level starts at 113, goes down to 98, goes up to 112 and then goes down to 108,
3) It is also apparent from FIG. 3E that two of the subjects [G and I] in the Aphoeline Group had slightly elevated insulin levels at time 0; in both of these cases, the insulin levels decreased by time 10.
4) FIG. 5E (Further Examples) shows the average concentrations of GLP1, GLP2, IGF-3, IGF-II, Glucose, Insulin, C-Peptide and PYY plotted against time of measurement for the Aphoeline-0 Group (concentrations at each time averaged over the subjects A-E), and FIG. 6E shows these averages for the Aphoeline Group (concentrations at each time averaged over the subjects F-J). We can see from FIGS. 5E and 6E that the average concentrations of Glucose and insulin decrease with time.
5) Mann-Kendall nonparametric test for trend was used to determine if both insulin and glucose levels decrease over time for Aphoeline-0 and Aphoeline Groups. These results are shown in Table 2, below

TABLE 2
Results of Mann - Kendall nonparametric test for trend

| | | | P - value for | | P - value for |
|---|---|---|---|---|---|
| | | Mann - | the alter- | Mann - | the alter- |
| | | Kendall | native | Kendall | native |
| | | Statistic τ | hypothesis of | Statistic τ | hypothesis of |
| | Sub- | for | decreasing | for | decreasing |
| Product | ject | Glucose | trend | Insulin | trend |
| Aphoeline -0 | A | −.5 | .02* | −.299 | .12 |
| Aphoeline -0 | B | −.64 | .005* | −.441 | .055** |
| Aphoeline -0 | B | −.524 | .015* | −.554 | .015* |
| Aphoeline -0 | D | .82 | .0003* | .04 | .94 |
| Aphoeline -0 | E | −.496 | .025* | −.93 | .00005* |
| Aphoeline | F | −.774 | .0007* | .0556 | .44 |
| Aphoeline | G | −.112 | .35 | −.33 | .09** |
| Aphoeline | H | −.389 | .06** | .11 | .35 |
| Aphoeline | I | −.624 | .005* | −.352 | .08** |
| Aphoeline | J | −.61 | .007* | — | — |

*Downward trend significant at test size 0.05,
**downward trend significant at test size 0.1

TABLE 3
Results of Mann-Kendall nonparametric test for trend

| | | Mann-Kendall Statistic τ for | | | | | |
|---|---|---|---|---|---|---|---|
| | | Glucose | | C-Peptide | | Insulin | |
| Product | Subject | τ | P-value | τ | P- | τ | P-value |
| Aphoeline-0 | A | −.66 | .003* | −.673 | .003 | −.636 | .004* |
| Aphoeline-0 | F | −.86 | .0002* | −.722 | .002 | −.807 | .0004* |
| Aphoeline-0 | G | −.697 | .002* | −.66 | .003 | −.236 | .35 |
| Aphoeline-0 | H | −.648 | .004* | −.74 | .002 | −.6 | .006* |
| Aphoeline-0 | I | −.611 | .006* | −.785 | .000 | −.455 | .03* |
| Aphoeline | J | −.623 | .005* | −.648 | .004 | −.309 | .10** |
| Aphoeline | K | −.597 | .01 | −.472 | .03 | −.236 | .17 |
| Aphoeline | P | −.908 | .0001* | −.785 | .000 | −.382 | .06** |
| Aphoeline | U | −.572 | .01* | −.86 | .000 | −.855 | .0002* |
| Aphoeline | E | −.785 | .006* | −.927 | .000 | −.527 | .015* |
| Aphoeline | K | −.917 | .00006 | −.85 | .000 | −.341 | .10** |
| Aphoeline | N | −.774 | .0007* | −.88 | .000 | −.782 | .0005* |
| Aphoeline | F | −.774 | .0007* | −.812 | .000 | .06 | .88 |
| Aphoeline | G | −.112 | .35 | −.587 | .007 | −.33 | .09** |

*Downward trend significant at test size 0.05,
**downward trend signification at test size 0.1

Results for Subjects with Elevated Glucose and/or Insulin Levels

The levels of Glucose, C-Peptide and Insulin were plotted against time for a subset of the data set generated during testing, for which initial Glucose and/or Insulin levels are elevated. The levels of Glucose, C-Peptide and Insulin all return to normal for subjects taking any of the throe Aphoeline formulations [Alpholine-0, Alpholine-1, and Alpholine2].

Weight Loss Associated with Positive Side Effects

FIG. 10E shows the total weight loss observed for a patient (50 year old female) as a function of days between measurements, and FIG. 11E shows levels of liver enzymes in the same patient at the times of measurements. For this subject, Aphoeline clearly has a positive and significant effect on liver enzymes.

### Discussion

Injection of analogue of GLP-1 peripherally is a familiar approach in the treatment of diabetes, and produces appetite suppression in a manner similar to Aphoeline treatment. However, the properties of peripheral GLP-1 include a different distribution pattern and a short half-life of approximately 3 minutes. The majority of the dose docs not enter the portal system as it would if GLP-1 was induced by GI tract stimulation and with peripheral administration less than 15% will go through the liver to the periphery. While exogenous use of enteric ileal brake hormones is demonstrated to have an effect on appetite suppression, the idea of resetting the endogenous ileal brake in the lumen of the GI tract has not been tried before, other than by bariatric surgery. The ileal brake pathway is optimally activated LOCALLY in the distal small bowel, and when stimulated properly these ileal brake hormones act synergistically and in a highly complementary rammer, which both avoids side effects associated with only one of them administered parenterally. The drawback to the peripheral injection approach of GLP-1, although proven to have appetite suppression, is partly a delivery site problem. For example, subcutaneous injection of GLP-1 mimetic, at supra-physiological levels, does not allow the advantages of portal application of lowered amounts. Thus the liver and pancreas effects are not beneficial; only the brain appetite suppression axis is activated. Furthermore there are GLP-1 receptors in non-target organs like the heart and kidney, and these may explain some of the recently noted side effects of Exenatide. Thus the portal system is where most of the action is taking place, and activation of the local ileal brake pathways lead to the full complement of benefits beyond appetite suppression. With oral administration of Aphoeline, there is appetite suppression, but also beneficial effects on glucose control, insulin pathways, re-set pancreatic glucose sensors, hepatic glycogen storage and glucose release, and mobilization of adipose tissue.

The actions controlled by Aphoeline are in the GI tract itself all the way from the esophagus to the rectum. Another problem with peripheral GLP-1 is the development of antibodies to the peptide within one year and up to 40% of the treated patients with Exenatide. The other side effects of Exenatide include pancreatitis and renal failure associated with the treatment.

On reviewing the literature in regard to appetite control and obesity the mainstream approach has been caloric counting and exercise. Excessive caloric intake has been linked to a psychological problem. As a consequence, from the patient viewpoint they are either addicted to food without will power or the patient is not sufficiently active to compensate for the intake of calories (49). Though valid, these statements do not give an accurate picture of the problem afflicting the large proportion of patients that appear to be very balanced psychologically and despite their best efforts are not capable of losing weight. Some reviews suggest that people under stress tend to lose less weight than people under less stressful situations, ascribing cortisol as the etiological factor. Other studies using a rat model (48) suggest that obesity is predetermined and one will tend to go back to the genetic curve with age.

We do know that certain conditions, including diabetes, hypertension, insulin resistance, commonly used antidepressants and anti-psychotics are associated with weight gain. The effect of bariatric surgery on patients with obesity and concomitant diabetes also seem to be mediated thru the suppression of appetite centrally after local GI activation of the ileal brake pathway. The mechanism of action is not psychological as oral caloric intake and energy expenditure, since patients with a bypass surgery for obesity have improved appetite control compared to people that undergo a lap band surgery. The effectiveness of bariatric surgery is also related to the connection site of the bypass. Make it too short and severe malabsorption results, while if the loop is too long the patient does not lose weight. Another consistent observation is the favorable weight loss action of Liraglutide in spite of no major changes in patient behavior or lifestyle (29).

The other approach to the treatment of obesity is to use medications that work elsewhere than on the appetite center, evoking actions by different pathways. The different side effects that will have to be dealt with include hypertension, stroke, addiction, seizures, cardiac arrhythmias and coronary events, pulmonary hypertension, severe depression, suicide, and insomnia. Even when the patient loses weight, there is a rebound off medications associated with binge eating and the patient ends up being either recycled in the system for another course of therapy in weight control centers, or gaining more weight than he started with, putting him at risk that could be higher than the baseline due to the severe weight fluctuations over short periods of time.

Vildagliptin is a selective dipeptidyl peptidase IV inhibitor that augments meal-stimulated levels of biologically active GLP-j. Chronic Vildagliptin treatment decreases postprandial glucose levels and reduces hemoglobin A1C in type 2 diabetic patients. However, little is known about the mechanism(s) by which Vildagliptin promotes reduction in plasma glucose concentration. METHODS: Sixteen patients with type 2 diabetes (age, 48+A3 yr.; body mass index, 34.4+/−1.7 kg/m2; hemoglobin A1c, 9.0+A0.3%) participated in a randomized, double-blind, placebo-controlled trial. On separate days patients received 100 mg Vildagliptin or placebo at 1730 h followed 30 min later by a meal tolerance test (MTT) performed with double tracer technique (3-(3)H-glucose iv and 1-(14)C-glucose orally). RESULTS: After Vildagliptin, suppression of endogenous glucose production (EGP) during 6-h MTT was greater than with placebo (1.02+/−0.06 vs. 0.74+/−0.06 mg·kg-1 min-1; P=0.004), and insulin secretion rate increased by 21% (P=0.003) despite significant reduction in mean plasma glucose (213+/−4 vs. 230+/−4 mg/dl; P=0.006). Consequently, insulin secretion rate (area under the curve) divided by plasma glucose (area under the curve) increased by 29% (P=0.01). Suppression of plasma glucagon during MTT was 5-fold greater with Vildagliptin (P<0.02). The decline in EGP was positively correlated (r=0.55; P<0.03) with the decrease in fasting plasma glucose (change=14 mg/dl). CONCLUSIONS: During MTT, Vildagliptin augments insulin secretion and inhibits glucagon release, leading to enhanced suppression of EGP. During the postprandial period, a single dose of Vildagliptin reduced plasma glucose levels by enhancing suppression of EGP.(40)

Other approaches to weight loss target absorption, create stales of malabsorption, produce stool incontinence, and may result in fatty liver and other undesirable effects (51).

Based on these premises leaders in the field started to emphasize a more natural GI tract based approach to weight loss that would involve all the endogenous mechanisms that regulate caloric intake and body weight. The goal was to lose more weight with fewer side effects, and the standard is Bariatric Surgery. A recent review of approaches to this problem eloquently summarizes the field (17, 41-44). The focus is shifting to the ileal brake pathways that are using the body natural signals: the gut hormones for future research of obesity pharmacotherapy (45, 46).

Based-on our clinical observations, there is a component of hunger and obesity that is visceral and unconscious. To a certain extent, these effects are unknown to the patient, making it very difficult for the person to control appetite. The person at the time will be trying to replace the lack of visceral perception with an alternative voluntary conscious awareness resulting in continuous monitoring of the calories and input output as well as calories used and activity at all time to control the weight. This is difficult, and often causes frustration to those attempting to lose weight in this manner.

Low-glycemic index (GI) foods and foods rich in whole grain are associated with reduced risk of type 2 diabetes and cardiovascular disease. Nilsson and Holst examined the effect of cereal-based bread evening meals (50 g available starch) that varied in content of indigestible carbohydrates, on glucose tolerance and related variables after a subsequent standardized breakfast in healthy subjects (n=15). At breakfast, blood was sampled for 3 h for analysis of blood glucose, serum insulin, serum FFA, serum triglycerides, plasma glucagon, plasma gastric-inhibitory peptide, plasma GLP-1, serum interleukin (IL)-6, serum IL-8, and plasma adiponectin. Satiety was subjectively rated after breakfast and the gastric emptying rate (GER) was determined using paracetamol as a marker. Breath hydrogen was measured as an indicator of colonic fermentation. Evening meals with barley kernel based bread (ordinary, high-amylose- or beta-glucan-rich genotypes) or an evening meal with white wheat flour bread (WWB) enriched with a mixture of barley fiber and resistant starch improved glucose tolerance at the subsequent breakfast compared with unsupplemented WWB (P<0.05). At breakfast, the glucose response was inversely correlated with colonic fermentation (r=−0.25; P<0.05) and GLP-1 (r=−0.26; P<0.05) and positively correlated with FFA (r=0.37; P<0.001). IL-6 was lower (P<0.01) md adiponectin, was higher (P<0.05) at breakfast following an evening meal with barley-kernel bread compared with WWB. Breath hydrogen correlated positively with satiety (r=0.27; P<0.01) and inversely with GER (r=−0.23; P<0.05). The authors concluded from these experiments that composition of indigestible carbohydrates of the evening meal may affect glycemic excursions and related-metabolic risk variables at breakfast through a mechanism involving colonic fermentation. The results provide evidence for a link between gut microbial metabolism and key factors associated with insulin resistance.(47)

Going back to the literature trying to figure out the different responses of fee body to food between normal and overweight or obese patients, the only significant abnormality that was reported, is fee response of the ileal brake to the intake of the mixed meal (17,22), and more specifically to carbohydrates. Therefore it seems fee natural appetite control pathways become tolerant to the intake of carbohydrates. This partially explains the success of the Adkins diet, even though in this case there are no demonstrable differences in the anatomy or histology of those two groups, except in rare cases of severe morbid long term obesity associated with atrophy of the ileum. Given the fact that food delivered to that part of the intestine is capable of stimulating those hormones independently of oral intake and the fact that fee ileal stimulation during a mixed meal can be inhibited by suppressing fee neurotransmission raise the possibility that the problem seems to be about the transmission of the signal from gut to brain. It is possible feat resets of a carbohydrate tolerant ileal brake pathway, will re-set the appetite center and renew the feedback, loop that interrupts eating, all without progression to a metabolic syndrome. Therefore if we are able to directly stimulate fee ileum we should be able to restore fee ileal brake signal and at least give the patient some help In restoring visceral signals that measures the food intake.

These visceral signals are not only important to signal satiety but as per reported reviews (34,44) these hormones are extremely beneficial to the patient. Their absence during down-regulation could be what the patients are seeking unconsciously when they overeat, energy improve muscle, liver, intestine stomach, nerve and heart. Since these hormones are also very important in fee homeostasis of fee insulin and glucose levels they will help tremendously in the use is of the reserves that are already present.

By stimulating the hormones naturally with Aphoeline/Brake™ we are delivering the majority of the hormones where they belong in the portal system, where they have the most powerful impact on inflammation that leads to metabolic syndrome complications. We were also encouraged by the fact that the bypass surgery for obesity is capable of stimulating those hormones in all patients, indicating that the innate ability of these hormones to respond is still present.

We set a goal to stimulate the ileal hormones with an oral natural agent consistent of a ileal brake hormone releasing substance. The data are compelling and the stimulation of the ileal brake pathway seems independent of age or weight or presence of type 2 diabetes. This demonstrates that the intestine still functions despite obesity, and the problem seems to be in the down-regulation of the signaling from the jejunum, which we interpret as a need to wake up the ileal brake. The ileal brake can be awakened either by RYGB surgery or oral administration of Brake™.

What we discovered from these stimulation that it have a very powerful effect on the glucose and insulin homeostasis not consistent with the assumption that these peptides work only by stimulation of the insulin but mainly through, reducing insulin resistance as well long before they achieve weight loss. This is also consistent with the data from bypass surgery.

The more powerful effect on steato-hepatitis seen by decrease of the hepatic enzyme levels to normal within 3-4 weeks need to be studied on a much longer duration to confirm the trend and the gains, but it seems that activation of the ileal brake produces many beneficial effects on metabolic syndrome including the decrease in inflammation, insulin resistance, the trend to normalize triglyceride and cholesterol as well as surprising improvement of all parameters including platelets. Similar platelets trend is seen in cirrhotic patients (non-published data).

Based on the recent publication of Liraglutide and weight loss(29), the GLP-1 family of gut hormones will induce weight loss but in a different way than expected, the weight loss is slow and happens after other parameters start to improve. Weight loss is insidious, just like weight gain, and occurs on a rather unconscious level. The pathway is re-activated after being dormant and the distal caloric signals are now once again respond to ileal brake signals from the ileum.

The advantage of having an oral stimulation of all the ileal hormones is the synergistic effect of the hormones that were meant to be stimulated together in a broad pathway beyond any individual component. The fact that these hormones are released In the portal system that seems to be the center of all metabolism except the muscles and the brain, the fact that the highest concentration of these hormones is hi the portal system make our stimulation much less intrusive and more efficient than the peripheral administration of such hormones.

The suppression of insulin resistance need further investigation even though we showed that the IGF system is stimulated, we do not feel this is the only answer to the question; other peptides as well as other cellular receptors such as the RR receptors need to be investigated as part of the equation. In the next section, we prioritize our future work in this direction.

The stimulation of the ileal hormones with Brake™: present opportunities and challenges.
1, A continuing priority is to improve the ileal brake stimulation potency with further adjustments of the formulation content and the ileal delivery system.
2. Another priority is to develop more practical tests to document the anticipated down-regulation of the ileal brake pathway in the obese, and to demonstrate the impact of Aphoeline/Brake™ in the resetting of this pathway. This testing should be applied to study of a variety of GI diseases such as irritable bowel, and to examine the relationships between hormones and intestinal permeability, immune system and bacterial flora.
3. Third priority is to check on the long term effects of the oral stimulation on improving muscles, pancreas, suppression of acid of the stomach to reported, and determine if the epidemic of reflux and adenocarcinoma increase could be explained on the basis of these hormones deficiency or abnormal responses as reported PYY and GLP1 inhibit together gastric acid secretion 100%.
4. It is necessary to examine the effects of Aphoeline on GI motility including the esophagus and achalasia since these hormones were reported to be neurotrophic. The effect on the lung has not been studied yet, but since it improves the function of other muscles it should have a beneficial impact on the costal muscles, as well as those of the bronchi and the diaphragm.
5. Diabetes is a major target and its innocuous profile should be considered its a first line of treatment, large study and long term effect should be targeted including HbA1c, all indications that the ileal brake pathway does improve diabetes. Because of its effect on insulin resistance, other circumstances of insulin resistance should be checked as well including but not limited to polycystic ovaries.
6. We also would study the effect on the liver. Even though it helps fatty liver it seems that it effect should be checked in different conditions as well including different hepatitis as a co-adjuvant therapy.
7. We would also investigate the use of Aphoeline as a co-adjuvant therapy in bypass surgery. Assessment; of action prior to surgery to study the ileal response or to stabilize die patient and improve their gut or post op as a salvage therapy, or co-adjuvant, should be considered.

The to-do list and the excitement are limitless, especially considering that these effects were produced by a benign orally administered natural product. Reactivation of a dormant gut peptide mechanism is a means of examining the gut as well as obesity from a new perspective.

### Examples 1-4: Further Assessment of Experimental Implications

We have demonstrated the feasibility of a benign food substance delivered orally to stimulate the ileal hormones. The response appears to be sufficient to standardize the stimulation of the ileal brake hormones. Some unusual effects of that stimulation included suppression of insulin resistance, improvement in blood glucose levels, and significant early improvement in liver enzymes and lipid levels. While these beneficial effects were sustained in short term experiments, further large scale clinical testing and longer term clinical studies will be needed to confirm the persistence of these effects.

Based on our open trials, the long term effect of the Aphoeline formulation is an increase in energy levels. There was an unconscious awareness of the calorie intake and a resetting of appetite which then resulted in significant weight loss. Long term double-blind placebo controlled trials, similar to the one conducted with Liraglutide, are being planned.

### Longer Term Studies

A number of patients above were followed for six months to a year period following the initial studies described above (therapy continued at 7 pills—about 10 g. glucose per day via Aphoeline-2 with blood work performed weekly) to determine, what effects would be present or manifest during that time period. The following results and general trends were obtained and/or observed:

1. Insulin resistance continued to be suppressed;

2. Insulin, pro-insulin and c-peptide were brought back to normal levels;

3. Patient's weight decreased substantially;

4. Decreased triglyceride levels to normal (from 400 mg/dl to about 100-120 mg/dl);

5. Decrease liver enzymes from about 300 IU/L down to a normal level (0-85 IU/L);

6. Decreased hepatitis C-virus titers.

7. Substantially decreased α-fetal protein (from 30 ng/ml to less than 6 ng/ml).

The effects of the present invention are long-lasting and therapy may be continued for extended periods of time, resulting in favorable responses in all patients tested.

The terms and expressions that have been employed in this application are used as terms of description and nm of limitation, and there is no intent in the use of such terms and expressions to exclude any equivalent of the features shown and described or portions thereof, but it is recognized that various modifications are possible within the scope of the invention as claimed.

Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and genetically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, diose skilled in the art will recognize that the invention is also thereby described in terms of any individual member or subgroup of members of the Markush group.

### Example 5

Reduction in Endotoxemia, Oxidative and Inflammatory Stress, and Insulin Resistance Following Roux-En-Y Gastric Bypass Surgery in Patients with Morbid Obesity and Type 2 Diabetes Mellitus.

Background: Roux-en-Y gastric bypass (RYGB) results in profound weight loss and resolution of Type 2 Diabetes Mellitis (T2DM). The mechanism of this remarkable transition remains poorly defined. It has been proposed that endotoxin (LPS) sets inflammatory tone, triggers weight gain, and initiates T2DM. Because RYGB may diminish LPS from endogenous and exogenous sources, we hypothesized that LPS and the associated cascade of oxidative and inflammatory stress would diminish after RYGB.

Methods: Fifteen adults with morbid obesity and T2DM undergoing RYGB were studied. Following an overnight fast, a baseline blood sample was collected the morning of surgery and at 180-days to assess changes in glycemia, insulin resistance, LPS, mononuclear cell (MNC) NFκB binding and mRNA expression of CD14, TLR-2, TLR-4, and markers of inflammatory stress.

Results: 180-days following RYGB, subjects had a significant fall in BMI (52.1±3.0 to 40.4±11.1), plasma glucose (148±4 to 101±4 mg/dl), insulin (18.5±2.2 to 8.6±1.0 mμ/ml) and HOMA-IR (7.1±1.1 to 2.1±0.3). Plasma LPS significantly reduced by 20±5% (0.567±0.033 to 0.443±0.022 EU/ml). NFκB DNA binding fell significantly by 21±8%, while TLR-4, TLR-2 and CD-14 expression fell significantly by 25±9%, 42±8%, and 27±10%, respectively. Inflammatory mediators CRP, MMP-9 and MCP-1 fell significantly by 47±7% (10.7±1.6 to 5.8±1.0 mg/L), 15±6% (492±42 to 356±26 ng/ml) and 11±4% (522±35 to 466±35 ng/ml), respectively.

Conclusions: LPS/NFκB DNA binding, TLR-4, TLR-2, and CD 14 expression, CRP, MMP-9 and MCP-1 fall significantly after RYGB. The mechanism underlying resolution of insulin resistance and 12DM following RYGB may be attributable, at least in part, to the reduction of endotoxemia and associated pro-inflammatory mediators.

### BACKGROUND

Obesity, insulin resistance, and type 2 diabetes mellitus (T2DM) are associated with low-grade chronic inflammation.¹⁻⁵ The inciting event linking the activation of the chronic inflammatory state to the development and/or maintenance of obesity and T2DM has not been well defined. In 2007, Cani et al demonstrated an animal model for the pathogenesis of obesity, insulin resistance, and T2DM wherein elevated circulating endotoxin, or bacterial cell wall lipopolysaccharide (LPS), exposure may set inflammatory tone, trigger weight gain, and initiate T2DM.⁶ LPS exposure is continuous from endogenous sources (gut microbiota)⁷⁻⁸ and intermittent from exogenous sources (high-fat, high-carbohydrate meals and saturated fat).⁹⁻¹⁰ The binding of LPS to the complex of CD14 and toll-tike receptor-4 (TLR-4) at the surface of innate immune cells lead to the activation of inflammatory pathways mediated by the pro-inflammatory transcription factor, nuclear factor kappa B (NFκB) and the secretion of pro-inflammatory cytokines and other mediators.¹¹ It is thus possible that LPS may be a significant contributor to the induction and maintenance of the chronic inflammatory state hallmark to obesity and T2DM.

Roux-en-Y gastric bypass (RYGB) results in profound weight loss in a majority of patients which is accompanied by a high resolution rate of T2DM.¹²⁻¹⁵ The resolution of the diabetic state is observed within days of the procedure and well before clinically significant weight loss has occurred.⁷ This time course of resolution provides important evidence dial the chronic inflammatory state may be mediated by a source other titan the adipose tissue. Since LPS is a potential source of the persistent chronic inflammatory state and RYGB ‘curative’ of the insulin resistant diabetic state, we hypothesized that plasma LPS concentration would be reduced following RYGB and that this reduction would be accompanied by a similar reduction in the expression of mononuclear cell (MNC) CD14 and TLR-4 along with a reduction in NFkB binding and other markers of oxidative and inflammatory stress.

### Subjects and Methods

Subjects: Fifteen adult subjects with morbid obesity (body mass index≥40 kg/m²) and T2DM scheduled to undergo RYGB were included in the study. The operative technique has been described previously.¹⁶ Subjects were required to have a minimum of three months of stable ACEI/ARB, statin, and T2DM therapy, defined as no greater than a one-step dose increase or decrease (i.e. metformin from 1000 mg to 500 mg or glyburide from 10 mg to 5 mg). Insulin requirements were not permitted to change greater than 25%. Subjects were: excluded if they required chronic aspirin. NSAID'S or systemic corticosteroids. Baseline characteristics for the subjects are presented in Table 1. After an overnight fast, a baseline blood sample was collected the morning of the RYGB procedure and at 180-days to assess change in glycemia, insulin resistance (HOMA-IR), plasma LPS, MNC NFκB binding and mRNA expression of CD1:4, TLR-2, TLR-4, and other markers of oxidative and inflammatory stress (G-reactive protein [CRP], monocyte chemoattractant protein-1 [MCP-1], and matrix metalloproteinase-9 [MMP-9]). The study was approved by the Catholic Health Institutional Review Board (Buffalo, N.Y.). Each participant signed informed consent (NCT00960765).

MNC Isolation: Blood samples were collected in Na-EDTA and carefully layered on Lympholyie medium (Cedarlane Laboratories, Hornby, ON). Samples were centrifuged and two bands separated out at the top of the RBC pellet. The MNC band was harvested and washed twice with Hank's balanced salt solution (HBSS). This method provides yields greater than 95% MNC preparation.

NFkB DNA binding activity: Nuclear NFkB DNA binding activity was measured by electrophoretic mobility shift assay (EMSA). Nuclear extracts were prepared from MNC and by high salt extraction as previously described.⁵⁻¹⁷ The Active NFkB complex band was determined by incubating nuclear extract from one sample with and without antibodies against p65 or p50 (Santa Cruz Biotechnology, CA), the 2 major components of the active NFκB complex. Specific NFκB bands will be supershifted (SS) (totally or partially) and will appear at a higher molecular weight in the gel while bands that are not affected by the addition of the antibodies are considered nonspecific (NS).

Quantification of TLR4, TLR2, CD14 mid MyD88 Expression: The mRNA expression of TLR4, TLR2, CD14 mid MyD88 was measured in MNC by RT-PCR: Total RNA was isolated using commercially available RNAqueous®-4PCR Kit (Ambion, Austin, Tex.). Real Time RT-PCR was performed using Stratagene Mx3000P QPCR System (La Jolla, Calif.), Sybergreen master mix (Qiagen, CA) and gene specific primers (Life Technologies, MD), All values were normalized to a reference value calculated by GeneNorm software based on the expression of a group of housekeeping genes including actin, ubiquitin C and cyclophilin A.

Plasma Measurements: Glucose concentrations were measured in plasma by YSI 2300 STAT Plus glucose analyzer (Yellow Springs, Ohio). ELISA was used to measure plasma concentrations of insulin (Diagnostic Systems Laboratories Inc., Webster, Tex.), MMP-9 and MCP-1 (R&D Systems, MN) and CRP (American Diagnostica Inc. Stamford, Conn.). Plasma endotoxin concentrations were measured by a commercially available kit (Cambrex Limulus Amebocyte Lysate (LAL) kit, Lonza Inc. Walkersville, Md.). This assay has a sensitivity range of 0.1 EU/ml-1.0 EU/ml. Normal values from lean subjects measured in our laboratory ranged from 0.15-0.35 EU/ml. Inter and intra-assay variations for this test is <10%. Plasma samples used for LPS determination were stored in LPS-free glass tubes to prevent loss of endotoxin to plastic tubes wall. All materials used for the assay were rendered LPS-free. Plasma was diluted 10 folds and heated to 75° C. for 5 min prior to LPS measurement.

Statistical Analysis: Statistical analysis was conducted using SigmaStat software (SPSS Inc., Chicago, IL). All data are represented as mean±S.E. Change from baseline was calculated and statistical analysis was carried out using Paired t-test or Wilcoxon Signed Rank Test, where appropriate. Correlation analysis was performed using Spearman rank-order correlation between change in weight and LPS.

Results of the experiments of this example are presented in FIGS. 1EX5-4EX5 as follows.

FIG. 1EX5 illustrates the change in plasma concentrations of glucose and insulin mid calculated HOMA-IR in obese T2DM patients before and six months following RYGB (N=15). Data are presented as Mean±SE. *P<0.05 by Paired t-test.

FIG. 2EX5 illustrates the change In TLR4, TLR2. CD14 and MyD88 expression In MNC from obese T2DM patients before and six months following RYGB (N=12). Data are presented as Mean±SE, ±P<0.05 by Paired t-test.

FIG. 3EX5 illustrates representative EMSA (A) mid percent change (B) for NFκB DNA binding activity in MNC from 3 obese T2DM patients (Pt) before (B) and six months after (A; RYGB (N=12). Data are presented as Mean±SE. *P<0.05 by Paired t-test. Active NFκB complex band was determined by the addition of anti-p65 or anti-p50 (components of the active NFκB complex) to the reaction mixture containing nuclear extracts from Pt1-B sample causing the supershifting (SS) of the NFκB complex (NFκB) band but no other nonspecific (NS) bands.

FIG. 4EX5 illustrates representative EMS A (A) and percent change (B) for NF □B DNA binding activity in MNC from obese T2DM patients (Pt) before (B) and six months after (A) RYGB (N=12). Data are presented as Mean±SE. *P<0.05 by Paired V test.

FIG. 5EX5 illustrates results of additional regression analyses of data taken from bariatric surgery patients and Brake™ treated patients. More specifically, FIG. 5EX5 provides the results of additional regression analyses of data taken from the bariatric surgery patients. The data compilations presented in the FIG. 5 illustrate that a dosage of approximately 10 grams of active ingredient of a pharmaceutical composition of the invention can have an aggregatepositive effect on ileal brake parameters equal to approximately 25% to approximately 40% of the aggregate positive effect on such parameters realized by Bariatric Surgery.

### Results

Anthropometric and Metabolic Changes Following RYGB: Six months following RYGB, BMI fell from 52.1±13.0 to 40.4±11.1 kg/m² and there were significant improvements in the HbA₁C and lipid profile (Table L, below). There was a significant fall in plasma concentrations of glucose (148±8 to 101±4 mg/dl), insulin (18.5±2.2 to 8.6±1.0 mμ/ml) and HOMA-IR (7.1±1.1 to 2.1±0.3) (FIG. 1EX5, P<0.05 for all). In addition, free fatty acid (FFA) concentration fell significantly by 24% (0.68±0.16 to 0.51±0.17 mM; p<0.05) and plasma transaminase concentrations (AST and ALT) fell 42% (35.6±15.0 to 20.8±9.6; U/L p<0.05) and 49% (36.5±12.8 to 18.6±13.4 U/L; p<0.05), respectively.

Medication Requirements Following RYGB: The use of antidiabetic medication was reduced over the six month follow-up period with fewer subjects requiring metformin (73 vs. 33%; p=0.036) and thiazolidinediones (47 vs. 7%; p0.036). Secretagogue (27 vs. 0%; p=0.1) and insulin-based regimens (33 vs, 20%; p=0.371), ACEI/ARB (33 vs, 20%; p=0.465), and statin (53 vs. 33%; p==0.181) use were not significantly reduced.

Effect of RYGB on Plasma LPS and Proinflammatory Mediators: The plasma concentration of LPS was reduced by 20±5% (0.567±0.033 to 0.443±0.022 EU/ml, FIG. 2EX5, P<0.05) following RYGB, The change in LPS was significantly correlated with the change in weight (r²=0.298; p=0.041). Proinflammatory mediators including CRP by 47±7% (10.7±1.6 to 5.8±1.0 mg/L), MMP-9 by 15±6% (492±42 to 356±26 ng/ml) and MCP-1 by 11±4% (522±35 to 466±37 ng/ml) also fell significantly following RYGB (FIG. 2, P<0.05).

Effect of RYGB on the Expression of TLRs and CD14 in MNC: The mRNA expression of TLR4, TLR2 and CD14 fell significantly by 25&9%, 42±8% and 27±10% over a six month period following RYGB (FIG. 3EX5, P<0.05), There was no significant change in MyD88 gene expression in MNC

Effect of RYGB on NFκB DNA binding: Supershifting assay confirmed the presence of a specific active NFκB complex band (NFκB) and at least 2 nonspecific bands (NS) in the MNC nuclear extracts (FIG. 4EX5). There was a significant reduction in intranuclear NFκB DNA binding in MNC measured by the intensity of the specific band in PMS A. It fell by 21±8% below the baseline at six months following RYGB (FIG. 4EX5, P<0.05).

### Discussion

Our data show clearly that in association with weight toss following RYGB there is a marked reduction in plasma LPS concentration and the mRNA expression of TLR-4 and CD14 in addition to a diminution in inflammation. Since LPS binds to CD14 and TLR-4, the reduction in all three factors potentially orchestrates a reduction in LPS induced inflammation. The activation of the TLR-4 by LPS leads to downstream signaling which leads to the activation of NFkB and to increased transcription of pro-inflammatory genes. Thus, the observed reduction in LPS concentration and life associated expression of TLRs and CD14 and intranuclear NFkB binding represent a reversal of the chronic inflammatory state which characterizes obesity and T2DM. In addition to these findings, we also observed a reduction in the expression of TLR-2 which is the receptor for lipopeptides and peptidoglycans from Gram positive bacteria. In contrast, the expression of MyD88, which mediates downstream inflammatory changes following the binding of TLR ligands, was not altered.

Our previous work has demonstrated that in humans, a single high-fat, high-carbohydrate meal (910 calories; 41% carbohydrate, 42% (at, 17% protein) significantly increases plasma LPS, MNC TLR-2, and TLR-4 expressions and markers of oxidative and inflammatory stress in comparison to an isocaloric meal rich, in fruit and fiber (58% carbohydrate, 27% fat, 15% protein) over a 5 hour period.⁹ We have also demonstrated that it is saturated fat rather than carbohydrate that induces an increase in LPS concentration and TLR-4 expression.¹⁰ The restriction of fat consumption induced by RYGB is likely to be a significant contributor to the long-term diminution of the chronic inflammatory state, in this context, it is important to note that the indicators of oxidative and inflammatory stress increase prior to a significant increase in LPS concentration, CD14 and TLR-4 expression following the intake of a pro-inflammatory meal. Such initial increases may increase intestinal permeability and facilitate the absorption of LPS from the gut. Thus, the role of LPS-CD14-TLR-4 increment following the intake of such a meal would be in the latter phases of post prandial inflammation and also in the context of chronic excessive macronutrient intake.⁶ It should be noted however, that because, our findings were observed in the fasting phase we cannot definitively determine if the changes observed in LPS and inflammatory markers are derived from the interruption of the chronic excessive macronutrient intake, a persistent shift in the endogenous microbiota, or a combination of these factors. Indeed, large gastrointestinal bacterial population shifts have been demonstrated following RYGB and may further contribute to alterations in gut permeability.⁸ To provide greater insight as to the contribution of macronutrient intake and the endogenous flora in the maintenance of the chronic inflammatory state it is of interest to investigate if die pro-inflammatory effect of a meal alters after RYGB.

To date, bariatric surgery is the only treatment known to ‘cure’T2DM.¹⁸ It is also relevant that bariatric surgery has been shown to reduce the risk of cardiovascular events.¹⁵ Our observations following RYGB are relevant in relation to the mechanisms underlying this benefit and thus the pathogenesis of these conditions, larger studies will be required to link each of the specific factors altered by RYGB independently to insulin resistance and T2DM on the one hand and to atherosclerosis on the other. Consistent with this, there was a reversal of insulin resistance as reflected in HOMA-IR with a fall in plasma concentrations of insulin, glucose and triglycerides. These effects along with marked weight loss signal a reversal of die metabolic syndrome¹⁹ and would potentially contribute to the partial or total resolution of T2DM known to occur following RYGB. A recent study from Italy has demonstrated not only a resolution of diabetes in patients subjected to gastric bypass surgery but also a significant reduction in cardiovascular events.²⁰ Previous studies have also shown a tendency for the resolution of T2DM or a marked reduction in the dose of insulin and other antidiabetic medications.²¹⁻²² However, it should be noted that there are other potential mechanisms, possibly involving changes in incretin physiology and behavioral response which may also contribute to the resolution of T2DM²³⁻²⁵ Indeed, in a recent study, it was demonstrated that following RYGB there was a significant sequential increase in GLP-1 and G1P concentrations.²⁷ This area is fertile for further investigation.

In addition to our findings on LPS, CD14, TLR-4, and intranuclear NFkB binding, we also observed a significant reduction in plasma FFA and transaminase concentrations at six months following RYGB. Increased FFA concentrations have been shown to induce inflammatory and oxidative stress including NFkB binding while also inducing insulin resistance.²⁸ RYGB has been shown to significantly improve characteristic histological changes of non-alcoholic fatty liver disease; (NAFLD) including steatosis, inflammation, and fibrosis.²⁹ This is of interest because lifestyle modification and weight reduction is not uniformly accepted as an effective treatment strategy for NAFLD.³⁰ Our observations that plasma LPS and the associated inflammatory cascade diminish following RYGB may also be relevant in relation to the pathogenesis of NAFLD and its complications of cirrhosis or liver cancer.

There are some limitations inherent in this work. We do not have appropriate controls for comparison with the patients who underwent surgery, Since patients referred for surgery and approved by their insurance companies undergo the appropriate dietary protocols and surgery almost immediately, it is very difficult to obtain parallel controls. However, the consistency of the reduction of various indices described ensures that the data are biologically significant. The other shortcoming of our work is the absence of sequential data during the 6 month period so that the evolution of the changes, described can be better understood. Such a detailed study is planned for the future.

### CONCLUSIONS

RYGB is associated with a marked weight loss and a striking reduction in insulin resistance and indices of chronic inflammation. In addition, these improvements are accompanied by reduction in plasma LPS exposure, MNC CD14. TLR-2, and TLR-4 expression and NFkB DNA binding. The reduction in LPS exposure and the expression of pro-inflammatory mediators following RYGB may contribute significantly to the resolution of insulin resistance and T2DM. These effects may potentially protect against atherosclerotic complications.

TABLE 1
Patients' demographic and biochemical data
at baseline and at six months following surgery.

| | Before Surgery | At Six Months |
|---|---|---|
| Females, % | 11, 73.3 | — |
| Age (years) | 44.9 ± 8.7 | — |
| Duration of T2DM (years) | 7.5 ± 4.0 | — |
| Systolic Blood Pressure | 132.9 ± 19.0 | 123.0 ± 11.1 |
| Diastolic Blood Pressure | 71.4 ± 12.3 | 78.5 ± 9.9 |
| Weight | 328.9 ± 72.8 | 255.3 ± 63.7* |
| Body Mass Index | 52.1 ± 13.0 | 40.4 ± 11.1* |
| HbA₁C (%) | 7.9 ± 1.4 | 6.3 ± 0.8* |
| Total Cholesterol (mg/dl) | 175 ± 36 | 165 ± 46 |
| LDL-Cholesterol (mg/dl) | 109.5 ± 30.4 | 96.2 ± 32.6* |
| HDL-Cholesterol (mg/dl) | 41.5 ± 8.2 | 48.5 ± 8.1* |
| Triglycerides (mg/dl) | 209.7 ± 158.5 | 131.6 ± 58.9 |
| Free Fatty Acids (mM) | 0.68 ± 0.16 | 0.51 ± 0.17* |
| Medications: n, % | | |
| ACEI/ARB | 5, 33.3 | 3, 20.0 |
| Statin | 8, 53.3 | 5, 33.3 |
| Exenatide | 1, 6.7 | 0, 0.0 |
| Insulin | 5, 33.3 | 3, 20.0 |
| Metformin | 11, 73.3 | 5, 33.3* |
| Sitagliptin | 4, 26.7 | 2, 13.3 |
| Sulfonylurea | 4, 26.7 | 0, 0.0 |
| Thiazolidinedione | 7, 46.7 | 1, 6.7* |

Data are presented as Mean ± SD,
*= P < 0.05 by paired t-test or Wilcoxon Signed Rank Test.

TABLE 2
Change in plasma concentrations of endotoxin
(LPS), CRP and MMP-9 in obese T2DM patients
at six months following RYGB (N = 15).

| | Before Surgery | At Six Months |
|---|---|---|
| LPS (EU/ml) | 0.567 ± 0.033 | 0.443 ± 0.022* |
| CRP (mg/L) | 10.7 ± 1.6 | 5.8 ± 1.0* |
| MMP-9 (ng/ml) | 492 ± 42 | 356 ± 26* |
| MCP-1(ng/ml) | 522 ± 35 | 466 ± 37* |

Data are presented as Mean ± SE
*P < 0.05 by Paired t- test.

### Example 6

Long-Term Stimulation of the Ileal Hormones by an Oral GRAS Compliant Agent Aphoeline. Effects on Metabolic Syndrome, Fatty Liver, Type II Diabetes and Hepatitis C.

The experiment of this example shows decreasing insulin resistance, triglycerides, liver enzymes, signaling caloric intake, using caloric reserve, and tuning body to health with every meal.

More specifically, the results show that compositions and methods' of the invention can decrease insulin resistance; maintain glucose homeostasis; decrease proinsulin, (that at times seems to be the only signal to insulin resistance); decrease liver enzymes (mainly ALT, AST, SGOT, and SGPT), either directly or secondary to decrease insulin resistance; decrease alpha-fetoprotein, likely secondary to a decrease in liver inflammation; decrease hepatitis C virus levels (direct effect by improving the immune system vs. via decrease in triglycerides), (see FIG. 23EX6); decreases triglyceride; decrease weight likely as a result of decreasing insulin resistance, improving energy and therefore activities and improve signaling to the brain; and provide a good way to approach fatty liver, prediabetes, hypertriglyceridemia obesity, insulin resistance state, metabolic syndrome in general.

The physiological response to oral chronic stimulation of the ileal hormones has not been well studied. We report the results of a pilot retrospective study on 18 patients with the following conditions, obese, prediabetes, hyperlipidemia, fatty liver with elevated liver enzymes, Hepatitis C with cirrhosis, metabolic syndrome, with normal anatomy (i.e. without intestinal or gastric surgeries) followed during chronic oral daily stimulation of the ileal hormones with Aphoeline from 4 to 16 months.

Oral chronic stimulation of the ileal hormones m all patients studied appear to help decrease the average baseline levels of insulin, proinsulin, AST, ALT, Triglycerides, HBA1c, and weight, in all cases approaching normal in a statistically significant manner. When only patients with abnormally elevated baseline levels were averaged the improvement was even more pronounced. Changes in most cases approached diose from a surgical procedure, Roux-en-Y gastric bypass (RYGB), which is considered the gold standard for cure of metabolic syndromes such as diabetes, obesity and hyperlipidemia.

Our study suggests that oral stimulation of the ileal brake hormones with Aphoeline Brake seems to be a promising way to approach problems of insulin resistance, fatty liver, prediabetes, early diabetes type II, hypertriglyceridemia obesity and metabolic syndrome in general, in recent years, the favored treatment for obesity has been RYGB surgery, although only recently has direct comparison established the superiority of this approach over conventional diabetes drug therapy (1). However, few studies have been conducted on die isolated contribution of orally enhanced ileal hormone stimulation based on the decreased gastric volume, intestinal malabsorption that restores such stimulation. We report these results, from a retrospective study on 18 patients with normal anatomy, i.e. without intestinal or gastric surgeries followed during chronic oral stimulation of the ileal hormones with Aphoeline Brake™. Chronic oral stimulation of the ileal hormones appears to help decrease insulin resistance and help in glucose homeostasis. It also decreases proinsulin, liver enzymes, mainly SGOT, SGPT (AST, ALT), alpha-fetoprotein, and triglycerides and reduces weight.

Our study suggests oral Brake™ precisely mimics RYGB and is therefore a promising way to approach metabolic syndrome problems of appetite control, fatty liver, prediabetes, associated hypertriglyceridemia, obesity, Type 2 diabetes (T2D), inflammation mediated loss of pancreatic function such as Type 1 diabetes (T1D), atherosclerosis, Hepatitis C, CHF, CORD, and metabolic syndrome in general.

The novel discovery from the 18 patients treated with Brake™ is that the effects of oral Brake™ treatment persist beyond the cessation of the Brake™ therapy for a considerable period of time (at least 3 months), implying for the first lime that chronic treatment with the oral ileal brake hormone releasing substance was creating a renewed function in visceral organs such as the GI tract, liver and pancreas. Their type 2 diabetes was not immediately returning even when the patients were no longer taking the medication. Indeed it is known or suspected that the hormone mediators of the ileal brake are capable of regenerating pancreatic beta cells and even hepatocytes, but it was completely novel to observe these effects after oral mimetics of RYGB surgery like Brake™.

### Introduction

Blood levels of ileal hormones like gastrin, secretin, gastric inhibitory polypeptide (G1P) and cholecystokinin (CCK-8), as well as GLP-1, glucagon like peptide PYY and Oxyntomodutin, are known to increase after a meal in a healthy individual but GLP1 and ileal hormones levels fail to increase normally in obese and T2D patients(2). L-cells are the major cells of the intestinal mucosa involved in releasing the ileal hormones, following stimulation by simple carbohydrates and emulsified fat content of food in the intestinal lumen. L-cells are mainly concentrated in the ileum in most species with very few cells present proximal to the ligament of Treitz in humans and other primates (3-5). A considerable number of ileal cells are also present in the proximal colon, in glicentin granules. Ileal brake hormones play a key role in regulating insulin secretion and glucose homeostasis, as well as reducing food intake and body weight (3, 6-13). Because various commercial products considered GLP-1 analogs, such as Exenatide and Liraglutide, stimulate insulin secretion in T2D patients after peripheral injection, it was concluded that the main action of GLP-1 (5) and the ileal hormones was a backup insulin hormone that stimulates insulin as a response to food in a physiological setting. Acute food stimulation of the Ileal hormones will, in fact, suppress insulin resistance and thus help in decreasing plasma glucose levels protecting pancreatic exhaustion, as well as preventing reactive hypoglycemia rather than stimulating insulin secretion. It is however, considerably more complicated, because the ileal brake hormones clearly regulate chronic inflammatory processes that lead to fatty liver and pancreatic insufficiency, thus being responsible for both optimal nutrition but also for maintenance functions on the enteric organs themselves

As pointed out by Drucker, peptide hormones are secreted from endocrine cells and neurons and exert their actions through activation of G protein-coupled receptors to regulate a diverse number of physiological systems including control of energy homeostasis, gastrointestinal motility, neuroendocrine circuits, and hormone secretion. The glucagon-like peptides, GLP-1 and GLP-2 are prototype peptide hormones released from gut endocrine cells in response to nutrient ingestion that regulate not only energy absorption and disposal, but also cell proliferation and survival. GLP-1 expands islet mass by stimulating pancreatic beta-cell proliferation and induction of islet neogenesis. GLP-1 also promotes cell differentiation, from exocrine cells or immature islet progenitors, toward a more differentiated beta-cell phenotype, GLP-2 stimulates cell proliferation in the gastrointestinal mucosa, leading to expansion of the normal mucosal epithelium, or attenuation of intestinal injury in experimental models of intestinal disease. Both GLP-1 and GLP-2 exert antiapoptotic actions in vivo, resulting in preservation of beta-cell mass and gut epithelium, respectively. Furthermore, GLP-1 and GLP-2 promote direct resistance to apoptosis in cells expressing GLP-1 or GLP-2 receptors. Moreover, an increasing number of structurally related peptide hormones and neuropeptides exert cytoprotective effects through G protein-coupled receptor activation in diverse cell types. Hence, peptide hormones, as exemplified by GLP-1 and GLP-2, may prove to be useful adjunctive tools for enhancement of cell differentiation, tissue regeneration, and cytoprotection for the treatment of human disease(14-29). These effects are only documented well in animal systems, yet they are all potentially linked as beneficial actions of RYGB surgery, since this procedure elicits the full response of die ileal brake and causes regeneration of pancreatic beta cells by virtue of creating an absence of need for insulin treatment within 3-6 months after insulin requiring patients undergo the RYGB surgery, and before they have lost much body weight (1)

A definitive cure for type 1 diabetes is currently being pursued with enormous effort by the scientific community. Different strategies are followed to restore physiologic production of insulin in diabetic patients. Restoration of self-tolerance remains the milestone that must be reached in order to move a step further and recover a cell source capable of independent and functional insulin production. Multiple strategies aimed at modulation of both central and peripheral immunity must be considered. Promising results now show that the immune system can be modulated in a way that acquisition of a “diabetes-suppressive” phenotype is possible. Once self-tolerance is achieved, reversal of the disease may be obtained by simply allowing physiologic rescue and/or regeneration of the beta cells to take place. Given that these outcomes have already been confirmed in humans, refinement of existing protocols along with novel methods adapted to T1D reversal will allow translation into clinical trials.(30) We believe that the time is at hand to consider oral stimulation of ileal brake hormones as the method of choice for regeneration of pancreatic beta cells in patients with both T2D and T1D, using oral mimetics of RYGB surgery that produce the full range of effects.

Accumulating data from animal models of T1D and some findings from Clinical studies suggest that autoimmune destruction of islet beta cells is associated with enhanced beta cell regeneration. These authors observe that successful immune therapies, aimed at preservation of islet cell mass, result in a remarkable reduction of beta cell regeneration. Treated or not, as long as the task of treatment is limited by “making peace” with autoimmunity, the process of beta cell loss continues, so current approaches to T1D pancreas regeneration are sub-optimal. Additional therapeutic modalities capable of stimulating beta cell regeneration in the absence of active autoimmune destruction are urgently needed.(31) Brake and RYGB may become the preferred approach since they are immunomodulators but not immunosuppressive. In fact, both Brake and RYGB enhance the immune resistance overall, given their beneficial impact on viruses that evade the immune system like Hepatitis C.

The issue of beta cell regeneration in human pancreas is probably one of the most controversial aspects of T1D research. These authors review the prospects for regeneration in T1D patients, and begin their review by first describing the known mechanisms underlying beta cell development and expansion in normal human pancreatic development, as they observe that it is likely that such mechanisms might also play a role in beta cell regeneration. The sensu strictiori definition of beta cells implies replacement of lost beta cell mass by new beta cells. In their discussion, however, they use the term in a more general way, defining as regeneration the formation of new beta cells, whether or not a loss of beta cells has actually occurred. The potential mechanisms of beta cell regeneration in the human pancreas were discussed in the second part of the review. In particular, they analyzed the processes of beta cell regeneration through proliferation of beta cells, neogenesis from non-beta cell precursors, and trans-differentiation from alpha cells. In the third part of this review, they explore the arguments for and against the ability of the human pancreas to regenerate functional beta cells in the context of T1D and in other pathological conditions.(32) This review establishes the rationale for oral mimetics of ileal brake hormones as a means of regenerating pancreatic beta cells, and supports our clinical observations that this process occurs in patients with diabetes.

T1D patients rely on cumbersome chronic injections of insulin, making the development of alternate durable treatments a priority. The ability of the pancreas to generate new beta-cells has been described in experimental diabetes models and, importantly, in infants with T1D. In this review, the authors discuss recent advances in identifying the origin of new beta-cells after pancreatic injury, with and without inflammation, revealing a surprising degree of cell plasticity in the mature pancreas. In particular, the inducible selective near-total destruction of beta-cells in healthy adult mice uncovers the intrinsic capacity of differentiated pancreatic cells to spontaneously reprogram to produce insulin. This opens new therapeutic possibilities because it implies that beta-cells can differentiate endogenously, in depleted adults, from heterologous origins.(33). Some of the stimuli capable of stimulating beta cell differentiation are ileal brake hormones, supporting the use of RYGB or oral Brake™ for this purpose

The mechanisms that regulate pancreatic beta cell mass are poorly understood. While autoimmune and pharmacological destruction of insulin-producing beta cells is often irreversible, adult beta cell mass does fluctuate in response to physiological cues including pregnancy and insulin resistance. This plasticity points to the possibility of harnessing the regenerative capacity of the beta cell to treat diabetes. These authors developed a transgenic mouse model to study the dynamics of beta cell regeneration from a diabetic state. Following doxycycline administration, transgenic mice expressed diphtheria toxin in beta cells, resulting in apoptosis of 70%-80% of beta cells, destruction of islet architecture, and diabetes. Withdrawal of doxycycline resulted in a spontaneous normalization of blood glucose levels and islet architecture and a significant regeneration of beta cell mass with no apparent toxicity of transient hyperglycemia. Lineage tracing analysis indicated that enhanced proliferation of surviving beta cells played the major role in regeneration. Surprisingly, treatment with Sirolimus and Tacrolimus, immunosuppressants used in the Edmonton protocol for human islet transplantation, inhibited beta cell regeneration and prevented the normalization of glucose homeostasis. These results suggest that regenerative therapy for T1D may be achieved if autoimmunity is halted using regeneration-compatible drugs(34). RYGB and oral Brake treatment appeals to act in this manner, as were shown in the 18 patients presented here as evidence of beneficial actions on diabetes and pre-diabetes. Recent studies have revealed a surprising plasticity of pancreatic beta-cell mass. Beta-cell mass is now recognized to increase and decrease in response to physiological demand, for example during pregnancy and in insulin-resistant states. The authors and others have shown that mice recover spontaneously from diabetes induced by killing of 70-80% of beta-cells, by beta-cell regeneration. The major cellular source for new beta-cells following specific ablation, as well as during normal homeostatic maintenance of adult beta-cells, is proliferation of differentiated beta-cells. More recently, it was shown that one form of severe pancreatic injury, ligation of the main pancreatic duct, activates a population of embryonic-type endocrine progenitor cells, which can differentiate into new beta-cells. The molecular triggers for enhanced beta-cell proliferation during recovery front diabetes and for activation of embryonic-type endocrine progenitors remain unknown and represent key challenges for future research. Taken together, recent data suggest that regenerative therapy for diabetes may be a realistic goal(35). This work points to the need for oral treatments that could regenerate cells in the pancreas, and establishes why an oral mimetic of RYGB could be beneficial.

Several studies have shown that the adult pancreas possesses a potential for beta-cell regeneration upon tissue injury. One of the difficulties in studying beta-cell regeneration has been the lack of a robust, synchronized animal model system that would allow controlled regulation of beta-cell loss and subsequent proliferation in adult pancreas. The investigators present a transgenic mouse regeneration model in which the c-Myc transcription factor/mutant estrogen receptor (cMycER(TAM)) fusion protein can be specifically activated in mature beta-cells. We have studied these transgenic mice by immunohistochemical and biochemical methods to assess the ablation and posterior regeneration of beta-cells. Activation of the cMycER(TAM) fusion protein results in synchronous and selective beta-cell apoptosis followed by the onset of acute diabetes, Inactivation of c-Myc leads to gradual regeneration of insulin-expressing cells and reversal of diabetes. These results demonstrate that the mature pancreas has the ability to fully recover from almost complete ablation of all existing beta-cells. These results also suggest the regeneration of beta-cells is mediated by replication of beta-cells rather than neogenesis from pancreatic duets.(36) Combination therapy with a dipeptidyl peptidase-4 inhibitor (DPP-IV) and a proton pump inhibitor (PPI) raises endogenous levels of GLP-1 and gastrin, respectively, and restores pancreatic beta-cell mass and normoglycemia in nonobese diabetic (NOD) mice with autoimmune diabetes(37). The aim of this study was to determine whether a DPP-IV and PPI combination could increase beta-cell mass in the adult human pancreas. Pancreatic cells from adult human pancreas donors were implanted in NOD-severe combined immunodeficient (NOD-scid) mice and the mice were treated with a DPP-IV and a PPI for 16 weeks. Human grafts were examined for insulin content and insulin-stained cells. Graft beta-cell function was assessed by intravenous glucose tolerance tests (IVGTT) and by; glucose control in human cell-engrafted mice treated with streptozotocin (STZ) to delete mouse pancreatic beta-cells. Plasma GLP-1 and gastrin levels were raised to two- to threefold in DPP-IV and PPI-treated mice. Insulin content and insulin-stained cells in human pancreatic cell grafts were increased 9- to 13-fold in DPP-IV and PPI-treated mice and insulin-stained cells were co-localized with pancreatic exocrine duct cells. Plasma human G-peptide responses to IVGTT were significantly higher and STZ-induced hyperglycemia was move completely prevented in DPP-TV and PPI-treated mice with grafts than in vehicle-treated mice with grafts. In conclusion, DPP-IV and PPI combination therapy raises endogenous levels of GLP-1 and gastrin and greatly expands the functional beta-cell mass in adult human pancreatic cells implanted in immunodeficient mice, largely from pancreatic duct cells. This suggests that a DPP-IV and PPI combination treatment may provide a pharmacologic therapy to correct the beta-cell deficit in type 1 diabetes(37). As this combination of oral drugs is producing similar effects on pancreatic regeneration as RYGB or oral Brake™, and is being used in a clinical trial to treat T1D patients, the existence of this study adds validation to the use of ileal brake hormone regulators in the treatment of T1D patients who would clearly and dramatically benefit from regeneration of beta cell mass.

Aphoeline is a composition which is used in the present application and comprises dextrose and a number of other components (Aphoeline/Aphoeline II/Brake™) as described above and in U.S. patent application Ser. No. 12/932,633, filed March 2011, which incorporated by reference in its entirety herein.

While even our earliest short term studies in patients demonstrated a rapid decline in insulin resistance, it was hot initially clear whether these observed acute regulatory mechanism of suppressing insulin resistance is maintained long-term. Durable response on pancreatic insulin producing capability was desirable for long term control of T2D, and the data incorporated hemin show that is occurring in both RYGB patients and Oral Brake™ patients. Thus both have impact by creating beneficial tissue remodeling patterns in visceral organs and tissues, and of course the primary advantage of Brake™ over RYGB is that it produces the same biomarker effects as the surgery, without need for the surgery except hr cases of extreme obesity where more weight must be lost for the health of the patient. To answer these remaining questions, we investigate in this study the effect of long term stimulation of ileal hormones with Aphoeline 2, an early formulation of Brake™, on a number of metabolic problems including fatty liver, triglyceride, weight, HBA1c, and insulin levels.

Methods: Eighteen patients who participated and agreed to share their findings anonymously for publication purposes, were followed in our practice for different diseases. Nine were female and 0.9 were male, ranging in age front 26 to 71, with an average age of 55, The ethnic breakdown included one African-American, one Asian, one from the Philippines and 2 Hispanics, the rest were Caucasians. Eleven patients were pre or early diabetic with elevated pro-insulin or insulin levels or HBA1c less or equal to 7.5, but not yet taking diabetes medications. Nine were diagnosed with fatty liver and abnormal liver enzymes ALT, AST. At least 2 diagnosed with liver biopsies, 7 of these also belonged to the pre diabetic/diabetic group, consistent with the reported comorbidity of both diseases; an additional 3 had hepatitis C but were not currently taking anti-viral drugs, of these, 2 had biopsy proven cirrhosis. All patients took Aphoeline Brake™ daily orally. Aphoeline tablets contain simple carbohydrates and herbs coated with a special pH-time dependent delivery system that delivers the content of the tablet primarily to the ileum. Daily dosing consisted of a dose of 7 pills taken once a day at the same time 4 hours before the major meal. This dose delivers to the ileum a carbohydrate content equivalent to 10.5 grams of glucose. All 18 patients were encouraged to exercise and follow a healthy diet. Patients were followed monthly for periods ranging between 4 months to 16 months, with a fatty liver profile, consisting of blood level of: glucose, insulin, proinsulin, c peptide, albumin, total protein, BUN, creatinine, alpha-fetoprotein, triglyceride, cholesterol, liver enzymes, bilirubin and LDH as well as thyroid profile. Body weight and BMI were also recorded during the each visit. The metabolic profile, liver and (insulin resistance) IR 2 changes, as well as the alpha-fetoprotein, (given the presence of liver disorder in the patients studied) were recorded during the period reported.

### Statistical Analysis

The two-sample paired t-test⁷ was used to determine if (i) there was a significant decrease in the mean profiles (fatty liver, weight, triglyceride and diabetes); this was done in two ways:

(a) using data for all 18 patients, and (b) for patients with initial reading out of normal range, and
(ii) If the percent decrease was significant, for patients with initial reading out of normal range.

In addition, we also computed (iii) the 95% confidence intervals for the parameter p, the true proportion of patients for which out of range reading became normal during the period of taking the medication. This was done using the confidence interval formula for the binomial proportion⁷

Since the amount of decrease towards normal was proportional to the abnormal initial values we divided the patient in 2 categories one with abnormal initial values the other one with a normal starting values on the parameters of SGOT, SGPT, insulin, proinsulin, triglyceride and cholesterol, and compare initial to final values, (iii)

### Results

### (i) Results of T_Tests for Difference in Mean Profiles (Before and After Taking Aphoeline)

The results from the paired t-tests show that, at the error rate of 5% or the confidence level of 95%, patients on Aphoeline experienced a significant decrease was observed in the mean profiles of:

| | Using the data for patients |
|---|---|
| Using the data for all 18 patients | with initial reading out of |
| (n = 18 in each case) - numbers | normal range) -numbers |
| are mean ± SE of mean | are mean ± SE of mean |
| SGOT by an average of | SGOT by an average of |
| 26.67 ± 8.59, | 39.46 ± 9.59 (n = 13), |
| SGPT by 40.94 12.90, and | SGPT by 78.0 ± 12.9 (n = 10), |
| GGTP by 36.82 ± 11.42, | INSULIN by an average of |
| INSULIN by an average of | 16.21 ± 7.49 (n = 9), |
| 9.92 ± 3.99, | Proinsulin by an average of |
| Proinsulin by an average of | 16.15 ± 8.16, (n = 10), |
| 10.31 ± 4.28, | Triglyceride by an average of |
| HBA1cC by 0.32 ± .11, | 100.2 ± 29.7, (n = 10), and |
| CPEPTIDE by 1.07 ± .34. | Cholesterol by an average of |
| ALPHAPHETOPROTEIN by | 24.9 ± 11.4 (n = 7). |
| 1.46 ± .70, | |
| TRIGLYCERIDE by | |
| 39.67 ± 20.16, and | |
| CREATININE by 0.06 ± .03. | |

### (ii) Results of T_Tests for Percent Decrease in Mean Profiles (Before and After Taking Aphoeline)

In this section, we present the results obtained for percent decrease in SCOT, SGPT, INSULIN, PROINSULIN, CHOLESTEROL, and, TRIGLYCRIDE The percent decrease was, calculated using the following formula:

Percent Decrease=100×(Final reading−Initial Reading)/Initial Reading

| | Number of | | |
|---|---|---|---|
| | patients with | | |
| | initial reading | Mean Percent | 95% Confidence |
| | out of normal | Decrease ± | Interval for Mean |
| Parameter | range (n) | SE of mean | Percent Decrease |
| SGOT | 13 | 47.57 ± 17.6 | (36.93, 58.21)* |
| SGPT | 10 | 59.07 ± 15.02 | (48.32, 69.81)* |
| INSULIN | 9 | 34.8 ± 13.0 | (4.8, 64.8)* |
| PROINSULIN | 10 | 16.15 ± 8.16 | (−2.31, 34.61) |
| TRIGLYCERIDE | 10 | 100.2 ± 29.7 | (32.9, 167.5)* |
| CHOLESTEROL | 7 | 10.8 ± 4.15 | (.65, 20.94)* |

*Percent decrease is statistically significant at 95% confidence, since 0 is not included in the confidence interval.

The results from the paired t-tests using the data for-patients with initial reading out of normal range show that, at the error rate of 5% or the confidence level of 95%, patients on Aphoeline experienced a statistically significant decrease in (see Table 2):

TABLE XX
Results of paired t-tests on final to initial readings

| Parameter | N | Mean | StDev | SEMean | 95UCL | T | P |
|---|---|---|---|---|---|---|---|
| WEIGHT | 18 | −33.94 | 62.81 | 14.80 | −8.19 | −2.29 | 0.017* |
| BMI | 18 | −7.50 | 13.62 | 3.21 | −1.91 | −2.34 | 0.016* |
| SGOT | 18 | −26.67 | 36.46 | 8.59 | −11.72 | −3.1 | 0.003* |
| SGPT | 18 | −40.94 | 54.73 | 12.90 | −18.50 | −3.17 | 0.003* |
| ALKALINEPHOSPHATASE | 18 | −6.83 | 25.66 | 6.05 | 3.69 | −1.13 | 0.137 |
| GGTP | 17 | −36.82 | 47.10 | 11.42 | −16.88 | −3.22 | 0.003* |
| INSULIN | 18 | −9.92 | 16.94 | 3.99 | −1.50 | −2.98 | 0.012* |
| PROINSULIN | 18 | −10.31 | 18.14 | 4.28 | −2.87 | −2.41 | 0.014* |
| HGB1AC | 18 | −0.32 | 0.46 | 0.11 | −0.13 | −2.93 | 0.005* |
| CPEPTIDE | 17 | −1.07 | 1.39 | 0.34 | −0.48 | −3.17 | 0.003* |
| ALPHAFETOPROTEIN | 17 | −1.46 | 2.88 | 0.70 | −0.24 | −2.09 | 0.026* |
| TRIGLYCERIDE | 18 | −39.67 | 85.52 | 20.16 | −4.60 | −1.97 | 0.033* |
| CHOLESTEROL | 18 | 0.11 | 67.25 | 15.85 | 27.68 | 0.01 | 0.503 |
| ALBUMIN | 17 | 0.03 | 0.27 | 0.06 | 0.14 | 0.46 | 0.672 |
| HDL | 17 | 6.35 | 22.07 | 5.35 | 15.70 | 1.19 | 0.874 |
| LDL | 17 | −13.27 | 80.33 | 19.48 | 20.75 | −0.68 | 0.253 |
| CREATININE | 17 | −0.06 | 0.14 | 0.03 | −0.01 | −1.93 | 0.036* |
| HGB | 18 | 0.73 | 3.71 | 0.87 | 2.25 | 0.84 | 0.793 |
| WBC | 18 | 0.24 | 1.78 | 0.42 | 0.97 | 0.58 | 0.716 |
| BILRUBIN | 17 | −0.16 | 0.89 | 0.22 | 0.22 | −0.73 | 0.237 |
| PLATELETCOUNT | 18 | 23.72 | 78.35 | 18.47 | 55.85 | 1.28 | 0.892 |

(iii) Confidence Intervals for Proportion of Patients Who Show Improvement

TABLE 2
95% Confidence Intervals for p (N = total number of
patients with initial reading out of normal range, X =
number of patients with final reading inside the normal range)

| | 95% Confidence Interval for p |
|---|---|
| ALT | 42% to 92% (N = 14, X = 10) |
| AST | 48% to 98% (N = 11, X = 9) |
| ALKALINE PHOSPHATASE | 1% to 99% (N = 2, X = 1) |
| GGTP | 19% to 99% (N = 4, X = 3) |
| C-PEPTIDE | 23% to 83% (N = 11, X = 6) |
| INSULIN | 21% to 86$ (N = 9, X = 5) |
| TRIGLYCERIDE | 35% to 93% (N = 10, X = 7) |
| CHOLESTEROL | 2% to 52% (N = 11, X = 2) |
| HDL | 0% to 63% (N = 3, X = 0) |
| LDL | 1% to 81% (N = 4, X = 1) |

We also plotted these measurements vs; TIME, measured as the number of days the medication was taken orally (see FIGS. 1-16). The monotonically decreasing behavior of WEIGHT and BMI with TIME can be seen from FIGS. 1-2.

iii) Confidence Intervals for Proportion of Patients Who Show Improvement

We also computed the 95% confidence intervals for the parameter p, the true proportion of patients for which out of range reading became normal during the period of taking the medication. These calculations showed that (see Table 2):
SGOT improved in 42%-92% of these patients.
SGPT improved in 48%-98% of these patients,
GGTP improved in 19%-99% of these patients,
INSULIN improved in 21%-86% of these patients,
C-PEPTIDE improved in 23%-83% of these patients, and
TRIGLYCERIDE improved in 35%-93% of these patients,

We also plotted these measurements vs. time, measured as the number of days the medication was taken orally (see FIGS. 1EX6-16EX6). The monotonically decreasing behavior of WEIGHT and BMI with TIME can be seen from FIGS. 1EX6-2EX6.

(iii) Comparison of Subgroups with Initially Elevated Vs. Initially Normal Starting Values:

The two categories one with abnormal initial values the other one with normal shirting values on the parameters of SCOT, SGPT, insulin, proinsulin, triglyceride and cholesterol, and compare initial averages to final averages. The results are dramatic, showing that the average changes in all patients went back to normal ranges, and that effectively the patients brought ail parameters to normal range. It also showed the more dramatic response proportional to the initial value deviation from normal.

Normal ranges for the values are as follow:

### SGOT (AST): 10-35; SGPT (ALT):9-60; INSULIN: 0-17; PROINSULIN: 0-18;

### TRIGLYCERIDE: 0-150;

### CHOLESTEROL: 125-200

For the group with the average elevated initial baseline levels,
the average abnormal initial values were as follow:

### SGOT(AST):72.23; SGPT (ALT): 126.80; INSULIN:36.58 PROINSULIN:44.50; TRIGLYCERTDE:243.40;

### CHOLESTEROL: 228.14

The final averages values for the same group were as follow:

### SGOT (AST):32.77; SGPT (ALT):48.8; INSULIN: 20.81; PROINSULIN: 28.35;

### TRIGLYCERIDE: 149.2:

### CHOLESTEROL: 203.29

The percentage decrease from initial to final for the same group was as follow:

### SGOT (AST):54. 53%; SGPT (ALT):61.52%; INSULIN: 42%; PROINSULIN: 363%;

### TRIGLYCERIDE: 40.18%;

### CHOLESTEROL: 10.90%

In terms of HOMA 2 the average decrease in insulin resistance was 2.1-23, making it a decrease of 43.6% in insulin resistance.

Graph that represents the initial, final of each group as well as the normal range value is included.¹⁵

TABLE 1
Averages Normal vs. Not-Normal patients

| | SGOT | SGPT | INSULIN | PROINSULIN | TRIGLYCERIDE | CHOLESTEROL |
|---|---|---|---|---|---|---|
| abnormal Initial | 72.23 | 126.80 | 36.58 | 44.50 | 249.40 | 228.14 |
| abnormal Final | 32.77 | 48.80 | 20.81 | 28.35 | 149.20 | 203.29 |
| percent | 54.53% | 61.52% | 42% | 36.30% | 40.18% | 10.90% |
| decrease | | | | | | |
| Normal Initial | 28.00 | 39.00 | 9.74 | 11.63 | 117.38 | 173.45 |
| Normal Final | 24.00 | 26.50 | 6.11 | 7.23 | 102.13 | 168.18 |
| percent | 14.29% | 35.05% | 37.29% | 37.85% | 13% | 3.04% |
| decrease | | | | | | |
| LOW | 10.00 | 9.00 | 0.00 | 0.00 | 0.00 | 125.00 |
| HIGH | 35.00 | 60.00 | 17.00 | 18.00 | 150.00 | 200.00 |

TABLE 1A
Results of Paired T-Tests on FINAL-INITIAL readings.

| | | Mean | | | 95% | | |
|---|---|---|---|---|---|---|---|
| Variable | N | Reduction | StDev | SEMean | UCL | T | P |
| BMI | 18 | −7.50 | 13.62 | 3.21 | −1.91 | −2.34 | 0.016* |
| SGOT | 18 | −26.67 | 36.46 | 8.59 | −11.72 | −3.1 | 0.003* |
| SGPT | 18 | −40.94 | 54.73 | 12.90 | −18.50 | −3.17 | 0.003* |
| Alkaline Phosphatase | 18 | −6.83 | 25.66 | 6.05 | 3.69 | −1.13 | 0.137 |
| GGTP | 17 | −36.82 | 47.10 | 11.42 | −16.88 | −3.22 | 0.003* |
| INSULIN | 18 | −9.92 | 16.94 | 3.99 | −1.50 | −2.98 | 0.012* |
| PROINSULIN | 18 | −10.31 | 18.14 | 4.28 | −2.87 | −2.41 | 0.014* |
| HGB1AC | 18 | −0.32 | 0.46 | 0.11 | −0.13 | −2.93 | 0.005* |
| CPEPTIDE | 17 | −1.07 | 1.39 | 0.34 | −0.48 | −3.17 | 0.003* |
| Alpha Fetoprotein | 17 | −1.46 | 2.88 | 0.70 | −0.24 | −2.09 | 0.026* |
| TRIGLYCERIDE | 18 | −39.67 | 85.52 | 20.16 | −4.60 | −1.97 | 0.033* |
| CHOLESTEROL | 18 | 0.11 | 67.25 | 15.85 | 27.68 | 0.01 | 0.503 |
| ALBUMIN | 17 | 0.03 | 0.27 | 0.06 | 0.14 | 0.46 | 0.672 |
| HDL | 17 | 6.35 | 22.07 | 5.35 | 15.70 | 1.19 | 0.874 |
| LDL | 17 | −13.27 | 80.33 | 19.48 | 20.75 | −0.68 | 0.253 |
| CREATININE | 17 | −0.06 | 0.14 | 0.03 | −0.01 | −1.93 | 0.036* |
| HGB | 18 | 0.73 | 3.71 | 0.87 | 2.25 | 0.84 | 0.793 |
| WBC | 18 | 0.24 | 1.78 | 0.42 | 0.97 | 0.58 | 0.716 |
| BILRUBIN | 17 | −0.16 | 0.89 | 0.22 | 0.22 | −0.73 | 0.237 |
| Platelet count | 18 | 23.72 | 78.35 | 18.47 | 55.85 | 1.28 | 0.892 |

TABLE 2
95% Confidence Intervals for p (N = total number of
patients with initial reading out of normal range, X =
number of patients with final reading inside the normal range)

| | 95% Confidence Interval for p values |
|---|---|
| SGOT | 42% to 92% (N = 14, X = 10) |
| SGPT | 48% to 98% (N = 11, X = 9) |
| ALKALINE PHOSPHATASE | 1% to 99% (N = 2, X = 1) |
| GGTP | 19% to 99% (N = 4, X = 3) |
| CPEPTIDE | 23% to 83% (N = 11, X = 6) |
| INSULIN | 21% to 86$ (N = 9, X = 5) |
| TRIGLYCERIDE | 35% to 93% (N = 10, X = 7) |
| CHOLESTEROL | 2% to 52% (N = 11, X = 2) |
| HDL | 0% to 63% (N = 3, X = 0) |
| LDL | 1% to 81% (N = 4, X = 1) |

### Example 7

RYGB (N=T5) Comparison with Brake (N=18) from the Perspective of Change in HOMA-IR Vs. Changes in Biomarkers and Manifestations of Metabolic Syndrome.

We used available data from the literature and our own analysis of patient data for normals, obese, obese T2D, obese T2D given DPP-IV inhibitors, Byetta 10 mcg, post RYGB, and post single dose of Brake to compare the relative potency of GLP-1 after challenge. The purposes were to examine the apparently sleeping ileal brake pathway, in T2D and the obese, which was shown, and to compare the relative increase in GLP-1 from the interventions performed. The illustrated analysis is below as FIG. 1EX7.

It was clear that there was an important homology between the use of oral formulations of Brake and RYGB surgery, and in fact the questions could be precisely examined as relative potency if we used our data on biomarkers of the response of the various metabolic syndrome manifestations such as insulin resistance, liver enzymes, triglycerides, and body weight itself.

Thus a comparison of RYGB patients (N=15) and Aphoeline/Brake treated patients (N=18) was made from the two studies presented in the body of this application.

All available patients were used in these comparisons unless they did not have a value. In some analyses, only patients with abnormal values at baseline were considered. Data were taken from the studies of patients performed by the investigators. Patient demographics are described earlier.

The purpose of this combined analysis was to define common mechanisms of action between RYGB surgery and oral use of Brake, with a reliance on biomarkers for the definition of relative potency between Brake and RYGB, Data were plotted vs. HOMA-IR change, because this parameter is the first to change and show's an overall dramatic and unexpected response to both RYGB and Brake administration.

### Results

Shown below (FIGS. 2EX7 A-E) are the combined data from RYGB patients and Broke treated patients, with the before values compared to 6 months post start of the monitoring period. Each group of patients is displayed with different symbols so that the similarities and differences can be appreciated. Parameters compared between the populations and presented include HOMA-IR changes, Weight changes, HBA1c changes, AST changes, ALT changes and Triglyceride changes. Many other biomarkers were measured in both studies but it is believed that the chosen biomarkers tell the metabolic syndrome story in sufficient detail to illustrate the discovery of ileal brake mimicry between the formulation of Aphoeline/Brake and RYGB surgery.

Overall, these results show that Brake, and RYGB are acting in nearly identical fashion on the chosen biomarkers, albeit with a shift in relative potency. The statistical analysis allows that potency comparison to be made, and the outcomes are in Table 1EX7 below.

### Discussion of Experimental Results (Examples 6 and 7)

The results of this study show that chronic daily stimulation of the ileal hormones with Aphoeline/Brake™, delivered directly into the ileum, tends to stabilize and maintain the body homeostasis, as well as decrease in the fasting state the abnormal levels of insulin, glucose, triglycerides and all of the measured liver enzymes. Also the significant decrease in alpha-fetoprotein seems to indicate a decrease in inflammation of the liver. Even though we expect some decrease in triglyceride levels with decreasing insulin resistance; it seems that it does decrease to a large extent independently. Combining the decrease in insulin resistance, triglyceride and liver inflammation with decrease in liver enzymes indicates a significant improvement in liver health and signals a role for these hormones to play in regeneration of hepatocytes and maintaining liver health. Even though one can argue that the improvement in insulin resistance per se can induce all the other changes, it is to note that these hormones even though short lived exert their action by combining with receptors at the levels of the organ including the liver. Given the recent finding of an increase role of the miRNA in liver cells to decrease insulin resistance there is a possibility that: these hormones might exert (here effect: through miRNA induction. Another possibility is the relative increase of IGF-1 and 2 that is observed as well during such stimulation, and their well-known effect on decreasing insulin resistance by activating their own cellular receptor's.

The results of these studies, alone and combined, show that chronic daily stimulation of the ileal hormones with Aphoeline delivered directly into the ileum, tends to stabilize and maintain the body homeostasis, as well as decrease the abnormal levels of insulin, glucose, triglyceride and liver enzymes. These are all beneficial effects on the common manifestations of metabolic syndrome in the western world, and it was very surprising to have similar activity produced by both RYGB surgery and the oral formulation. The only significant difference was in the amount of weight lost, and we account for the greater weight loss of RYGB surgery as an effect of shrinking the size of the stomach, a clearly additive effect with the actions on the ileal brake that is absent from the patients given the oral formulation alone.

The decline in liver enzymes was remarkable and similar between the two study populations, in this case Brake performing better than the RYGB surgery, ft should be pointed out that some of the patients given Brake had hepatic abnormalities, while the RYGB patients did not. However, in both cases, the conclusion is that there is an insulin resistance associated increase in fatty liver disease occurring with obesity and T2D, and in both cases the decline in fatty liver condition was associated with RYGB surgery or the oral formulation used for treatment in both cases the decline in liver enzymes to normal occurred in the first month after starting treatment or performing surgery. Also the significant decrease in alpha-fetoprotein seems to indicate a decrease in inflammation of the liver. Even though-we expect some decrease in triglyceride levels with decreasing insulin resistance; it seems that it does decrease to a large extent earlier and independently. Combining the decrease in insulin resistance, triglyceride and liver inflammation with decrease in liver enzymes indicates a significant improvement in liver health and signals an important role for the intra portal release of these ileal brake hormones resulting from RYGB surgery or use of Brake in maintaining liver health.

One might argue that the newly discovered and remarkable improvement in insulin resistance per se can induce all the other changes. However, it is notable that these hormones, even though short lived, exert their action by combining with receptors at the levels of the organ including the liver. Given the recent finding of an increased role of the miRNA in liver cells⁸ to decrease insulin resistance there is a possibility that these hormones might exert there effect through miRNA induction. Another possibility is the relative increase of IGF-1 and IGF-2 that is observed as well during such stimulation, and their well-known effect on decreasing insulin resistance by activating their own cellular receptors⁶.

The decrease in weight was significant, but slow, and lags behind the laboratory parameters of metabolic syndrome. This indicates that weight loss is the net result of an improving system health, resolving inflammation and metabolic syndrome manifestations and a beneficial consequence of reactivated signaling originating in the ileum, rather than art independent or a leading factor preceding the other parameters. To note that metabolic parameters do not all move in a very strict linear fashion, reflecting real life variation in both individuals, real living, life style and measurements and suggesting any short-term measurements in those analyses, especially the weight loss, will not likely reflect the long term trend in these studies. Until these pathways are completely understood, it will be necessary and sufficient to use biomarkers to define relative potency and to differentiate between means of activating the ileal brake in health and disease.

Ileal brake hormones play a key role in regulating insulin secretion and glucose homeostasis, as well as reducing food intake and body weight^{2,4,11,12}. We have previously shown⁶ that a single dose of Aphoeline/Brake significantly decreases glucose, c-peptide and insulin levels up to 10 hours vs, baseline in healthy volunteers. A statistically significant increase in plasma levels of PYY, GLP-1, and GLP-2 was also observed from 0 to peak hours white leptin was not significantly increased. The subjects with baseline elevated insulin and or fasting glucose experienced a much more dramatic decrease in both blood insulin and glucose levels with ileal hormone stimulation. This suggests that in normal metabolism, the balance between absorption and signaling of appetite and maintenance of the body weight is in equilibrium (FIG. 16, FIG. 17). The controller that maintains this equilibrium is the ileal brake, and the signaling pathways are the hormones that are secreted by these gastrointestinal cells in response to food components that reach the ileal brake. It also suggests that at least some of the ileal hormones are secreted in the jejunum or even more proximal areas of absorption. Thus, is essential as a sensor-signaling dual action that senses mainly carbohydrates and fat and sends hormonal signals intra-portally via hormones secreted by the L cells, to maintain the digestive system and the overall nutritional balance of the body, allow it m use its reserves, as well as signaling to suppress appetite for substances not needed. A very elegant and efficient system that uses the food that is absorbed, to signal absorption and the amount being absorbed based on the segment that it stimulates, the more distal the more intense the signal and in normal condition it will be proportional to the amount of calories ingested, but the increase in the intensity of the segment is logarithmic based on cell distribution it will reach a plateau in the ileum (see FIG. 21) this figure representing a theoretical distribution of intensity that will change with individuals either by having different starting points or different slope and possible different starting plateau or different intensity of the plateau itself. This could explain the wide variety of appetite control patterns evident in the human population.

As a result of ileal brake signaling hormones, the end of the increased appetite will come more abruptly towards the end of the meal making the progression of the signal intensity non-linear. The more food that is rapidly ingested the more will be left for the distal segments and the strength of the appetite suppression signal will disproportionately increase. The absence or decrease of the signal in the jejunum associated with absorption as per obese and metabolic syndrome will mislead the measurement and the automatic maintenance that happen normally with absorption. Thus, in the obese and particularly in the obese type 2 diabetic, the ileal brake becomes less responsive, requiring increasingly more food to decrease the appetite for food. It might be thought that the ileal brake goes to sleep in increasing obesity, allowing weight gain in major proportions, all a consequence of a failure to suppress appetite. Because of the defect it will allow insulin and glucose to go higher, eventually triggering pancreatic exhaustion; This defect will be proportional to the lack of signaling, i.e. the less the signaling the more severe the insulin resistance and glucose levels, the more fatty liver and increase in triglycerides, the less body maintenance, the more possible intestinal leaks and depression of the immune system, fatty liver, reflux and less usage of fat reserves, less signaling of satiety. In short, all of the metabolic syndrome manifestations as detailed here develop in a step by step fashion as there is a decline in the hormonal signals from the L cells. Obesity and type 2 diabetes develop step by step all based on an initial relative or absolute lack of signaling from L cells at the level of the jejunum or ileum. It is apparent that patients with obesity and type 2 diabetes have very low amounts of ileal brake hormone release, as shown in FIG. 1EX7 above. As the L-cells are not abnormal, just asleep at this point, it was shown in our data that either RYGB surgery or oral administration of Aphoeline/Brake can restore the downtrend in output of the stimulating hormones, from the ileum, restore the suppression of appetite, and indeed, to create an overall wakeup of the ileal brake. In our data, it was demonstrated that the L-cells distally can substitute for the proximal signaling as well. They did indeed decrease both insulin levels as well as blood glucose acutely, especially in people with elevated baseline levels, showing that the stimulation of the more distal L-cells have the potential to reverse the defect in metabolic syndrome. What was left to prove at that point was that long term stimulation will maintain the same benefit and continue to reverse the defect of signaling and the beneficial effect can be maintained long term.

In this pilot study of patients given Aphoeline/Brake compared with RYGB surgery, results suggest that, long-term stimulation of the ileal hormones with either of these interventions can wake up the otherwise normal but sleeping ileal brake, and thereby suppress insulin resistance as well as lower blood glucose, the decrease being more pronounced in patients with a higher baseline levels. Brake treated patients had similar profiles of biomediators as patients with RYGB surgery, showing the homology between these approaches to ileal brake management of metabolic syndrome manifestations for the first time. Also the expected increase of insulin as reported with GLP.1 analogs, when injected peripherally did not happen, with the oral ileal stimulation, indicating the ileal hormones increase in the portal system independent of absorption and stimulation, at the level of the jejunum, under physiological conditions, is likely to inhibit insulin resistance by lowering both insulin and blood glucose simultaneously⁵.

In addition to the multitude of effects that ileal hormones exert on different organs^{3,9} in healthy individuals, they also seem to enhance absorption and control of blood glucose and work in tandem with G1P, and other hormones (that stimulate insulin with meals, and enhance absorption) to decrease insulin resistance and move the glucose intracellularly. This prevents longer period of hyperinsulinemia, hyperglycemia, with subsequent hypoglycemia and beta cell exhaustion. All of these processes are involved and associated with conditions such as pre-diabetes, type II diabetes⁹, metabolic syndrome, and obesity^{10,11} all of these abnormalities are corrected in a similar manner, insulin resistance first, by RYGB or an oral treatment with Aphoeline/Brake.

This study also demonstrates that the short term effects and benefits observed with oral stimulation of the ileal hormones are sustained with long term stimulation resulting in similar benefits to RYGB. The benefits were not identical with respect to magnitude of weight loss, but oral use of Brake does not alter the size of the stomach so there is greater weight loss overall with RYGB surgery. This may suggest the pathology of abnormal signaling lies in the jejunum where early signaling is admixed with absorption. Permanent or temporary changes could have happened either to alter the stimulation secretion and/or action of the hormones or the production and/or differentiation of the cells from a stem cell in the crypt. Another possibility is that the long term deficiencies in those hormones might alter the post receptor signaling in the organ as per miRNA which will interfere with insulin resistance and glucose homeostasis. Therefore in this scenario it is possible to start with a somatic problem of food imbalance or bad food interfering with hormone release and signaling to trigger a permanent damage that will interfere with miRNA that in turn will make the changes from a pro-syndrome to a full blown irreversible disease. In this scenario prevention and early detection and intervention is the best and cheapest approach to the problem, and seems to agree with real life reality.

Because of the primordial importance of the L cells signaling in the ileum, a survival feature to prevent malabsorption and death, the L cells presence there is denser and more uniform. They form an emergency signaling or brake, present in most living creatures. This is in contrast to the more sparse heterogeneous distribution in the jejunum. The L cells in the ileum are more protected and the signaling more preserved and less easily damaged than the jejunal signaling, therefore even though the L cells jejunal signaling starts very early through food contact which happens to be the same area of absorption. The more intense signaling is the further down were normal amount of food does not get to and is absorbed before it reaches dial area. Since the main problem in obese, Type II diabetics and patients with metabolic syndrome seems to be a defect in the early response of L cells to meals, Ileal stimulation with Aphoeline/Brake acts similar to the stimulation induced with RYGB surgery,^{(FIG. 19)} bringing food down to the functioning L-cells signaling in the ileum. By bypassing the” dead zone signaling segment it helps reset the signaling process and allowing foe body to receive the signaling and maintenance required associated with absorption in the case of bypass and without absorption in the case of Aphoeline/Brake.

Beside the improvement in function, the ileal signaling brings about the true signaling that allows the brain to gage the status of the body as well as to determine and use the caloric reserve present. The GLP1 and PYY were shown to act on the hypothalamus with blood glucose to signal satiety¹². Without the ileal hormones there is no automatic sensor reading of the caloric status of the body available, and the brain has to rely on the conscious logical part to calculate the calories (like in conscious calorie count) and has to work contrary to what the faulty biological signal that is being sent to the brain, (not enough calories), making it very difficult for obese, diabetics and others to live their lives accordingly. Steady weight gain is the result of this down-regulation of the ileal brake signaling. The ileal hormones will also improve the intestine itself as recently demonstrated with GLP-2,¹⁵ as well as allow the body to use its reserve of fat as recently published with oxyntomodulin¹⁶.

The theoretical question whether prolonged treatment i.e. oral ileal stimulation, could reverse the original pathology in the Intestine, as well as to allow the body to restore normal signaling again. This will have to wait further testing. However, it is clear that RYGB surgery has that beneficial tong term effect, and if one compares our findings of RYGB patients with those given Aphoeline/Brake, the summary of results are found in table XX below.

TABLE 1EX7
Summary of Relative Potency comparisons between Brake and RYGB surgery

| | | | | | | | | Brake as a |
|---|---|---|---|---|---|---|---|---|
| | | | | | | | | % Change |
| | | Brake | | | RYGB | | p | versus |
| Parameter | N | Mean | SD | N | Mean | SD | value | RYGB Change |
| % Weight loss, total | 18 | 5.29 | 4.01 | 15 | 25.2 | 5.88 | 0.203 | 20.97 |
| in 6 mo. | | | | | | | | |
| % Weight loss as | 18 | 5.4 | 48 | 15 | 44.9 | 14.4 | 0.006 | 12.03 |
| excess kg in 6 mo | | | | | | | | |
| % chg. HOMA-IR | 18 | 38.3 | 17.8 | 15 | 60.8 | 18.6 | 0.002 | 62.99 |
| pre to post chg. in 6 mo. | | | | | | | | |
| % chg. HBA1c pre to | 6 | 11.2 | 4.35 | 15 | 20.5 | 12.2 | 0.019 | 54.63 |
| post chg. in 6 mo. | | | | | | | | |
| % chg. AST pre to | 15 | 41.3 | 21.7 | 15 | 26.0 | 22.9 | 0.071 | 158.0 |
| post change in 6 mo | | | | | | | | |
| % chg. ALT pre to | 16 | 50.5 | 20.5 | 13 | 26.9 | 31.0 | 0.028 | 187.0 |
| post change in 6 mo | | | | | | | | |
| % chg. Triglycerides | 11 | 32.5 | 15.2 | 6 | 40.3 | 24.0 | 0.498 | 81.0 |
| pre to post in 6 mo. | | | | | | | | |

In general, the results in Table 1EX7 show Brake to be at least 20% as active on the ileal brake over long term (6 months) as RYGB surgery. With respect to some key parameters like HOMA-IR, a measure of insulin resistance. Brake is as much as 62% as active as RYGB. Concerning the decline in HBA1c, a measure of long term glucose exposure, Brake is 54% as potent as RYGB surgery. Each of these findings shows similar slopes of response biomarkers between RYGB and Brake. This further indicates that the ileal brake is re-activated to the benefit of decreases in the associated metabolic syndrome biomarkers and adverse event pathways. Thus, both RYGB and Brake are capable of waking up the ileal brake on a long term basis, and both therefore act in a similar manner in the amelioration of metabolic syndrome and its complications. This is very novel and important, because long term studies have shown that RYGB surgery can reverse atherosclerosis and type 2 diabetes, and thus there is the potential for an oral medication to accomplish these same goals in the treatment of patients with metabolic syndromes. As to the relative potency of Brake vs. RYGB, the importance of these ratios will become clearer as the biomarkers linkage to both short and long term outcomes are studied.

Because the true signaling derived from the ileal hormones to the brain is triggered with fat and carbohydrate (that usually gives satisfaction as well as energy and signaling the body has enough energy to spend), it is not surprising that these two types of food are associated with fatigue, tiredness as well as with depression, it also explain the good taste associated with them. Is this the answer to food addiction¹³, the brain and the body looking for the right signaling?

We are predicting that a combination of oral stimulation and oral medication or injection or a combination should be added to the prospective clinical studies. Further consideration of using oral ileal stimulation in combination with other medications, similar to die one with Hepatitis C, and other viruses, combination treatments could be rationally designed, especially for diabetic treatment where brake would be given with DPP-IV inhibitors. Other drugs could be contribute to augmented response, inducing response or effectiveness. In altered metabolism the balance will shift toward the absorption, insulin production and poor or no stimulation of the ileal hormones, therefore poor signaling of satiety and body caloric reserve and usage, resulting in insulin resistance, fatty liver and obesity, instead of a smooth transition of food and signaling and coordinated secretion. (FIG. 2EX8). Both gastric bypass as well as oval ileal stimulation with Aphoeline will restore some physiological signaling.

Like in the acute stimulation of the ileal hormones by Aphoeline-II, the chronic daily stimulation of the ileal hormones showed again that these hormones in their natural physiological release in the portal system, tend to stabilize and maintain the body homeostasis, by decreasing in the fasting state the abnormal levels of insulin, glucose triglyceride, and by decreasing the liver enzymes directly or indirectly. Of note, even the alpha-fetoprotein seems to decrease significantly confirming a decrease in inflammation of the liver by a mechanism that does not involve immunosuppression. The decrease in triglyceride seems to be significant and may reflect an optimization of lipid handling by both the G1 tract and the liver. Even though we expect some decrease in triglyceride levels with decreasing insulin resistance, it seems that the triglyceride impact is earlier and independent of the impact on weight, and it was extremely novel to observe these long term benefits from an oral mimetic of RYGB surgery.

The decrease in weight was significant but slow and follows the other-parameters, indicating the weight loss is a result of an improving system and signaling, rather than what is often stated. Weight reduction in fact may be an independent factor or a leading one that follows the other parameters driven by the ileal brake hormone regulating pathways. To note that metabolic parameters do not all move in a very strict linear fashion, reflecting real life variation in both individuals and measurements and suggesting any short-term measurements in those analyses, especially the weight loss, will not likely reflect the long term trend of organ and tissue regeneration that was the novel finding of these studies.

Non-alcoholic fatty liver, disease (NAFLD) is the hepatic manifestation of metabolic syndrome and the leading cause of chronic liver disease in the Western world. Twenty percent of NAFLD individuals develop chronic hepatic inflammation (non-alcoholic steatohepatitis, NASH) associated with cirrhosis, portal hypertension and hepatocellular carcinoma, yet the causes of progression from NAFLD to NASH remain obscure. In recent publications, the authors show that the NLRP6 and NLRP3 inflammasomes and the effector protein IL-18 negatively regulate NAFLD/NASH progression, as well as multiple aspects of metabolic syndrome via modulation of the gut microbiota. Different mouse models reveal that inflammasome-deficiency-associated changes in the configuration of the gut microbiota are associated with exacerbated hepatic steatosis and inflammation through influx of TLR4 and TLR9 agonists into the portal circulation, leading to enhanced hepatic tumour-necrosis factor (TNF)-alpha expression that drives NASH progression. Furthermore, co-housing of inflammasome-deficient mice with wild-type mice results in exacerbation of hepatic steatosis and obesity. Thus, altered Interactions between the gut microbiota and the host, produced by defective NLRP3 and NLRP6 inflammasome sensing, may govern the rate of progression of multiple metabolic syndrome-associated abnormalities, highlighting the central role of the microbiota in the pathogenesis of heretofore seemingly unrelated systemic auto-inflammatory and metabolic disorders.(38-41). Of significance, our recent studies in RYGB patients(42) as well as the studies in these 18 patients, show that ileal brake hormones moderate these effects on NAFLD. Thus, the novel observation of RYGB and oral Brake is modulation of the G1 tract inflammasome process by ileal brake hormones, and the subsequent ability to use this novel treatment to decrease both hepatic inflammation and NAFLD. This is further beneficial in the treatment of hepatitis C.

With regard to the role of GLP-2 in the improvement of intestinal function and reabsorptive capability, several research groups have concluded that GLP-2 increases gut growth, reduces mucosal cell death, and augments mesenteric blood flow and nutrient absorption. Exogenous GLP-2(1-33) also stimulates glucagon secretion and enhances gut barrier function with implications for susceptibility to systemic inflammation and subsequent metabolic dysregulation, Bahrami and colleagues examined the importance of GLP-2 receptor (GLP-2R) signaling for glucose homeostasis in multiple models of metabolic stress, diabetes, and obesity. Body weight, islet function, glucose tolerance, and islet histology were studied in wild-type, high-fat fed, lean diabetic, Glp2r(−/−) and ob/ob:Glp2r(−/−) mice. They found that GLP-2 did not stimulate glucagon secretion from isolated pancreatic islets in vitro, and exogenous GLP-2 had no effect on the glucagon response to insulin-induced hypoglycemia in vivo. Glp2r(−/−) mice exhibit no change in glycemia, and plasma glucagon levels were similar in Glp2r(−/−) and Glp2r(+/+) mice after hypoglycemia or after oral or intraperitoneal glucose challenge. Moreover, glucose homeostasis was comparable in Glp2r(−/−) and Glp2r(+/+) mice fed a high-fat diet for 5 months or after induction of streptozotocin-induced diabetes. In contrast, loss of the GLP-2R leads to increased glucagon secretion and alpha-cell mass, impaired intraperitoneal glucose tolerance and hyperglycemia, reduced hem-cell mass, and decreased islet proliferation in ob/ob:Glp2r(−/−) mice. CONCLUSIONS: Our results show that, although the GLP-2R is not critical for the stimulation or suppression of glucagon secretion or glucose homeostasis in normal or lean diabetic mice, elimination of GLP-2R signaling in obese mice impairs the normal islet adaptive response required to maintain glucose homeostasis(43). Clearly, GLP-2 does not act alone even though it is beneficial to cellular regeneration. This points to the novel importance of stimulating the Is-cells to produce the ileal brake regulatory hormones as opposed to the current strategy to purity each one and administer it by injection. The full response is necessary, as are all die ileal brake hormones as released by either oral Brake or RYGB surgery.

The actions of the structurally related proglucagon-derived peptides (PGDPs)-glucagon, glucagon-like peptide (GLP)-1 and GLP-2-are focused on complementary aspects of energy homeostasis. Glucagon opposes insulin action, regulates hepatic glucose production, and is a primary hormonal defense against hypoglycemia. Conversely, attenuation of glucagon action markedly improves experimental diabetes, hence glucagon antagonists may prove useful for the treatment of type 2 diabetes. GLP-1 controls blood glucose through regulation of glucose-dependent insulin secretion, inhibition of glucagon secretion and gastric emptying, and reduction of food intake. GLP-1-receptor activation also augments insulin biosynthesis, restores beta-cell sensitivity to glucose, increases beta-cell proliferation, and reduces apoptosis, leading to expansion of the beta-cell mass. Administration of GLP-1 is highly effective in reducing blood glucose in subjects with type 2 diabetes but native GLP-1 rapidly degraded by dipeptidyl peptidase IV. A GLP-1-receptor agonist, exendin 4, has recently been approved for the treatment of type 2 diabetes in the US. Dipeptidyl-peptidase-IV inhibitors, currently in phase III clinical trials, stabilize the postprandial levels of GLP-1 and gastric inhibitory polypeptide and lower blood glucose in diabetic patients via inhibition of glucagon secretion and enhancement of glucose-stimulated insulin secretion. GLP-2 acts proximally to control energy intake by enhancing nutrient absorption and attenuating mucosal injury and is currently in phase III clinical trials for the treatment of short bowel syndrome. Thus the modulation of proglucagon-derived peptides has therapeutic potential for the treatment of diabetes and intestinal disease(44).

Gut peptides exert diverse effects regulating satiety, gastrointestinal motility and acid secretion, epithelial integrity, and both nutrient absorption and disposal. These actions are initiated by activation of specific G protein-coupled receptors and may be mediated by direct or indirect effects on target cells. More recent evidence demonstrates that gut peptides, exemplified by glucagon-like peptides-1 and 2 (GLP-1 and GLP-2), directly regulate signaling pathways coupled to cell proliferation and apoptosis. GLP-1 receptor activation enhances beta-cell proliferation and promotes islet neogenesis via activation of pdx-1 expression. The proliferative effects of GLP-1 appear to involve multiple intracellular pathways, including stimulation of Akt, activation of protein kinase Czeta, and transactivation of the epidermal growth factor receptor through the c-src kinase. GLP-1 receptor activation also promotes cell survival in beta-cells and neurons via increased levels of cAMP leading to cAMP response element binding protein activation, enhanced insulin receptor substrate-2 activity and, ultimately, activation of Akt. These actions of GLP-1 are reflected by expansion of beta-cell mass and enhanced resistance to beta-cell injury in experimental models of diabetes in vivo. GLP-2 also promotes intestinal cell proliferation and confers resistance to cellular injury in a variety of cell types. Administration of GLP-2 to animals with experimental intestinal injury promotes regeneration of the gastrointestinal epithelial mucosa and confers resistance to apoptosis in an indirect manner via yet-to-be identified GLP-2 receptor-dependent regulators of mucosal growth and cell survival. These proliferative and antiapoptotic actions of GLP-1 and GLP-2 may contribute to protective and regenerative actions of these peptides fit human subjects with diabetes and intestinal disorders, respectively (45).

BACKGROUND & AIMS: Gut-derived peptides including ghrelin, cholecystokinin (CCK), peptide YY (PYY), glucagon-like peptide (GLP-1), and GLP-2 exert overlapping actions on energy homeostasis through defined G-protein-coupled receptors (GPCRs). The proglucagon-derived peptide (PGDP) oxyntomodulin (OXM) is cosecreted with GLP-1 and inhibits feeding in rodents and humans; however, a distinct receptor for OXM has not been identified.

METHODS; We examined the mechanisms mediating oxyntomodulin action using stable cell lines expressing specific PGDP receptors in vitro and both wild-type and knockout mice in vivo. RESULTS: OXM activates signaling pathways in cells through glucagon or GLP-1 receptors (GLP-1R) but transiently inhibits food intake in vivo exclusively through the GLP-IR. Both OXM and the GLP-1R agonist exendin-4 (Ex-4) activated neuronal c-fos expression in the paraventricular nucleus of the hypothalamus, the area postrema, and the nucleus of the solitary tract following intraperitoneal (i.p.) injection. However, OXM transiently inhibited food intake in wild-type mice following intracerebroventricular (i.c.v.) but not i.p. administration, whereas Ex-4 produced a more potent and sustained inhibition of food intake following both i.c.v. and i.p. administration. The anorectic effects of OXM were preserved in Gcgr(−/−) mice but abolished in GLP-1R(−/−) mice. Although central Ex-4 and OXM inhibited feeding via a GLP-1R-dependent mechanism, Ex-4 but not OXM reduced VO2 and respiratory quotient in wild-type mice. Conclusions: These findings demonstrate that structurally distinct. PGDPs differentially regulate food intake and energy expenditure by interacting with a GLP-1R-dependent pathway. Hence ligand-specific activation of a common GLP-1R increases the complexity of gut-central nervous system pathways regulating energy homeostasis and metabolic expenditure (46).

There is also abundant evidence that oral RYGB mimetics could improve lipid metabolism. For example, excessive postprandial lipemia is a prevalent condition that results from intestinal oversecretion of apolipoprotein B48 (apoB48)-containing lipoproteins. GLP-2 is a gastrointestinal-derived intestinotrapic hormone that links nutrient absorption to intestinal structure and function. The effects of GLP-2 on intestinal lipid absorption and lipoprotein production were studied in hamsters, and intestinal lipid absorption and chylomicron production were quantified in hamsters, wild-type mice, and Cd36(−/−) mice infused with exogenous GLP-2. Newly synthesized apoB48 was metabolically labeled in primary hamster jejunal fragments. Fatty acid absorption was measured, and putative fatty acid transporters were assessed by immune-blotting. In these animals, human GLP-2 increased secretion of the triglyceride (TG)-rich lipoprotein (TRL)-apoB48 following oral administration of olive oil to hamsters: TRL and cholesterol mass each: increased 3-fold. Fast protein liquid chromatography profiling indicated that GLP-2 stimulated secretion of chylomicron/very low-density lipoprotein-sized particles. Moreover, GLP-2 directly stimulated apoB48 secretion in jejunal fragments cultured ex vivo, increased expression of fully glycosylated cluster of differentiation 36/fatty acid translocase (CD36), and induced intestinal absorption of [(3)H]triolein. The ability of GLP-2 to increase intestinal lipoprotein production was lost in Cd36(−/−) mice. CONCLUSIONS: GLP-2 stimulates intestinal apoB48-containing lipoprotein secretion, possibly through increased lipid uptake, via a pathway that requires CD36. These findings suggest that GLP-2 represents a nutrient-dependent signal that regulates intestinal lipid absorption and the assembly and secretion of TRLs from intestinal enterocytes (47).

The research group of Tsujimoto in Japan has been examining the GPR-120 receptor on the L-cell surface, which detects lipids in the distal ileum and activates the ileal brake in response to lipids at that site (48, 49). As free fatty acids provide an important energy source as nutrients, and act as signalling molecules in various cellular processes, several G-protein-coupled receptors have been identified as free-Tatty-acid receptors important in physiology as well as in several diseases. GPR120 (also known as O3FAR1) functions as a receptor for unsaturated long-chain free fatty acids and has a critical role in various physiological homeostasis mechanisms such as adipogenesis, regulation of appetite and food preference. They show that GPR120-deficient mice fed a high-fat diet develop obesity, glucose intolerance and fatty liver with decreased adipocyte differentiation and lipogenesis and enhanced hepatic lipogenesis. Insulin resistance in such mice is associated with reduced insulin signalling and enhanced inflammation in adipose tissue. In humans, they determined that GPR120 expression in adipose tissue is significantly higher in obese individuals than in lean controls. GPR120 exon sequencing in obese subjects reveals a deleterious non-synonymous mutation (p.R270H) that inhibits GPR120 signalling activity. Furthermore, the p.R270H variant increases the risk of obesity in European populations. Overall, this study demonstrates that the lipid sensor GPR120 has a key role in sensing dietary fat and, therefore, in the control of energy balance in both humans and rodents.(48, 49). Hie novel finding in our patients is that the luminal surface receptor is doubtless stimulated by lipid content, in oral Brake™ or in diet by RYGB diversion of lipids to the ileum.

Ileal brake hormones play a key role in regulating insulin secretion and glucose homeostasis, as well as reducing food intake and body weight (3, 5, 9). We previously have studied the effect of an ileal delivery formulation made of carbohydrates and natural herbs on die levels of these hormones and their associated biomarkers in healthy volunteers. Results show a single dose of Aphoeline-1 significantly decreased glucose, c-peptide and insulin levels up to 10 hours vs. baseline (filing 2010 and incorporated into October 2011 as well). A statistically significant increase in plasma levels of PYY, GLP1, and GLP2 was also observed from 0 to peak hours while leptin was not significantly increased. On the subjects found to have initially elevated insulin and or fasting glucose, the stimulation of the ileal hormones had a much more dramatic effect in decreasing both the insulin and the blood sugar. We postulated that in normal metabolism, the balance between absorption and signaling of satiety and maintenance of the body is in equilibrium. The balance among these factors is illustrated in FIG. 2EX8 below.

In altered metabolism the balance will shift toward the absorption, insulin production and poor or no stimulation of the ileal hormones, therefore poor signaling of satiety and body caloric reserve and usage, resulting in insulin resistance, fatty liver and obesity. Obesity is a natural state in a setting of excessive availability of readily absorbed, dense and high nutritional content foods, typical of the modern western diet. Even after obesity is fully developed it is reversible. Both RYGB and oval ileal stimulation of ileal hormones with Brake will restore some physiological signaling. This is shown in FIG. 2EX9.

From these studies in volunteers and patients, the following conclusions are drawn:
1. The isolated stimulation of the ileal hormones seems to suppress insulin levels as well as blood sugar (the decrease levels are more pronounced with a higher baseline).
2. The expected increase as observed with medication as Exenatide and Vildagliptin did not occur with oral ileal stimulation and release of ileal brake hormones, indicating the ileal hormone increase in the portal system with exclusion of absorption and jejunal stimulation under physiological parameters is likely to inhibit insulin resistance and lower both insulin and blood sugar simultaneously.
3. In normal people, besides the multitude of effects these hormones exert on different organs and part of the body(5), they enhance absorption and control of blood sugar and work in tandem with G1P and Others. Collectively, there is a release of appropriate insulin amounts with meals, and an overall enhancement of normoglycemia by decreasing the resistance to insulin and movement of glucose intracellularly, thus preventing longer period of hyperinsulinemia, hyperglycemia, with subsequent hypoglycemia and beta cell exhaustion.
4. These are the basic metabolic syndrome defects, associated with obesity(50), metabolic syndrome(2), prediabetes, and type 2 diabetes(13).

The inventors have demonstrated in the present invention that the short term oral stimulation of the ileal hormones continue to work the same in the long term chronic stimulation bringing all the benefits associated with it.

One might predict that the pathology of abnormal signaling lies in jejunum where the impact of early signaling defects is admixed with more efficient absorption. Permanent or temporary changes could have happened either to alter the, stimulation, secretion of the hormones or the production, or differentiation of the cells from a stem cell in the crypt.

Because of the primordial importance of the signaling in the ileum as a survival feature to prevent malabsorption and death, less heterogeneity more uniform L-cells (as an emergency signaling or brake, present in most living creatures) compared to the jejunum, the ileal break is more protected and less easily damaged than the jejunal signaling, and the signaling is preserved.

Therefore in our oral stimulation, we are using the ileal stimulation with an ileal brake hormone releasing substance (preferably, Brake™), a mimetic of RYGB, to reset the signaling process and allow the body to recover by regeneration of new cells and tissues. Besides the improvement in organ function, we bring about the true signaling that allows the brain to gage the status of the body as well as to determine and use the caloric reserve present. Without it there is no automatic reading of the caloric status of the body available, and one has to rely on the conscious logical part of the brain to calculate the calories and has to work contrary to what the faulty biological signal is sending to the brain, (not enough calories), making it very difficult for obese, diabetics and others to live their lives accordingly.

These hormones will improve the intestines, pancreas and liver, itself, as recently demonstrated with GLP-2 (51). They also permit the body to use its reserve of fat as recently published about oxyntomodulin(52). An interesting question is whether a prolonged treatment i.e. oral ileal stimulation, could reverse the original pathology in the intestine, as well as to allow normal signaling again. The data here suggest regeneration and restoration is possible across most of the organs and tissues of the G1 tract, pancreas, liver and blood vessels.

### Further Discussion Points and General Observations

1. The primary biological purpose of the ileal brake is to act as a sensor to food absorption, acting as a balancing act on the maintenance side of the equation and intervene when needed in case of emergency to maximize GI absorption of nutrients and food substances. The usual reason for activation is absorption of food, in extreme condition it is activated to detect malabsorption, which could happen if there is a defect in absorptive cells and surfaces in proximal segments of the intestine, or rapid transit as per infection or pancreatic in sufficiency or altered acid secetion as per Z.E.
2. As long as there is food in excess and malabsorption is not detected, the ileal brake are stimulated just enough to maintain and coordinate the sensing as well as the maintenance of the portal organs i.e the intestines, stomach, pancreas liver and visceral fat, the insulin sugar and also to improve the rest of the body to include satiety signaling, and nutrients not needed immediately are absorbed and processed into fat or hepatic storage areas in the viscera: Obesity is not opposed by the ileal brake so long as there is no signal of malabsorption. In fact as obesity progress to metabolic syndrome and diabetes type 2, the early functions as a sensing organ, of the ileal brake disappears, showing less output of regulatory hormones titan normal in the well fed state. The patient remains hungry in most cases.
3. In times of food deprivation the ileal brake is also quiet, the patient remains hungry and the ileal brake hormones are active to optimize the GI, liver and pancreas to extract and process airy food or nutrients. Meanwhile, via leptin and other factors like epinephrine fat cells and hepatocytes are instructed to release nutrients, glucose, and lipids as required to maintain normal energy and metabolic functions.
4. Malabsorption, administration of Oral Brake™ or RYGB surgery cause activation of the distal portion of the ileal brake reserved for emergency scenarios, in each case triggering GLP-2 to repair the intestine and restore proper absorption, slowing down the motility suppressing secretion. Also triggered are the same repair functions but at a much more intense level that usually happen during regular meals, in the pancreas and liver (to deal with optimal absorption and utilization of glucose and lipids). Pancreas regeneration is controlled by GLP-1, GLP-2, gastrin, Oxyntomodulin and PYY, and likely still more, unknown factors of the intestine
5. In normal fed time after fasting the ileal brake remodels the G1 tract, pancreas and liver to deal optimally with any food ingestedacting as signaling pathways responsible for control of fat reabsorption and gluconeogenesis from the liver, all in an attempt to maintain energy supply to organs and tissues of the body. Regulatory hormones are released in complex and highly organized and sequential patterns in order to use optimally the oral intake of nutrients to optimal recover of nutrients stored in fat cells and the liver. There is not one ileal brake hormone responsible for all of these beneficial effects, in fact there are many and some are doubtless yet to be discovered.
6. Oral use of Brake™ or RYGB surgery activates the non functional ileal brake in obese patients with metabolic syndromes, and diabetes type 2 or insulin resistance, allowing the the entire spectrum of GI tract remodeling, pancreas regeneration, removal of fat from liver and fat cells and reversal of atherosclerosis to be restarted
7. Gastric Banding, another form of bariatric surgery is less effective because it is restrictive only results in a smaller stomach and the ingestion of less food acting only on the pain neuroreceptor as a hindrance to more food without the benefit of any other maintenance or sensing or metabolic benefit the same is tru of other modalities that relies on decreasing stomach volume without restarting the ileal hormones stimulation
8. Via actions on the central appetite pathways, the ileal brake hormones released change appetite and food preferences. For example, RYGB and oral Brake™ change food preferences of obese patients away from sugar and fat and toward vegetables and protein.
9. Thus far patients studied before and after RYGB surgery and presented in the October 26^{th} filing are demonstrating nearly identical patterns of response to Brake treated patients presented in the October 26^{th} filing. The only difference is that RYGB patients lose more weight overall. This latter observation is to be expected, because RYGB creates a very small stomach and forces the ingestion of minimal meals, while Brake treated patients have a normal stomach.
10. In common, the RYGB patients and the Brake patients are demonstrating an early and rapid reversal of insulin resistance, a decline in liver enzymes and inflammation, a decline in elevated triglycerides and abnormal lipids, and a steady decline in weight (between 11b and 1 kg per week).
11. Inflammation markers like CRP, endotoxin and alpha-fetoprotein are declining steadily in all patients, with the timing of resolution of abnormal inflammation over 3-6 months, and in parallel to weight loss. One explanation for this has been that the inflammation associated with visceral obesity is declining along the trajectory of obesity itself. Clearly the patient notices weight loss from central areas of adiposity, considered beneficial to well-being,
12. In the pancreas, these markers indicate decline in insulin resistance and increased insulin output of the pancreas, which is associated with a decline in HBA1c to normal values that persist even after stopping Brake™ therapy. Hyperglycemia returns only alter the patient begins to gain excess weight again (1-3 months off Brake™), which shows that there are demonstrable residual benefits from the remodeling of the pancreas.
13. In the Liver, these markers indicate decline in hepatic inflammation, which is associated with a decline in ALT, AST, AP and AlphaFetoProtein to normal values that persist even after stopping Brake™ therapy. Hepatic inflammation and fatty liver does not return even after the patient begins to gain excess weight again (1-3 months off Brake™), which shows that there are demonstrable residual benefits from the remodeling of the liver.
14. Claim ileal brake hormone related regeneration of a persisting nature in pancreas, liver and arterioles, based on ileal Brake optimization of visceral organs and nutrient flow
15. Claim regeneration as net benefit of long lasting changes to ileal brake hormone mediated pathways; benefits of RYGB or oral Brake mimicry of RYGB surgery to CV system, Pancreas, liver, heart, lung, kidneys and brain.

### References (Examples 1-4)

1. Polak J M, Bloom S, Coulling I, Pearse A G. Immunofluorescent localization of enteroglucagon cells in the gastrointestinal tract of the dog. Gut. 1971; 12(4):311-8.
2. Polak J M, Coulling I, Bloom S, Pearse AG. Immunofluorescent localization of secretin and enteroglucagon in human intestinal mucosa. Scand J Gastroenterol. 1971; 6(8):739-44.
3. Maljaars P W, Peters H P, Mela D J, Masclee A A. Ileal brake: a sensible food target for appetite control. A review. Physiol Behav. 2008; 95(3):271-81.
4. Van Citters G W, Lin H C. Ileal brake: neuropeptidergic control of intestinal transit. Curr Gastroenterol Rep. 2006; 8(5):367-73.
5. Schirra J, Coke B. The physiological role of GLP-1 inhuman: in cretin, ileal brake or more? Regul Pept. 2005; 128(2): 109-15.
6. Dobson C L, Davis S S, Chauhan S, Sparrow R A, Wilding I R. The effect of ileal brake activators on the oral bioavailability of atenolol in man. Int J Pharm. 2002; 248(1-2); 61-70.
7. Van Citters G W. Lin H C. The ileal brake: a fifteen-year progress report. Curr Gastroenterol Rap. 1999; 1 (5); 404-9.
8. Vu M K, Nouwens M A, Biemond I, Lamers C B, Masclee A A. The osmotic laxative magnesium sulphate activates the ileal brake. Aliment Pharmacol Ther. 2000; 14(5):587-95.
9. Dobson C L, Davis S S, Chauhan S, Sparrow R A, Wilding I R. The effect of oleic acid on the human ileal brake and its implications for small intestinal transit of tablet formulations. Pharm Res. 1999; 16(1):92-6.
10. Pironi L, Stanghellini V, Miglioli M, et al. Fat-induced ileal brake in humans; a dose-dependent phenomenon correlated to the plasma levels of peptide YY. Gastroenterology. 1993; 105(3); 733-9.
11. Soper N J, Chapman N J, Kelly K A, Brown M L, Phillips S F, Go V L. The ‘ileal brake’ after ileal pouch-anal anastomosis. Gastroenterology. 1990; 98(1): 111-6.
12. Spiller R C, Trotman I F, Adrian T E, Bloom S R, Misiewicz I J, Silk D B. Further characterisation of the ‘ileal brake’ reflex in man-effect of ileal in fusion of partial digests of fat, protein, and starch on jejunal motility and release of neurotensin, enteroglucagon, and peptide YY. Gut. 1988; 29(8): 1042-51.
13. Milk fat, diarrhoea, and the ileal brake. Lancet. 1986; 1(8482):658.
14. Spiller R C, Tratman I F, Higgins B E, et al. The ileal brake-inhibition of jejunal mobility after ileal fat perfusion in man. Gut. 1.984, 25(4):365-74.
15. Tamburrano G, Mauceri M, Lala A, Tonelli F, Leonetti F, Andreani D. Plasma levels of glucagon-like polypeptides in gastrectomized patients transformed from Billroth II into Billroth I. Horm Metab Res. 1982; 14(12):642-5.
16. Tamburrano G, F. Tonelli, A. Lala, M. Mauceri, J. Fayad and D. Andreani, Plasma Levels of Extra pancreatic Glucagon (GLI) and Nesidioglucagon (IRG) in Patients with Esophagoplasty International Symposium on Hypoglycemia, p. 493, Serano public Rome, 1980.
17. Holst J J. Glucagonlike peptide 1: a newly discovered gastrointestinal hormone. Gastroenterology. 1994; 107(6): 1848-55.
18. Holst J J, Bersani M, Johnsen A H, Kofod H, Hartmann B, Orskov C. Proglucagon processing in porcine and human pancreas: J Biol Chem. 1994:269(29): 18827-33.
19. Williams D L. Minireview: finding the sweet spot peripheral versus central glucagon-like peptide 1 action in feeding and glucose homeostasis. Endocrinology. 2009; 150(7):2997-3001.
20. Melds. A V, Patterson B, Kamik A A, Shannon R P. Glucagon-like peptide-1 and myocardial protection: more than, glycemic control. Clin Cardiol. 2009; 32(5):236-43.
21. Abu-Hamdah R, Rabiee A, Meneilly G S, Shannon R P, Andersen D K, Elahi D. Clinical review: The extrapancreatic effects of glucagon-like peptide-1 and related peptides. J Clin Endocrinol Metab. 2009; 94(6): 1843-52.
22. Ranganath L R, Beety J M, Morgan L M, Wright J W, Howland R, Marks V. Attenuated GLP-1 secretion in obesity; cause or consequence? Gut. 1996; 38(6):916-9,
23. Field B C, Wren A M, Cooke D, Bloom S R. Gut hormones as potential new targets for appetite regulation and the treatment of obesity. Drugs. 2008; 68(2): 147-63.
24. Jayasena C N, Bloom S R. Role of gut hormones in obesity, Endocrinol Metab Clin North Am. 2008; 37(3):769-87, xi.
25. Damholt A B, Buchan A M, Kofod B. Glucagon-like-peptide-1 secretion from canine L-cells is increased by glucose-dependent-insulinotropic peptide but unaffected by glucose. Endocrinology. 1998; 139(4):2085-91.
26. Holst J J. The physiology of glucagon-like peptide J. Physiol Rev. 2007; S7(4):1409-39.
27. Gutzwiller J P, Drewe J, Goke B, et al. Glucagon-like peptide-1 promotes satiety and reduces food intake in patients with diabetes mellitus type 2. Am J Physiol. 1999; 276(5 Pt 2):R1541-4.
28. Gtuzwiller J P, Goke B, Drewe J, et. al. Glucagon-like peptide-1: a potent regulator of food intake in humans. Gut. 1999; 44(1):81-6.
29. Astrup A, Rossner S, Van Gaal L, et al. Effects of liraglutide in the treatment of obesity: a randomised, double-blind, placebo-controlled study. Lancet. 2009:374(9701): 1606-16.
30. Peters C T, Choi Y H, Brubaker P L, Anderson G H. A glucagon-like peptide-1 receptor agonist and art antagonist modify macronutrient selection by rats, J Nutr. 2001; 131 (8):2164-70.
31. Choi Y H, Anderson G H. An interaction between hypothalamic glucagon-like peptide-1 and macronutrient composition determines food intake in rats. J Nutr. 2001; 131(6): 1819-25.
32. Burcelin R, Da Costa A, Drucker D, Thorens B. Glucose competence of the hepatoportal vein sensor requires the presence of an activated glucagon-like peptide-1 receptor. Diabetes. 2001; 50(8):1720-8.
33. Drucker D J. Development of glucagon-like peptide-1-based pharmaceuticals as therapeutic agents for the treatment of diabetes. Curr Pharm Des. 2001; 7(14): 1399-412.
34. Drucker D J. Glucagon-like peptide 2. J Clin Endocrinol Metab. 2001:86(4): 1759-64.
35. Lovshin J, Estali J, Yusta B, Brown T J, Drucker D J. Glucagon-like peptide (GLP)-2 action in the murine central nervous system is enhanced by elimination of GLP-1 receptor signaling. J Biol Chem. 2001; 276(24):21489-99.
36. Boushey R P, Yusta B, Drucker D J. Glucagon-like peptide (GLP)-2 reduces chemotherapy-associated mortality and enhances cell survival in cells expressing a transfected GLP-2 receptor. Cancer Res. 2001; 61(2):61(2); 687-93.
37. Drucker D J. Minireview: the glucagon-like peptides. Endocrinology. 2001; 142(2):521-7.
38. Adrian T B, Savage A P, Fuessl H S, Wolfe K, Besterman H S, Bloom S R. Release of peptide YY (PYY) after resection of small bowel, colon, or pancreas in man. Surgery. 1987; 101(6); 715-9.
39. Savage A P, Adrian T E, Cardan G, Chatterjee V K, Bloom S R. Effects of peptide YY (PYY) on mouth to caecum intestinal transit time and on the rate of gastric emptying in healthy volunteers. Gut. 1987; 28(2):166-70.
40. Balas B, Baig M R, Watson C, et al. The dipeptidyl peptidase IV inhibitor vildagliptin suppresses endogenous glucose production and enhances islet function after single-dose administration in type 2 diabetic patients. J Clin Endocrinol Metab. 2007; 92(4): 1249-55,
41. Qualmann C, Nauck M A, Hoist J J, Orskov C, Creufczfeldt W. Insulinotropic actions of intravenous glucagon-like peptide-1 (GLP-1) [7-36 amide] in the fasting state in healthy subjects, Acta Diabetol. 1995; 32(1):13-6.
42. Holst J J. Glucagon-like peptide-1, a gastrointestinal hormone with a pharmaceutical potential. Curr Med Chem, 1999; 6(11): 1005-17.
43. Meier J J, Gallwitz B, Salmen S, et al. Normalization of glucose concentrations and deceleration of gastric emptying after solid meals during intravenous glucagon-like peptide 1 in patients with type 2 diabetes. J Clin Endocrinol Metab. 2003; 88(6); 2719-25.
44. Baynes K C, Dhillo W S, Bloom S R. Regulation of food intake by gastrointestinal hormones. Curr Opin Gastroenterol. 2006; 22(6):626-31.
45. Chaudhri O B, Wynne K, Bloom S R. Can gut hormones control appetite and prevent obesity? Diabetes Care. 2008; 31 Suppl 2:S284-9.
46. Chaudhri O, Small C, Bloom S, Gastrointestinal hormones regulating appetite. Philos Trans R Soc Lond B Biol Sci. 2006; 361(1471): 1187-209.
47. Nilsson A C, Ostman E M, Holst J J, Bjorck I M. Including indigestible carbohydrates in the evening meal of healthy subjects improves glucose tolerance, lowers inflammatory markers, and increases satiety after a subsequent standardized breakfast. J Nutr. 2008; 138(4):732-9.
48. Maclean P S, Energy balance with weight loss and relapse Am.J.Physiology, 2004.
49. Thomas A. Warden, et al. One year weight losses in the look-ahead study: Factors associated with success. Obesity (2009) 17, 713-722.
50. FDA COMMUNICATION Aug. 24, 2009 Early Communication about an Ongoing Safety Review Orlistat (marketed as Alli and Xenical).
51. Hipel, K. W. & McLeod, A. I. (2005). Time Series Modeling of Water Resources and Environmental Systems.
52. Computing, Vienna, Austria. R Development Core Team (2009), R; A language and environment for statistical computing. R Foundation for Statistics ISBN 3-900051-07-0, URL http://www.R-project.org.

### References: Example 5

1. Crook M A, Tutt P, Pickup J C. Elevated serum sialic acid concentration in NIDDM and its relationship to blood pressure and retinopathy. Diabetes Care. January 1993; 16(1):57-60.
2. Dandona P, Aljada A, Bandyopadhyay A. Inflammation: the link between insulin resistance, obesity and diabetes. Trends Immunol. January 2004; 25(1):4-7,
3. Pickup J C, Matlock M B, Chusney G D, Burt D. NIDDM as a disease of the innate immune system: association of acute-phase reactants and interleukin-6 with metabolic syndrome X. Diabetologia. November 1997; 40(11): 1286-1292.
4. Wellen K E, Hotamisligil G S. Inflammation, stress, and diabetes. J Clin Invest. May 2005; 115(5):1111-1119.
5. Ghanim H, Aljada A, Hofmeyer D, Syed T, Mohanty P, Dandona P. Circulating mononuclear cells in the obese are in a proinflammatory stale. Circulation, Sep. 21 2004; 110(12): 1564-1571.
6. Cani P D, Amar J, Iglesias M A, et al. Metabolic endotoxemia initiates obesity and insulin resistance. Diabetes. July 2007; 56(7): 1761-1772.
7. Cummings D E, Overduin J, Foster-Schubert K E. Gastric bypass for obesity: mechanisms of weight loss and diabetes resolution. J Clin Endocrinol Metab. June 2004; 89(6):2608-2615.
8. Zhang H, DiBaise J K, Zuccolo A, et al Human gut microbiota in obesity and after gastric bypass. Proc Natl Acad Sci USA. Feb. 17 2009; 106(7)-2365-2370.
9. Ghanim H, Abuaysheh S, Sia C L, et al. Increase in plasma endotoxin concentrations and the expression of Toll-like receptors and suppressor of cytokine signaling-3 in mononuclear cells after a high-fat, high-carbohydrate meat; implications for insulin resistance. Diabetes Care. December 2009; 32(12):2281-2287.
10. Deopurkar R, Ghanim H, Friedman J, et al. Differential effects of cream, glucose, and orange juice on inflammation, endotoxin, and the expression of Toll-like receptor-4 and suppressor of cytokine signaling-3; Diabetes Care, May 2010; 33 (5); 991-997.
11. Chow J C, Young D W, Golenbock D T, Christ. W J, Gusovsky F. Toll-like receptor-4 mediates lipopolysaccharide-induced signal transduction. J Biol Chem. Apr. 16 1999; 274(16): 10689-10692.
12. Buchwald H, Avidor Y, Braunwald E, et al. Bariatric surgery: a systematic review and meta-analysis. JAMA. Oct. 13 2004; 292(14): 1724-1737.
13. Pories W J, Swanson M S, MacDonald K G, et al. Who would have thought it? An operation proves to be the most effective therapy for adult-onset diabetes mellitus. Ann Surg. September 1995; 222(3):339-350; discussion 350-332.
14. Schauer P R, Burguera B, Ikramuddin S, et al. Effect of laparoscopic Roux-en Y gastric bypass on type 2 diabetes mellitus, Ann Surg. October 2003; 238(4):467-484; discussion 484-465.
15. Sjostrom L, Lindroos A K, Pelfonen M, et al. Lifestyle, diabetes, and cardiovascular risk factors 10 years after bariatric surgery, N Engl J Med Dec. 23 2004; 351(26):2683-2693.
16. Caruanua J A M, M N, Smith A D, Stawiasz K A, Kabakov E, Kabakov J M, Roux-en-Y gastric bypass by single incision mini-laparotomy: outcomes in 3300 consecutive patients. Obes Surg. 201.1; 2.1:820-824.
17. Andrews N C, Faller D V. A rapid micropreparation technique for extraction of DNA-binding proteins from limiting numbers of mammalian cells. Nucleic Acids Res. 199; 19(9):2499.
18. Poumaras D J, Osborne A, Hawkins S C, et al. Remission of type 2 diabetes after gastric bypass and banding: mechanisms and 2 year outcomes. Ann Surg. December 2010; 252(6):966-971.
19. Dandona P, Aljada A, Chaudhuri A, Mohanty P, Garg R. Metabolic syndrome: a comprehensive perspective based on interactions between obesity, diabetes, and inflammation. Circulation. Mar. 22 2005; 111(11): 1448-1454,
20. Iaconetii A, Pammzi S, De Gaetano A, et al. Effects of bilio-pancreatic diversion on diabetic complications; a 10-year follow-up. Diabetes Care. March 2011:34(3):561-567.
21. Campos G M, Rabl C; Roll G R, et al. Better weight loss, resolution of diabetes, and quality of life for laparoscopic gastric bypass vs banding: results of a 2-cohort pair-matched study. Arch Surg. February 2011; 146(2): 149-155.
22. Lee W J, Chong K, Ser K H, et al. Gastric bypass vs sleeve gastrectomy for type 2 diabetes mellitus; a randomized controlled trial. Arch Surg. February 2011; 146(2): 143-148.
23. Ochner C N, Gibson C, Shaoik M, Goel V, Geliebter A. Changes in neurohormonal gut peptides following bariatric surgery. Int J Obes (Loud). February 2011; 35(2): 153-166.
24. Orlando F A, Goncalves C G, George Z M, Halverson J D, Cunningham P R, Meguid M M. Neurohormonal pathways regulating food intake and changes after Roux-en-Y gastric bypass. Surg Obes Relat Dis. September-October 2005; 1(5):486-495,
25. Batterham R L, ffytche D H, Rosenthal J M, et al. PYY modulation of cortical and hypothalamic brain areas predicts feeding behaviour in humans. Nature. Nov. 1 2007; 450(7166); 106-109.
26. Doucet E, Cameron J. Appetite control after weight loss: what is the role of bloodborne peptides? Appl Physiol Nutr Metab, June 2007; 32(3); 523-532.
27. Falken Y, Hellstrom P M, Holst J J, Naslund E. Changes in Glucose Homeostasis after Roux-en-Y Gastric Bypass Surgery for Obesity at Day Three, Two Months, and One Year after Surgery: Role of Out Peptides: J Clin Endocrinol Metab. July 2011; 96(7):2227-2235.
28. Tripathy D, Mohanty P, D hinds a S, et al. Elevation of free fatty acids induces inflammation and impairs vascular reactivity in healthy subjects. Diabetes. December 2003; 52(12):2882-2887.
29. Clark J M, Alkhuraishi A R, Solga, Alli P, Diehl A M, Magnuson T H. Roux-en-Y gastric bypass improves liver histology in patients with non-alcoholic fatty liver disease. Obes Res. July 2005; 13(7): 1180-1186.
30. Wang R T, Koretz R L, Yee H F, Jr. Is weight reduction an effective therapy for nonalcoholic fatty Liver? A systematic review. Am J Med. November 2003; 115(7):554-559.

### References (Example 6)

1. Schauer P R, Kashyap S R, Wolskl K, Brethauer S A, Kiiwao I P, Pothier C B, et al. Bariatric surgery versus intensive medical therapy in obese patients with diabetes. N Engl J Med. 2012; 366(17); 1567-76.
2. Ranganath L R, Beety J M, Morgan L M, Wright J W, Howland R, Marks V. Attenuated GLP-1 secretion in obesity: cause or consequence? Gut. 1996; 38(6):916-9.
3. Baynes K C, Dhillo W S, Bloom S R, Regulation of food intake by gastrointestinal hormones. Curr Opin Gastroenterol. 2006; 22(6):626-31.
4. Drucker D J, Glucagon-like peptide 2. J Clin Endocrinol Metab. 2001; 86(4):1759-64.
5. Holst J J. The physiology of glucagon-like peptide 1. Physiol Rev, 2007; 87(4): 1409-39.
6. Chaudhri O, Small C, Bloom S, Gastrointestinal hormones regulating appetite. Philos Trans R Soc Lond B Biol Sci. 2006; 361(1471):1187-209.
7. Chaudhri O B, Field B C, Bloom S R, Editorial: from gut to mind-hormonal satiety signals and anorexia nervosa. J Clin Endocrinol Metab, 2006; 91 (3):797-8.
8. Chaudhri O B, Parkinson J R, Kuo Y T, Druce M R, Herlihy A H, Bell J D, et al. Differential Hypothalamic neuronal activation following peripheral injection of GLP-1 and oxyntomodulin in mice detected by manganese-enhanced magnetic resonance imaging. Biochem Biophys Res Common. 2006; 350(2):298-306.
9. Chaudhri O B, Wynne K, Bloom S R, Can gut hormones control appetite and prevent obesity? Diabetes Care. 2008; 31 Suppl 2:S2S4-9.
10. Field B C, Chaudhri O B, Bloom S R, Bowels control brain: gut hormones and obesity.

Nat Rev Endocrinol. 2010; 6(8):444-53.
11. Field B C, Wren A M, Cooke D, Bloom S R. Gut hormones as potential new targets for, appetite regulation and the treatment of obesity. Drugs. 2008; 68(2); 147-63,
12. Field B C, Wren A M, Peters V, Baynes K C, Martin N M, Patterson M, et al. PYY3-36 and oxyntomodulin can be additive in their effect on food intake in overweight and obese humans. Diabetes. 2010; 59(7): 1635-9,
13. Jayasena C N, Bloom S R. Role of gut hormones in obesity. Endocrinol Metab Clin North Am. 2008; 37(3):769-87, xi.
14. Boushey R P, Abadir A, Flames D, Baggio L L, Li Y, Berger V, et al. Hypoglycemia, defective islet glucagon secretion, but normal islet mass in mice with a disruption of the gastrin gene. Gastroenterology, 2003; 125(4); 1164-74.
15. Cao X, Flock G, Choi C, Irwin D M, Drucker D J. Aberrant regulation of human intestinal proglucagon gene expression in the NCI-H716 cell line. Endocrinology. 2003; 144(5):2025-33.
16. De Leon D D, Deng S, Madam R, Ahima R S, Drucker D J, Staffers D A. Role of endogenous glucagon-like peptide-1 in islet regeneration after partial pancreatectomy. Diabetes. 2003; 52(2):365-71.
17. Drucker D J. Glucagon-like peptide-1 and the islet beta-cell: augmentation of cell proliferation and inhibition of apoptosis. Endocrinology. 2003; 144(12):5145-8.
18. Drucker D J. Enhancing incretin action for the treatment of type 2 diabetes. Diabetes Care. 2003; 26(10):2929-40.
19. Drucker D J. Glucagon-like peptides: regulators of cell proliferation, differentiation, and apoptosis. Mol Endocrinol. 2003; 17(2):161-71.
20. Drucker D J. Therapeutic potential of dipeptidyl peptidase IV inhibitors for the treatment of type 2 diabetes. Expert Opin Investig Drugs. 2003; 12(1):87-100.
21. During M J, Cao L, Zuzga D S, Francis J S, Fitzsimons H L, Jiao X, et al. Glucagon-like peptide-1 receptor is involved in learning and neuroprotection. Nat Med. 2003; 9(9):1173-9.
22. Estall J L, Drucker D J. Dual regulation of cell proliferation and survival via activation of glucagon-like peptide-2 receptor signaling. J Nutr, 2003; 133(11):3708-11.
23. Gros R, You X, Baggio L L, Kabir M G, Sadi A M, Mungrue I N, et al Cardiac function in mice lacking the glucagon-like peptide-1 receptor, Endocrinology. 2003; 144(6):2242-52.
24. Kim J G, Baggio L L, Bridon D P, Castaigne I P, Robitaille M F, Jette L, et al. Development and characterization of a glucagon-like peptide 1-albumin conjugate: the ability to activate the glucagon-like peptide 1 receptor in vivo. Diabetes. 2003; 52(3):751-9.
25. Li Y, Hansotia T, Yusta B, Ris F, Halban P A, Drucker D J. Glucagon-like peptide-1 receptor signaling modulates beta cell apoptosis. J Biol Chem. 2003; 278(1):471-8.
26. Mayo K E, Miller U, Bataille D, Dalle S, Goke B, Thorens B, et al. International Union of Pharmacology. XXXV. The glucagon receptor family. Pharmacol Rev. 2003; 55(1):167-94.
27. Trinh D K, Zhang K, Hossain M, Brubaker P L, Drucker D J. Pax-6 activates endogenous proglucagon gene expression in the rodent gastrointestinal epithelium. Diabetes. 2003; 52(2); 425-33.
28. Walsh N A, Yusta B, DaCambra M P, Anini Y, Drucker D J, Brubaker P L. Glucagon-like peptide-2 receptor activation in the rat intestinal mucosa. Endocrinology. 2003; 144(10):4385-92.
29. Yamamoto H, Kishi T, Lee C E, Choi B J, Fang H, Hollenberg A N, et al. Glucagon-like peptide-1-responsive catecholamine neurons in the area postrema link peripheral glucagon-like peptide-1 with central autonomic control sites. J Neurosci. 2003; 23(7):2939-46.
30. Pasquali L, Giannoukakis N, Trucco M. Induction of immune tolerance to facilitate beta cell regeneration in type 1 diabetes. Adv Drug Deliv Rev. 2008; 60(2):106-13.
31. Ablamunits V, Sherry N A, Kushner J A, Herold K C, Autoimmunity and beta cell regeneration in mouse and human type 1 diabetes: the peace is not enough. Ann N Y Acad Sci 2007; 1103:19-32.
32. Gianani R. Beta cell regeneration in human pancreas. Semin Immunopathol. 2011; 33(1):23-7.
33. Desgraz R, Bonal C, Herrera P L, beta-cell regeneration: the pancreatic intrinsic faculty. Trends Endocrinol Metab. 2011; 22(1); 34-43.
34. Nir T, Melton D A, Dor Y. Recovery from diabetes in mice by beta cell regeneration. J Clin Invest. 2007; 117(9); 2553-61.
35. Khalaileh A, Gonen-Gross T, Magenheim J, Nir T, Porat S, Salpeter S, et al Determinants of pancreatic beta-cell regeneration. Diabetes Obes Metab. 2008; 10 Suppl 4:128-35.
36. Cano D A, Rulifson I C. Reiser P W, Swigart L B, Pelengaris S, German M, et al. Regulated beta-cell regeneration in the adult mouse pancreas. Diabetes. 2008; 57(4):958-66.
37. Suarez-Pinzon W L, Rabinovitch A, Combination therapy with a dipeptidyl peptidase-4 inhibitor and a proton pump inhibitor induces beta-cell neogenesis from adult human pancreatic duct cells implanted in immunodeficient mice. Cell Transplant 2011; 20(9): 1343-9.
38. Eisenbarth S C, Williams A, Colegio O R, Meng H, Stvoxvig T, Rongvaux A, et al. NLRP10 is a NOD-like receptor essential to initiate adaptive immunity by dendritic cells. Nature, 2012; 484(7395):510-3.
39. Henao-Mejia J, Elinav E, Strowig T, Flavell R A. Inflammasomes: far beyond inflammation. Nat Immunol. 2012; 13(4):321-4.
40. Henao-Mejia J, Elinav E, Jin-C, Hao L, Mehal W Z, Strowig T, et al. Inflammasome-mediated dysbiosis regulates progression of NAFLD and obesity. Nature, 2012; 482(7384): 179-85.
41. Strowig T, Henao-Mejia J, Elinav E, Havel R. Inflammasomes in health and disease. Nature?. 2012; 48117381); 278-86.
42. Monte S V, Caruana J A, Ghanim H, Sia C L, Korzeniewski K, Schentag J J, et al. Reduction in endotoxemia, oxidative and inflammatory stress, and insulin resistance after Roux-en-Y gastric bypass surgery in patients with morbid obesity and type 2 diabetes mellitus. Surgery. 2011.
43. Bahrami J, Longuet C, Baggio L L, Li K, Drucker D J Glucagon-like peptide-2 receptor modulates islet adaptation to metabolic stress in the ob/ob mouse. Gastroenterology. 2010; 139(3):857-68.
44. Drucker D J Biologic actions and therapeutic potential of the proglucagon-derived peptides, Nat Clin Pract Endocrinol Metab, 2005; 1(1):22-31.
45. Brubaker P L, Drucker D J. Minireview: Glucagon-like peptides regulate cell proliferation and apoptosis in the pancreas, gut, and central nervous system. Endocrinology. 2004; 145(6):2653-9.
46. Baggio L L, Huang Q, Brown T J, Drucker D J. Oxyntomodulin and glucagon-like peptide-1 differentially regulate murine food intake and energy expenditure. Gastroenterology. 2004; 127(2):546-58.
47. Hsieh J, Longuet C, Malda A, Bahrami J, Xu E, Baker C L, et al. Glucagon-like peptide-2 increases intestinal lipid absorption and chylomicron production via CD36; Gastroenterology. 2009; 137(3):997-1005, e1-4.
48. Hara T, Hirasawa A, Ichimura A, Kimura I, Tsujimoto G. Free fatty acid receptors FFAR1 and GPR120 as novel therapeutic targets for metabolic disorders. J Pharm Sci. 2011; 100(9):3594-601,
49. Ichimura A, Hirasawa A, Poulain-Godefroy O, Bonneford A, Hara T, Yengo L et al. Dysfunction of lipid sensor GPR120 leads to obesity in both mouse and human. Nature. 2012; 483(7389):350-4.
50. Burcelin R, Da Costa A, Drucker D, Thorens B. Glucose competence of the hepatoportal vein sensor requires the presence of an activated glucagon-like peptide-1 receptor. Diabetes, 2001; 50(8): 1720-8.
51. Jeppesen P B, Sanguinetti E L, Buchman A, Howard L, Scolapio J S, Ziegler T R, et al. Teduglutide (ALX-0600), a dipeptidyl peptidase IV resistant glucagon-like Peptide 2 analogue, improves intestinal function in short bowel syndrome patients. Gut. 2005; 54(9): 1224-31.
52. Wynne K, Park A J, Small C J, Patterson M; Ellis S M, Murphy K G, et al. Subcutaneous oxyntomodulin reduces body weight in overweight and obese subjects: a double-blind, randomized, controlled trial. Diabetes. 2005; 54(8); 2390-5.

1. A method of treating a plurality of laboratory test biomarkers in a human patient or subject in need comprising administering a single daily orally administered medicament for a period of at least three months to effect a response of improvement or resolution in all of said laboratory test biomarkers, wherein said medicament comprises D-glucose as an ileal brake hormone releasing substance which is released in an effective amount in the ileum of said patient or subject, thereby releasing ileal brake hormones and lowering insulin resistance, elevated blood glucose, elevated triglycerides, elevated AST and ALT, elevated LDL and elevated total cholesterol and raising HDL levels, wherein said changes in these laboratory test biomarkers after said 3 months of treatment is at least 20% and as great as 80% of the changes which occur at 3 months after Roux-en-Y surgery.

2. The method of claim 1, wherein said response of said patient or subject to treatment after 3 months results in a decline in insulin resistance as evidenced by laboratory tests of glucose, insulin and HbA1c which is at least about 50% to about 80% as great as is the effect of Roux-en-Y surgery on insulin resistance.

3. The method of claim 1, wherein said response of said patient or subject to treatment after 3 months on said laboratory test biomarkers of triglycerides, HDL, LDL and total cholesterol results in a decline in hyperlipidemia, which is at least about 50% to about 80% as great as is the effect of Roux-en-Y surgery on hyperlipidemia.

4. The method of claim 1, wherein said response of said patient or subject to treatment after 3 months on laboratory tests of glucose and HbA1c levels evidences a decline in hyperglycemia, wherein said decline is at least about 50% to about 80% as great as is the effect of Roux-en-Y surgery on hyperglycemia, and wherein said patient has type 2 diabetes.

5. The method of claim 1 wherein said patient or subject has metabolic syndrome, said treatment resulting in improvement or resolution of said metabolic syndrome.

6. The method of claim 1, wherein said daily dosage of D-glucose is between 5000 mg and 20,000 mg per day.

7. The method according to claim 1, wherein said medicament includes an animal or vegetable oil or fat.

8. The method according to claim 7, wherein said oil is olive oil, corn oil or an oil or fat from a seed or nut.

9. The treatment method according to claim 1 wherein said medicament releases the majority of the ileal brake hormone releasing substance upon reaching the subject's ileum, wherein the substance activates or re-activates the L-cells of the ileum of said patient or subject.

10. The method according to claim 1 wherein said medicament which comprises D-glucose is enteric coated.

11. The method of claim 10, wherein said enterically-coated medicament comprises a tablet, a hard or soft capsule, each coated for releasing the majority of the ileal brake hormone releasing substance in vivo upon reaching the patient's ileum.

12. The method of claim 1, wherein the medicament is made by coating the ileal brake hormone releasing substance with a material which has a pH dissolution or time delayed profile that delays the release in vivo of the majority of the ileal brake hormone releasing substance until the dosage form reaches the subject's ileum, wherein the coating releases the ileal brake hormone releasing substance at pH values specific to the coating within the range of about 6.8 to about 7.5.

13. The method according to 1 wherein a majority of the ileal brake hormone releasing substance is released from the dosage form when the dosage form reaches the subject's ileum, whereupon the compositional formulation may either activate or re-activate the L-cells of the ileum and thereby mimic the chemical and physiological characteristics of an activated ileal brake in a manner similar to Roux-en-Y surgery.

14. The method of claim 1 wherein said medicament contains the ileal brake hormone releasing substance in combination or over-coated with at least one active agent selected from the group consisting of DPP-IV inhibitors, statins, biguanides, ACE inhibitors, AII inhibitors, Thiazolidinediones, insulin or insulin-like drugs, serotonin H3 blockers, tranquilizers, compounds with immunoregulatory actions, compounds that lower beta amyloid in the brain, and compounds that act on PDE-5 receptors to improve erectile dysfunction in an effective amount wherein said ileal brake hormone releasing substance comprises a core having a coating defined pH release characteristic of about 6.8 to about 7.5 in combination with said active agent in immediate release form, whereupon the dosage form releases a majority of said substance in the ileum which is effective to activate or re-activate the L-cells of the ileum.

15. The method according to claim 1, wherein said ileal brake hormone releasing substance comprises D-glucose in an amount ranging from about 5 grams to about 20 grams.

16. The method according to claim 1 wherein said substance further includes at least one additional component selected from the group consisting of alfalfa leaf, chlorella algae, chlorophyllin, barley grass juice concentrate and mixtures thereof.

17. A method of treating metabolic syndrome in a human patient or subject in need comprising orally administering an effective amount of an enteric coated, ileum hormone stimulating amount of an ileal brake hormone releasing substance comprising an effective amount of glucose in combination with an animal or vegetable oil or fat in oral dosage form wherein at least 50% of said substance is released from the dosage form in the ileum of said patient or subject after oral administration, said substance stimulating the release of ileal hormones in the ileum of said patient or subject effective to produce the following effects in said patient or subject:
1) a reduction of insulin resistance whereby blood glucose and blood insulin levels of said patient or subject are normalized;
2) a reduction in hyperlipidemia;
3) a lowering of blood and hepatic glucose and triglycerides; and
4) an amelioration of hepatic inflammation and fatty liver; wherein the effect of said administration of said substance over time on at least insulin resistance and hyperlipidemia is at least 50% to 80% as effective as Roux-en-Y (RYGB) surgery is on insulin resistance and hyperlipidemia in a patient or subject with metabolic syndrome.
